(19) United States
(12) Patent Application Publication
(10) Pub. No.: US 2004/0049022 A1
(43) Pub. Date: Mar. 11, 2004

(54) Composition & methods for treatment and screening

(76) Inventors: Jonathan W. Nyce, Titusville, NJ (US); Anthony Sandrasagra, South Brunswick, NJ (US); Lei Tang, Princeton, NJ (US); Douglas Aguilar, Jersey City, NJ (US); Shoreh Miller, Plainsboro, NJ (US); Syed Shahabuddin, Newtown, PA (US); Hong Lu, Plainsboro, NJ (US); Hui Cong, Cranbury, NJ (US)

Correspondence Address: HOWREY SIMON ARNOLD & WHITE, LLP, BOX 34, 301 RAVENSWOOD AVE., MENLO PARK, CA, 94025, US

(21) Appl. No.: 10/627,930

(22) Filed: Jul. 25, 2003

Related U.S. Application Data
(63) Continuation-in-part of application No. PCT/US02/13135, filed on Apr. 23, 2002.
(63) Continuation-in-part of application No. PCT/US02/13143, filed on Apr. 23, 2002.

(51) Int. Cl. C07H 21/04; A61K 48/00

(52) U.S. Cl. 536/23.2; 514/44

(57) ABSTRACT

This invention relates to single or multiple target anti-sense oligonucleotides (STA or MTA oligos) of low or no adenosine content for respiratory disease-relevant genes, composition thereof and method for manufacturing the composition. The compositions are effective in the prophylaxis and treatment of diseases and conditions associated with the up-regulated expression of one or more different combination of the genes, including airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, and bronchoconstriction, among others. This invention further relates to a method for screening candidate compounds useful for the prevention and/or treatment of respiratory diseases which binds to gene(s), EST(s), cDNA(s), mRNA(s), or their expresed product(s).

### BACKGROUND OF THE INVENTION

### RELATED APPLICATIONS

This application is a continuation-in-part of PCT Application No. PCT/US02/13135 (EPI-0629), entitled COMPOSITIONS, FORMULATIONS & KIT WITH ANTI-SENSE OLIGONUCLEOTIDE & ANTI-INFLAMMATORY STEROID AND/OR UBIQUINONE FOR TREATMENT OF RESPIRATORY & LUNG DISEASE, and PCT/US02/13143 (EPI-0529), entitled COMPOSITION, FORMULATIONS & KITS FOR TREATMENT OF RESPIRATORY & LUNG DISEASE WITH ANTI-SENSE OLIGONUCLEOTIDES & A BRONCHODILATING AGENT, both filed Apr. 24, 2002, by Jonathan W. Nyce et al.

### INCORPORATION OF SEQUENCE LISTING

The substitute Sequence Listing submitted on compact disc, created on Jun. 12, 2002 as file entitled, “EPI-00673 seqlist st25.txt” containing 827K bytes of data, is hereby incorporated by reference.

### FIELD OF THE INVENTION

This invention relates to single and multiple target anti-sense (STA or MTA) oligonucleotides (oligos) targeted to certain genes, compositions and formulations thereof, mRNAs and proteins, that are useful in the prophylaxis and treatment of various diseases and conditions associated with the gene(s) up-regulated expression, and for screening compounds active at the gene(s), mRNA(s), or gene product(s).

### BACKGROUND OF THE INVENTION

Respiratory diseases, such as allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, and bronchoconstriction, are extremely common in the general population. for example, asthma in the United States affects more than 3% of the population, and accounts for about 1% of all health care costs.

During the last decade, anti-sense oligonucleotides (oligos) have received considerable theoretical consideration and experimental validation as potential pharmacological agents in human diseases. A significant advance has been their direct administration to respiratory tissues and the respiratory tract, which not only targeted localized tissues but decreased the required dosage. Anti-sense therapy thus has significant advantages for increasing target specificity and decreasing systemic side effects. The application of anti-sense therapy to the prevention and treatment of respiratory diseases is particularly suitable for those diseases associated with gene up-regulation. The Human Genome Project has provided a plethora of new nucleic acid sequences, many of which correspond to genes of known activities. Others, however, are novel sequences, whose Expressed Sequence Tags (ESTs) may be applied to the discovery of new genes, to the elucidation of their functions and the invention of new treatments custom tailored to each specific gene or to combinations of genes.

This knowledge may also be combined with known treatments in the pursuit of better prophylatic and therapeutic regimes for various diseases.

Currently, there is a need for oligos and other types of compounds that are effective in the prevention and therapy of respiratory and other lung diseases, and for the discovery of new genes and their functions. In addition, anti-sense oligos to certain targets associated with specific diseases or conditions, are helpful in screening libraries of small molecules that are active at the gene(s), mRNA(s), or gene product(s).

### SUMMARY OF THE INVENTION

This invention relates to an agent, comprising a STA (single target anti-sense) or MTA (multiple target anti-sense) oligo(s) that is anti-sense to an initiation codon, a coding region or a 5′ or 3′ intron-exon junction of a nucleic acid target, or anti-sense to their corresponding mRNA; pharmaceutically and veterinarily acceptable salts of the oligo(s) or mixtures thereof; and a surfactant that may be operatively linked to the oligo(s). Examples of nucleic acid targets include interleukin-4 receptor (IL4R), interleukin-5 receptor (IL5R), chemokine receptors CCR1 and CCR3, chemokines Eotaxin-1, RANTES and MCP4, CD23, ICAM, VCAM, tryptase a or b or PDE4 (phosphodiesterase 4 A, B, Cor D subtypes). The composition of the invention comprise(s) the above oligo, and a pharmaceutically or veterinarily acceptable carrier, in an amount effective to attenuate or inhibit symptoms associated with a disease such as airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, and bronchoconstriction, among others.

The carrier is selected preferably from gaseous, liquid and solid carriers. More preferably, the composition comprises one or more therapeutic or diagnostic agent(s), and/or surfactants. The present composition is provided in a variety of formulation and may comprise a solid powder or liquid carrier, or a formulation may include lipid particles or lipid vesicles and contains, more preferably, liposomes, and/or the particles comprising micro-crystals. The formulation may be provided in a form of a respirable formulation, or an aerosol, that is manufactured in bulk or in single or multiple unit form, and may be included in a capsule or cartridge. The composition may be manufactured by combining one or more oligos with a pharmaceutically or veterinarily acceptable carrier and formulation ingredients and other bioactive agents and stored, or the ingredients may be combined just prior to use. The composition of the invention may further comprise other diagnostic or therapeutic compounds, surfactants, antioxidants, flavoring and coloring agents, fillers, volatile oils, buffering agents, dispersants, RNA inactivating agents, antioxidants, flavoring agents, propellants, preservatives, or surfactants. The oligonucleotide has a sequence of at least 4 contiguous nucleotides selected from full length sequence. This invention further relates to a vector carrying the oligo(s), and to a cell, comprising the above oligonucleotide. The oligonucleotide(s) (oligo(s)) is (are) also provided as a kit, comprising in separate containers, a delivery device, the above composition and instructions for its use, and optionally the oligo(s) and a carrier for preparation of the composition. The delivery device may comprise a nebulizer that delivers single metered doses of the formulation, an insufflator, a pressurized inhaler, or a dry powder delivery device.

The oligo(s) of the invention may be used for preventing or treating airway inflammation and other respiratory diseases by administration to a subject affected by an airway inflammation, other respiratory disease or cancer of an effective amount of an oligo targeted to one or more gene(s), mRNA(s), or gene product(s), or a composition thereof as disclosed in this specification. In another application, the oligos of the invention may be used for screening candidate compounds from a library of small molecules, or to anti-sense oligo(s) that is antisense to one or more gene(s), or mRNA(s), as disclosed in this specification. The candidate compounds may be contacted with, or introduced into a cell expressing, one or more genes, mRNAs, or gene product(s), detecting the binding of the compound and the gene(s), or mRNA(s) and/or a change in the mRNA levels, or in the expressed protein levels, characteristics, or function. The candidate compound may be an inhibitor, an agonist, or an inverse agonist of the target.

### DETAILED DESCRIPTION OF THE INVENTION

This invention arose from a desire by the inventors to improve on prior discoveries relating to the preventative or therapeutic utilization of anti-sense oligos in the treatment of diseases or conditions that may have multiple contributing pathways. The inventors reasoned that they could improve on the prior art by attenuating or enhancing the effects of one or more novel genes and/or pathways with anti-sense oligo(s) directed to those target(s) associated with a specific disease or condition. They, thus, set out to attempt a novel and unobvious strategy and overcame numerous obstacles, particularly the extensive searching and selection of the specifically active targets and the elucidation of the targets' sequences, be it genomic DNA and small molecules RNAs or proteins involved in specific diseases or conditions and designing appropriate drugs in the form of anti-sense oligos suitable for the selected targets. The inventors provide below various preferred embodiments of this invention, and exemplify specifically designed STA and MTA oligo sequences.

Glossary

The term “adenosine-free”, as used herein, means that no adenosine residue is contained in an oligonucleotide. An adenosine-free oligonucleotide is devoid of adenosine. If any of adenosine residues are substituted or replaced with des-adenosine (desA) to give an oligonucleotide having desA but not adenosine, this oligonucleotide is deemed as an adenosine-free oligonucleotide. The term “agent”, as used herein, means a chemical compound, a mixture of chemical compounds, a synthesized compound, a therapeutic compound, an organic compound, an inorganic compound, a nucleic acid, a protein, a biological molecule, a macromolecule, lipid, oil, fillers, solution, a cell or a tissue. Agents may be added to prepare a formulation comprising an inhibitor or an oligonucleotide and used in a composition or a kit in a pharmaceutical or veterinary use. The term “airway”, as used herein, means part of or the whole respiratory system of a subject which exposes to air. The airway includes throat, a windpipe, a respiratory tract, a lung, and lung lining. The airway also includes trachea, bronchi, bronchioles, terminal bronchioles, respiratory bronchioles, alveolar ducts, and alveolar sacs. The term “airway inflammation”, as used herein, means a disease or condition related to inflammation on airway of subject. The airway inflammation may be caused or accompanied by allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, and bronchoconstriction. The term “an anti-sense oligonucleotide (oligo)”, as used herein, means an oligonucleotide which, in this invention, is applied to the reduction or inhibition of gene expression by inhibition of a target nucleic acid. Preferably, the target nucleic acid is messenger RNA (mRNA) or gene. For example, the oligonucleotide generally means a sequence of synthetic or naturally derived nucleotide that (1) hybridizes or is antisense to any segment of an mRNA encoding a target protein under appropriate hybridization conditions, and which (2) upon hybridization causes the reduction in gene expression of the target protein. See, Milligan, J. F. et al., J. Med. Chem. 36(14), 1923-1937 (1993), the relevant portion of which is hereby incorporated in its entirety by reference.

The term “a candidate compound”, as used herein, means a sample compound used for screening to identify a candidate with an activity. The candidate compounds are not limited to their source and useful as therapeutics of respiratory diseases. The term “a carrier”, as used herein, means a biologically acceptable carrier in the form of a gaseous, liquid, solid carriers, and mixtures thereof, which are suitable for the different routes of administration intended. Preferably, the carrier is pharmaceutically or veterinarily acceptable. The composition may optionally comprise other agents such as other therapeutic compounds known in the art for the treatment of the condition or disease, antioxidants, flavoring agents, coloring agents, fillers, volatile oils, buffering agents, dispersants, surfactants, RNA inactivating agents, propellants and preservatives, as well as other agents known to be utilized in therapeutic compositions. The term “a cell-internalized agent”, as used herein, means an agent that enhances or facilitates the internalization of a desired compound or composition into a cell. Preferably, examples of cell-internalized agents are transferrin, asialoglycoprotein, streptavidin, or sperimine. The term “chimeric” oligonucleotides or “chimeras”, as used herein, means oligonucleotides which contain two or more chemically distinct regions, each made up of at least one nucleotide. The term “complementary,” as used herein, means the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other.

The term “a composition”, as used herein, means a mixture containing an inhibitor used in this invention and a carrier. The composition also means a mixture containing an oligonucleotide of this invention and a carrier. The composition may contain other agents. The composition is preferably a pharmaceutical or veterinary composition. The terms “des-adenosine (desA)” and “des-thymidine (desT”)”, as used herein, mean oligonucleotides substantially lacking either adenosine or thymidine, respectively. In some instances, the desT sequences are naturally occurring, and in others they may result from substitution of an undesirable nucleotide (A) by another one lacking its undesirable activity. In the present context, the substitution is generally accomplished by substitution of A with a “universal base”, as is known in the art. The term “down-regulation” as used herein, means a decrease in production, secretion, expression or availability (and thus a decrease in concentration) of the targeted protein or nucleic acids. The term “an effective amount” as used herein, means an amount which provides a therapeutic or prophylactic benefit. The term “fixed” as used herein, means that the non-homologous nucleotide may be replaced with a universal base that may base-pair with similar or equal affinity with two or more of the four nucleotide present in natural DNA: A (adenine), G (guanine), C (cytosine), and T (thymidine). This step generates a further novel sequence, different from the one found in nature, that permits the oligonucleotide to bind, preferably equally well, with the primary target, the secondary target, the tertiary target, etc. The term “a fragment”, as used herein, means a single-stranded nucleic acid having a desired sequence. The fragment has at least four contiguous nucleotides having a sequence derived from desired source. The term “homology”, as used herein, means the identity of residues in nucleic acid or amino acid sequences. When the identity is one hundred percent comparing two or more sequences, those sequences have identical residues in their sequences. The term “homologous”, as used herein, means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later. Preferably the region of identity is greater than about 5 base pair (bp), more preferably the region of identity is greater than 10 bp. “Homologous”, thus, means the level of the identity of sequences, preferably, 60% or more, preferably 70% or more, preferably 80% or more, more preferably 90% or more, or most preferably any one of 95%, 96%, 97%, 98% or 99%. Other residues that are not identical are mismatches.

The term “hybridize”, as used herein, means that a nucleic acid including an oligonucleotide binds or is antisense to its complementary chain of a nucleic acid and maintains binding under an appropriate condition. Hydrogen bonding, which may be Hoogsteen hydrogen bonding or Watson-Crick hydrogen bonding, is formed between complementary nucleoside or nucleotide bases. For example, adenine and thymidine are complementary nucleotide bases, and cytosine and guanine are complementary nucleotide bases which pair through the formation of hydrogen bonds. If a complementary chain is not homologous, a nucleic acid may not bind to and form a bonding. The term “an inhibitor”, as used herein, means a substance which inhibits the activity of the protein or genes encoding therefore selected from interleukin-4 receptor, interleukin-5 receptor, chemokine receptors CCR1 and CCR3, chemokines Eotaxin-1, RANTES and MCP4, CD23, ICAM, VCAM, tryptase a or b, PDE4 (A, B, C, D subtypes). The inhibitor may be a compound or substance binding to one or more gene(s), mRNA(s), or gene product(s), such as gene product(s) and inhibits the activity of the gene(s), mRNA(s), or gene product(s). Additionally, the inhibitor can be a compound or substance which suppresses the expression of one or more gene(s), mRNA(s) or gene product(s). Examples of the inhibitors may be, but not limited to, a chemical compound, an antibody and an oligonucleotide. The term “methylated cytosine”, as used herein, means a cytosine base that is substituted for cytosine to create at least one methylated CpG dinucleotide present in an oligonucleotide. Methylated cytosine is depicted as ^{me}C or ^{m}C. The term “a multi-targeted anti-sense (MTA) oligonucleotide (oligo)”, as used herein, means an oligonucleotide that is antisense to at least two different nucleic acids and is capable of attenuating the expression of more than one target mRNA, or to enhance or attenuate the activity of one or more pathways.

The term “naturally-occurring”, as used herein, means the fact that an object can be found in nature. For example, a nucleic acid or a nucleic acid sequence that is present in an organism (including viruses) that can be isolated from a source in nature and which has not been intentionally modified by man in the laboratory is naturally-occurring. Generally, the term naturally-occurring refers to an object as present in a non-pathological (undiseased) individual, such as would be typical for the species. The term “a non-fully desA sequence”, as used herein, means a sequence may have a content of adenosine of less than about 15%, more preferably less than about 10%, and still more preferably less than 5%, and some even less than 2% adenosine. The term “an oligonucleotide (oligo)”, as used herein, means an oligomer or polymer of ribonucleic acid or deoxyribonucleic acid, or mimetics thereof. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases. Preferably, an oligonucleotide is about 4 to 70, 7 to 70, 7 to 60, 10 to 50, 20 to 40, 20 to 30, 21, 22, 23, 24, 25, 26, 27, 28, or 29, in length. The oligonucleotide may be preferably an anti-sense oligonucleotide.

The term “operatively (operably) linked”, as used herein, means that a nucleic acid is placed into a functional relationship with another nucleic acid sequence including a presequence, secretory leader sequence, promoter, enhancer, ribosome binding site, expression control sequence, or reporter gene, etc. Generally, “operatively linked” means that the DNA sequences being linked are contiguous, for some sequences and, not for other sequences. Linking is accomplished by ligation at convenient restriction sites. If such sites do not exist, the synthetic oligonucleotide adaptors or linkers are used in accordance with conventional practice. The terms “preventing” or “prevention”, as used herein, mean a prophylactic treatment made before a subject obtains a disease or ailing condition such that it can have a subject avoid having a disease or condition related thereto. The term “reducing”, as used herein, means decreasing or preventing the translation or expression of a gene by an oligonucleotide that binds specifically with a target mRNA. The term “respiratory diseases”, as used herein, means diseases or conditions related to the respiratory system. Examples include, but not limited to, airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, and bronchoconstriction. The terms “a segment”, as used herein, means at least four contiguous nucleotides having a sequence derived from any part of mRNA. The term “sequence identity”, as used herein, means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein means a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison.

The term “prodrug” as used herein, means a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention may be prepared as SATE [(S-acetyl-2-thioethyl) phosphate] derivatives according to the methods disclosed in WO 93/24510. The term “a spacer”, as used herein, means a molecule or a group of molecules that connects two molecules, such as a nucleotide and a random nucleotide, and serves to place the two molecules in a preferred configuration. The terms “stringent conditions” or “semi stringent conditions”, as herein used mean conditions under which a test nucleic acid molecule will hybridize to a target nucleotide sequence, to a detectably greater degree than other sequences (e.g., at least two-fold over background). Stringent and semi-stringent conditions are sequence-dependent and will differ in experimental contexts. For example, longer sequences hybridize specifically at higher temperatures. Generally, stringent conditions are selected to be about 5° C. to about 20° C. lower, and preferably, 5° C. lower, than the thermal melting point (Tm) for the specific target sequence at a defined ionic strength and pH. The Tm is the temperature (under defined ionic strength and pH) at which 50% of a complementary target sequence hybridizes to a perfectly matched probe. Typically, stringent conditions will be those in which the salt concentration is less than about 1.0 M Na ion concentration (or other salts), typically about 0.01 to 1.0 M Na ion concentration (or other salts), at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary semi stringenct conditions include hybridization with a buffer solution of 30 % formamide, 1 M NaCl, 1% SDS at 37° C., and a wash in 2×SSC at 50° C. Exemplary high stringency conditions include hybridization in 50% formamide, 1 M NaCl, 1% SDS at 37° C, and a wash in 0.1×SSC at 60° C. The term “a target”, as used herein, means a nucleic acid, such as a gene(s), EST(s), cDNA(s), mRNA(s), or gene product(s), their expressed product(s) or protein to which an inhibitor used in this invention acts on. For example, an oligonucleotide targeting to a specific nucleic acid that is antisense to its target nucleic acid and suppresses the expression of a target gene, thereby production of the target protein is inhibited. The terms “treat” or “treating”, as used herein, mean a treatment which decreases the likelihood that the subject administered such treatment will manifest symptoms of disease or other conditions. The term “a universal base”, as used herein, means a substitute base used for adenosine in its position in a nucleic acid which forms a hydrogen bond and binds to thymidine but lacks the ability to activate adenosine receptors and otherwise exercise the constricting effect of adenosine in the lungs. The term “up-regulation”, as used herein, means an increase in production, secretion, expression, function or availability (and thus an increase in concentration) of the targeted protein or nucleic acids. The term “an up-taken agent”, as used herein, means an agent which helps a cell take up a substance into a cell. It is used to take an exogenous substance into a cell to passively give a different genotype and/or phenotype. Preferably, the uptaken agents are transferrin, asialoglycoprotein, streptavidin, or sperinine.

This invention provides oligonucleotides (oligos) that is antisense to a nucleic acid target(s) such as G-alphaH, or other genes, or mRNAs to the genes' initiation codons, genomic flanking regions, intron-exon borders, their 5′-end, 3′-end, and regions within 2 to 10 nucleotides of the 5′-end and the 3′-end, the border sections between their coding and non-coding regions, or coding and non-coding regions of RNAs corresponding to the target genes.

The oligos of this invention may be obtained by first selecting fragments of a target nucleic acid having at least 4 contiguous nucleic acids selected from the group consisting of G and C, and then obtaining a first oligonucleotide 4 to 70 nucleotides long which comprises the selected fragment and preferably has a C and G nucleic acid content of up to and including about 20%, about 15%. A second complementary oligonucleotide 4 to 70 nucleotides long is then obtained comprising a sequence which is anti-sense to the selected fragment, the second oligonucleotide having an adenosine base content of up to and including about 20%, about 15%. When the first selected fragment comprises at least one thymidine base, the corresponding adenosine base in the second anti-sense oligonucleotide may be substituted with a universal base selected from heteroaromatic bases which bind to a thymidine base but have antagonist activity and less than about 0.3 of the adenosine base agonist activity at the adenosine A₁, A_{2b} and A₃ receptors, or heteroaromatic bases which have no activity or have an agonist activity at the adenosine A₂ₐ receptor.

A “nucleoside” is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligos that do not have a phosphorus atom in their internucleoside backbone are also called oligonucleosides. Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. These modifications may increase the in vivo stability of the oligonucleotide are particularly preferred. The naturally occurring phosphodiester linkages of oligonucleotides are susceptible to some degree of degradation by cellular nucleases. Many of the residues proposed herein, on the contrary, are highly resistant to nuclease degradation. See Milligan et al., and Cohen, J. S. D., supra. In another preferred embodiment, the oligo(s) may be protected from degradation by adding a “3′-end cap” by which nuclease-resistant linkages are substituted for phosphodiester linkages at the 3′ end of the oligonucleotide. See, Tidd, D. M. and Warenius, H. M., Be. J. Cancer 60: 343-350 (1989); Shaw, J. P. et al., Nucleic Acids Res. 19: 747-750 (1991), the relevant section of which are incorporated in their entireties herein by reference. Phosphoramidates, phosphorothioates, and methylphosphonate linkages all function adequately in this manner for the purposes of this invention. The more extensive the modification of the phosphodiester backbone the more stable the resulting agent, and in many instances the higher their RNA affinity and cellular permeation. See Milligan, et al., supra. The number of residues that may be modified or substituted will vary depending on the need, target, and route of administration, and may be from 1 to all the residues, to any number in between. Many different methods for replacing the entire phosphodiester backbone with novel linkages are known. See, Millikan et al, supra. Phosphorothioate and methylphosphonate-modified oligonucleotides are particularly preferred due to their availability through automated oligonucleotide synthesis. See, Millikan et al, supra. Where appropriate, the agent of this invention may be administered in the form of their pharmaceutically acceptable salts, or as a mixture of the oligonucleotide and its salt In another embodiment of this invention, a mixture of different oligonucleotides or their pharmaceutically acceptable slats is administered. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide (oligo) or oligo mimetic backbones that do not include a phosphorus atom therein have backbones that are formed by short chain alkyl or cycloalkyl internucleoside linkages, mixed heteroatom or alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; formacetal and thioformacetal backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Other preferred modified oligonucleotide backbones have thioether, carbonate, carbamate, sulfate, sulfite, hydroxylamine, methylene(methyimino) (MMI), methyleneoxy (methylimino) (MOMI), 2′-O-methyl, phosphoramidate backbones and combination thereof. Representative United States patents that teach oligomimetic preparation include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other preferred oligomimetics or modified oligos of the invention, both the sugar and the internucleoside linkage, i.e. the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and may be bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500). Most preferred embodiments of the invention are oligonucleotides with phosphorothioate backbones and oligonucleosides with heteroatom backbones, and in particular, —CH₂NHOCH₂—, —CH₂N(CH₃)OCH₂—(methylene (methylimino) or MMI backbone), —CH₂ON(CH₃)CH₂, —CH₂N(CH₃)N(CH₃)CH₂— and —ON(CH₃)CH₂CH₂— (wherein the native phosphodiester backbone is represented as —OPOCH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides (oligos) may also contain one or more substituted sugar moieties. Preferred modified oligos comprise one of the following at the 2′ position: OH; F; O—, S—, or N-alkyl, O-alkyl-O-alkyl, O—, S—, or N-alkenyl, or O—, S—, or N-alkynyl, wherein the alkyl, alkenyl and alkynyl may be substituted or unsubstituted C₁ to C₁₀ alkyl or C₂ to C₁₀ alkenyl and allynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OC₃, SOCH₃, SO₂CH₃, ONO₂, NO₂, N₃, NH₂, heterocycloalkyl, heterocycloalkaryl, aminoalkylamino, poly-alkylamino, substituted silyl an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O—(2-methoxyethyl) or 2′-MOE) (Martin et al., Helv. Chim. Acta 1995, 78, 486-504) i.e., an alkoxyalkoxy group. Further preferred modifications include 2′-dimethylaminooxyethoxy, i.e., a O(CH₂)₂ON(CH₃)₂ group, also known as 2′-DMAOE, and 2′-dimethylaminoethoxyethoxy (2′-DMAEOE) as described in examples hereinbelow. Other preferred modifications include 2′-methoxy (2-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Also Locked Nucleic Acid (LNA) and morpholino may be applicable for sugar mimetics. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920.

Oligos may also include nucleobase (“base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (^{me}C or ^{m}C), 5-hydroxymethyl cytosine, xanthine and its derivatives (e.g., theophylline, caffeine, dyphylline, etophylline, acephylline piperazine, bamifyllne, and enprofylline), hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of polymer Science And Engineering 1990, pages 858-859, Kroschwitz, J. I., ed. John Wiley & Sons, those disclosed by Englisch et al., Angewandte Chemie, International Edition 30: 613-722 (1991), and those disclosed by Sanghvi, Y. S., Chapter 15, Antisense Research and Applications, pp. 289-302, Crooke, S. T. and Lebleu, B., Eds., CRC Press (1993). Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by about 0.6 to about 1.2 degree. C. Sanghvi, Y. S., Crooke, S. T. and Lebleu, B., Eds., Antisense Research and Application, CRC press, Boca Raton, pp. 276-278 (1993), and are presently preferred base substitutions, even more particularly when combined with 2′-O-methoxyethyl sugar modifications. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

Another modification of the oligos of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Nat2. Acad. Sci. USA 1989, 86, 6553-6556), cholic acid (Manoharan et al., Bioorg. Med. Chem. Lett. 1994, 4, 1053-1059), a thioether, e.g., hexyl-S-tritylthiol (Manoharan et al., Ann. N.Y. Acad. Sci. 1992, 660, 306-309; Manoharan et al., Bioorg. Med. Chem Let 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. pharmacol. Exp. Ther., 1996, 277, 923-937). Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The present invention also includes oligos which are chimeric oligos. These oligonucleotides typically contain at least one region wherein the oligo is modified so as to have increased resistance to nuclease degradation, increased cellular uptake, and/or increased binding affinity for the target nucleic acid. As the present oligos may be single or double stranded RNAs, DNAs or RNA/DNAs, an additional region of the oligo may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of an RNA target may be routinely detected by gel electrophoresis and, if necessary, by associated nucleic acid hybridization techniques known in the art. Chimeric oligos of the invention include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 540 and 3′ “wings”) are modified to have greater affinity for the target RNA molecule but are unable to support nuclease activity (e.g. fluoro- or 2′-O-methoxyethyl-substituted). Chimeric oligos are not limited to being modified at the sugar moiety, but may also include oligonucleosides or oligonucleotides with modified backbones, e.g. with regions of phosphorothioate and phosphodiester backbone linkages, or with regions of MMI and phosphorothioate backbone linkages, among others. Other chimeras include “wingmers,” also known in the art as “hemimers,” that is, oligos with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the oligonucleotides of the present invention contain a 2′-O-methoxyethyl (2′-O—CH₂CH₂OCH₃) modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (OCH₂CHOCH₂) modification. Oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also preferred. Chimeric oligonucleotides comprising one or more such modifications are presently preferred. The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is well known to use similar techniques to prepare oligonucleotides such as the phosphorothioates and 2′-alkoxy or 2′-alkoxyalkoxy derivatives, including 2′-O-methoxyethyl oligonucleotides (Martin, P., Helv. Chim. Acta 78: 486-504 (1995)). Similar techniques and commercially available modified amidites and controlled-pore glass (CPG) products, such as biotin, fluorescein, acridine, psoralen-modified amidites and CPG (available from Glen Research, Sterling, Va.) may be employed to synthesize fluorescently labeled, biotinylated or other conjugated oligos. The antisense oligos of the invention include bioequivalent compounds, such as pharmaceutically acceptable salts and prodrugs of the oligos. This is intended to encompass any pharmaceutically acceptable salts, esters, or salts of such esters, or any other compound that, upon administration, is capable of providing (directly or indirectly) the biologically active metabolites of the compounds and residues thereof. Accordingly, for example, the invention also includes pharmaceutically acceptable salts of the nucleic acids of the invention and their prodrugs of such nucleic acids. “Pharmaceutically acceptable salts” are physiologically and pharmaceutically acceptable salts of the nucleic acids of the invention, such as salts that retain the desired biological activity of the parent compound and do not exhibit undesirable toxicological effects. See, for example, Berge et al., J. Pharm. Sci. 66: 1-19 (1977).

Examples of pharmaceutically acceptable salts of the oligos include, but are not limited to, (a) salts formed with cations such as sodium, potassium, ammonium, magnesium, calcium, polyamines such as spermine and spermidine, and the like; (b) acid addition salts formed with inorganic acids, e.g. hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The oligos of the invention may additionally or alternatively be prepared to be delivered as a prodrug. When no first oligo segments having the desired T content are found or where desirable segments contain T, it is possible to reduce the adenosine content of the second oligos corresponding to the thymidines (T) present in the target RNA to less than about 20%, about 15%, or fully eliminate A from the oligonucleotide sequence as a means for preventing their breakdown products from freeing adenosine into the lung tissue environment and, thereby, aggravating the subject's ailment and/or countering the beneficial effect of the administered agent The STA and MTA oligos used in this invention have the capacity to attenuate the expression of one or more target mRNA(s), or to enhance or attenuate the activity of one or more pathways. By means of example, all possible anti-sense sequences of about 7, about 10, about 12, about 15, about 18, about 21 to about 28, about 30, about 35, about 40, about 45, about 50, about 60, about 70 or more mononucleotides may be identified in a target mRNA, for example by searching for segments that are 7 or more nucleotides long within a target sequence, the segments being low in, or lacking thymidine (T), a nucleotide which is complementary to adenosine (A). This search typically results in about 10 to 30 such desT segments, i.e. naturally lacking thymidine, or segments with low T content, e.g. up to and including about 20%, about 15% T, from which oligonucleotides of varying lengths may be designed for a typical target mRNA of average length, i.e. about 1800 nucleotides long. The sense sequence for each strictly complementary desA anti-sense oligo sequence obtained for a specific target may be then deduced. The deduced sense sequence, thus, may be then used to search for sequences of preferred secondary targets. Alternatively, one or more sequence databases, e.g., GENBANK, and the like, may be searched for alternative secondary sequences. Thus, the targeting may be undertaken in several manners, one being the selection of specific targets associated with one or more related diseases. Alternatively, a primary target may be selected first, and an oligonucleotide found, preferably, a desA oligonucleotide and, then, secondary, tertiary or more targets searched for if an MTA is desired. In a typical search, either the list of preferred secondary targets or of a data base, multiple instances of homologous secondary targets of interest are identified. That is, the present technology is directed to finding the instances where there are natural homologies between primary, secondary, and other target sequences, and utilizing the finding for designing anti-sense oligos for preventative and therapeutic treatment of specific diseases or conditions associated with the target macromolecules from which the MTAs are obtained.

In the present invention, the oligos targeted to mRNAs associated with ailments involving lung airway pathology(ies), and their modification may be designed to reduce undesirable side effects caused by adenosine release upon breakdown, while preserving their activity and efficacy for their intended purpose. In this manner, the inventor targets a specific gene to design one or more oligo(s) that selectively bind(s) to the corresponding mRNA, and then reduces, if necessary, their content of adenosine via substitution with universal base or an adenosine analog incapable of activating adenosine A₁A_{2b} or A₃ receptors. Based on the prior experience in the field, the inventors reasoned that in addition to “down-regulating” specific genes, they could increase the effect of the oligo(s) administered by either selecting segments of RNA that are devoid, or have a low content, of thymidine (T) or, alternatively, substitute one or more adenosine(s) present in the designed oligo(s) with other nucleotide bases, so called universal bases, which bind to thymidine but lack the ability to activate adenosine receptors and otherwise exercise the constricting effect of adenosine in the lungs, etc. Given that adenosine (A) is a nucleotide base complementary to thymidine (T), when a T appears in the RNA, the oligo will have an A at the same position. For consistency's sake, all RNAs and oligos are represented in this patent by a single strand in the 5′ to 3′ direction, when read from left to right, although their complementary sequence(s) is (are) also encompassed within the four corners of the invention. In addition, all nucleotide bases and amino acids are represented utilizing the recommendations of the IUPAC-IUB Biochemical Nomenclature Commission, or by the known 3-letter code (for amino acids). The oligo(s) of this invention may be used to treat ailments associated with airway inflammation which may be accompanied by reduced airway function in a subject, whatever its cause. The oligo(s) used in the invention may have a reduced A content to prevent its liberation upon in vivo degradation of the oligo(s). Examples of airway diseases that may be treated by the method of this invention include airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), and/or bronchoconstriction. By means of example, the G-alphaH gene may be selected as a primary target and searched for low or T-free (desthymidine (desT)) segments. When a number of desT segments are found, their anti-sense segments may be deduced, and perhaps about 20 or even more desA anti-sense oligo sequences obtained. In some cases, these anti-sense sequences may represent all desA anti-sense sequences found within the mRNA of this primary target and, for MTAs it may be utilized to start the search for homologous sequences within a preferred list of secondary targets such as the one shown in Table 1 above or Table 2 below, or within a sequence data base, such as GENBANK. For each of the about 20 original desA anti-sense sequences found for the G-alphaH gene, typically about 10 to 30 homologous sequences may be found among the other members of the group shown in Table 1 below (secondary, tertiary, and the like targets). The SEQ ID NOS and the GENBANK ACCESSION numbers of the nucleic acids for the selected respiratory genes are shown in Table 1 below.

TABLE 1
Exemplary Genes

| | | SEQ ID NO of the | SEQ ID NOS of | |
|---|---|---|---|---|
| Target Human Gene | Genbank Accession No. | Gene | Oligo(s) | SEQ ID of Concatemer |
| IL4R (interleukin 4 receptor) | NM_000418 | 1 | 2-157 | 158 |
| IL5R (interleukin 5 receptor) | X61177 | 159 | 160-319 | 320 |
| CCR1 | XM_003248 | 321 | 322-353 | 354 |
| CCR3 | NM_001837 | 355 | 356-527 | 528 |
| eotaxin-D49372 | D49372 | 529 | 530-565 | 566 |
| eotaxin-U46573 | U46573 | 567 | 568 | 569 |
| eotaxin-U46572 | U46572 | 570 | 571-605 | 606 |
| RANTES | NM_002985 | 607 | 608-736 | 737 |
| MCP4 | Z77650 | 738 | 739-860 | 861 |
| CD23-X04772 | X04772 | 862 | 863-1072 | 1073 |
| CD23-M23562 | M23562 | 1074 | 1075-1077 | 1078 |
| CD23-M14766 | M14766 | 1079 | 1080 | 1081 |
| ICAM | J03132 | 1082 | 1083-1291 | 1292 |
| VCAM | X53051 | 1293 | 1294-1318 | 1319 |
| Tryptase-a | M33494 | 1320 | 1321-1485 | 1486 |
| Tryptase-b | M37488 | 1487 | 1488-1571 | 1572 |
| PDE4A | U97584 | 1573 | 1574-1781 | 1782 |
| PDE4B | NM_002600 | 1783 | 1784-1788 | 1789 |
| PDE4C | Z46632 | 1790 | 1791-2152 | 2153 |
| PDE4D | NM_006203 | 2154 | 2155-2162 | 2163 |
| IL5R-X61176 | X61176 | 2164 | 2165-2496 | 2497 |
| concatemer | — | 2498 | 2499 | — |

In some instances, the search produces homologies for the primary target with not only secondary targets (homology between primary target and the sequence from one other target), but also with tertiary targets (homology between primary target and sequences from, e.g. three other target mRNA). When this occurs, the oligos found are said to be 100% homologous. More typically, however, the sequences found contain one or more non-fully homologous nucleotides within the secondary, tertiary or quaternary sequences. In many cases, this mismatch might generally suffice to render the oligonucleotide less active or even inactive against the target(s). A degree of mismatch between “homologous” sequences may be up to about 40%, about 30%, about 20%, about 10%, about 5%. mismatched nucleotides and even more preferred no more than about 5 %. In some instances, higher % mismatch is acceptable, and the oligos still are active since the non-homologous nucleotide may be “fixed” or replaced with a “universal” base that may base-pair with similar or equal affinity with two or more of the four nucleotide present in natural DNA: A, G, C, and T, whichever it must hybridize or is antisense to. This “fixing” step generates a further novel sequence, different from the one found in nature, that permits the oligo(s) to bind, preferably equally well, with the primary, secondary and tertiary target, etc.

When a respiratory gene is selected as a target, its mRNA or DNA is searched for low uridine (U) or uridine-free (des U) fragments, or thymidine or thymidine-free (desT) fragments. Only U or T and des U or desT segments of the mRNA or DNA are selected which, in turn, will produce low A or desA anti-sense as their complementary strand. When a number of RNA desT segments are found, the sequence of the anti-sense segments may be synthesized. Typically, about 10 to about 30, and even larger numbers, of desA anti-sense sequences may be obtained by this method. These anti-sense sequences may include some or all desA oligonucleotide sequences corresponding to low U or T or des U or desT segments of the target mRNA or DNA, such as any one of those shown in Table 1 above or Table 2 below. When this occurs, the oligos found are said to be low A or 100% A-free. For each of the original desA oligos corresponding to the target gene, typically about 10 to 30 sequences may be found within the target gene or RNA that have a low U content (RNA) or T content (DNA). In accordance with this invention, the selected fragment sequences may also contain a small number of uridine nucleotides (RNA) within the secondary or tertiary or quaternary sequences. A replacement of nucleotides may be done to decrease the A content of the anti-sense oligo and/or to increase hybridization to a plurality of targets.

In this invention, these so called “non-fully desA” sequences may preferably have a content of adenosine of less than about 15%, about 10%, about 5%, and some even less than 2% adenosine. In some instances a higher content of adenosine is acceptable and the oligonucleotides are still active, particularly where the adenosine nucleotide may be “fixed” or replaced with a “universal” base that may base-pair with similar or equal affinity to two or more of the four nucleotide present in natural DNA: A, G, C, and T. A “universal base” is defined in this patent as any compound, more commonly a pyrimidine or purine analogue, having the capacity to hybridize to one or more of A, T, C, U or G. In another embodiment, the universal base has substantially reduced, or substantially lacking, ability to bind adenosine receptors. Adenosine analogs which do not activate adenosine receptors, such as the adenosine A₁, A_{2b} and/or A₃ receptors, may be used.

This “fixing” step generates a novel sequence(s), different from the one(s) found in nature, that permits the oligonucleotide(s) to bind, preferably equally well, with the target RNA. Examples of universal bases are 1(2′-deoxy-β-D-ribofuranosyl)-5-nitroindole, 1-(2′-deoxy-β-D-ribofuranosyl)-3-nitropyrrole, 7-(2′-deoxy-β-D-ribofuranosyl)inosine, 7-(2′-deoxy-β-D-ribofuranosyl)nebularine, 6H, 8H-3,4-dihydropyrimido[4,5-c]oxazine-7-one-2′-deoxyribose and 2-amino-6-methoxyaminopurine (Glen Research, Sterling, Va.). In addition to the above, universal bases which may be substituted for any other base although with somewhat reduced hybridization potential, include 1-(2′-deoxy-β-D-ribofuranosyl)-3-nitropyrrole, 1-(2′-deoxy-β-D-ribofuranosyl)-5-nitroindole, 7-(2′-deoxy-β-D-ribofuranosyl)inosine, 7-(2′-deoxy-β-D-ribofuranosyl)nebularine, 7-(2′-deoxy-β-D-ribofuranosyl)isoguanosine, 7-(2′-deoxy-β-D-ribofuranosyl)-4-methylindole, 7-(2′-deoxy-β-D-ribofuranosyl)-6-phenylinosine, 7-(2′-deoxy-β-D-ribofuranosyl)-2,6-diamine-purine (TriLink BioTechnologies, San Diego, Calif.). More specific mismatch repairs may be made using “P” nucleotide, 6H, 8H-3,4-dihydropyrimido[4,5-c][1,2]oxazin-7-one-2′deoxyribose, which base pairs with either guanine (G) or adenine (A) and “K” nucleotide, 2-amino-6-methoxyaminopurine, which base pairs with either cytidine (C) or thymidine (T), among others. An artisan will know how to select or find others. Moreover, others that are known in the art are also suitable. See, for example, Loakes, D. and Brown, D. M., Nucl. Acids Res. 22:4039-4043 (1994); Ohtsuka, E. et al., J. Biol. Chem.260(5):2605-2608 (1985); Lin, P. K. T. and Brown, D. M., Nucleic Acids Res. 20(19):5149-5152 (1992; Nichols, R. et al., Nature 369(6480): 492-493 (1994); Rahmon, M. S. and Humayun, N. Z., Mutation Research 377 (2): 263-8 (1997); Amosova, O., et al., Nucleic Acids Res. 25 (10): 1930-1934 (1997); Loakes D. & Brown, D. M., Nucleic Acids Res. 22 (20): 4039-4043 (1994), the entire sections relating to universal bases and their preparation and use in nucleic acid binding are incorporated herein by reference. When non-fully des U or desT sequences are found in the naturally occurring mRNA or target, they are selected typically so that about 1 to 3 universal base substitutions will suffice to obtain a 100% “desA” oligonucleotide. Thus, the present method provides oligonucleotides to different targets which are low in, or devoid of, A content, and oligonucleotides wherein one or more adenosines, or other bases may be “fixed” by replacement with a “universal” base. Universal bases are known in the art and need not be listed herein. An artisan will know which compounds may act as universal bases, and replace them for A or any of the other bases.

The present approach in the design of oligonucleotides produces oligos suitable for application to a variety of diseases or conditions, e.g. respiratory and lung diseases including inflammatory diseases, such as airway inflammation, lung allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, and bronchoconstriction, among others.

The present invention is concerned primarily with the treatment of vertebrates, and within this group, of mammals, including human and non-human simians, wild and domesticated animals, marine and land animals, household pets, and zoo animals, for example, felines, canines, equines, pachiderms, cetaceans, and still more preferably to human subjects. One particularly suitable application of this technology, however, is for veterinary purposes, and includes all types of small and large animals in the care of a veterinarian, including wild animals, marine animals, household animals, zoo animals, and the like. Targeted genes and proteins are preferably mammalian, and the sequences targeted for producing the oligos of the invention are preferably of the same species as the subject being treated. Although in many instances, targets of a different species are also suitable, particularly those segments of the target RNA or gene that display greater than about 25%, about 45%, about 85%, about 95% homology, with the recipient's sequence. A preferable group of compositions is composed of des-A anti-sense oligos. Another preferred group is composed of non-fully desA oligonucleotides, where one or more adenosine or other bases are replaced with universal bases.

The present composition and formulations reduce gene expression of the target genes and/or mRNA(s), such as those of the interleukin-4 receptor and related genes listed in Table 1. This is generally attained by hybridization of the oligonucleotides to the coding (sense) sequence of a targeted messenger RNA (mRNA) as is known in the art. The exogenously administered compositions of the invention decrease the levels of mRNA and/or protein encoded by the target gene. They may also cause changes in the growth characteristics or shapes of the thus treated cells. See, Milligan et al. (1993); Helene, C. and Toulme, 3. Biochim. Biophys. Acta 1049, 99-125 (1990); Cohen, J. S. D., Ed., Oligodeoxynucleotides as Anti-sense Inhibitors of Gene Expression; CRC Press: Boca Raton, Fla. (1987), the relevant portion of which is hereby incorporated in its entirety by reference. Many protein, RNA and gene sequences are in the public domain. Others may be deduced from known information. The mRNA sequence of a targeted protein may be derived from the nucleotide sequence of the gene expressing the protein. For example, the sequence of the genomic human adenosine A₁ receptor and that of the rat and human adenosine A₃ receptors are known. See, U.S. Pat. No. 5,320,962; Zhou, F., et al., Proc. Nat'l Acad. Sci. (USA) 89: 7432 (1992); Jacobson, M. A., et al., U.K. Pat. Appl. No. 9304582.1. The sequence of the adenosine A_{2b} receptor gene is also known. See, Salvatore, C. A., Luneau, C. J., Johnson, R. G. and Jacobson, M., Genomics (1995), the relevant portion of which is hereby incorporated in its entirety by reference. The sequences of many of the exemplary target genes are also known. See, GENBANK Data Base, NIH, Rockville, Md. The sequences of unavailable genes may be obtained by isolating target segments and by applying technology known in the art. Once the sequence of the gene, EST(s), cDNA(s), mRNA(s) and/or the protein are known, an oligonucleotide(s) may be produced as described above according to this invention in accordance with standard techniques.

In one aspect of this invention, the oligo(s) has (have) a sequence(s) that specifically bind(s) to a portion or segment of an mRNA molecule which encodes a protein(s) associated with a disease or condition that may be associated with airway and/or lung inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, and bronchoconstriction, and the like. One effect of this binding is to reduce or even prevent the translation of the corresponding mRNA and, thereby, reduce the available amount of target protein in the subject's lung.

The oligo(s) of this invention have the capacity to attenuate the expression of one or more target genes or mRNAs and/or to attenuate the activity of one or more pathways. By means of example, the present method may be practiced by identifying all possible deoxyribonucleotide segments which are low in uridine (U) (RNA), or thymidine (T) (DNA) or deoxynucleotide segments low in adenosine (A) (oligos) of about 7 or more mononucleotides, preferably up to about 60 mononucleotides, more preferably about 10 to about 36 mononucleotides, and still more preferably about 12 to about 21 mononucleotides, in a target mRNA or a gene, respectively. This may be attained by searching for mononucleotide segments within a target sequence which are low in, or lack uridine or other bases (RNA) or thymidine (T) or other bases (DNA), a nucleotide which is complementary to adenosine, or that are low in adenosine (gene), cytosine, guanidine and thymidine. In most cases, this search typically results in about 10 to 30 oligos of varying lengths for a typical target mRNA of average length, i.e., about 1800 nucleotides long. Those oligo sequences selected are then compared with other target segments for hybridizable portions. If complete homology is not found, the oligo sequences may be fixed by substitution of a universal base for one or more of the unmatched bases. The oligo(s) of this invention may be of any suitable length, including but not limited to, about 7, to about 60 nucleotides long, preferably about 12 to about 45, more preferably up to about 30 nucleotides long, and still more preferably up to about 21, although they may be of other lengths as well, depending on the particular target and the mode of delivery. The oligonucleotide(s) of the invention may be directed to any and all segments of a target RNA or DNA, and may be single or double stranded DNA or RNA oligos. One preferred group of oligo(s) includes those directed to a mRNA region containing an intron-exon junction. Where the oligo is directed to an intron-exon junction, it may either entirely overlie the junction or it may be sufficiently close to the junction to inhibit the splicing-out of the intervening exon during processing of precursor mRNA to mature mRNA, e.g. with the 3′ or 5′ terminus of the oligonucleotide being positioned within about, for example, within about 2 to 10, preferably about 3 to 5, nucleotide of the intron-exon junction. Also preferred are oligos that overlap the initiation codon, and those near the 5′ and 3′ termini of the coding region, among others.

This invention thus provides a composition, comprises an oligo(s) anti-sense to a single target (STA), or to multiple targets, (MTA) including target genes, coding and non-coding regions of mRNA, initiation codons of the genes, genomic flanking regions including the gene, intron-exon borders, 5′-end regions, 3′-end regions, regions within 2 to 10 nucleotides in length of the 5′-end or 3′-end, and regions overlapping the coding and non-coding regions, the entire sequence of precursor RNAs, poly-A segment, at least 4 contiguous nucleotides selected from RNA segments and RNAs encoding proteins known to be associated with one or more diseases or conditions or mixtures thereof.

The compositions in accordance with this invention are RNA, DNA or hybrids thereof and they may be single or double stranded. They are preferably designed to be anti-sense to target genes, ESTs, cDNAs, and/or mRNAs related in origin to the species to which it is to be administered. When treating humans, the agents are preferably anti-sense to a human gene or RNA. The compositions of the invention encompass oligos that are anti-sense to naturally occurring DNA and/or RNA sequences, fragments thereof of up to a length of one (1) base less than the targeted sequence, preferably at least about 7 nucleotides long, oligos having only over about 0.02%, about 0.1%, about 1%, and about 4% adenosine nucleotides, and up to about 30%, about 15%, about 10% and about 5%, adenosine nucleotide, or lacking adenosine altogether, and oligos, in which one or more of the adenosine nucleotides have been replaced with so-called universal bases that may pair up with thymidine nucleotides but fail to substantially trigger adenosine receptor activity. Examples of human sequences and fragments, which are not limiting, of oligonucleotide of the invention are the following fragments as well as shorter segments of the fragments and of the full gene or mRNA coding and non-coding sequences, exons and intron-exon junctions encompassing preferably 7, 10, 15, 18 to 21, 24, 27, 30, n-1 nucleotides for each sequence, where n is the sequence's total number of nucleotides. These fragments may be any portion of the longer oligo(s), for example, from the middle, 5′-end, 3′-end or starting at any other site of the original sequence. Of particular importance are fragments of low adenosine nucleotide content, that is, those fragments containing less than or about 30%, less than or about 15%, less than or about 10%, less than or about 5%, and devoid of adenosine nucleotides, either by choice or by replacement with a universal base in accordance with this invention. Similarly, other bases may be replaced to form an MTA as discussed above. The composition of the invention includes as a most preferred group of sequences and their fragments, where one or more adenosines or other bases present in the sequence have been replaced by a universal base (B), as exemplified here. Similarly, also encompassed are all shorter fragments of the B-containing fragments designed by substitution of B(s) for adenosine(s) (A(s)) contained in the sequences, fragments thereof or segments thereof, as described above. Similar substitutions may be made with a universal base of any of the other bases. Examples of the oligonucleotide sequences of this invention are provided in Table 1 above.

The following are examples of sequences corresponding to the targets exemplified in this invention. An annotation is made to oligo sequences in the order of SEQ ID NO, Code, Genbank Accession NO, and Sequence. The code is used as fragment numbers and the GENBANK Accession number are shown before the actual sequence. For example, the first oligonucleotide sequence for the interleukin-4 receptor gene listed below has SEQ ID NO: 2, the code (or fragment No) is MIL4R12, Genbank Accession No is NM_{—}000418, and its sequence is CTC-CAC-TCA-CTC-CAG-GTG. All nucleic acid sequences shown in this patent begin with their 5′ terminus, and all the amino acid sequences begin with their amino-acid terminus.

IL4R Nucleic Acid Sequences (GENBANK ACCESSION NO. NM 000418)

| (SEQ ID NO: 1) | | | | |
|---|---|---|---|---|
| GGCGAATGGAGCAGGGGCGCGCAGTATAATTAAAGATTTACACCAGCTGGAAGAAATCATAGAGAAGCCGGGCGTGGTGGCTCATGCCTATAATCCCAGCACTTTTGGAGGCTGAGGCGGGCAGATCACTTGAG | | | | |
| ATCAGGAGTTCGAGACCAGCCTGGTGCCTTGGCATCTCCCAATGGGGTGGCTTTGCTCTGGGCTCCTGTTCCCTGTGAGCTGCCTGGTCCTGCTGCAGGTGGCAAGCTCTGGGAACATGAAGGTCTTGCAGGAG | | | | |
| CCCACCTGCGTCTCCGACTACATGAGCATCTCTACTTGCGAGTGGAAGATGAATGGTCCCACCAATTGCAGCACCGAGCTCCGCCTGTTGTACCAGCTGGTTTTTCTGCTCTCCGAAGCCCACACGTGTATCCC | | | | |
| TGAGAACAACGGAGGCGCGGGGTGCGTGTGCCACCTGCTCATGGATGACGTGGTCAGTGCGGATAACTATACACTGGACCTGTGGGCTGGGCAGCAGCTGCTGTGGAAGGGCTCCTTCAAGCCCAGCGAGCATG | | | | |
| TGAAACCCAGGGCCCCAGGAAACCTGACAGTTCACACCAATGTCTCCGACACTCTGCTGCTGACCTGGAGCAACCCGTATCCCCCTGACAATTACCTGTATAATCATCTCACCTATGCAGTCAACATTTGGAGT | | | | |
| GAAAACGACCCGGCAGATTTCAGAATCTATAACGTGACCTACCTAGAACCCTCCCTCCGCATCGCAGCCAGCACCCTGAAGTCTGGGATTTCCTACAGGGCACGGGTGAGGGCCTGGGCTCAGTGCTATAACAC | | | | |
| CACCTGGAGTGAGTGGAGCCCCAGCACCAAGTGGCACAACTCCTACAGGGAGCCCTTCGAGCAGCACCTCCTGCTGGGCGTCAGCGTTTCCTGCATTGTCATCCTGGCCGTCTGCCTGTTGTGCTATGTCAGCA | | | | |
| TCACCAAGATTAAGAAAGAATGGTGGGATCAGATTCCCAACCCAGCCCGCAGCCGCCTCGTGGCTATAATAATCCAGGATGCTCAGGGGTCACAGTGGGAGAAGCGGTCCCGAGGCCAGGAACCAGCCAAGTGC | | | | |
| CCACACTGGAAGAATTGTCTTACCAAGCTCTTGCCCTGTTTTCTGGAGCACAACATGAAAAGGGATGAAGATCCTCACAAGGCTGCCAAAGAGATGCCTTTCCAGGGCTCTGGAAAATCAGCATGGTGCCCAGT | | | | |
| GGAGATCAGCAAGACAGTCCTCTGGCCAGAGAGCATCAGCGTGGTGCGATGTGTGGAGTTGTTTGAGGCCCCGGTGGAGTGTGAGGAGGAGGAGGAGGTAGAGGAAGAAAAAGGGAGCTTCTGTGCATCGCCTG | | | | |
| AGAGCAGCAGGGATGACTTCCAGGAGGGAAGGGAGGGCATTGTGGCCCGGCTAACAGAGAGCCTGTTCCTGGACCTGCTCGGAGAGGAGAATGGGGGCTTTTGCCAGCAGGACATGGGGGAGTCATGCCTTCTT | | | | |
| CCACCTTCGGGAAGTACGAGTGCTCACATGCCCTGGGATGAGTTCCCAAGTGCAGGGCCCAAGGAGGCACCTCCCTGGGGCAAGGAGCAGCCTCTCCACCTGGAGCCAAGTCCTCCTGCCAGCCCGACCCAGAG | | | | |
| TCCAGACAACCTGACTTGCACAGAGACGCCCCTCGTCATCGCAGGCAACCCTGCTTACCGCAGCTTCAGCAACTCCCTGAGCCAGTCACCGTGTCCCAGAGAGCTGGGTCCAGACCCACTGCTGGCCAGACACC | | | | |
| TGGAGGAAGTAGAACCCGAGATGCCCTGTGTCCCCCAGCTCTCTGAGCCAACCACTGTGCCCCAACCTGAGCCAGAAACCTGGGAGCAGATCCTCCGCCGAAATGTCCTCCAGCATGGGGCAGCTGCAGCCCCC | | | | |
| GTCTCGGCCCCCACCAGTGGCTATCAGGAGTTTGTACATGCGGTGGAGCAGGGTGGCACCCAGGCCAGTGCGGTGGTGGGCTTGGGTCCCCCAGGAGAGGCTGGTTACAAGGCCTTCTCAAGCCTGCTTGCCAG | | | | |
| CAGTGCTGTGTCCCCAGAGAAATGTGGGTTTGGGGCTAGCAGTGGGGAAGAGGGGTATAAGCCTTTCCAAGACCTCATTCCTGGCTGCCCTGGGGACCCTGCCCCAGTCCCTGTCCCCTTGTTCACCTTTGGAC | | | | |
| TGGACAGGGAGCCACCTCGCAGTCCGCAGAGCTCACATCTCCCAAGCAGCTCCCCAGAGCACCTGGGTCTGGAGCCGGGGAAAAGGTAGAGGACATGCCAAAGCCCCCACTTCCCCCAGGAGCAGGCCACAGAC | | | | |
| CCCCTTGTGGACAGCCTGGGCAGTGGCATTGTCTACTCAGCCCTTACCTGCCACCTGTGCGGCCACCTGAAACAGTGTCATGGCCAGGAGGATGGTGGCCAGACCCCTGTCATGGCCAGTCCTTGCTGTGGCTG | | | | |
| CTGCTGTGGAGACAGGTCCTCGCCCCCTACAACCCCCCTGAGGGCCCCAGACCCCTCTCCAGGTGGGGTTCCACTGGAGGCCAGTCTGTGTCCGGCCTCCCTGGCACCCTCGGGCATCTCAGAGAAGAGTAAAT | | | | |
| CCTCATCATCCTTCCATCCTGCCCCTGGCAATGCTCAGAGCTCAAGCCAGACCCCCAAAATCGTGAACTTTGTCTCCGTGGGACCCACATACATGAGGGTCTCTTAGGTGCATGTCCTCTTGTTGCTGAGTCTG | | | | |
| CAGATGAGGACTAGGGCTTATCCATGCCTGGGAAATGCCACCTCCTGGAAGGCAGCCAGGCTGGCAGATTTCCAAAAGACTTGAAGAACCATGGTATGAAGGTGATTGGCCCCACTGACGTTGGCCTAACACTG | | | | |
| GGCTGCAGAGACTGGACCCCGCCCAGCATTGGGCTGGGCTCGCCACATCCCATGAGAGTAGAGGGCACTGGGTCGCCGTGCCCCACGGCAGGCCCCTGCAGGAAAACTGAGGCCCTTGGGCACCTCGACTTGTG | | | | |
| AACGAGTTGTTGGCTGCTCCCTCCACAGCTTCTGCAGCAGACTGTCCCTGTTGTAACTGCCCAAGGCATGTTTTGCCCACCAGATCATGGCCCACGTGGAGGCCCACCTGCCTCTGTCTCACTGAACTAGAAGC | | | | |
| CGAGCCTAGAAACTAACACAGCCATCAAGGGAATGACTTGGGCGGCCTTGGGAAATCGATGAGAAATTGAACTTCAGGGAGGGTGGTCATTGCCTAGAGGTGCTCATTCATTTAACAGAGCTTCCTTAGGTTGA | | | | |
| TGCTGGAGGCAGAATCCCGGCTGTCAAGGGGTGTTCAGTTAAGGGGAGCAACAGAGGACATGAAAAATTGCTATGACTAAAGCAGGGACAATTTGCTGCCAAACACCCATGCCCAGCTGTATGGCTGGGGGCTC | | | | |
| CTCGTATGCATGGAACCCCCAGAATAAATATGCTCAGCCACCCTGTGGGCCGGGCAATCCAGACAGCAGGCATAAGGCACCAGTTACCCTGCATGTTGGCCCAGACCTCAGGTGCTAGGGAAGGCGGGAACCTT | | | | |
| GGGTTGAGTAATGCTCGTCTGTGTGTTTTAGTTTCATCACCTGTTATCTGTGTTTGCTGAGGAGAGTGGAACAGAAGGGGTGGAGTTTTGTATAAATAAAGTTTCTTTGTCTC | | | | |
| SEQ ID NO. | Code, | Genbank Accession NO, | Sequence | |
| 2, | MIL4R12, | NM_000418, | CTC-CAC-TCA-CTC-CAG-GTG, | |
| 3, | MIL4R13, | NM_00418, | CTC-CAC-TCA-CTC-CAG, | |
| 4, | MIL4R16, | NM_00418, | GCA-GCT-GCC-CCA-TGC-TG, | |
| 5, | MIL4R17, | NM_00418, | GAG-AAG-GCC-TTG-TAA-CC, | |
| 6, | HIL4R1, | NM_00418, | GCG-CCC-CTG-CTC-CAT-TCG-CC, | |
| 7, | HIL4R2, | NM_00418, | TTT-CTT-CCA-GCT-GTG-TGT, | |
| 8, | HIL4R3, | NM_00418, | CAC-CAC-GCC-CGG-CTT-CTC-T, | |
| 9, | HIL4R4, | NM_00418, | TCT-GCC-CGC-CTC-AGC-CTC-C, | |
| 10, | HIL4R5, | NM_00418, | GGC-ACC-AGG-CTG-GTC-TCG, | |
| 11, | HIL4R6, | NM_00418, | TGG-GAG-ATG-CCA-AGG-CAC, | |
| 12, | HIL4R7, | NM_00418, | GCC-ACC-CCA-TTG-GGA-GAT, | |
| 13, | HIL4R8, | NM_00418, | GCA-AAG-CCA-CCC-CAT-TGG, | |
| 14, | HIL4R9, | NM_00418, | GTT-CCC-AGA-GCT-TGC-CAC-CT, | |
| 15, | HIL4R10, | NM_00418, | GGA-GCA-CGG-TGC-TGC-AAT-TG, | |
| 16, | HIL4R11, | NM_00418, | GAT-ACA-CGT-GTG-GGC-TTC-GG, | |
| 17, | HIL4R12, | NM_00418, | GCG-CCT-CCG-TTG-TTC-TCA-GG, | |
| 18, | HIL4R13, | NM_00418, | GCA-GCT-GCT-GCC-CAG-CCC, | |
| 19, | HIL4R14, | NM_00418, | GGT-TTC-CTG-GGG-CCC-TGG-GT, | |
| 20, | HIL4R15, | NM_00418, | GGG-ATA-CGG-GTT-GCT-CCA-G, | |
| 21, | HIL4R16, | NM_00418, | TCT-GCC-GGG-TCG-TTT-TCA-CT, | |
| 22, | HIL4R17, | NM_00418, | TTC-AGG-GTG-CTG-GCT-GCG, | |
| 23, | HIL4R18, | NM_00418, | GGC-CCT-CAC-CCG-TGC-CCT-GT, | |
| 24, | HIL4R19, | NM_00418, | CCA-CTC-ACT-CCA-GGT-GGT-GT, | |
| 25, | HIL4R20, | NM_00418, | TGT-GCC-ACT-TGG-TGC-TGG, | |
| 26, | HIL4R21, | NM_00418, | TGC-TGC-TCG-AAG-GGC-TCC-CT, | |
| 27, | HIL4R22, | NM_00418, | GGC-GGC-TGC-GGG-CTG-GGT, | |
| 28, | HIL4R23, | NM_00418, | CGG-GAC-CGC-TTC-TCC-CAC-TG, | |
| 29, | HIL4R24, | NM_00418, | CTT-GGC-TGG-TTC-CTG-GCC-T, | |
| 30, | HIL4R25, | NM_00418, | TGG-CAG-CCT-TGT-GAG-GAT-CT, | |
| 31, | HIL4R26, | NM_00418, | TCT-TGC-TGA-TCT-CCA-CTG-GG, | |
| 32, | HIL4R27, | NM_00418, | GTC-ATC-CCT-GCT-GCT-CTC, | |
| 33, | HIL4R28, | NM_00418, | TCT-TAC-TTC-CCG-AAG-GTG-G, | |
| 34, | HIL4R29, | NM_00418, | GGT-TGT-CTG-GAC-TCT-GGG-T, | |
| 35, | HIL4R30, | NM_00418, | GTT-GCT-GAA-GCT-GCG-GT, | |
| 36, | HIL4R31, | NM_00418, | CGG-GAC-CGC-TTC-TCC-CAC-TG, | |
| 37, | HIL4R32, | NM_00418, | TGC-TCC-CAG-GTT-TCT-GGC-TC, | |
| 38, | HIL4R33, | NM_00418, | CCC-TGC-TCC-ACC-GCA-TGT, | |
| 39, | HIL4R34, | NM_00418, | GGC-TTA-TAC-CCC-TCT-TCC-C, | |
| 40, | HIL4R35, | NM_00418, | TGA-GCT-CTG-CGG-ACT-GCG, | |
| 41, | HIL4R36, | NM_00418, | CTG-TTT-CAG-GTG-GCC-GC, | |
| 42, | HIL4R37, | NM_00418, | GGA-TTT-ACT-CTT-CTC-TG, | |
| 43, | HIL4R38, | NM_00418, | GGG-TCT-GGC-TTG-AGC-TCT-G, | |
| 44, | HIL4R39, | NM_00418, | GCC-CTA-GTC-CTC-ATC-TGC, | |
| 45, | HIL4R40, | NM_00418, | TGC-CAG-CCT-GGC-TGC-CTT-CC, | |
| 46, | HIL4R41, | NM_00418, | GCG-ACC-CAG-TGC-CCT-CTA-CT, | |
| 47, | HIL4R42, | NM_00418, | GTC-TGC-TGC-AGA-AGC-TGT-GG, | |
| 48, | HIL4R43, | NM_00418, | GGC-TCG-GCT-TCT-AGT-TCA-G, | |
| 49, | HIL4R44, | NM_00418, | GTC-TGC-TGC-AGA-AGC-TGT-GG, | |
| 50, | HIL4R45, | NM_00418, | TCG-ATT-TCC-CAA-GGC-CGC-CC, | |
| 51, | HIL4R46, | NM_00418, | TTC-ATG-TCC-TCT-GTT-GCT-CC, | |
| 52, | HIL4R47, | NM_00418, | GGC-ATG-GGT-GTT-TGG-CAG-C, | |
| 53, | HIL4R48, | NM_00418, | GTG-CCT-TAT-GCC-TGC-TGT-CT, | |
| 54, | HIL4R49, | NM_00418, | TTC-TGT-TCC-ACT-CTC-CTC, | |
| 55, | HIL4R50, | NM_00418, | AGA-AAC-TTT-ATT-TAT-ACA-A, | |
| 56, | HIL4R51, | NM_00418, | GGC-TCC-ACT-CAC-TCC-AG, | |
| 57, | HIL4R52, | NM_00418, | GGC-TCC-ACT-CAC-TCC-A, | |
| 58, | HIL4R53, | NM_00418, | GCT GGG ATT ATA GGC ATG AG, | |
| 59, | HIL4R54, | NM_00418, | CCT GAT CTC AAG TGA TCT GC, | |
| 60, | HIL4R55, | NM_00418, | ACA GGG AAC AGG AGC CCA GA, | |
| 61, | HIL4R56, | NM_00418, | GCA GCA GGA CCA GGC AGC TC, | |
| 62, | HIL4R57, | NM_00418, | GGC TCC TGC AAG ACC TTC AT, | |
| 63, | HIL4R58, | NM_00418, | TGT AGT CGG AGA CGC AGG TG, | |
| 64, | HIL4R59, | NM_00418, | CTC GCA AGT AGA GAT GCT CA, | |
| 65, | HIL4R60, | NM_00418, | GTG GGA CCA TTC ATC TTC CA, | |
| 66, | HIL4R61, | NM_00418, | AAC CAG CGT GTG GGC TTC GG, | |
| 67, | HIL4R62, | NM_00418, | AGA GCA GAA AAA CCA GCT GG, | |
| 68, | HIL4R63, | NM_00418, | GAT ACA CGT GTG GGC TTC GG, | |
| 69, | HIL4R64, | NM_00418, | ACT GAC CAC GTC ATC CAT GA, | |
| 70, | HIL4R65, | NM_00418, | TCC AGT GTA TAG TTA TCC GC, | |
| 71, | HIL4R66, | NM_00418, | GAA GGA GCC CTT CCA CAG CA, | |
| 72, | HIL4R67, | NM_00418, | TTC ACA TGC TCG CTG GGC TT, | |
| 73, | HIL4R68, | NM_00418, | GAC ATT GGT GTG AAC TGT CA, | |
| 74, | HIL4R69, | NM_00418, | GTC AGC AGC AGA GTG TCG GA, | |
| 75, | HIL4R70, | NM_00418, | ATT ATA CAG GTA ATT GTC AG, | |
| 76, | HIL4R71, | NM_00418, | TTG ACTGCA TAG GTG AGA TG, | |
| 77, | HIL4R72, | NM_00418, | ATA GAT TCT GAA ATC TGC CG, | |
| 78, | HIL4R73, | NM_00418, | GGT TCT AGG TAG GTC ACG TT, | |
| 79, | HIL4R74, | NM_00418, | TGG CTG CGA TGC GGA GGG AG, | |
| 80, | HIL4R75, | NM_00418, | CCC TGT AGG AAA TCC CAG AC, | |
| 81, | HIL4R76, | NM_00418, | TGG CTG CGA TGC GGA GGG AG, | |
| 82, | HIL4R77, | NM_00418, | TAT AGC CCT GAG CCC AGG CC, | |
| 83, | HIL4R78, | NM_00418, | CAG GAT GAC AAT GCA GGA AA, | |
| 84, | HIL4R79, | NM_00418, | TAG CAC AAC AGG CAG ACG GC, | |
| 85, | HIL4R80, | NM_00418, | TAA TCT TGG TGA TGC TGA AC, | |
| 86, | HIL4R81, | NM_00418, | CTG ATC CCA CGT AAG AAA GA, | |
| 87, | HIL4R82, | NM_00418, | TTA TTA TAG CCA CGA GGC GG, | |
| 88, | HIL4R83, | NM_00418, | ACA ATT CTT CCA GTG TGG GC, | |
| 89, | HIL4R84, | NM_00418, | CGG GAC CGC TTC TCC CAC TG, | |
| 90, | HIL4R85, | NM_00418, | ACT TGG CTG GTT CCT GGC CT, | |
| 91, | HIL4R86, | NM_00418, | TCA TCC CTT TTC ATG TTG TG, | |
| 92, | HIL4R87, | NM_00418, | GCC CTG GAA AGGCAT CTC TT, | |
| 93, | HIL4R88, | NM_00418, | GCT CTC TGG CCA GAG GAC TG, | |
| 94, | HIL4R89, | NM_00418, | ACA CAT CGC ACC ACG CTG AT, | |
| 95, | HIL4R90, | NM_00418, | CTC CTC CTC CTC ACA CTC CA, | |
| 96, | HIL4R91, | NM_00418, | CAG GCGATG CAC AGA AGC TC, | |
| 97, | HIL4R92, | NM_00418, | ATG CCC TCC CTT CCC TCC TG, | |
| 98, | HIL4R93, | NM_00418, | TCT CTG TTA GCC GGG CCA CA, | |
| 99, | HIL4R94, | NM_00418, | GAG CAG GTC CAG GAA CAG GC, | |
| 100, | HIL4R95, | NM_00418, | AGG TGG AAG AAG GCA TGA CT, | |
| 101, | HIL4R96, | NM_00418, | GGA ACT CAT CCC AGG GCA TG, | |
| 102, | HIL4R97, | NM_00418, | CTC CTT GGG CCC TGC ACT TG, | |
| 103, | HIL4R98, | NM_00418, | GCT CCA GGT GGA GAG GCT GC, | |
| 104, | HIL4R99, | NM_00418, | CGG GCT GGC AGG AGG ACT TG, | |
| 105, | HIL4R100, | NM_00418, | AGG GTT GCC TGC GAT GAC GA, | |
| 106, | HIL4R101, | NM_00418, | ACG GTG ACT GGC TCA GGG AG, | |
| 107, | HIL4R102, | NM_00418, | TGG ACC CAG CTC TCT GGG AC, | |
| 108, | HIL4R103, | NM_00418, | TGT CTG AAG CTG CGG TAA GC, | |
| 109, | HIL4R104, | NM_00418, | ACA GTG GTT GGC TCA GAG AG, | |
| 110, | HIL4R105, | NM_00418, | GGA CAT TTC GGC GGA GGA TC, | |
| 111, | HIL4R106, | NM_00418, | CAA ACT CCT GAT AGC CAC TG, | |
| 112, | HIL4R107, | NM_00418, | ACC GCA CTG GCC TGG GTG CC, | |
| 113, | HIL4R108, | NM_00418, | GGC CTT GTA ACC AGC CTC TC, | |
| 114, | HIL4R109, | NM_00418, | CTG GCA AGC AGG CTT GAG AA, | |
| 115, | HIL4R110, | NM_00418, | GAA TGA GGT CTT GGA AAG GC, | |
| 116, | HIL4R110, | NM_00418, | TGT CCA GTC CAA AGG TGA AC, | |
| 117, | HIL4R111, | NM_00418, | CTT GGG AGA TGT GAG CTC TG, | |
| 118, | HIL4R112, | NM_00418, | GGC TCC AGA CCC AGG TGC TC, | |
| 119, | HIL4R113, | NM_00418, | GGC TTT GGC ATG TCC TCT AC, | |
| 120, | HIL4R114, | NM_00418, | GGG TCT GTG GCC TGC TCC TG, | |
| 121, | HIL4R115, | NM_00418, | TGC CCA GGC TGT CCA CAA GG, | |
| 122, | HIL4R116, | NM_00418, | GGC TGA GTA GAC AAT GCC AC, | |
| 123, | HIL4R117, | NM_00418, | CCG CAC AGG TGG CAG GTA AG, | |
| 124, | HIL4R118, | NM_00418, | TCC TCC TGG CCA TGA CAC TG, | |
| 125, | HIL4R119, | NM_00418, | GCC ACA GCA AGG ACT GGC CA, | |
| 126, | HIL4R120, | NM_00418, | GAC CTG TCT CCA CAG CAG CA, | |
| 127, | HIL4R121, | NM_00418, | CAG ACT GGC CTC CAG TGG AA, | |
| 128, | HIL4R122, | NM_00418, | GGT GCC AGG GAG GCC GGA CA, | |
| 129, | HIL4R123, | NM_00418, | GGG CAG GAT GGA AGG ATG AT, | |
| 130, | HIL4R124, | NM_00418, | GTC CCA CGG AGA CAA AGT TC, | |
| 131, | HIL4R125, | NM_00418, | AGA GAC CCT CAT GTA TGT GG, | |
| 132, | HIL4R126, | NM_00418, | CAG GCA TGG ATA AGC CCT AG, | |
| 133, | HIL4R127, | NM_00418, | TTC CAG GAG GTG GCA TTT CC, | |
| 134, | HIL4R128, | NM_00418, | GCC AAT CAC CTT CAT ACC AT, | |
| 135, | HIL4R129, | NM_00418, | TCC AGT CTC TGC AGC CCA GT, | |
| 136, | HIL4R130, | NM_00418, | GTG GCG AGC CCA GCC CAA TG, | |
| 137, | HIL4R131, | NM_00418, | GCC CTC TAC TCT CAT GGG AT, | |
| 138, | HIL4R132, | NM_00418, | GAG GTG CCC AAG GGC CTC AG, | |
| 139, | HIL4R133, | NM_00418, | CAA CAA CTC GTT CAC AAG TC, | |
| 140, | HIL4R134, | NM_00418, | GAA GCT GTG GAG GGA GCA GC, | |
| 141, | HIL4R135, | NM_00418, | AAC AGG GAC AGT CTG CTG CA, | |
| 142, | HIL4R136, | NM_00418, | AAC ATG CCT TGG GCA GTT AC, | |
| 143, | HIL4R137, | NM_00418, | GGC CAT GAT CTG GTG GGC AA, | |
| 144, | HIL4R138, | NM_00418, | GGC AGG TGG GCC TCC ACG TG, | |
| 145, | HIL4R139, | NM_00418, | CCT GAA GTT CAA TTT CTC AT, | |
| 146, | HIL4R140, | NM_00418, | TCT AGG CAA TGA CCA CCC TC, | |
| 147, | HIL4R141, | NM_00418, | CGA TTT CCC AAG GCC GCC CA, | |
| 148, | HIL4R142, | NM_00418, | ACA GCC GGG ATT CTG CCT CC, | |
| 149, | HIL4R142, | NM_00418, | TGC TTT AGT CAT AGC AAT TT, | |
| 150, | HIL4R143, | NM_00418, | GTT TGG CAG CAA ATT GTC CC, | |
| 151, | HIL4R144, | NM_00418, | TGG CTG AGC ATA TTT ATT CT, | |
| 152, | HIL4R145, | NM_00418, | GCC CAC AGG GTG GCT GAG CA, | |
| 153, | HIL4R146, | NM_00418, | CCT GCT GTC TGG ATT GCC CG, | |
| 154, | HIL4R147, | NM_00418, | GCC AAC ATG CAG GGT AAC TG, | |
| 155, | HIL4R148, | NM_00418, | CCC TAG CAC CTG AGG TCT GG, | |
| 156, | HIL4R149, | NM_00418, | CAA CCC AAG GTT CCC GCC TT, | |
| 157, | HIL4R150, | NM_00418, | ACA CAC AGA CGA GCA TTA CT, | |
| Concatemer Nucleic Acid Sequences of IL4R gene oligo sequences | | | | |
| (SEQ ID NO: 158) | | | | |
| CTCCACTCACTCCAGGTGCTCCACTCACTCCAGGCAGCTGCCCCATGCTGGAGAAGGCCTTGTAACCGCGCCCCTGCTCCATTCGCCTTTCTTCCAGCTGTGTGTCACCACGCCCGGCTTCTCTTCTGCCCGCC | | | | |
| TCAGCCTCCGGCACCAGGCTGGTCTCGTGGGAGATGCCAAGGCACGCCACCCCATTGGGAGATGCAAAGCCACCCCATTGGGTTCCCAGAGCTTGCCACCTGGAGCTCGGTGCTGCAATTGGATACACGTGTGG | | | | |
| GCTTCGGGCGCCTCCGTTGGTTCTCAGGGCAGCTGCTGCCCAGCCCGGTTTCCTGGGGCCCTGGGTGGGATACGGGTTGCTCCAGTCTGCCGGGTCGTTTCACTTTCAGGGTGCTGGCTGCGGGCCCTCACCCG | | | | |
| TGCCCTGTCCACTCACTCCAGGTGGTGTTGTGCCACTTGGTGCTGGTGCTGCTCGAAGGGCTCCCTGGCGGCTGCGGGCTGGGTCGGGACCGCTTCTCCCACTGCTTGGCTGGTTCCTGGCCTTGGCAGCCTTG | | | | |
| TGAGGATCTTCTTGCTGATCTCCACTGGGGTCATCCCTGCTGCTCTCTCGTACTTCCCGAAGGTGGGGTTGTCTGGACTCTGGGTGTTGCTGAAGCTGCGGTCGGGTTCTACTTCCTCCAGGTGCTCCCAGGTT | | | | |
| TCTGGCTCCCCTGCTCCACCGCATGTGGCTTATACCCCTCTTCCCTGAGCTCTGCGGACTGCGCTGTTTCAGGTGGCCGCGGATTTACTCTTCTCTGGGGTCTGGCTTGAGCTCTGGCCCTAGTCCTCATCTGC | | | | |
| TGCCAGCCTGGCTGCCTTCCGCGACCCAGTGCCCTCTACTGTCTGCTGCAGAAGCTGTGGGGCTCGGCTTCTAGTTCAGGTCATTCCCTTGATGGCTGTCGATTTCCCAAGGCCGCCCTTCATGTCCTCTGTTG | | | | |
| CTCCGGCATGGGTGTTTGGCAGCGTGCCTTATGCCTGCTGTCTTTCTGTTCCACTCTCCTCAGAAACTTTATTTATACAAGGCTCCACTCACTCCAGGGCTCCACTCACTCCAGCTGGGATTATAGGCATGAGC | | | | |
| CTGATCTCAAGTGATCTGCACAGGGAACAGGAGCCCAGAGCAGCAGGACCAGGCAGCTCGGCTCCTGCAAGACCTTCATTGTAGTCGGAGACGCAGGTGCTCGCAAGTAGAGATGCTCAGTGGGACCATTCATC | | | | |
| TTCCAAACCAGCGTGTGGGCTTCGGAGAGCAGAAAAACCAGCTGGGATACACGTGTGGGCTTCGGACTGACCACGTCATCCATGATCCAGTGTATAGTTATCCGCGAAGGAGCCCTTCCACAGCATTCACATGC | | | | |
| TCGCTGGGCTTGACATTGGTGTGAACTGTCAGTCAGCAGCAGAGTGTCGGAATTATACAGGTAATTGTCAGTTGACTGCATAGGTGAGATGATAGATTCTGAAATCTGCCGGGTTCTAGGTAGGTCACGTTTGG | | | | |
| CTGCGATGCGGAGGGAGCCCTGTAGGAAATCCCAGACTGGCTGCGATGCGGAGGGAGTATAGCCCTGAGCCCAGGCCCAGGATGACAATGCAGGAAATAGCACAACAGGCAGACGGCTAATCTTGGTGATGCTG | | | | |
| AACCTGATCCCACGTAAGAAAGATTATTATAGCCACGAGGCGGACAATTCTTCCAGTGTGGGCCGGGACCGCTTCTCCCACTGACTTGGCTGGTTCCTGGCCTTCATCCCTTTTCATGTTGTGGCCCTGGAAAG | | | | |
| GCATCTCTTGCTCTCTGGCCAGAGGACTGACACATCGCACCACGCTGATCTCCTCCTCCTCACACTCCACAGGCGATGCACAGAAGCTCATGCCCTCCCTTCCCTCCTGTCTCTGTTAGCCGGGCCACAGAGCA | | | | |
| GGTCCAGGAACAGGCAGGTGGAAGAAGGCATGACTGGAACTCATCCCAGGGCATGCTCCTTGGGCCCTGCACTTGGCTCCAGGTGGAGAGGCTGCCGGGCTGGCAGGAGGACTTGAGGGTTGCCTGCGATGACG | | | | |
| AACGGTGACTGGCTCAGGGAGTGGACCCAGCTCTCTGGGACTGTCTGAAGCTGCGGTAAGCACAGTGGTTGGCTCAGAGAGGGACATTTCGGCGGAGGATCCAAACTCCTGATAGCCACTGACCGCACTGGCCT | | | | |
| GGGTGCCGGCCTTGTAACCAGCCTCTCCTGGCAAGCAGGCTTGAGAAGAATGAGGTCTTGGAAAGGCTGTCCAGTCCAAAGGTGAACCTTGGGAGATGTGAGCTCTGGGCTCCAGACCCAGGTGCTCGGCTTTG | | | | |
| GCATGTCCTCTACGGGTCTGTGGCCTGCTCCTGTGCCCAGGCTGTCCACAAGGGGCTGAGTAGACAATGCCACCCGCACAGGTGGCAGGTAAGTCCTCCTGGCCATGACACTGGCCACAGCAAGGACTGGCCAG | | | | |
| ACCTGTCTCCACAGCAGCACAGACTGGCCTCCAGTGGAAGGTGCCAGGGAGGCCGGACAGGGCAGGATGGAAGGATGATGTCCCACGGAGACAAAGTTCAGAGACCCTCATGTATGTGGCAGGCATGGATAAGC | | | | |
| CCTAGTTCCAGGAGGTGGCATTTCCGCCAATCACCTTCATACCATTCCAGTCTCTGCAGCCCAGTGTGGCGAGCCCAGCCCAATGGCCCTCTACTCTCATGGGATGAGGTGCCCAAGGGCCTCAGCAACAACTC | | | | |
| GTTCACAAGTCGAAGCTGTGGAGGGAGCAGCAACAGGGACAGTCTGCTGCAAACATGCCTTGGGCAGTTACGGCCATGATCTGGTGGGCAAGGCAGGTGGGCCTCCACGTGCCTGAAGTTCAATTTCTCATTCT | | | | |
| AGGCAATGACCACCCTCCGATTTCCCAAGGCCGCCCAACAGCCGGGATTCTGCCTCCTGCTTTAGTCATAGCAATTTGTTTGGCAGCAAATTGTCCCTGGCTGAGCATATTTATTCTGCCCACAGGGTGGCTGA | | | | |
| GCACCTGCTGTCTGGATTGCCCGGCCAACATGCAGGGTAACTGCCCTAGCACCTGAGGTCTGGCAACCCAAGGTTCCCGCCTTACACACAGACGAGCATTACT | | | | |
| IL5R Nucleic Acid Sequences (GENBANK ACCESSION NO.X61177) | | | | |
| (SEQ ID NO: 159) | | | | |
| TAGATGCTGGGGTTGCAGCCACGAGCATAGACACGACAGACACGGTCCTCGCCATCTTCTGTTGAGTACTGGTCGGAACAAGAGGATCGTCTGTAGACAGGATATGATCATCGTGGCGCATGTATTACTCATCC | | | | |
| TTTTGGGGGCCACTGAGATACTGCAAGCTGACTTACTTCCTGATGAAAAGATTTCACTTCTCCCACCTGTCAATTTCACCATTAAAGTTACTGGTTTGGCTCAAGTTCTTTTACAATGGAAACCAAATCCTGAT | | | | |
| CAAGAGCAAAGGAATGTTAATCTAGAATATCAAGTGAAAATAAACGCTCCAAAAGAAGATGACTATGAAACCAGAATCACTGAAAGCAAATGTGTAACCATCCTCCACAAAGGCTTTTCAGCAAGTGTGCGGAC | | | | |
| CATCCTGCAGAACGACCACTCACTACTGGCCAGCAGCTGGGCTTCTGCTGAACTTCATGCCCCACCAGGGTCTCCTGGAACCTCAATTGTGAATTTAACTTGCACCACAAACACTACAGAAGACAATTATTCAC | | | | |
| GTTTAAGGTCATACCAAGTTTCCCTTCACTGCACCTGGCTTGTTGGCACAGATGCCCCTGAGGACACGCAGTATTTTCTCTACTATAGGTATGGCTCTTGGACTGAAGAATGCCAAGAATACAGCAAAGACACA | | | | |
| CTGGGGAGAAATATCGCATGCTGGTTTCCCAGGACTTTTATCCTCAGCAAAGGGCGTGACTGGCTTGCGGTGCTTGTTAACGGCTCCAGCAAGCACTCTGCTATCAGGCCCTTTGATCAGCTGTTTGCCCTTCA | | | | |
| CGCCATTGATCAAATAAATCCTCCACTGAATGTCACAGCAGAGATTGAAGGAACTCGTCTCTCTATCCAATGGGAGAAACCAGTGTCTGCTTTTCCAATCCATTGCTTTGATTATGAAGTAAAAATACACAATA | | | | |
| CAAGGAATGGATATTTGCAGATAGAAAAATTGATGACCAATGCATTCATCTCAATAATTGATGATCTTTCTAAGTACGATGTTCAAGTGAGAGCAGCAGTGAGCTCCATGTGCAGAGAGGCAGGGCTCTGGAGT | | | | |
| GAGTGGAGCCAACCTATTTATGTGGGAAATGATGAACACAAGCCCTTGAGAGAGTGGTTTGTCATTGTGATTATGGCAACCATCTGCTTCATCTTGTTAATTCTCTCGCTTATCTGTAAAATATGTCATTTATG | | | | |
| GATCAAGTTGTTTCCACCAATTCCAGCACCAAAAAGTAATATCAAAGATCTCTTTGTAACCACTAACTATGAGAAAGCTGGAATTTAAATTCAAGCATGTTTTAACTTTTGGTTTAAGGTACTTGGGTGTACCT | | | | |
| GGCAGTGTTGTAAGCTCTTTACATTAATTAATTAACTCTCTAGGTACTGTTATCTTCATTTTATAAACAAGGCAGCTGAAGTTGAGAGAAATAAGTAACCTGTCCTAGGTCACACAATTAGGAAATGACAGATC | | | | |
| TGGCAGTCTATTTCCAGGCAGTCTATTTCCACGAGGTCATGAGTGCGAAAGAGGGACTAGGGGAAGAATGATTAACTCCAGGGAGCTGACTTTTCTAGTGTGCTTACCTGTTTTGCATCTCTCAAGGATGTGCC | | | | |
| ATGAAGCTGTAGCCAGGTGGAATTGTACCACAGCCCTGACATGAACACCTGATGGCAGCTGCTGGGTTGGAGCCTAGACAAAAACATGAAGAACCATGGCTGCTGCCTGAGCCCATCGTGCTGTAATTATAGAA | | | | |
| AACCTTCTAAGGGAAGAATATGCTGATATTTTTCAGATAAGTACCCCTTTTATAAAAATCCTCCAAGTTAGCCCTCGATTTTCCATGTAAGGAAACAGAGGCTTTGAGATAATGTCTGTCTCCTAAGGGACAAA | | | | |
| GCCAGGACTTGATCCTGTCTTAAAAATGCAAAATGTAGTACTTCTTCCATCAAAGGTAGACATGCACTAAGGGACAGGTTTTGGCTTGGTATCAGAATACATTTTTAAAAGCTGTGTAAGAATTGAACGGGCTG | | | | |
| TACTAGGGGGTATA | | | | |
| 160, | EPI-06-001, | X61177, | CCATGCGATGAGAAGCAGCGG, | |
| 161, | EPI-06-002, | X61177, | GGTGGCCATGCGATGAGAAGC, | |
| 162, | EPI-06-003, | X61177, | CAATGTGCCTGGCCTGAG, | |
| 163, | EPI-06-004, | X61177, | TCAGGCACAGGACCAATGCT, | |
| 164, | EPI-06-005, | X61177, | ACCAATGCTCAATGTGCC, | |
| 165, | EPI-06-006, | X61177, | TAGCGTCAGGCACAGGACC, | |
| 166, | EPI-06-007, | X61177, | TCTAGCATAGCGTCAGGCAC, | |
| 167, | EPI-06-008, | X61177, | CAGCATCTAGCATAGCGTC, | |
| 168, | EPI-06-009, | X61177, | CTATGCTCGTGGCTGCAAC, | |
| 169, | EPI-06-010, | X61177, | CGTGTCTATGCTCGTGGCT, | |
| 170, | EPI-06-011, | X61177, | CGTGTCTATGCTCGTGGC, | |
| 171, | EPI-06-012, | X61177, | GTGTCTGTCGTGTCTATGCTC, | |
| 172, | EPI-06-013, | X61177, | GGCGAGGACCGTGTCTGTCG, | |
| 173, | EPI-06-014, | X61177, | GGCGAGGACCGTGTCTGT, | |
| 174, | EPI-06-015, | X61177, | CAGAAGATGGCGAGGACCGTG, | |
| 175, | EPI-06-016, | X61177, | CAGAAGATGGCGAGGACCG, | |
| 176, | EPI-06-017, | X61177, | CTCAACAGAAGATGGCGAGG, | |
| 177, | EPI-06-018, | X61177, | CCAGTACTCAACAGAAGATGG, | |
| 178, | EPI-06-019, | X61177, | TTCCGACCAGTACTCAACAG, | |
| 179, | EPI-06-020, | X61177, | CTCTTGTTCCGACCAGTACTC, | |
| 180, | EPI-06-021, | X61177, | TCCTCTTGTTCCGACCAG, | |
| 181, | EPI-06-022, | X61177, | GACGATCCTCTTGTTCCG, | |
| 182, | EPI-06-023, | X61177, | CTACAGACGATCCTCTTG, | |
| 183, | EPI-06-024, | X61177, | GCCTGTCTACAGACGATCC, | |
| 184, | EPI-06-025, | X61177, | CTGTAGCCTGTCTACAGACG, | |
| 185, | EPI-06-026, | X61177, | AATCTGTAGCCTGTCTAC, | |
| 186, | EPI-06-027, | X61177, | GATGAGTGAACATGACAGG, | |
| 187, | EPI-06-028, | X61177, | TTACTATGAGGATTTAA, | |
| 188, | EPI-06-029, | X61177, | GCCACGATGATCATATCCTT, | |
| 189, | EPI-06-030, | X61177, | GCCACGATGATCATATCC, | |
| 190, | EPI-06-031, | X61177, | GCGCCACGATGATCATAT, | |
| 191, | EPI-06-032, | X61177, | ATGCGCCACGATGATCAT, | |
| 192, | EPI-06-033, | X61177, | TGCGCCACGATTGATCATA, | |
| 193, | EPI-06-034, | X61177, | ATACATGCGCCACGATG, | |
| 194, | EPI-06-035, | X61177, | GAGTAATACATGCGCCAC, | |
| 195, | EPI-06-036, | X61177, | GGATGAGTAATACATGCG, | |
| 196, | EPI-06-037, | X61177, | TTGCAGTATCTCAGTGGC, | |
| 197, | EPI-06-038, | X61177, | GCTTGCAGTATCTCAGTG, | |
| 198, | EPI-06-039, | X61177, | GTCAGCTTGCAGTATCTC, | |
| 199, | EPI-06-040, | X61177, | GGAAGTAAGTCAGCTTGCAG, | |
| 200, | EPI-06-041, | X61177, | TTGACAGGTGGGAGAAGTGA, | |
| 201, | EPI-06-042, | X61177, | CCAGTAACTTTAATGGTG, | |
| 202, | EPI-06-043, | X61177, | GATCAGGATTTGGTTTCC, | |
| 203, | EPI-06-044, | X61177, | GCTCTTGATCAGGATTTGG, | |
| 204, | EPI-06-045, | X61177, | CCTTTGCTCTTGATCAGG, | |
| 205, | EPI-06-046, | X61177, | GATTAACATTCCTTTGCTC, | |
| 206, | EPI-06-047, | X61177, | CTGGTTTCATAGTCATCTTC, | |
| 207, | EPI-06-048, | X61177, | GGTTACACATTTGCTTTCAG, | |
| 208, | EPI-06-049, | X61177, | TGTGGAGGATGGTTACAC, | |
| 209, | EPI-06-050, | X61177, | GGTTACACATTTGCTTTCAG, | |
| 210, | EPI-06-051, | X61177, | GTCGTTCTGCAGGATGGTCCG, | |
| 211, | EPI-06-052, | X61177, | GTGGTCGTTCTGCAGGATG, | |
| 212, | EPI-06-053, | X61177, | AGTGAGTGGTCGTTCTGC, | |
| 213, | EPI-06-054, | X61177, | GCCAGTAGTGAGTGGTCGT, | |
| 214, | EPI-06-055, | X61177, | GCTGGCCAGTAGTGAGTG, | |
| 215, | EPI-06-056, | X61177, | GCCCAGCTGCTGGCCAGTAGT, | |
| 216, | EPI-06-057, | X61177, | GAAGCCCAGCTGCTGGCCA, | |
| 217, | EPI-06-058, | X61177, | CAGCAGAAGCCCAGCTGC, | |
| 218, | EPI-06-059, | X61177, | GAAGTTCAGCAGAAGCCCA, | |
| 219, | EPI-06-060, | X61177, | GGCATGAAGTTCAGCAGAAG, | |
| 220, | EPI-06-061, | X61177, | TTCCAGGAGACCCTGGTG, | |
| 221, | EPI-06-062, | X61177, | TGAGGTTCCAGGAGACCC, | |
| 222, | EPI-06-063, | X61177, | CACAATTGAGGTTCCAGG, | |
| 223, | EPI-06-064, | X61177, | GTGTTTGTGGTGCAAGTTA, | |
| 224, | EPI-06-065, | X61177, | TGTCTTCTGTAGTGTTTGTGG, | |
| 225, | EPI-06-066, | X61177, | GCCAGGTGCAGTGAAGGG, | |
| 226, | EPI-06-067, | X61177, | ACAAGCCAGGTGCAGTGA, | |
| 227, | EPI-06-068, | X61177, | TGCCAACAAGCCAGGTGC, | |
| 228, | EPI-06-069, | X61177, | ATCTGTGCCAACAAGCCA, | |
| 229, | EPI-06-070, | X61177, | GGCATCTGTGCCAACAAGCC, | |
| 230, | EPI-06-071, | X61177, | CCAAGAGCCATACCTATAG, | |
| 231, | EPI-06-072, | X61177, | CAGTCCAAGAGCCATACC, | |
| 232, | EPI-06-073, | X61177, | GCATTCTTCAGTCCAAGAGCC, | |
| 233, | EPI-06-074, | X61177, | CTTGGCATTCTTCAGTCC, | |
| 234, | EPI-06-075, | X61177, | TGCTGTATTCTTGGCATTCTT, | |
| 235, | EPI-06-076, | X61177, | CTTTGCTGTATTCTTGGC, | |
| 236, | EPI-06-077, | X61177, | CTCCCCAGTGTGTCTTTGCTG, | |
| 237, | EPI-06-078, | X61177, | TTCTCCCCAGTGTGTCTT, | |
| 238, | EPI-06-079, | X61177, | GATATTTCTCCCCAGTGT, | |
| 239, | EPI-06-080, | X61177, | CATGCGATATTTCTCCCC, | |
| 240, | EPI-06-081, | X61177, | CCAGCATGCGATATTTCT, | |
| 241, | EPI-06-082, | X61177, | GCCAGTCACGCCCTTTGCTG, | |
| 242, | EPI-06-083, | X61177, | GCCAGTCACGCCCTTTGC, | |
| 243, | EPI-06-084, | X61177, | AGCCGTTAACAAGCACCG, | |
| 244, | EPI-06-085, | X61177, | GCTGGAGCCGTTAACAAG, | |
| 245, | EPI-06-086, | X61177, | TGCTTGCTGGAGCCGTTA, | |
| 246, | EPI-06-087, | X61177, | GCAGAGTGCTTGCTGGAGC, | |
| 247, | EPI-06-088, | X61177, | GATAGCAGAGTGCTTGCT, | |
| 248, | EPI-06-089, | X61177, | GGGCCTGATAGCAGAGTGC, | |
| 249, | EPI-06-090, | X61177, | ATCAATGGCGTGAAGGGC, | |
| 250, | EPI-06-091, | X61177, | ATTTGATCAATGGCGTGA, | |
| 251, | EPI-06-092, | X61177, | TGTGACATTCAGTGGAGG, | |
| 252, | EPI-06-093, | X61177, | TCTCTGCTGTGACATTCAGT, | |
| 253, | EPI-06-094, | X61177, | TCAATCTCTGCTGTGACA, | |
| 254, | EPI-06-095, | X61177, | GTTCCTTCAATCTCTGCTG, | |
| 255, | EPI-06-096, | X61177, | CGAGTTCCTTCAATCTCTGCTG, | |
| 256, | EPI-06-097, | X61177, | GAGAGACGAGTTCCTTCA, | |
| 257, | EPI-06-098, | X61177, | GGATAGAGAGACGAGTTC, | |
| 258, | EPI-06-099, | X61177, | CTCCCATTGGATAGAGAGACG, | |
| 259, | EPI-06-100, | X61177, | GTTTCTCCCATTGGATAGAG, | |
| 260, | EPI-06-101, | X61177, | CACTGGTTTCTCCCATTGG, | |
| 261 | EPI-06-102, | X61177, | GAATGCATTGGTCATCAA, | |
| 262, | EPI-06-103, | X61177, | GAGATGAATGCATTGGTC, | |
| 263, | EPI-06-104, | X61177, | CTTGAACATCGTACTTAG, | |
| 264, | EPI-06-105, | X61177, | GCTCTCACTTGAACATCGTAC, | |
| 265, | EPI-06-106, | X61177, | CTGCTCTCACTTGAACATCG, | |
| 266, | EPI-06-107, | X61177, | CTGCTGCTCTCACTTGAAC, | |
| 267, | EPI-06-108, | X61177, | TCTCTGCACATGGAGCTC, | |
| 268, | EPI-06-109, | X61177, | CTGCCTCTCTGCACATGG, | |
| 269, | EPI-06-110, | X61177, | GAGCCCTGCCTCTCTGCAC, | |
| 270, | EPI-06-111, | X61177, | CTCCAGAGCCCTGCCTCTCT, | |
| 271, | EPI-06-112, | X61177, | CTCACTCCAGAGCCCTGCC, | |
| 272, | EPI-06-113, | X61177, | CTCCACTCACTCCAGAGCC, | |
| 273, | EPI-06-114, | X61177, | TGGCTCCACTCACTCCAGAG, | |
| 274, | EPI-06-115, | X61177, | GGTTGGCTCCACTCACTCCAG, | |
| 275, | EPI-06-116, | X61177, | TGTTCATCATTTCCCACATA, | |
| 276, | EPI-06-117, | X61177, | AAGGGCTTGTGTTCATCA, | |
| 277, | EPI-06-118, | X61177, | AACCACTCTCTCAAGGGCT, | |
| 278, | EPI-06-119, | X61177, | CAGTAACACTAATACCGT, | |
| 279, | EPI-06-120, | X61177, | CAGATGGTTGCCATAATCAC, | |
| 280, | EPI-06-121, | X61177, | TGAAGCAGATGGTTGCCAT, | |
| 281, | EPI-06-122, | X61177, | CTCATAGTTAGTGGTTAC, | |
| 282, | EPI-06-123, | X61177, | GCTTTCTCATAGTTAGTG, | |
| 283, | EPI-06-124, | X61177, | GGACCCAGCTTTCTCATAG, | |
| 284, | EPI-06-125, | X61177, | GACTTCAATTTCCGTCTC, | |
| 285, | EPI-06-126, | X61177, | CAGATGACTTCAATTTCCG, | |
| 286, | EPI-06-127, | X61177, | CAACTCCAGGCTTCTCTATA, | |
| 287, | EPI-06-128, | X61177, | CAACTCCAGGCTTCTCTAT, | |
| 288, | EPI-06-129, | X61177, | GGGTCTCAACTCCAGGCTTC, | |
| 289, | EPI-06-130, | X61177, | CCAGGGTCTCAACTCCAGGC, | |
| 290, | EPI-06-131, | X61177, | ATCCTCCAGGGTCTCAAC, | |
| 291, | EPI-06-132, | X61177, | CACAGAATCCTCCAGGGT, | |
| 292, | EPI-06-133, | X61177, | GGATGCCAAAGTGACAGTCA, | |
| 293, | EPI-06-134, | X61177, | GGATGCCAAAGTGACAGTC, | |
| 294, | EPI-06-135, | X61177, | TCATCAGAGGATGCCAAAGT, | |
| 295, | EPI-06-136, | X61177, | GTGTGAGTTCATCAGAGGAT, | |
| 296, | EPI-06-137, | X61177, | GGCATGTGTGAGTTCATCAG, | |
| 297, | EPI-06-138, | X61177, | CTGAGGCATGTGTGAGTTC, | |
| 298, | EPI-06-139, | X61177, | GAGCCAGCATCCCTGTTCTT, | |
| 299, | EPI-06-140, | X61177, | GAGCCAGCATCCCCTGTTC, | |
| 300, | EPI-06-141, | X61177, | AGCCAAGAGCCAGCATCCCTGT, | |
| 301, | EPI-06-142, | X61177, | GCCAAGAGCCAGCATCCCTGT, | |
| 302, | EPI-06-143, | X61177, | TAGCCAAGAGCCAGCATCCC, | |
| 303, | EPI-06,144, | X61177, | CCTCTTAGCCAAGAGCCAGC, | |
| 304, | EPI-06-145, | X61177, | ACACCTCTTAGCCAAGAGCC, | |
| 305, | EPI-06-146, | X61177, | TCTGAACACCTCTTAGCCAAG, | |
| 306, | EPI-06-147, | X61177, | TTCTGAACACCTCTTAGC, | |
| 307, | EPI-06-148, | X61177, | CTGGGTGTATTGCTTCGCAG, | |
| 308, | EPI-06-149, | X61177, | GGATGAAGCATCCATACTTT, | |
| 309, | EPI-06-150, | X61177, | TGAGGCGATTTGGATGAAGC, | |
| 310, | EPI-06-151, | X61177, | GTCAACTTCCCTGCTGTAGG, | |
| 311, | EPI-06-152, | X61177, | TGCTTGGATGAGTCAACTTC, | |
| 312, | EPI-06-153, | X61177, | GTGCTACCCTGTACGGCATG, | |
| 313, | EPI-06-154, | X61177, | TTGGCAGGTGAGGAGGTGCT, | |
| 314, | EPI-06-155, | X61177, | GTCTGAGGTGAGTCAAGC, | |
| 315, | EPI-06-156, | X61177, | ACGGCACAGCCAGAAGTA, | |
| 316, | EPI-06-157, | X61177, | ACAGCCAAACGGCACAGCCAG, | |
| 317, | EPI-06-158, | X61177, | GTGCTACAATTGGCAGCTT, | |
| 318, | EPI-06-159, | X61177, | TGGTTCACTCCAGGCTGATG, | |
| 319, | EPI-06-160, | X61177, | CAGTCTTGAATCCAAGTTC, | |
| Concatemer Nucleic Acid Sequences of IL5R gene oligo sequences | | | | |
| (SEQ ID NO: 320) | | | | |
| CCATGCGATGAGAAGCAGCGGGGTGGCCATGCGATGAGAAGCCAATGTGCCTGGCCTGAGTCAGGCACAGGACCAATGCTACCAATGCTCAATGTGCCTAGCGTCAGGCACAGGACCTCTAGCATAGCGTCAGG | | | | |
| CACCAGCATCTAGCATAGCGTCCTATGCTCGTGGCTGCAACCGTGTCTATGCTCGTGGCTCGTGTCTATGCTCGTGGCGTGTCTGTCGTGTCTATGCTCGGCGAGGACCGTGTCTGTCGGGCGAGGACCGTGTC | | | | |
| TGTCAGAAGATGGCGAGGACCGTGCAGAAGATGGCGAGGACCGCTCAACAGAAGATGGCGAGGCCAGTACTCAACAGAAGATGGTTCCGACCAGTACTCAACAGCTCTTGTTCCGACCAGTACTCTCCTCTTGT | | | | |
| TCCGACCAGGACGATCCTCTTGTTCCGCTACAGACGATCCTCTTGGCCTGTCTACAGACGATCCCTGTAGCCTGTCTACAGACGAATCTGTAGCCTGTCTACGATGAGTGAACATGACAGGTTACTATGAGGAT | | | | |
| TTAAGCCACGATGATCATATCCTTGCCACGATGATCATATCCGCGCCACGATGATCATATATGCGCCACGATGATCATTGCGCCACGATGATCATAATACATGCGCCACGATGGAGTAATACATGCGCCACGGA | | | | |
| TGAGTAATACATGCGTTGCAGTATCTCAGTGGCGCTTGCAGTATCTCAGTGGTCAGCTTGCAGTATCTCGGAAGTAAGTCAGCTTGCAGTTGACAGGTGGGAGAAGTGACCAGTAACTTTAATGGTGGATCAGG | | | | |
| ATTTGGTTTCCGCTCTTGATCAGGATTTGGCCTTTGCTCTTGATCAGGGATTAACATTCCTTTGCTCCTGGTTTCATAGTCATCTTCGGTTACACATTTGCTTTCAGTGTGGAGGATGGTTACACGGTTACACA | | | | |
| TTTGCTTTCAGGTCGTTCTGCAGGATGGTCCGGTGGTCGTTCTGCAGGATGAGTGAGTGGTCGTTCTGCGCCAGTAGTGAGTGGTCGTGCTGGCCAGTAGTGAGTGGCCCAGCTGCTGGCCAGTAGTGAAGCCC | | | | |
| AGCTGCTGGCCACAGCAGAAGCCCAGCTGCGAAGTTCAGCAGAAGCCCAGGCATGAAGTTCAGCAGAAGTTCCAGGAGACCCTGGTGTGAGGTTCCAGGAGACCCCACAATTGAGGTTCCAGGGTGTTTGTGGT | | | | |
| GCAAGTTATGTCTTCTGTAGTGTTTGTGGGCCAGGTGCAGTGAAGGGACAAGCCAGGTGCAGTGATGCCAACAAGCCAGGTGCATCTGTGCCAACAAGCCAGGCATCTGTGCCAACAAGCCCCAAGAGCCATAC | | | | |
| CTATAGCAGTCCAAGAGCCATACCGCATTCTTCAGTCCAAGAGCCCTTGGCATTCTTCAGTCCTGCTGTATTCTTGGCATTCTTCTTTGCTGTATTCTTGGCCTCCCCAGTGTGTCTTTGCTGTTCTCCCCAGT | | | | |
| GTGTCTTGATATTTCTCCCCAGTGTCATGCGATATTTCTCCCCCCAGCATGCGATATTTCTGCCAGTCACGCCCTTTGCTGGCCAGTCACGCCCTTTGCAGCCGTTAACAAGCACCGGCTGGAGCCGTTAACAA | | | | |
| GTGCTTGCTGGAGCCGTTAGCAGAGTGCTTGCTGGAGCGATAGCAGAGTGCTTGCTGGGCCTGATAGCAGAGTGCATCAATGGCGTGAAGGGCATTTGATCAATGGCGTGATGTGACATTCAGTGGAGGTCTCT | | | | |
| GCTGTGACATTCAGTTCAATCTCTGCTGTGACAGTTCCTTCAATCTCTGCTGCGAGTTCCTTCAATCTCTGCTGGAGAGACGAGTTCCTTCAGGATAGAGAGACGAGTTCCTCCCATTGGATAGAGAGACGGTT | | | | |
| CCACTCACTCCAGAGGGTTGGCTCCACTCACTCCAGTGTTCATCATTTCCCACATAAAGGGCTTGTGTTCATCAAACCACTCTCTCAAGGGCTCAGTAACACTAATACCGTCAGATGGTTGCCATAATCACTGA | | | | |
| AGCAGATGGTTGCCATCTCATAGTTAGTGGTTACGCTTTCTCATAGTTAGTGGGACCCAGCTTTCTCATAGGACTTCAATTTCCGTCTCCAGATGACTTCAATTTCCGCAACTCCAGGCTTCTCTATACAACTC | | | | |
| CAGGCTTCTCTATGGGTCTCAACTCCAGGCTTCCCAGGGTCTCAACTCCAGGCATCCTCCAGGGTCTCAACCACAGAATCCTCCAGGGTGGATGCCAAAGTGACAGTCAGGATGCCAAAGTGACAGTCTCATCA | | | | |
| GAGGATGCCAAAGTGTGTGAGTTCATCAGAGGATGGCATGTGTGAGTTCATCAGCTGAGGCATGTGTGAGTTCGAGCCAGCATCCCTGTTCTTGAGCCAGCATCCCCTGTTCAGCCAAGAGCCAGCATCCCTGT | | | | |
| GCCAAGAGCCAGCATCCCTGTTAGCCAAGAGCCAGCATCCCCCTCTTAGCCAAGAGCCAGCACACCTCTTAGCCAAGAGCCTCTGAACACCTCTTAGCCAAGTTCTGAACACCTCTTAGCCTGGGTGTATTGCT | | | | |
| TCGCAGGGATGAAGCATCCATACTTTTGAGGCGATTTGGATGAAGCGTCAACTTCCCTGCTGTAGGTGCTTGGATGAGTCAACTTCGTGCTACCCTGTACGGCATGTTGGCAGGTGAGGAGGTGCTGTCTGAGG | | | | |
| TGAGTCAAGCACGGCACAGCCAGAAGTAACAGCCAAACGGCACAGCCAGGTGCTACAATTGGCAGCTTTGGTTCACTCCAGGCTGATGCAGTCTTGAATCCAAGTTC | | | | |
| CCR1 Nucleic Acid Sequences (GENBANK ACCESSION NO, X61177) | | | | |
| (SEQ ID NO: 321) | | | | |
| GAGAAGCCGGGATGGAAACTCCAAACACCACAGAGGACTATGACACGACCACAGAGTTTGACTATGGGGATGCAACTCCGTGCCAGAAGGTGAACGAGAGGGCCTTTGGGGCCCAACTGCTGCCCCCTCTGTAC | | | | |
| TCCTTGGTATTTGTCATTGGCCTGGTTGGAAACATCCTGGTGGTCCTGGTCCTTGTGCAATACAAGAGGCTAAAAAACATGACCAGCATCTACCTCCTGAACCTGGCCATTTCTGACCTGCTCTTCCTGTTCAC | | | | |
| GCTTCCCTTCTGGATCGACTACAAGTTGAAGGATGACTGGGTTTTTGGTGATGCCATGTGTAAGATCCTCTCTGGGTTTTATTACACAGGCTTGTACAGCGAGATCTTTTTCATCATCCTGCTGACGATTGACA | | | | |
| GGTACCTGGCCATCGTCCACGCCGTGTTTGCCTTGCGGGCACGGACCGTCACTTTTGGTGTCATCACCAGCATCATCATTTGGGCCCTGGCCATCTTGGCTTCCATGCCAGGCTTATACTTTTCCAAGACCCAA | | | | |
| TGGGAATTCACTCACCACACCTGCAGCCTTCACTTTCCTCACGAAAGCCTACGAGAGTGGAAGCTGTTTCAGGCTCTGAAACTGAACCTCTTTGGGCTGGTATTGCCTTTGTTGGTCATGATCATCTGCTACAC | | | | |
| AGGGATTATAAAGATTCTGCTAAGACGACCAAATGAGAAGAAATCCAAAGCTGTCCGTTTGATTTTTGTCATCATGATCATCTTTTTTCTCTTTTGGACCCCCTACAATTTGACTATACTTATTTCTGTTTTCC | | | | |
| AAGACTTCCTGTTCACCCATGAGTGTGAGCAGAGCAGACATTTGGACCTGGCTGTGCAAGTGACGGAGGTGATCGCCTACACGCACTGCTGTGTCAACCCAGTGATCTACGCCTTCGTTGGTGAGAGGTTCCGG | | | | |
| AAGTACCTGCGGCAGTTGTTCCACAGGCGTGTGGCTGTGCACCTGGTTAAATGGCTCCCCTTCCTCTCCGTGGACAGGCTGGAGAGGGTCAGCTCCACATCTCCCTCCACAGGGGAGCATGAACTCTCTGCTGG | | | | |
| GTTCTGACTCAGACCATAGGAGGCCAACCCAAAATAAGCAGGCGTGACCTGCCAGGCACACTGAGCCAGCAGCCTGGCTCTCCCAGCCAGGTTCTGACTCTTGGCACAGCATGGAGTCACAGCCACTTGGGATA | | | | |
| GAGAGGGAATGTAATGGTGGCCTGGGGCTTCTGAGGCTTCTGGGGCTTCAGTCTTTTCCATGAACTTCTCCCCTGGTAGAAAGAAGATGAATGAGCAAAACCAAATATTCCAGAGACTGGGACTAAGTGTACCA | | | | |
| GAGAAGGGCTTGGACTCAAGCAAGATTTCAGATTTGTGACCATTAGCATTTGTCAACAAAGTCACCCACTTCCCACTATTGCTTGCACAAACCAATTAAACCCAGTAGTGGTGACTGTGGGCTCCATTCAAAGT | | | | |
| GAGCTCCTAAGCCATGGGAGACACTGATGTATGAGGAATTTCTGTTCTTCCATCACCTCCCCCCCCCCGCCACCCTCCCACTGCCAAAGAACTTGGAAATAGTGATTTCCACAGTGACTCCACTCTGAGTCCCA | | | | |
| GAGCCAATCAGTAGCCAGCATCTGCCTCCCCTTCACTCCCACCGCAGGATTTGGGCTCTTGGAATCCTGGGGAACATAGAACTCATGACGGAAGAGTTGAGACCTAACGAGAAATAGAAATGGGGAACTACTGC | | | | |
| TGGCAGTGGAACTAAGAAAGCCCTTAGGAAGAATTTTTATATCCACTAAAATCAAACAATTCAGGGAGTGGGCTAAGCACGGGCCATATGAATAACATGGTGTGCTTCTTAAAATAGCCATAAAGGGGAGGGAC | | | | |
| TCATCATTTCCATTTACCCTTCTTTTCTGACTATTTTTCAGAATCTCTCTTCTTTTCAAGTTGGGTGATATGTTGGTAGATTCTAATGGCTTTATTGCAGCGATTAATAACAGGCAAAAGGAAGCAGGGTTGGT | | | | |
| TTCCCTTCTTTTTGTTCTTCATCTAAGCCTTCTGGTTTTATGGGTCAGAGTTCCGACTGCCATCTTGGACTTGTCAGCAAAAAAAAAAAATAATAATAATAATAAGGCCTGCTGTGTAAGCTGACAGTATTTGT | | | | |
| AGCTGATAGGGGGTTGGGAGGAAAGTGTCTACTAGGAGGGTGGGGTGAGATTCTGTGTTGATGT | | | | |
| 322, | EPI-1-71, | XM_003248, | AGGTAGATGNTGGTCAT, | |
| 323, | EPI-1-72, | XM_003248, | GGTCNGAAATGGCCAGGTT, | |
| 324, | EPI-1-73, | XM_003248, | AGGAAGAGCAGGTCNGAAAT, | |
| 325, | EPI-1-74, | XM_003248, | GTNATAAAACCCAGAGAGGA, | |
| 326, | EPI-1-75, | XM_003248, | CCTGTGTNATAAAACCCAGA, | |
| 327, | EPI-1-76, | XM_003248, | ACAAGCCTGTGTNATAAAAC, | |
| 328, | EPI-1-77, | XM_003248, | GCTGTACAAGCCTGTGTNAT, | |
| 329, | EPI-1-78, | XM_003248, | CTCGCTGTACAAGCCTGTGT, | |
| 330, | EPI-1-79, | XM_003248, | AAGATCTCGCTGTACAAGCC, | |
| 331, | EPI-1-80, | XM_003248, | AAAGAGGCTTGTACAGCGAG, | |
| 332, | EPI-1-81, | XM_003248, | ATGAAAAAGATCTCGCTGT, | |
| 333, | EPI-1-82, | XM_003248, | AGGATNATGAAAAAGATCTC, | |
| 334, | EPI-1-83, | XM_003248, | CAGCAGGATNATGAAAAAGC, | |
| 335, | EPI-1-84, | XM_003248, | ATCGTCAGCAGGATNATGAA, | |
| 336, | EPI-1-85, | XM_003248, | GTCAATCGTCAGCAGGATNA, | |
| 337, | EPI-1-86, | XM_003248, | ACCTGTCAATCGTCAGCAGG, | |
| 338, | EPI-1-87, | XM_003248, | AGGTACCTGTCAATCGTCAG, | |
| 339, | EPI-1-88, | XM_003248, | GGCCAGGTACCTGTCAATCG, | |
| 340, | EPI-1-89, | XM_003248, | CNATCGATTGACAGGTACCT, | |
| 341, | EPI-1-90, | XM_003248, | TGGACNATCGATTGACAGGT, | |
| 342, | EPI-1-91, | XM_003248, | GTGATGACACCAAAAGTGAC, | |
| 343, | EPI-1-92, | XM_003248, | GCTGGTGATGACACCAAAAG, | |
| 344, | EPI-1-93, | XM_003248, | GATGCTGGTGATGACACCA, | |
| 345, | EPI-1-94, | XM_003248, | CTGTACAAGCCTGTGNGA, | |
| 346, | EPI-1-95, | XM_003248, | ATNCCTGTGTAGCAGATG, | |
| 347, | EPI-1-96, | XM_003248, | GTAGGCGATCACCTCNGTCAC, | |
| 348, | EPI-1-97, | XM_003248, | AAGGCGTAGATCACNGGGTT, | |
| 349, | EPI-1-98, | XM_003248, | CCAACNAAGGCGTAGATCAC, | |
| 350, | EPI-1-99, | XM_003248, | GTTGGAGAGAGGTTCCGGAA, | |
| 351, | EPI-1-100, | XM_003248, | GAGAGAGGTTCCGGAAGTAC, | |
| 352, | EPI-1-101, | XM_003248, | CGCAGGTACTTCCGGAACCTC, | |
| 353, | EPI-1-102, | XM_003248, | TGNCGCAGGTACTTCCGGAA, | |
| Concatemer Nucleic Acid Sequences of CCR1 gene oligo sequenqes | | | | |
| (SEQ ID NO: 354) | | | | |
| AGGTAGATGNTGGTCATGGTCNGAAATGGCCAGGTTAGGAAGAGCAGGTCNGAAATGTNATAAAACCCAGAGAGGACCTGTGTNATAAAACCCAGAACAAGCCTGTGTNATAAAACGCTGTACAAGCCTGTGTN | | | | |
| ATCTCGCTGTACAAGCCTGTGTAAGATCTCGCTGTACAAGCCAAAGAGGCTTGTACAGCGAGATGAAAAAGATCTCGCTGTAGGATNATGAAAAAGATCTCCAGCAGGATNATGAAAAAGCATCGTCAGCAGGA | | | | |
| TNATGAAGTCAATCGTCAGCAGGATNAACCTGTCAATCGTCAGCAGGAGGTACCTGTCAATCGTCAGGGCCAGGTACCTGTCAATCGCNATCGATTGACAGGTACCTTGGACNATCGATTGACAGGTGTGATGA | | | | |
| CACCAAAAGTGACGCTGGTGATGACACCAAAAGGATGCTGGTGATGACACCACTGTACAAGCCTGTGNGAATNCCTGTGTAGCAGATGGTAGGCGATCACCTCNGTCACAAGGCGTAGATCACNGGGTTCCAAC | | | | |
| NAAGGCGTAGATCACGTTGGAGAGAGGTTCCGGAAGAGAGAGGTTCCGGAAGTACCGCAGGTACTTCCGGAACCTCTGNCGCAGGTACTTCCGGAA | | | | |
| CCR3 Nucleic Acid Sequences (GENBANK ACCESSION NO, X61177) | | | | |
| (SEQ ID NO: 355) | | | | |
| TTTTTCTTCTTCTATCACAGGGAGAAGTGAAATGACAACCTCACTAGATACAGTTGAGACCTTTGGTACCACATCCTACTATGATGACGTGGGCCTGCTCTGTGAAAAAGCTGATACCAGAGCACTGATGGCCC | | | | |
| AGTTTGTGCCCCCGCTGTACTCCCTGGTGTTCACTGTGGGCCTCTTGGGCAATGTGGTGGTGGTGATGATCCTCATAAAATACAGGAGGCTCCGAATTATGACCAACATCTACCTGCTCAACCTGGCCATTTCG | | | | |
| GACCTGCTCTTCCTCGTCACCCTTCCATTCTGGATCCACTATGTCAGGGGGCATAACTGGGTTTTTGGCCATGGCATGTGTAAGCTCCTCTCAGGGTTTTATCACACAGGCTTGTACAGCGAGATCTTTTTCAT | | | | |
| AATCCTGCTGACAATCGACAGGTACCTGGCCATTGTCCATGCTGTGTTTGCCCTTCGAGCCCGGACTGTCACTTTTGGTGTCATCACCAGCATCGTCACCTGGGGCCTGGCAGTGCTAGCAGCTCTTCCTGAAT | | | | |
| TTATCTTCTATGAGACTGAAGAGTTGTTTGAAGAGACTCTTTGCAGTGCTCTTTACCCAGAGGATACAGTATATAGCTGGAGGCATTTCCACACTCTGAGAATGACCATCTTCTGTCTCGTTCTCCCTCTGCTC | | | | |
| GTTATGGCCATCTGCTACACAGGAATCATCAAAACGCTGCTGAGGTGCCCCAGTAAAAAAAAGTACAAGGCCATCCGGCTCATTTTTGTCATCATGGCGGTGTTTTTCATTTTCTGGACACCCTACAATGTGGC | | | | |
| TATCCTTCTCTCTTCCTATCAATCCATCTTATTTGGAAATGACTGTGAGCGGAGCAAGCATCTGGACCTGGTCATGCTGGTGACAGAGGTGATCGCCTACTCCCACTGCTGCATGAACCCGGTGATCTACGCCT | | | | |
| TTGTTGGAGAGAGGTTCCGGAAGTACCTGCGCCACTTCTTCCACAGGCACTTGCTCATGCACCTGGGCAGATACATCCCATTCCTTCCTAGTGAGAAGCTGGAAAGAACCAGCTCTGTCTCTCCATCCACAGCA | | | | |
| GAGCCGGAACTCTCTATTGTGTTTTAGGTCAGATGCAGAAAATTGCCTAAAGAGGAAGGACCAAGGAGATGAAGCAAACACATTAAGCCTTCCACACTCACCTCTAAAACAGTCCTTCAAACTTCCAGT | | | | |
| 356, | EPI-1-1, | NM_001837, | TTTTAGAGGTGAGTGTGGAA, | |
| 357, | EPI-1-2, | NM_001837, | GAGGTGAGTGTGGAAGGCTT, | |
| 358, | EPI-1-3, | NM_001837, | AATGTGTTTGCTTCATCTCC, | |
| 359, | EPI-1-4, | NM_001837, | GTTTGCTTCATCTCCTTGGT, | |
| 360, | EPI-1-5, | NM_001837, | CTTCATCTCCTTGGTCCTTC, | |
| 361, | EPI-1-6, | NM_001837, | TCTCCTTGGTCCTTCCTCTT, | |
| 362, | EPI-1-7, | NM_001837, | TTGGTCCTTCCTCTTTAGGC, | |
| 363, | EPI-1-8, | NM_001837, | CCTTCCTCTTTAGGCAATTT, | |
| 364, | EPI-1-9, | NM_001837, | CTCTTTAGGCAATTTTCTGC, | |
| 365, | EPI-1-10, | NM_001837, | TAGGCAATTTTCTGCATCTG, | |
| 366, | EPI-1-11, | NM_001837, | AATTTTCTGCATCTGACCTA, | |
| 367, | EPI-1-12, | NM_001837, | CAATAGAGAGTTCCGGCTCT, | |
| 368, | EPI-1-13, | NM_001837, | GAGAGTTCCGGCTCTGCTGT, | |
| 369, | EPI-1-14, | NM_001837, | TTCCGGCTCTGCTGTGGATG, | |
| 370, | EPI-1-15, | NM_001837, | GCTCTGCTGTGGATGGAGAG, | |
| 371, | EPI-1-16, | NM_001837, | GCTGTGGATGGAGAGACAGA, | |
| 372, | EPI-1-17, | NM_001837, | GGATGGAGAGACAGAGCTGG, | |
| 373, | EPI-1-18, | NM_001837, | GAGAGACAGAGCTGGTTCTT, | |
| 374, | EPI-1-19, | NM_001837, | ACAGAGCTGGTTCTTTCCAG, | |
| 375, | EPI-1-20, | NM_001837, | GCTGGTTCTTTCCAGCTTCT, | |
| 376, | EPI-1-21, | NM_001837, | TTCTTTCCAGCTTCTCACTA, | |
| 377, | EPI-1-22, | NM_001837, | TCCAGCTTCTCACTAGGAAG, | |
| 378, | EPI-1-23, | NM_001837, | CTTCTCACTAGGAAGGAATG, | |
| 379, | EPI-1-24, | NM_001837, | CACTAGGAAGGAATGGGATG, | |
| 380, | EPI-1-25, | NM_001837, | GGAAGGAATGGGATGTATCT, | |
| 381, | EPI-1-26, | NM_001837, | GAATGGGATGTATCTGCCCA, | |
| 382, | EPI-1-27, | NM_001837, | GGATGTATCTGCCCAGGTGC, | |
| 383, | EPI-1-28, | NM_001837, | TATCTGCCCAGGTGCATGAG, | |
| 384, | EPI-1-29, | NM_001837, | GCCCAGGTGCATGAGCAAGT, | |
| 385, | EPI-1-30, | NM_001837, | GGTGCATGAGCAAGTGCCTG, | |
| 386, | EPI-1-31, | NM_001837, | ATGAGCAAGTGCCTGTGGAA, | |
| 387, | EPI-1-32, | NM_001837, | CAAGTGCCTGTGGAAGAAGT, | |
| 388, | EPI-1-33, | NM_001837, | GCCTGTGGAAGAAGTGGCGC, | |
| 389, | EPI-1-34, | NM_001837, | TGGAAGAAGTGGCGCAGGTA, | |
| 390, | EPI-1-35, | NM_001837, | GAAGTGGCGCAGGTACTTCC, | |
| 391, | EPI-1-36, | NM_001837, | GGCGCAGGTACTTCCGGAAC, | |
| 392, | EPI-1-37, | NM_001837, | AGGTACTTCCGGAACCTCTC, | |
| 393, | EPI-1-38, | NM_001837, | CTTCCGGAACCTCTCTCCAA, | |
| 394, | EPI-1-39, | NM_001837, | GCGTAGATCACCGGGTTCAT, | |
| 395, | EPI-1-40, | NM_001837, | GATCACCGGGTTCATGCAGC, | |
| 396, | EPI-1-41, | NM_001837, | CCGGGTTCATGCAGCAGTGG, | |
| 397, | EPI-1-42, | NM_001837, | TTCATGCAGCAGTGGGAGTA, | |
| 398, | EPI-1-43, | NM_001837, | GCAGCAGTGGGAGTAGGCGA, | |
| 399, | EPI-1-44, | NM_001837, | AGTGGGAGTAGGCGATCACC, | |
| 400, | EPI-1-45, | NM_001837, | GAGTAGGCGATCACCTCTGT, | |
| 401, | EPI-1-46, | NM_001837, | GGCGATCACCTCTGTCACCA, | |
| 402, | EPI-1-47, | NM_001837, | TCACCTCTGTCACCAGCATG, | |
| 403, | EPI-1-48, | NM_001837, | TCTGTCACCAGCATGACCAG, | |
| 404, | EPI-1-49, | NM_001837, | CACCAGCATGACCAGGTCCA, | |
| 405, | EPI-1-50, | NM_001837, | GCATGACCAGGTCCAGATGC, | |
| 406, | EPI-1-51, | NM_001837, | ACCAGGTCCAGATGCTTGCT, | |
| 407, | EPI-1-52, | NM_001837, | GTCCAGATGCTTGCTCCGCT, | |
| 408, | EPI-1-53, | NM_001837, | GATGCTTGCTCCGCTCACAG, | |
| 409, | EPI-1-54, | NM_001837, | TTGCTCCGCTCACAGTCATT, | |
| 410, | EPI-1-55, | NM_001837, | CCGCTCACAGTCATTTCCAA, | |
| 411, | EPI-1-56, | NM_001837, | ATGGATTGATAGGAAGAGAG, | |
| 412, | EPI-1-57, | NM_001837, | TTGATAGGAAGAGAGAAGGA, | |
| 413, | EPI-1-58, | NM_001837, | AGGAAGAGAGAAGGATAGCC, | |
| 414, | EPI-1-59, | NM_001837, | GAGAGAAGGATAGCCACATT, | |
| 415, | EPI-1-60, | NM_001837, | AAGGATAGCCACATTGTAGG, | |
| 416, | EPI-1-61, | NM_001837, | TAGCCACATTGTAGGGTGTC, | |
| 417, | EPI-1-62, | NM_001837, | ACATTGTAGGGTGTCCAGAA, | |
| 418, | EPI-1-63, | NM_001837, | GAGCCGGATGGCCTTGTACT, | |
| 419, | EPI-1-64, | NM_001837, | GGATGGCCTTGTACTTTTTT, | |
| 420, | EPI-1-65, | NM_001837, | GCCTTGTACTTTTTTTTACT, | |
| 421, | EPI-1-65A, | NM_001837, | ACCTCAGCACGTTTTTGATG, | |
| 422, | EPI-1-66, | NM_001837, | AGCAGCGTTTTGATGATTCC, | |
| 423, | EPI-1-67, | NM_001837, | CGTTTTGATGATTCCTGTGT, | |
| 424, | EPI-1-68, | NM_001837, | TGATGATTCCTGTGTAGCAG, | |
| 425, | EPI-1-69, | NM_001837, | ATTCCTGTGTAGCAGATGGC, | |
| 426, | EPI-1-70, | NM_001837, | CGAGCAGAGGGAGAACGAGA, | |
| 427, | EPI-1-71, | NM_001837, | AGGTAGATGNTGGTCAT, | |
| 428, | EPI-1-72, | NM_001837, | GGTCNGAAATGGCCAGGTT, | |
| 429, | EPI-1-73, | NM_001837, | AGGAAGAGCAGGTCNGAAAT, | |
| 430, | EPI-1-74, | NM_001837, | GTNATAAAACCCAGAGAGGA, | |
| 431, | EPI-1-75, | NM_001837, | CCTGTGTNATAAAACCCAGGA, | |
| 432, | EPI-1-76, | NM_001837, | ACAAGCCTGTGTNATAAAAC, | |
| 433, | EPI-1-77, | NM_001837, | GCTGTACAAGCCTGTGTNAT, | |
| 434, | EPI-1-78, | NM_001837, | CTCGCTGTACAAGCCTGTGT, | |
| 435, | EPI-1-78A, | NM_001837, | AAGATCTCGCTGTACAAGCC, | |
| 436, | EPI-1-80, | NM_001837, | AAAGAGGCTTGTACAGCGAG, | |
| 437, | EPI-1-81, | NM_001837, | ATGAAAAAAGATCTCGCTGT, | |
| 438, | EPI-1-82, | NM_001837, | AGGATNATGAAAAAGATCTC, | |
| 439, | EPI-1-83, | NM_001837, | CAGCAGGATNATGAAAAAGC, | |
| 440, | EPI-1-84, | NM_001837, | ATCGTCAGCAGGATNATGAA, | |
| 441, | EPI-1-85, | NM_001837, | GTCAATCGTCAGCAGGATNA, | |
| 442, | EPI-1-86, | NM_001837, | ACCTGTCAATCGTCAGCAGG, | |
| 443, | EPI-1-87, | NM_001837, | AGGTACCTGTCAATCGTCAG, | |
| 444, | EPI-1-88, | NM_001837, | GGCCAGGTACCTGTCAATCG, | |
| 445, | EPI-1-89, | NM_001837, | CNATCGATTGACAGGTACCT, | |
| 446, | EPI-1-90, | NM_001837, | TGGACNATCGATTGACAGGT, | |
| 447, | EPI-1-91, | NM_001837, | GTGATGACACCAAAAGTGAC, | |
| 448, | EPI-1-92, | NM_001837, | GCTGGTGATGACACCAAAAG, | |
| 449, | EPI-1-93, | NM_001837, | GATGCTGGTGATGACACCA, | |
| 450, | EPI-1-94, | NM_001837, | CTGTACAAGCCTGTGNGA, | |
| 451, | EPI-1-95, | NM_001837, | ATNCCTGTGTAGCAGATG, | |
| 452, | EPI-1-96, | NM_001837, | GTAGGCGATCACCTCNGTCAC, | |
| 453, | EPI-1-97, | NM_001837, | AAGGCGTAGATCACNGGGTT, | |
| 454, | EPI-1-98, | NM_001837, | CCAACNAAGGCGTAGATCAC, | |
| 455, | EPI-1-99, | NM_001837, | GTTGGAGAGAGGTTCCGGAA, | |
| 456, | EPI-1-100, | NM_001837, | GAGAGAGGTTCCGGAAGTAC, | |
| 457, | EPI-1-101, | NM_001837, | CGAGACAGAAGATGGTCATT, | |
| 458, | EPI-1-102, | NM_001837, | TGNCGCAGGTACTTCCGGAA, | , |
| 459, | EPI-1-103, | NM_001837, | AGAGGGAGAACGAGACAGAA, | |
| 460, | EPI-1-104, | NM_001837, | GAGAACGAGACAGAAGATGG, | |
| 461, | EPI-1-105, | NM_001837, | CGAGACAGAAGATGGTCATT, | |
| 462, | EPI-1-106, | NM_001837, | CAGAAGATGGTCATTCTCAG, | |
| 463, | EPI-1-107, | NM_001837, | GATGGTCATTCTCAGAGTGT, | |
| 464, | EPI-1-108, | NM_001837, | AACTCTTCAGTCTCATAGAA, | |
| 465, | EPI-1-109, | NM_001837, | ATTCAGGAAGAGCTGCTAGC, | |
| 466, | EPI-1-110, | NM_001837, | CAGGCCCCAGGTGACGATGC, | |
| 467, | EPI-1-111, | NM_001837, | CCCAGGTGACGATGCTGGTG, | |
| 468, | EPI-1-112, | NM_001837, | GTGACGATGCTGGTGATGAC, | |
| 469, | EPI-1-113, | NM_001837, | AAGTGACAGTCCGGGCTCGA, | |
| 470, | EPI-1-114, | NM_001837, | ATTCAGGAAGAGCTGCTAGC, | |
| 471, | EPI-1-115, | NM_001837, | CAGGCCCCAGGTGACGATGC, | |
| 472, | EPI-1-116, | NM_001837, | TGGACAATGGCCAGGTACCT, | |
| 473, | EPI-1-117, | NM_001837, | AATGGCCAGGTACCTGTCGA, | |
| 474, | EPI-1-118, | NM_001837, | CCAGGTACCTGTCGATTGTC, | |
| 475, | EPI-1-119, | NM_001837, | TACCTGTCGATTGTCAGCAG, | |
| 476, | EPI-1-120, | NM_001837, | GTCGATTGTCAGCAGGATTA, | |
| 477, | EPI-1-121, | NM_001837, | AAGATCTCGCTGTACAAGCC, | |
| 478, | EPI-1-122, | NM_001837, | CTCGCTGTACAAGCCTGTGT, | |
| 479, | EPI-1-123, | NM_001837, | AACCCTGAGAGGAGCTTACA, | |
| 480, | EPI-1-124, | NM_001837, | TGAGAGGAGCTTACACATGC, | |
| 481, | EPI-1-125, | NM_001837, | GGAGCTTACACATGCCATGG, | |
| 482, | EPI-1-126, | NM_001837, | AACCCAGTTATGCCCCCTGA, | |
| 483, | EPI-1-127, | NM_001837, | AGTTATGCCCCCTGACATAG, | |
| 484, | EPI-1-128, | NM_001837, | TGCCCCCTGACATAGTGGAT, | |
| 485, | EPI-1-129, | NM_001837, | CCTGACATAGTGGATCCAGA, | |
| 486, | EPI-1-130, | NM_001837, | CATAGTGGATCCAGAATGGA, | |
| 487, | EPI-1-131, | NM_001837, | TGGATCCAGAATGGAAGGGT, | |
| 488, | EPI-1-132, | NM_001837, | CCAGAATGGAAGGGTGACGA, | |
| 489, | EPI-1-133, | NM_001837, | ATGGAAGGGTGACGAGGAAG, | |
| 490, | EPI-1-133A, | NM_001837, | AGGGTGACGAGGAAGAGCAG, | |
| 491, | EPI-1-134, | NM_001837, | GACGAGGAAGAGCAGGTCCG, | |
| 492, | EPI-1-135, | NM_001837, | GCCAGGTTGAGCAGGTAGAT, | |
| 493, | EPI-1-136, | NM_001837, | GTTGAGCAGGTAGATGTTGG, | |
| 494, | EPI-1-137, | NM_001837, | GCAGGTAGATGTTGGTCATA, | |
| 495, | EPI-1-138, | NM_001837, | ATTCGGAGCCTCCTGTATTT, | |
| 496, | EPI-1-139, | NM_001837, | GAGCCTCCTGTATTTTATGA, | |
| 497, | EPI-1-140, | NM_001837, | TCCTGTATTTTATGAGGATC, | |
| 498, | EPI-1-141, | NM_001837, | TATTTTATGAGGATCATCAC, | |
| 499, | EPI-1-142, | NM_001837, | TATGAGGATCATCACCACCA, | |
| 500, | EPI-1-143, | NM_001837, | GGATCATCACCACCACCACA, | |
| 501, | EPI-1-144, | NM_001837, | ATCACCACCACCACATTGCC, | |
| 502, | EPI-1-145, | NM_001837, | CACCACCACATTGCCCAAGA, | |
| 503, | EPI-1-146, | NM_001837, | CCACATTGCCCAAGAGGCCC, | |
| 504, | EPI-1-147, | NM_001837, | TTGCCCAAGAGGCCCACAGT, | |
| 505, | EPI-1-148, | NM_001837, | CAAGAGGCCCACAGTGAACA, | |
| 506, | EPI-1-149, | NM_001837, | GGCCCACAGTGAACACCAGG, | |
| 507, | EPI-1-150, | NM_001837, | ACAGTGAACACCAGGGAGTA, | |
| 508, | EPI-1-150A, | NM_001837, | GAACACCAGGGAGTACAGCG, | |
| 509, | EPI-1-151, | NM_001837, | CTGGGCCATCAGTGCTCTGG, | |
| 510, | EPI-1-152, | NM_001837, | CCATCAGTGCTCTGGTATCA, | |
| 511, | EPI-1-153, | NM_001837, | AGTGCTCTGGTATCAGCTTT, | |
| 512, | EPI-1-154, | NM_001837, | TCTGGTATCAGCTTTTTCAC, | |
| 513, | EPI-1-155, | NM_001837, | TATCAGCTTTTTCACAGAGC, | |
| 514, | EPI-1-156, | NM_001837, | GCTTTTTCACAGAGCAGGCC, | |
| 515, | EPI-1-157, | NM_001837, | TTCACAGAGCAGGCCCACGT, | |
| 516, | EPI-1-158, | NM_001837, | AGAGCAGGCCCACGTCATCA, | |
| 517, | EPI-1-159, | NM_001837, | AGGCCCACGTCATCATAGTA, | |
| 518, | EPI-1-160, | NM_001837, | CACGTCATCATAGTAGGATG, | |
| 519, | EPI-1-161, | NM_001837, | CATCATAGTAGGATGTGGTA, | |
| 520, | EPI-1-162, | NM_001837, | GTATCTAGTGAGGTTGTCAT, | |
| 521, | EPI-1-163, | NM_001837, | TAGTGAGGTTGTCATTTCAC, | |
| 522, | EPI-1-164, | NM_001837, | AGGTTGTCATTTCACTTCTC, | |
| 523, | EPI-1-165, | NM_001837, | GTCATTTCACTTCTCCCTGT, | |
| 524, | EPI-1-166, | NM_001837, | TTCACTTCTCCCTGTGATA, | |
| 525, | EPI-1-167, | NM_001837, | TTCTCCCTGTGATAGAAGAA, | |
| 526, | EPI-1-168, | NM_001837, | TTCACTTCTCCCTGTGATAG, | |
| 527, | EPI-1-169, | NM_001837, | TTCTCCCTGTGATAGAAGAA, | |
| Concatemer Nucleic Acid Sequences of CCR3 gene oligo sequences | | | | |
| (SEQ ID NO: 528) | | | | |
| TTTTAGAGGTGAGTGTGGAAGAGGTGAGTGTGGAAGGCTTAATGTGTTTGCTTCATCTCCGTTTGCTTCATCTCCTTGGTCTTCATCTCCTTGGTCCTTCTCTCCTTGGTCCTTCCTCTTTTGGTCCTTCCTCT | | | | |
| TTAGGCCCTTCCTCTTTAGGCAATTTCTCTTTAGGCAATTTTCTGCTAGGCAATTTTCTGCATCTGAATTTTCTGCATCTGACCTACAATAGAGAGTTCCGGCTCTGAGAGTTCCGGCTCTGCTGTTTCCGGCT | | | | |
| CTGCTGTGGATGGCTCTGCTGTGGATGGAGAGGCTGTGGATGGAGAGACAGAGGATGGAGAGACAGAGCTGGGAGACAGAGACTGGTTCTTACAGAGCTGGTTCTTTCCAGGCGTGGTTCTTTCCAGCTTCTTT | | | | |
| CTTTCCAGCTTCTCACTATCCAGCTTCTCACTAGGAAGCTTCTCACTAGGAAGGAATGCACTAGGAAGGAATGGGATGGGAAGGAATGGGATGTATCTGAATGGGATGTATCTGCCCAGGATGTATCTGCCCAG | | | | |
| GTGCTATCTGCCCAGGTGCATGAGGCCCAGGTGCATGAGCAAGTGGTGCATGAGCAAGTGCCTGATGAGCAAGTGCCTGTGGAACAAGTGCCTGTGGAAGAAGTGCCTGTGGAAGAAGTGGCGCTGGAAGAAGT | | | | |
| GGCGCAGGTAGAAGTGGCGCAGGTACTTCCGGCGCAGGTACTTCCGGAACAGGTACTTCCGGAACCTCTCCTTCCGGAACCTCTCTCCAAGCGTAGATCACCGGGTTCATGATCACCGGGTTCATGCAGCCCGG | | | | |
| GTTCATGCAGCAGTGGTTCATGCAGCAGTGGGAGTAGCAGCAGTGGGAGTAGGCGAAGTGGGAGTAGGCGATCACCGAGTAGGCGATCACCTCTGTGGCGATCACCTCTGTCACCATCACCTCTGTCACCAGCA | | | | |
| TGTCTGTCACCAGCATGACCAGCACCAGCATGACCAGGTCCAGCATGACCAGGTCCAGATGCACCAGGTCCAGATGCTTGCTGTCCAGATGCTTGCTCCGCTGATGCTTGCTCCGCTCACAGTTGCTCCGCTCA | | | | |
| CAGTCATTCCGCTCACAGTCATTTCCAAATGGATTGATAGGAAGAGAGTTGATAGGAAGAGAGAAGGAAGGAAGAGAGAAGGATAGCCGAGAGAAGGATAGCCACATTAAGGATAGCCACATTGTAGGTAGCCA | | | | |
| CATTGTAGGGTGTCACATTGTAGGGTGTCCAGAAGAGCCGGATGGCCTTGTACTGGATGGCCTTGTACTTTTTTGCCTTGTACTTTTTTTTACTACCTCAGCAGCGTTTTGATGAGCAGCGTTTTGATGATTCC | | | | |
| CGTTTTGATGATTCCTGTGTTGATGATTCCTGTGTAGCAGATTCCTGTGTAGCAGATGGCCGAGCAGAGGGAGAACGAGAAGGTAGATGNTGGTCATGGTCNGAAATGGCCAGGTTAGGAAGAGCAGGTCNGAA | | | | |
| ATGTNATAAAACCCAGAGAGGACCTGTGTNATAAAACCCAGAACAAGCCTGTGTNATAAAACGCTGTACAAGCCTGTGTNATCTCGCTGTACAAGCCTGTGTAAGATCTCGCTGTACAAGCCAAAGAGGCTTGT | | | | |
| ACAGCGAGATGAAAAAGATCTCGCTGTAGGATNATGAAAAAGATCTCCAGCAGGATNATGAAAAAGCATCGTCAGCAGGATNATGAAGTCAATCGTCAGCAGGATNAACCTGTCAATCGTCAGCAGGAGGTACC | | | | |
| TGTCAATCGTCAGGGCCAGGTACCTGTCAATCGCNATCGATTGACAGGTACCTTGGACNATCGATTGACAGGTGTGATGACACCAAAAGTGACGCTGGTGATGACACCAAAAGGATGCTGGTGATGACACCACT | | | | |
| GTACAAGCCTGTGNGAATNCCTGTGTAGCAGATGGTAGGCGATCACCTCNGTCACAAGGCGTAGATCACNGGGTTCCAACNAAGGCGTAGATCACGTTGGAGAGAGGTTCCGGAAGAGAGAGGTTCCGGAAGTA | | | | |
| CCGCAGGTACTTCCGGAACCTCTGNCGCAGGTACTTCCGGAAAGAGGGAGAACGAGACAGAAGAGAACGAGACAGAAGATGGCGAGACAGAAGATGGTCATTCAGAAGATGGTCATTCTCAGGATGGTCATTCT | | | | |
| CAGAGTGTAACTCTTCAGTCTCATAGAAATTCAGGAAGAGCTGCTAGCCAGGCCCCAGGTGACGATGCCCCAGGTGACGATGCTGGTGGTGACGATGCTGGTGATGACAAGTGACAGTCCGGGCTCGAACAGTC | | | | |
| CGGGCTCGAAGGGCCAGCATGGACAATGGCCAGGTGGACAATGGCCAGGTACCTAATGGCCAGGTACCTGTCGACCAGGTACCTGTCGATTGTCTACCTGTCGATTGTCAGCAGGTCGATTGTCAGCAGGATTA | | | | |
| AAGATCTCGCTGTACAAGCCCTCGCTGTACAAGCCTGTGTAACCCTGAGAGGAGCTTACATGAGAGGAGCTTACACATGCGGAGCTTACACATGCCATGGAACCCAGTTATGCCCCCTGAAGTTATGCCCCCTG | | | | |
| ACATAGTGCCCCCTGACATAGTGGATCCTGACATAGTGGATCCAGACATAGTGGATCCAGAATGGATGGATCCAGAATGGAAGGGTCCAGAATGGAAGGGTGACGAATGGAAGGGTGACGAGGAAGAGGGTGAC | | | | |
| GAGGAAGAGCAGGACGAGGAAGAGCAGGTCCGGCCAGGTTGAGCAGGTAGATGTTGAGCAGGTAGATGTTGGGCAGGTAGATGTTGGTCATAATTCGGAGCCTCCTGTATTTGAGCCTCCTGTATTTTATGATC | | | | |
| CTGTATTTTATGAGGATCTATTTTATGAGGATCATCACTATGAGGATCATCACCACCAGGATCATCACCACCACCACAATCACCACCACCACATTGCCCACCACCACATTGCCCAAGACCACATTGCCCAAGAG | | | | |
| GCCCTTGCCCAAGAGGCCCACAGTCAAGAGGCCCACAGTGAACAGGCCCACAGTGAACACCAGGACAGTGAACACCAGGGAGTAGAACACCAGGGAGTACAGCGCTGGGCCATCAGTGCTCTGGCCATCAGTGC | | | | |
| TCTGGTATCAAGTGCTCTGGTATCAGCTTTTCTGGTATCAGCTTTTTCACTATCAGCTTTTTCACAGAGCGCTTTTTCACAGAGCAGGCCTTCACAGAGCAGGCCCACGTAGAGCAGGCCCACGTCATCAAGGC | | | | |
| CCACGTCATCATAGTACACGTCATCATAGTAGGATGCATCATAGTAGGATGTGGTAGTATCTAGTGAGGTTGTCATTAGTGAGGTTGTCATTTCACAGGTTGTCATTTCACTTCTCGTCATTTCACTTCTCCCT | | | | |
| GTTTCACTTCTCCCTGTGATATTCTCCCTGTGATAGAAGAATTCACTTCTCCCTGTGATAGTTCTCCCTGTGATAGAAGAA | | | | |
| Eotaxin-D49372 Nucleic Aid Sequences (GENBANK ACCESSION NO.X61177) | | | | |
| (SEQ ID NO: 529) | | | | |
| GCATTTTTTCAAGTTTTATGATTTATTTAACTTGTGGAACAAAAATAAACCAGAAACCACCACCTCTCACGCCAAAGCTCACACCTTCAGCCTCCAACATGAAGGTCTCCGCAGCACTTCTGTGGCTGCTGCTC | | | | |
| ATAGCAGCTGCCTTCAGCCCCCAGGGGCTCGCTGGGCCAGCTTCTGTCCCAACCACCTGCTGCTTTAACCTGGCCAATAGGAAGATACCCCTTCAGCGACTAGAGAGCTACAGGAGAATCACCAGTGGCAAATG | | | | |
| TCCCCAGAAAGCTGTGATCTTCAAGACCAAACTGGCCAAGGATATCTGTGCCGACCCCAAGAAGAAGTGGGTGCAGGATTCCATGAAGTATCTGGACCAAAAATCTCCAACTCCAAAGCCATAAATAATCACCA | | | | |
| TTTTTGAAACCAAACCAGAGCCTGAGTGTTGCCTAATTTGTTTTCCCTTCTTACAATGCATTCTGAGGTAACCTCATTATCAGTCCAAAGGGCATGGGTTTTATTATATATATATATATTTTTTTTTTAAAAAA | | | | |
| AAACGTATTGCATTTAATTTATTGAGGCTTTAAAACTTATCCTCCATGAATATCAGTTATTTTTAAACTGTAAAGCTTTGTGCAGATTCTTTACCCCCTGGGAGCCCCAATTCGATCCCCTGTCACGTGTGGGC | | | | |
| AATGTTCCCCCTCTCCTCTCTTCCTCCCTGGAATCTTGTAAAGGTCCTGGCAAAGATGATCAGTATGAAAATGTCATTGTTCTTGTGAACCCAAAGTGTGACTCATTAAATGGAAGTAAATGTTGTTTTAGGAA | | | | |
| TAC | | | | |
| 530, | EOTAXIN 1, | D49372, | GGT-GGT-GGT-TTC-TGG-GTT-GGT, | |
| 531, | EOTAXIN 2, | D49372, | GGT-GGT-GGT-TTC-TGG-GTT-G, | |
| 532, | EOTAXIN 3, | D49372, | TGT-TGG-AGG-CTG-AAG-GTG-TG, | |
| 533, | EOTAXIN 4, | D49372, | CTG-CGG-AGA-CCT-TCA-TGT-TGG, | |
| 534, | EOTAXIN 5, | D49372, | TGC-TGC-GGA-GAC-CTT-CAT-G, | |
| 535, | EOTAXIN 6, | D49372, | GTG-CTG-CGG-AGA-CCT-TCA-TGT, | |
| 536, | EOTAXIN 7, | D49372, | GGG-CTG-AAG-GCA-GCT-GCT, | |
| 537, | EOTAXIN 8, | D49372, | CTG-GCC-CAG-CGA-GCC-CCT-GG, | |
| 538, | EOTAXIN 9, | D49372, | GCA-GGT-GGT-TGG-GAC-AGA-AG, | |
| 539, | EOTAXIN 10, | D49372, | TCT-TCC-TAT-TGG-CCA-GGT-T, | |
| 540, | EOTAXIN 11, | D49372, | CCT-GTA-GCT-CTC-TAG-TCG-CTG, | |
| 541, | EOTAXIN 12, | D49372, | GGG-ACA-TTT-GCC-ACT-GGT-G, | |
| 542, | EOTAXIN 13, | D49372, | ATC-ACA-GCT-TTC-TGG-GGA-C, | |
| 543, | EOTAXIN 14, | D49372, | GGC-CAG-TTT-GGT-CTT-GAA-G, | |
| 544, | EOTAXIN 15, | D49372, | CCT-GCA-CCC-ACT-TCT-TCT-TG, | |
| 545, | EOTAXIN 16, | D49372, | TTG-GTC-CAG-ATA-CTT-CAT-GG, | |
| 546, | EOTAXIN 17, | D49372, | TAT-TTA-TGG-CTT-TGG-AGT-TG, | |
| 547, | EOTAXIN 18, | D49372, | TCA-GGC-TCT-GGT-TTG-GTT-TC, | |
| 548, | EOTAXIN 19, | D49372, | CCC-ATG-CCC-TTT-GGA-CTG, | |
| 549, | EOTAXIN 20, | D49372, | CTG-ATA-TTC-ATG-GAG-GAT, | |
| 550, | EOTAXIN 21, | D49372, | TTG-CCC-ACA-CGT-GAC-AGG-GG, | |
| 551, | EOTAXIN 22, | D49372, | TGA-TCA-TCT-TTG-CCA-GGA-CC, | |
| 552, | EOTAXIN 23, | D49372, | GGG-TTC-ACA-AGA-ACA-ATG-AC, | |
| 553, | EOTAXIN 24, | D49372, | CTT-CCA-TTT-AAT-GAG-TCA-CAC, | |
| 554, | EOTAXIN 57, | D49372, | GGT GGT TGG GAC AGA AGC TG, | |
| 555, | EOTAXIN 58, | D49372, | GGG TAT CTT CCT ATT GGC C, | |
| 556, | EOTAXIN 59, | D49372, | TGA TTC TCC TGT AGC TCT CT, | |
| 557, | EOTAXIN 64, | D49372, | ACC CAC TTC TTC TTG GGG TC, | |
| 558, | EOTAXIN 65, | D49372, | TGG TCC AGA TAC TTC ATG G, | |
| 559, | EOTAXIN 66, | D49372, | CTC AGG CTC TGG TTT GGT TTC, | |
| 560, | EOTAXIN 67, | D49372, | CCC ATG CCC TTT GGA CTG, | |
| 561, | EOTAXIN 68, | D49372, | TAA CTG ATA TTC ATG GAG G, | |
| 562, | EOTAXIN 69, | D49372, | TTG CCC ACA CGT GAC AGG G, | |
| 563, | EOTAXIN 70, | D49372, | GAT TCC AGG GAG GAA GAG, | |
| 564, | EOTAXIN 71, | D49372, | TAC TGA TCA TCT TTG CCA GG, | |
| 565, | EOTAXIN 72, | D49372, | TGA GTC ACA CTT TGG GTT C, | |
| Concatemer Nucleic Acid Sequences of Eotaxin-D49372 gene oligo sequences | | | | |
| (SEQ ID NO: 566) | | | | |
| GGTGGTGGTTTCTGGGTTGGTGGTGGTGGTTTCTGGGTTGTGTTGGAGGCTGAAGGTGTGCTGCGGAGACCTTCATGTTGGTGCTGCGGAGACCTTCATGGTGCTGCGGAGACCTTCATGTGGGCTGAAGGCAG | | | | |
| CTGCTCTGGCCCAGCGAGCCCCTGGGCAGGTGGTTGGGACAGAAGTCTTCCTATTGGCCAGGTTCCTGTAGCTCTCTAGTCGCTGGGGACATTTGCCACTGGTGATCACAGCTTTCTGGGGACGGCCAGTTTGG | | | | |
| TCTTGAAGCCTGCACCCACTTCTTCTTGTTGGTCCAGATACTTCATGGTATTTATGGCTTTGGAGTTGTCAGGCTCTGGTTTGGTTTCCCCATGCCCTTTGGACTGCTGATATTCATGGAGGATTTGCCCACAC | | | | |
| GTGACAGGGGTGATCATCTTTGCCAGGACCGGGTTCACAAGAACAATGACCTTCCATTTAATGAGTCACACGGTGGTTGGGACAGAAGCTGGGGTATCTTCCTATTGGCCTGATTCTCCTGTAGCTCTCTACCC | | | | |
| ACTTCTTCTTGGGGTCTGGTCCAGATACTTCATGGCTCAGGCTCTGGTTTGGTTTCCCCATGCCCTTTGGACTGTAACTGATATTCATGGAGGTTGCCCACACGTGACAGGGGATTCCAGGGAGGAAGAGTACT | | | | |
| GATCATCTTTGCCAGGTGAGTCACACTTTGGGTTC | | | | |
| Eoxtaxin-U46573 Nucleic Acid Sequences (GENBANK ACCESSION NO.X61177) | | | | |
| (SEQ ID NO: 567) | | | | |
| CAACCCAGAAACCACCACCTCTCACGCCAAAGCTCACACCTTCAGCCTCCAACATGAAGGTCTCCGCAGCACTTCTGTGGCTGCTGCTCATAGCAGCTGCCTTCAGCCCCCAGGGGCTCGCTGGGCCAGCTTCT | | | | |
| GTCCCAACCACCTGCTGCTTTAACCTGGCCAATAGGAAGATACCCCTTCAGCGACTAGAGAGCTACAGGAGAATCACCAGTGGCAAATGTCCCCAGAAAGCTGTGATCTTCAAGACCAAACTGGCCAAGGATAT | | | | |
| CTGTGCCGACCCCAAGAAGAAGTGGGTGCAGGATTCCATGAAGTATCTGGACCCAAAAATCTCCAACTCCAAAGCCATAAATAATCACCATTTTTGAAACCAAACCAGAGCCTGAGTGTTGCCTAATTTGTTTC | | | | |
| CCTTCTTACAATGCATTCTGAGGTAACCTCATTATCAGTCCAAAGGGCATGGGTTTTATTATATATATATATATATATTTTTTTTTTAAAAAAAAACGTATTGCATTTAATTTATTGAGGCTTTAAACTTATCC | | | | |
| TCCATGAATATCAGTTATTTTAAACTGTAAAGCTTTGTGCAGATTCTTTACCCCCTGGGATGCCCCAATTCGATCCCCTGTCACGTGTGGGCAATGTTCCCCCTCTCCTCTCTTCCTCCCTGGAATCTTGTAAA | | | | |
| GGTCCTGGCAAAGATGATCAGTATGAAAATGTCATTGTTCTTGTGAACCCAAAGTGTGACTCATTAAATGGAAGTAATGTTGTTTTAGGAATACATAAAGTATGTGCATATTTTATTATAGTCACTAGTTGTAA | | | | |
| TTTTTTTGTGGGAAATCCACACTGAGCTGAGGGGG | | | | |
| 568, | EOTAXIN 25, | U46673, | CCC-CTC-AGC-TCA-GTG-TGG, | |
| Concatemer Nucleic Acid Sequences of Exotaxin-U46573 gene oligo sequences | | | | |
| (SEQ ID NO: 569) | | | | |
| CCCCTCAGCTCAGTGTGG | | | | |
| Eotaxin-U46572 Nucleic Acid Sequences (GENBANK ACCESSION NO.X61177) | | | | |
| (SEQ ID NO: 570) | | | | |
| CCACATATTCCCCTCCTTTTCCAAGGCAAGATCCAGATGGATTAAAAAATGTACCAAGTCCCTCCTACTAGCTTGCCTCTCTTCTGTTCTGCTTGACTTCCTAGGATCTGGAATCTGGTCAGCAATCAGGAATC | | | | |
| CCTTCATCGTGACCCCCGCATGGGCAAAGGCTTCCCTGGAATCTCCCACACTGTCTGCTCCCTATAAAAGGCAGGCAGATGGGCCAGAGGAGCAGAGAGGCTGAGACCAACCCAGAAACCACCACCTCTCACGC | | | | |
| CAAAGCTCACACCTTCAGCCTCCAACATGAAGGTCTCCGCAGCACTTCTGTGGCTGCTGCTCATAGCAGCTGCCTTCAGCCCCCAGGGGCTCGCTGGGCCAGGTAAGCCCCCCAACTCCTTACAGGAAAGGTAA | | | | |
| GGTAACCACCTCCAGGCTACTAGGTCAGCAAGAATCTTTACAGACTCACTGCAAATTCTCCATTTGAAAAATAGGGAAACAGGTTTTGTGGGTGGACAAGAAATGCCTCAACCGTCACATCCAGTCACTGGAAG | | | | |
| AGCCAGAACTAGAAAGCTCCCGAGTCTTTTCCCCACATTCAAGAGGGCCGCTGGGTGCATCCTTACCCAGCTATCCTTACAGTGTTTGGGAATGGGGAATGGCTCTGTCTTACTGTGGGCATGGTGGGCATTTT | | | | |
| TGGCAGTGGGAGAGAAGGAAAATCTGTTGATTAGAAGCTCAGTATGTTAATTCGACTCCAGGACAGCTTTCAGAGACAGTGGCTAAGAGAAGAACGAGGTCCCAGGGGATCTCTTGAGGTGACTTATTTTGACA | | | | |
| CTCTTTGGGAAAGTTATCTAGGAGATTTGTTCCATAACTCATTTTCCCATACTCTGGTGACAAATTTACTGAGTGTATCGGTCCCACTGAGCCAGTGCATAGCATGGTAACAAACAGTTCTAAATTATCAATGA | | | | |
| CTTAACAGAATTAACTAAATTAACAAAAGTTACTTTCTCACTTGTACTAAATATCTATAATGTATGGGCTCAGGCTTCTGCATTTTATACTCAGGATTCTAGACTGATGGAGAAGTTGCCATGTGGGGGAACAT | | | | |
| TGATGGATACTGTGATAAAGCAGAAGAAAGCTCTCAGGAGTCTTGCATAGGCAATGCACTGTGGCTCAAAAATGACACCCATCACTTTGTCTCCTTCTTTATTGATCAAAACTAATTAATGCCTCCAACCAAAC | | | | |
| AAAAGTGGCCAAGAAATGCAAGTCTACCTTGTGTCTCAAAACAGAGGATGGAGAATATTTGGTGAAAATTACCATGACCATCACATGGCCACGTAGGTCTTTATAATGACAGAGCTAGCATTTGTCACATTGAC | | | | |
| CAAGCTTTGTCCATACACTCTACAGTAATGATGAGTCCTCAGTGCACAGGGGAGGATGCTGAAGACACAGGACAGCATCCTCCAGACACATAAGACTTCAGAGCAGAGGGATTCTCCCTCCACCTCTCGCAATT | | | | |
| CCTTGCTTTCTCCTAACTTCCTTTACAAAGTCATGCTTGGAAATGTCTATGTATCATCATGTGGCTCATTTTTTTCTCTGTTCATTTTTTTTCCCCAAAATTCAGCTTCTGTCCCAACCACCTGCTGCTTTAAC | | | | |
| CTGGCCAATAGGAAGATACCCCTTCAGCGACTAGAGAGCTACAGGAGAATCACCAGTGGCAAATGTCCCCAGAAAGCTGTGATGTAAGTAAATAAAGTTCACCCTCCCCTAGACAAAAAAATAATGTCTAGGGC | | | | |
| ACAGAGTCAAGAACTGTGGGAGTCATAGACTCTGATAGTTTGACCTCTATGGTCCAATTCATTAATTTTCACAAGTGAGTGTTCACTCCCAGCTCCCTGCCTGGGAGATTGCTGTAGTCATATCAATTTCTTCA | | | | |
| AGTCAAGAGCAAAGATGGTTTTACTGGGCCTTTAAGAGCAGCAACTAACCCAAGAGTCTCATCCTTCCTCCTCTCCGTAGCAACCCTTTGTCCAGGGGCAGATGGTCCTTAAATATTTAGGGTCAAATGGGCAG | | | | |
| AATTTTCAAAAACAATCCTTCCAATTGCATCCTGATTCTCCCCACAGCTTCAAGACCAAACTGGCCAAGGATATCTGTGCCGACCCCAAGAAGAAGTGGGTGCAGGATTCCATGAAGTATCTGGACCAAAAATC | | | | |
| TCCAACTCCAAAGCCATAAATAATCACCATTTTTGAAACCAAACCAGAGCCTGAGTGTTGCCTAATTTGTTTTCCCTTCTTACAATGCATTCTGAGGTAACCTCATTATCAGTCCAAAGGGCATGGGTTTTATT | | | | |
| ATATATATATATATATATTTTTTTTTAAAAAAAAACGTATTGCATTTAATTTATTGAGGCTTTAAAACTTATCCTCCATGAATATCAGTTATTTTTAAACTGTAAAGCTTTGTGCAGATTCTTTACCCCCTGGG | | | | |
| AGCCCCAATTCGATCCCCTGTCACGTGTGGGCAATGTTCCCCCTCTCCTCTCTTCCTCCCTGGAATCTTGTAAAGGTCCTGGCAAAGATGATCAGTATGAAAATGTCATTGTTCTTGTGAACCCAAAGTGTGAC | | | | |
| TCATTAAATGGAAGTAATGTTGTTTTAGGAATACATAAAGTATGTGCATATTTTATTATAGTCACTAGTTGTAATTTTTTGTGGGAAATCCACACTGAGCTGAGGGGG | | | | |
| 571, | EOTAXIN 26, | U46572, | GGG CTT ACC TGG CC AGC, | |
| 572, | EOTAXIN 27, | U46572, | TGG TTA CCT TAC CTT TCC TG, | |
| 573, | EOTAXIN 28, | U46572, | CTG ACC TAG TAG CCT GGA GG, | |
| 574, | EOTAXIN 29, | U46572, | GAA TTT GCA GTG AGT CTG TA, | |
| 575, | EOTAXIN 30, | U46572, | GAG GCA TTT CTT GTC CAC CC, | |
| 576, | EOTAXIN 31, | U46572, | GGC TCT TCC AGT GAC TGG AT, | |
| 577, | EOTAXIN 32, | U46572, | GAC TCG GGA GCT TTC TAG TT, | |
| 578, | EOTAXIN 33, | U46572, | CCA GCG GCC CTC TTG AAT GT, | |
| 579, | EOTAXIN 34, | U46572, | CAC TGT AAG GAT AGC TGG GT, | |
| 580, | EOTAXIN 35, | U46572, | CCA TGC CCA CAG TAA GAC AG, | |
| 581, | EOTAXIN 36, | U46572, | CAG ATT TTC CTT CTC TCC CC, | |
| 582, | EOTAXIN 37, | U46572, | TCT CTT AGC CAC TGT CTC TG, | |
| 583, | EOTAXIN 38, | U46572, | TCC CCT GGG ACC TCG TTC TT, | |
| 584, | EOTAXIN 39, | U46572, | TCT CCT AGA TAA CTT TCC C, | |
| 585, | EOTAXIN 40, | U46572, | CAG AGT ATG GGA AAA TGA GT, | |
| 586, | EOTAXIN 41, | U46572, | CTG GCT CAG TGG GAC CGA T, | |
| 587, | EOTAXIN 42, | U46572, | TGT TTG TTA CCA TGC TAT GC, | |
| 588, | EOTAXIN 43, | U46572, | TGC AGA AGC CTG AGC CCA TA, | |
| 589, | EOTAXIN 44, | U46572, | CTC CAT CAG TCT AGA ATC CT, | |
| 590, | EOTAXIN 45, | U46572, | TCC ATC AAT GTT CCC CCA C, | |
| 591, | EOTAXIN 46, | U46572, | CCT GAG AGC TTT CTT CTG CT, | |
| 592, | EOTAXIN 47, | U46572, | TGA GCC ACA GTG CAT TGC CT, | |
| 593, | EOTAXIN 48, | U46572, | TCT TGG CCA CTT TTG TTT GG, | |
| 594, | EOTAXIN 49, | U46572, | GAC TTG CAT TTC TTG GCC AC, | |
| 595, | EOTAXIN 50, | U46572, | TCT CCA TCC TCT GTT TTG AG, | |
| 596, | EOTAXIN 51, | U46572, | CGT GGC CAT GTG ATG GTC, | |
| 597, | EOTAXIN 52, | U46572, | CAT TAC TGT AGA GTG TAT GG, | |
| 598, | EOTAXIN 53, | U46572, | TTG GAG GCA TTA ATT AGT TT, | |
| 599, | EOTAXIN 54, | U46572, | GCA TCC TCC CCT GTG CAC TG, | |
| 600, | EOTAXIN 55, | U46572, | GGA TGC TGT CCT GTG TCT TC, | |
| 601, | EOTAXIN 56, | U46572, | GGT GGA GGG AGA ATC CCT CT, | |
| 602, | EOTAXIN 57, | U46572, | CAC AGT TCT TGA CTC TGT GC, | |
| 603, | EOTAXIN 58, | U46572, | GGG AGC TGG GAG TGA ACA CT, | |
| 604, | EOTAXIN 59, | U46572, | CCA TCT TTG CTC TTG ACT TG, | |
| 605, | EOTAXIN 60, | U46572, | CTT GGG TTA GTT GCT GCT CT, | |
| Concatemer Nucleic Acid Sequences of Eoxtaxin-U46572 gene oligo sequences | | | | |
| (SEQ ID NO: 606) | | | | |
| GGGCTTACCTGGCCAGCTGGTTACCTTACCTTTCCTGCTGACCTAGTAGCCTGGAGGGAATTTGCAGTGAGTCTGTAGAGGCATTTCTTGTCCACCCGGTCTCTTCCAGTGACTGGATGATCGGGAGCTTTCTA | | | | |
| GTTCCAGCGGCCCTCTTGAATGTCACTGTAAGGATAGCTGGGTCCATGCCCACAGTAAGACAGCAGATTTTCCTTCTCTCCCCTCTCTTAGCCACTGTCTCTGTCCCCTGGGACCTCGTTCTTTCTCCTAGATA | | | | |
| ACTTTCCCCAGAGTATGGGAAAATGAGTCTGGCTCAGTGGGACCGATTGTTTGTTACCATGCTATGCTGCAGAAGCCTGACCCATACTCCATCAGTCTAGAATCCTTCCATCAATGTTCCCCCACCCTGAGGAG | | | | |
| CTTTCTTCTGCTTGAGCCACAGTGCATTGCCTTCTTGGCCACTTTTGTTTGGGACTTGCATTTCTTGGCCACTCTCCATCCTCTGTTTTGAGCGTGGCCATGTGATGGTCCATTACTGTAGAGTGTATGGTTGG | | | | |
| AGGCATTAATTAGTTTGCATCCTCCCCTGTGCACTGGGATGCTGTCCTGTGTCTTCGGTGGAGGGAGAATCCCTCTCACAGTTCTTGACTCTGTGCGGGAGCTGGGAGTGAACACTCCATCTTTGCTCTTGACT | | | | |
| TGCTTGGGTTAGTTGCTGCTCT | | | | |
| RANTES Nucleic Acid Sequences (GENBANK ACCESSION NO.X61177) | | | | |
| (SEQ ID NO: 607) | | | | |
| CCTCCGACAGCCTCTCCACAGGTACCATGAAGGTCTCCGCGGCACGCCTCGCTGTCATCCTCATTGCTACTGCCCTCTGCGCTCCTGCATCTGCCTCCCCATATTCCTCGGACACCACACCCTGCTGCTTTGCC | | | | |
| TACATTGCCCGCCCACTGCCCCGTGCCCACATCAAGGAGTATTTCTACACCAGTGGCAAGTGCTCCAACCCAGCAGTCGTCTTTGTCACCCGAAAGAACCGCCAAGTGTGTGCCAACCCAGAGAAGAAATGGGT | | | | |
| TCGGGAGTACATCAACTCTTTGGAGATGAGCTAGGATGGAGAGTCCTTGAACCTGAACTTACACAAATTTGCCTGTTTCTGCTTGCTCTTGTCCTAGCTTGGGAGGCTTCCCCTCACTATCCTACCCCACCCGC | | | | |
| TCCTTGAAGGGCCCAGATTCTGACCACGACGAGCAGCAGTTACAAAAACCTTCCCCAGGCTGGACGTGGTGGCTCAGCCTTGTAATCCCAGCACTTTGGGAGGCCAAGGTGGGTGGATCACTTGAGGTCAGGAG | | | | |
| TTCGAGACAGCCTGGCCAACATGATGAAACCCCATGTGTACTAAAAATACAAAAAATTAGCCGGGCGTGGTAGCGGGCGCCTGTAGTCCCAGCTACTCGGGAGGCTGAGGCAGGAGAATGGCGTGAACCCGGGA | | | | |
| GCGGAGCTTGCAGTGAGCCGAGATCGCGCCACTGCACTCCAGCCTGGGCGACAGAGCGAGACTCCGTCTCAAAAAAAAAAAAAAAAAAAAAAAAAATACAAAAATTAGCCGCGTGGTGGCCCACGCCTGTAATC | | | | |
| CCAGCTACTCGGGAGGCTAAGGCAGGAAAATTGTTTGAACCCAGGAGGTGGAGGCTGCAGTGAGCTGAGATTGTGCCACTTCACTCCAGCCTGGGTGACAAAGTGAGACTCCGTCACAACAACAACAACAAAAA | | | | |
| GCTTCCCCAACTAAAGCCTAGAAGAGCTTCTGAGGCGCTGCTTTGTCAAAAGGAAGTCTCTAGGTTCTGAGCTCTGGCTTTGCCTTGGCTTTGCAAGGGCTCTGTGACAAGGAAGGAAGTCAGCATGCCTCTAG | | | | |
| AGGCAAGGAAGGGAGGAACACTGCACTCTTAAGCTTCCGCCGTCTCAACCCCTCACAGGAGCTTACTGGCAAACATGAAAAATCGGGG | | | | |
| 608, | EPI-10-1, | NM_002985, | ATTTTTCATGTTTGCCAGTA, | |
| 609, | EPI-10-2, | NM_002985, | GAGTGCAGTGTTCCTCCCTT, | |
| 610, | EPI-10-3, | NM_002985, | CAGTGTTCCTCCCTTCCTTG, | |
| 611, | EPI-10-4, | NM_002985, | TTCCTCCCTTCCTTGCCTCT, | |
| 612, | EPI-10-5, | NM_002985, | CCCTTCCTTGCCTCTAGAGG, | |
| 613, | EPI-10-6, | NM_002985, | CCTTGCCTCTAGAGGCATGC, | |
| 614, | EPI-10-7, | NM_002985, | CCTCTAGAGGCATGCTGACT, | |
| 615, | EPI-10-8, | NM_002985, | AGCAGCGCCTCAGAAGCTCT, | |
| 616, | EPI-10-9, | NM_002985, | CGCCTCAGAAGCTCTTCTAG, | |
| 617, | EPI-10-10, | NM_002985, | CAGAAGCTCTTCTAGGCTTT, | |
| 618, | EPI-10-11, | NM_002985, | GCTCTTCTAGGCTTTAGTTG, | |
| 619, | EPI-10-12, | NM_002985, | AGCCTCCACCTCCTGGGTTC, | |
| 620, | EPI-10-13, | NM_002985, | CCTGCCTTAGCCTCCCGAGT, | |
| 621, | EPI-10-14, | NM_002985, | CTTAGCCTCCCGAGTAGCTG, | |
| 622, | EPI-10-15, | NM_002985, | CCTCCCGAGTAGCTGGGATT, | |
| 623, | EPI-10-16, | NM_002985, | GGATTACAGGCGTGGGCCAC, | |
| 624, | EPI-10-17, | NM_002985, | ACAGGCGTGGGCCACCACGC, | |
| 625, | EPI-10-18, | NM_002985, | CGTGGGCCACCACGCGGCTA, | |
| 626, | EPI-10-19, | NM_002985, | GCAGTGGCGCGATCTCGGCT, | |
| 627, | EPI-10-20, | NM_002985, | CAAGCTCCGCTCCCGGGTTC, | |
| 628, | EPI-10-21, | NM_002985, | TCCGCTCCCGGGTTCACGCC, | |
| 629, | EPI-10-22, | NM_002985, | CTCAGCCTCCCGAGTAGCTG, | |
| 630, | EPI-10-23, | NM_002985, | CCTCCCGAGTAGCTGGGACT, | |
| 631, | EPI-10-24, | NM_002985, | CGAGTAGCTGGGACTACAGG, | |
| 632, | EPI-10-25, | NM_002985, | AGCTGGGACTACAGGCGCCC, | |
| 633, | EPI-10-26, | NM_002985, | GGACTACAGGCGCCCGCTAC, | |
| 634, | EPI-10-27, | NM_002985, | CGCCCGCTACCACGCCCGGC, | |
| 635, | EPI-10-28, | NM_002985, | TTTTGTATTTTTAGTACACA, | |
| 636, | EPI-10-29, | NM_002985, | TTTCATCATGTTGGCCAGGC, | |
| 637, | EPI-10-30, | NM_002985, | TGTCTCGAACTCCTGACCTC, | |
| 638, | EPI-10-31, | NM_002985, | CGAACTCCTGACCTCAAGTG, | |
| 639, | EPI-10-32, | NM_002985, | ACCTCAAGTGATCCACCCAC, | |
| 640, | EPI-10-33, | NM_002985, | AAGTGATCCACCCACCTTGG, | |
| 641, | EPI-10-34, | NM_002985, | ATCCACCCACCTTGGCCTCC, | |
| 642, | EPI-10-35, | NM_002985, | TGCTGGGATTACAAGGCTGA, | |
| 643, | EPI-10-36, | NM_002985, | GGATTACAAGGCTGAGCCAC, | |
| 644, | EPI-10-37, | NM_002985, | ACAAGGCTGAGCCACCACGT, | |
| 645, | EPI-10-38, | NM_002985, | GCCACCACGTCCAGCCTGGG, | |
| 646, | EPI-10-39, | NM_002985, | TGCTGCTCGTCGTGGTCAGA, | |
| 647, | EPI-10-40, | NM_002985, | CTCGTCGTGGTCAGAATCTG, | |
| 648, | EPI-10-41, | NM_002985, | TCAGAATCTGGGCCCTTCAA, | |
| 649, | EPI-10-42, | NM_002985, | ATCTGGGCCCTTCAAGGAGC, | |
| 650, | EPI-10-43, | NM_002985, | GGCCCTTCAAGGAGCGGGTG, | |
| 651, | EPI-10-44, | NM_002985, | GGGAAGCCTCCCAAGCTAGG, | |
| 652, | EPI-10-45, | NM_002985, | GGCAGATGCAGGAGCGCAGA, | |
| 653, | EPI-10-46, | NM_002985, | ATGCAGGAGCGCAGAGGGCA, | |
| 654, | EPI-10-47, | NM_002985, | TCTCCATCCTAGCTCATCTC, | |
| 655, | EPI-10-48, | NM_002985, | AGTTGATGTACTCCCGAACC, | |
| 656, | EPI-10-49, | NM_002985, | GAACCCATTTCTTCTCTGGG, | |
| 657, | EPI-10-50, | NM_002985, | CATTTCTTCTCTGGGTTGGC, | |
| 658, | EPI-10-51, | NM_002985, | CTTCTCTGGGTTGGCACACA, | |
| 659, | EPI-10-52, | NM_002985, | TTGGCACACACTTGGCGGTT, | |
| 660, | EPI-10-53, | NM_002985, | ACACACTTGGCGGTTCTTTC, | |
| 661, | EPI-10-54, | NM_002985, | CTTGGCGGTTCTTTCGGGTG, | |
| 662, | EPI-10-55, | NM_002985, | CTGCTGGGTTGGAGCACTTG, | |
| 663, | EPI-10-56, | NM_002985, | GGGTTGGAGCACTTGCCACT, | |
| 664, | EPI-10-57, | NM_002985, | TACTCCTTGATGTGGGCACG, | |
| 665, | EPI-10-58, | NM_002985, | GCTGAGCCACCACGTCCAGC, | |
| 666, | EPI-10-59, | NM_002985, | CTGGGTTGGCACACACTTGG, | |
| 667, | EPI-10-60, | NM_002985, | ATTTTCCTGCCTTAGCCTCC, | |
| 668, | EPI-10-61, | NM_002985, | GCCTCCCAAGCTAGGACAAG, | |
| 669, | EPI-10-62, | NM_002985, | CCAAGCTAGGACAAGAGCAA, | |
| 670, | EPI-10-63, | NM_002985, | CTAGGACAAGAGCAAGCAGA, | |
| 671, | EPI-10-64, | NM_002985, | CCAAGCTAGGACAAGAGCAA, | |
| 672, | EPI-10-65, | NM_002985, | CTAGGACAAGAGCAAGCAGA, | |
| 673, | EPI-10-66, | NM_002985, | TTCAGGTTCAAGGACTCTCC, | |
| 674, | EPI-10-66A, | NM_002985, | GTTCAAGGACTCTCCATCCT, | |
| 675, | EPI-10-67, | NM_002985, | AGGACTCTCCATCCTAGCTC, | |
| 676, | EPI-10-68, | NM_002985, | ACAGGCGCCCGCTACCACGC, | |
| 677, | EPI-10-69, | NM_002985, | ATGTACTCCCGAACCCATTT, | |
| 678, | EPI-10-70, | NM_002985, | CTCCCGAACCCATTTCTTCT, | |
| 679, | EPI-10-71, | NM_002985, | TCATGTTGGCCAGGCTGTCT, | |
| 680, | EPI-10-72, | NM_002985, | TTGGCCAGGCTGTCTCGAAC, | |
| 681, | EPI-10-73, | NM_002985, | CAGGCTGTCTCGAACTCCTG, | |
| 682, | EPI-10-74, | NM_002985, | TCCCGGGTTCACGCCATTCT, | |
| 683, | EPI-10-75, | NM_002985, | GGTTCACGCCATTCTCCTGC, | |
| 684, | EPI-10-75, | NM_002985, | ACGCCATTCTCCTGCCTCAG, | |
| 685, | EPI-10-76, | NM_002985, | ATTCTCCTGCCTCAGCCTCC, | |
| 686, | EPI-10-77, | NM_002985, | CCTGCCTCAGCCTCCCGAGT, | |
| 687, | EPI-10-78, | NM_002985, | GGGCAGTGGGCGGGCAATGT, | |
| 688, | EPI-10-78, | NM_002985, | GTGGGCGGGCAATGTAGGCA, | |
| 689, | EPI-10-79, | NM_002985, | AAGCAGCAGGGTGTGGTGTC, | |
| 690, | EPI-10-80, | NM_002985, | GCAGGGTGTGGTGTCCGAGG, | |
| 691, | EPI-10-81, | NM_002985, | GTGTGGTGTCCGAGGAATAT, | |
| 692, | EPI-10-82, | NM_002985, | GGAGCGCAGAGGGCAGTAGC, | |
| 693, | EPI-10-83, | NM_002985, | GCAGAGGGCAGTAGCAATGA, | |
| 694, | EPI-10-84, | NM_002985, | GGGCAGTAGCAATGAGGATG, | |
| 695, | EPI-10-85, | NM_002985, | GTAGCAATGAGGATGACAGC, | |
| 696, | EPI-10-86, | NM_002985, | AATGAGGATGACAGCGAGGC, | |
| 697, | EPI-10-87, | NM_002985, | GGATGACAGCGAGGCGTGCC, | |
| 698, | EPI-10-88, | NM_002985, | ACAGCGAGGCGTGCCGCGGA, | |
| 699, | EPI-10-89, | NM_002985, | GAGGCGTGCCGCGGAGACCT, | |
| 700, | EPI-10-90, | NM_002985, | GTGCCGCGGAGACCTTCATG, | |
| 701, | EPI-10-91, | NM_002985, | GCGGAGACCTTCATGGTACC, | |
| 702, | EPI-10-92, | NM_002985, | GACCTTCATGGTACCTGTGG, | |
| 703, | EPI-10-93, | NM_002985, | TCATGGTACCTGTGGAGAGG, | |
| 704, | EPI-10-94, | NM_002985, | GTACCTGTGGAGAGGCTGTC, | |
| 705, | EPI-10-95, | NM_002985, | TGTGGAGAGGCTGTCGGAGG, | |
| 706, | EPI-10-96, | NM_002985, | AGAGGCATGCTGACTTCCTT, | |
| 707, | EPI-10-97, | NM_002985, | CATGCTGACTTCCTTCCTTG, | |
| 708, | EPI-10-98, | NM_002985, | TGACTTCCTTCCTTGTCACA, | |
| 709, | EPI-10-99, | NM_002985, | TCCTTCCTTGTCACAGAGCC, | |
| 710, | EPI-10-100, | NM_002985, | CCTTGTCACAGAGCCCTTGC, | |
| 711, | EPI-10-101, | NM_002985, | CCAGAGCTCAGAACCTAGAG, | |
| 712, | EPI-10-102, | NM_002985, | GCTCAGAACCTAGAGACTTC, | |
| 713, | EPI-10-103, | NM_002985, | GAACCTAGAGACTTCCTTTT, | |
| 714, | EPI-10-104, | NM_002985, | TAGAGACTTCCTTTTGACAA, | |
| 715, | EPI-10-105, | NM_002985, | GGGAAGCTTTTTGTTGTTGT, | |
| 716, | EPI-10-106, | NM_002985, | GCTTTTTGTTGTTGTTGTTG, | |
| 717, | EPI-10-107, | NM_002985, | TTGTTGTTGTTGTTGTGACG, | |
| 718, | EPI-10-108, | NM_002985, | GTTGTTGTTGTGACGGAGTC, | |
| 719, | EPI-10-109, | NM_002985, | TGTTGTGACGGAGTCTCACT, | |
| 720, | EPI-10-110, | NM_002985, | TGACGGAGTCTCACTTTGTC, | |
| 721, | EPI-10-111, | NM_002985, | GAGTCTCACTTTGTCACCCA, | |
| 722, | EPI-10-112, | NM_002985, | TCACTTTGTCACCCAGGCTG, | |
| 723, | EPI-10-113, | NM_002985, | TTGTCACCCAGGCTGGAGTG, | |
| 724, | EPI-10-114, | NM_002985, | ACCCAGGCTGGAGTGAAGTG, | |
| 725, | EPI-10-115, | NM_002985, | GGCTGGAGTGAAGTGGCACA, | |
| 726, | EPI-10-116, | NM_002985, | GAGTGAAGTGGCACAATCTC, | |
| 727, | EPI-10-117, | NM_002985, | AAGTGGCACAATCTCAGCTC, | |
| 728, | EPI-10-118, | NM_002985, | CGAGTAGCTGGGATTACAGG, | |
| 729, | EPI-10-119, | NM_002985, | AGCTGGGATTACAGGCGTGG, | |
| 730, | EPI-10-120, | NM_002985, | GAGACGGAGTCTCGCTCTGT, | |
| 731, | EPI-10-121, | NM_002985, | GGAGTCTCGCTCTGTCGCCC, | |
| 732, | EPI-10-122, | NM_002985, | CTCGCTCTGTCGCCCAGGCT, | |
| 733, | EPI-10-123, | NM_002985, | TCTGTCGCCCAGGCTGGAGT, | |
| 734, | EPI-10-124, | NM_002985, | CGCCCAGGCTGGAGTGCAGT, | |
| 735, | EPI-10-125, | NM_002985, | AGGCTGGAGTGCAGTGGCGC, | |
| 736, | EPI-10-126, | NM_002985, | GGAGTGCAGTGGCGCGATCT, | |
| Concatemer Nucleic Acid Sequences of RANTES gene oligo sequences | | | | |
| (SEQ ID NO: 737) | | | | |
| ATTTTTCATGTTTGCCAGTAGAGTGCAGTGTTCCTCCCTTCAGTGTTCCTCCCTTCCTTGTTCCTCCCTTCCTTGCCTCTCCCTTCCTTGCCTCTAGAGGCCTTGCCTCTAGAGGCATGCCCTCTAGAGGCATG | | | | |
| CTGACTAGCAGCGCCTCAGAAGCTCTCGCCTCAGAAGCTCTTCTAGCAGAAGCTCTTCTAGGCTTTGCTCTTCTAGGCTTTAGTTGAGCCTCCACCTCCTGGGTTCCCTGCCTTAGCCTCCCGAGTCTTAGCCT | | | | |
| CCCGAGTAGCTGCCTCCCGAGTAGCTGGGATTGGATTACAGGCGTGGGCCACACAGGCGTGGGCCACCACGCCGTGGGCCACCACGCGGCTAGCAGTGGCGCGATCTCGGCTCAAGCTCCGCTCCCGGGTTCTC | | | | |
| CGCTCCCGGGTTCACGCCCTCAGCCTCCCGAGTAGCTGCCTCCCGAGTAGCTGGGACTCGAGTAGCTGGGACTACAGGAGCTGGGACTACAGGCGCCCGGACTACAGGCGCCCGCTACCGCCCGCTACCACGCC | | | | |
| CGGCTTTTGTATTTTTAGTACACATTTCATCATGTTGGCCAGGCTGTCTCGAACTCCTGACCTCCGAACTCCTGACCTCAAGTGACCTCAAGTGATCCACCCACAAGTGATCCACCCACCTTGGATCCACCCAC | | | | |
| CTTGGCCTCCTGCTGGGATTACAAGGCTGAGGATTACAAGGCTGAGCCACACAAGGCTGAGCCACCACGTGCCACCACGTCCAGCCTGGGTGCTGCTCGTCGTGGTCAGACTCGTCGTGGTCAGAATCTGTCAG | | | | |
| AATCTGGGCCCTTCAAATCTGGGCCCTTCAAGGAGCGGCCCTTCAAGGAGCGGGTGGGGAAGCCTCCCAAGCTAGGGGCAGATGCAGGAGCGCAGAATGCAGGAGCGCAGAGGGCATCTCCATCCTAGCTCATC | | | | |
| TCAGTTGATGTACTCCCGAACCGAACCCATTTCTTCTCTGGGCATTTCTTCTCTGGGTTGGCCTTCTCTGGGTTGGCACACATTGGCACACACTTGGCGGTTACACACTTGGCGGTTCTTTCCTTGGCGGTTCT | | | | |
| TTCGGGTGCTGCTGGGTTGGAGCACTTGGGGTTGGAGCACTTGCCACTTACTCCTTGATGTGGGCACGGCTGAGCCACCACGTCCAGCCTGGGTTGGCACACACTTGGTCCTGACCTCAAGTGATCCACGTGGT | | | | |
| CAGAATCTGGGCCCATTTTCCTGCCTTAGCCTCCGCCTCCCAAGCTAGGACAAGCCAAGCTAGGACAAGAGCAACTAGGACAAGAGCAAGCAGATTCAGGTTCAAGGACTCTCCGTTCAAGGACTCTCCATCCT | | | | |
| AGGACTCTCCATCCTAGCTCACAGGCGCCCGCTACCACGCATGTACTCCCGAACCCATTTCTCCCGAACCCATTTCTTCTTCATGTTGGCCAGGCTGTCTTTGGCCAGGCTGTCTCGAACCAGGCTGTCTCGAA | | | | |
| CTCCTGTCCCGGGTTCACGCCATTCTGGTTCACGCCATTCTCCTGCACGCCATTCTCCTGCCTCAGATTCTCCTGCCTCAGCCTCCCCTGCCTCAGCCTCCCGAGTGGGCAGTGGGCGGGCAATGTGTGGGCGG | | | | |
| GCAATGTAGGCAAAGCAGCAGGGTGTGGTGTCGCAGGGTGTGGTGTCCGAGGGTGTGGTGTCCGAGGAATATGGAGCGCAGAGGGCAGTAGCGCAGAGGGCAGTAGCAATGAGGGCAGTAGCAATGAGGATGGT | | | | |
| AGCAATGAGGATGACAGCAATGAGGATGACAGCGAGGCGGATGACAGCGAGGCGTGCCACAGCGAGGCGTGCCGCGGAGAGGCGTGCCGCGGAGACCTGTGCCGCGGAGACCTTCATGGCGGAGACCTTCATGG | | | | |
| TACCGACCTTCATGGTACCTGTGGTCATGGTACCTGTGGAGAGGGTACCTGTGGAGAGGCTGTCTGTGGAGAGGCTGTCGGAGGAGAGGCATGCTGACTTCCTTCATGCTGACTTCCTTCCTTGTGACTTCCTT | | | | |
| CCTTGTCACATCCTTCCTTGTCACAGAGCCCCTTGTCACAGAGCCCTTGCCCAGAGCTCAGAACCTAGAGGCTCAGAACCTAGAGACTTCGAACCTAGAGACTTCCTTTTTAGAGACTTCCTTTTGACAAGGGA | | | | |
| AGCTTTTTGTTGTTGTGCTTTTTGTTGTTGTTGTTGTTGTTGTTGTTGTTGTGACGGTTGTTGTTGTGACGGAGTCTGTTGTGACGGAGTCTCACTTGACGGAGTCTCACTTTGTCGAGTCTCACTTTGTCACC | | | | |
| CATCACTTTGTCACCCAGGCTGTTGTCACCCAGGCTGGAGTGACCCAGGCTGGAGTGAAGTGGGCTGGAGTGAAGTGGCACAGAGTGAAGTGGCACAATCTCAAGTGGCACAATCTCAGCTCCGAGTAGCTGGC | | | | |
| ATTACAGGAGCTGGGATTACAGGCGTGGGAGACGGAGTCTCGCTCTGTGCAGTCTCGCTCTGTCGCCCCTCGCTCTGTCGCCCAGGCTTCTGTCGCCCAGGCTGGAGTCGCCCAGGCTGGAGTGCAGTAGGCTG | | | | |
| GAGTGCAGTGGCGCGGAGTGCAGTGGCGCGATCT | | | | |
| MCP4 Nucleic Acid Sequences (GENBANK ACCESSION NO.X61177) | | | | |
| (SEQ ID NO: 738) | | | | |
| TTCAACCCCCAGGGACTTGCTCAGCCAGATGCACTCAACGTCCCATCTACTTGCTGCTTCACATTTAGCAGTAAGAAGATCTCCTTGCAGAGGCTGAAGAGCTATGTGATCACCACCAGCAGGTGTCCCCAGAA | | | | |
| GGCTGTCATCTTCAGAACCAAACTGGGCAAGGAGATCTGTGCTGACCCAAAGGAGAAGTGGGTCCAGAATTATATGAAACACCTGGGCCGGAAGCTCACACCCTGAAGACTTGAACTCTGCTACCCCTACTGAA | | | | |
| GGCTATCAAGCTGGAGTACGTGAAATGACTTTTCCATTCTCCTCTGGCCTCCTCTTCTATGCTTTGGAATACTTCTACCATAATTTTCAAATAGGATGCATTCGGTTTTGTGATTCAAAATGTACTATGTGTTAAGTA | | | | |
| ATATTGGCTATTATTTGACTTGTTGCTGGTTTGGAGTTTATTTGAGTATTGCTGATCTTTTCTATAGCAAGGCCTTGAGCAAGTAGGTTGCTGTCTCTAAGCCCCCTTCCCTTCCACTATGAGCTGCTGGCAGT | | | | |
| GGGTTTGTATTCGGTTCCCAGGGGTTGAGAGCATGCCTGTGGGAGTCATGGACATGAAGGGATGCCGCAATGTAGGAAGGAGAGCTCTTTGTGAATGTGAGGTGTTGCTAAATATGTTATTGTGGAAAGATGAA | | | | |
| TGCAATAGTAGGACTGCTGACATTTTGCAGAAAATACATTTTATTTAAAAATCTCCTAAAAAAAAAAAAAAAAAAAAAAAAAAAAAAAAAAAAA | | | | |
| 739, | EPI-104-1, | 877650, | TCTGGCTGAGCAAGTCCCTG, | |
| 740, | EPI-104-2, | 877650, | TGCATTCATCTTTCCACAAT, | |
| 741, | EPI-104-3, | 877650, | AGAGCTCTCCTTCCTACATT, | |
| 742, | EPI-104-4, | 877650, | TTCCTACATTGCGGCATCCC, | |
| 743, | EPI-104-5, | 877650, | ACATTGCGGCATCCCTTCAT, | |
| 744, | EPI-104-6, | 877650, | GCGGCATCCCTTCATGTCCA, | |
| 745, | EPI-104-7, | 877650, | ATCCCTTCATGTCCATGACT, | |
| 746, | EPI-104-8, | 877650, | TTCATGTCCATGACTCCCAC, | |
| 747, | EPI-104-9, | 877650, | GTCCATGACTCCCACAGGCA, | |
| 748, | EPI-104-10, | 877650, | TGACTCCCACAGGCATGCTC, | |
| 749, | EPI-104-11, | 877650, | CCCACAGGCATGCTCTCAAC, | |
| 750, | EPI-104-12, | 877650, | AGGCATGCTCTCAACCCCTG, | |
| 751, | EPI-104-14, | 877650, | TGCTCTCAACCCCTGGGAAC, | |
| 752, | EPI-104-15, | 877650, | TGCCAGCAGCTCATAGTGGA, | |
| 753, | EPI-104-16, | 877650, | ATAGAAGAGGAGGCCAGAGG, | |
| 754, | EPI-104-17, | 877650, | AGAGGAGGCCAGAGGAGAAT, | |
| 755, | EPI-104-18, | 877650, | AGTCATTTCACGTACTCCAG, | |
| 756, | EPI-104-19, | 877650, | TTTCACGTACTCCAGCTTGA, | |
| 757, | EPI-104-20, | 877650, | CGTACTCCAGCTTGATTTCA, | |
| 758, | EPI-104-21, | 877650, | ATTCTGGACCCACTTCTCCT, | |
| 759, | EPI-104-22, | 877650, | GGACCCACTTCTCCTTTGGG, | |
| 760, | EPI-104-23, | 877650, | GGTGATCACATAGCTCTTCA, | |
| 761, | EPI-104-24, | 877650, | TCACATAGCTCTTCAGCCTC, | |
| 762, | EPI-104-25, | 877650, | TAGCTCTTCAGCCTCTGCAA, | |
| 763, | EPI-104-26, | 877650, | CTTCAGCCTCTGCAAGGAGA, | |
| 764, | EPI-104-27, | 877650, | GCCTCTGCAAGGAGATCTTC, | |
| 765, | EPI-104-28, | 877650, | ACCTGCTGGTGGTGATCACA, | |
| 766, | EPI-104-29, | 877650, | CTGGTGGTGATCACATAGCT, | |
| 767, | EPI-104-30, | 877650, | TCCAGCTTGATTTCAGTAGG, | |
| 768, | EPI-104-31, | 877650, | GGTAGCAGAGTTCAAGTCTT, | |
| 769, | EPI-104-32, | 877650, | CAGAGTTCAAGTCTTCAGGG, | |
| 770, | EPI-104-33, | 877650, | TTCAAGTCTTCAGGGTGTGA, | |
| 771, | EPI-104-34, | 877650, | GTCTTCAGGGTGTGAGCTTC, | |
| 772, | EPI-104-35, | 877650, | CAGGGTGTGAGCTTCCGGCC, | |
| 773, | EPI-104-36, | 877650, | TGTGAGCTTCCGGCCCAGGT, | |
| 774, | EPI-104-37, | 877650, | GCTTCCGGCCCAGGTGTTTC, | |
| 775, | EPI-104-38, | 877650, | CGGCCCAGGTGTTTCATATA, | |
| 776, | EPI-104-39, | 877650, | GCAGCTCATAGTGGAAGGGA, | |
| 777, | EPI-104-40, | 877650, | GCTTAGAGACAGCAACCTAC, | |
| 778, | EPI-104-41, | 877650, | GAGACAGCAACCTACTTGCT, | |
| 779, | EPI-104-42, | 877650, | AGCAACCTACTTGCTCAAGG, | |
| 780, | EPI-104-43, | 877650, | CCTACTTGCTCAAGGCCTTG, | |
| 781, | EPI-104-44, | 877650, | TTGCTCAAGGCCTTGCTATA, | |
| 782, | EPI-104-45, | 877650, | ACATAGTACATTTTGAATCA, | |
| 783, | EPI-104-46, | 877650, | CCGAATGCATCCTATTTGAA, | |
| 784, | EPI-104-47, | 877650, | AATTATGGTAGAAGTATTCC, | |
| 785, | EPI-104-48, | 877650, | TGGTTCTGAAGATGACAGCC, | |
| 786, | EPI-104-49, | 877650, | CTGAAGATGACAGCCTTCTG, | |
| 787, | EPI-104-50, | 877650, | GGGACACCTGCTGGTGGTGA, | |
| 788, | EPI-104-51, | 877650, | TCAACCCCTGGGAACCGAAT, | |
| 789, | EPI-104-52, | 877650, | TCTCCTTCCTACATTGCGGC, | |
| 790, | EPI-104-53, | 877650, | AATGTCAGCAGTCCTACTAT, | |
| 791, | EPI-104-54, | 877650, | CAGCAGTCCTACTATTGCAT, | |
| 792, | EPI-104-55, | 877650, | GTCCTACTATTGCATTCATC, | |
| 793, | EPI-104-56, | 877650, | ACTATTGCATTCATCTTTCC, | |
| 794, | EPI-104-57, | 877650, | AACATATTTAGCAACACCTC, | |
| 795, | EPI-104-58, | 877650, | ATTTAGCAACACCTCACATT, | |
| 796, | EPI-104-59, | 877650, | GCAACACCTCACATTCACAA, | |
| 797, | EPI-104-60, | 877650, | TGCAAGGAGATCTTCTTACT, | |
| 798, | EPI-104-61, | 877650, | ATGTGAAGCAGCAAGTAGAT, | |
| 799, | EPI-104-62, | 877650, | AAGCAGCAAGTAGATGGGAC, | |
| 800, | EPI-104-63, | 877650, | GCAAGTAGATGGGACGTTGA, | |
| 801, | EPI-104-64, | 877650, | TAGATGGGACGTTGAGTGCA, | |
| 802, | EPI-104-65, | 877650, | GGGACGTTGAGTGCATCTGG, | |
| 803, | EPI-104-66, | 877650, | GTTGAGTGCATCTGGCTGAG, | |
| 804, | EPI-104-67, | 877650, | GTGCATCTGGCTGAGCAAGT, | |
| 805, | EPI-104-68, | 877650, | CACTTCTCCTTTGGGTCAGC, | |
| 806, | EPI-104-69, | 877650, | CTCCTTTGGGTCAGCACAGA, | |
| 807, | EPI-104-70, | 877650, | TTGGGTCAGCACAGATCTCC, | |
| 808, | EPI-104-71, | 877650, | TCAGCACAGATCTCCTTGCC, | |
| 809, | EPI-104-72, | 877650, | ACAGATCTCCTTGCCCAGTT, | |
| 810, | EPI-104-73, | 877650, | TCTCCTTGCCCAGTTTGGTT, | |
| 811, | EPI-104-74, | 877650, | TTGCCCAGTTTGGTTCTGAA, | |
| 812, | EPI-104-75, | 877650, | CAGTTTGGTTCTGAAGATGA, | |
| 813, | EPI-104-76, | 877650, | TGGTTCTGAAGATGACAGCC, | |
| 814, | EPI-104-77, | 877650, | CTGAAGATGACAGCCTTCTG, | |
| 815, | EPI-104-78, | 877650, | GGGACACCTGCTGGTGGTGA, | |
| 816, | EPI-104-79, | 877650, | ACCTGCTGGTGGTGATCACA, | |
| 817, | EPI-104-80, | 877650, | CTGGTGGTGATCACATAGCT, | |
| 818, | EPI-104-81, | 877650, | TCCAGCTTGATTTCAGTAGG, | |
| 819, | EPI-104-82, | 877650, | GGTAGCAGAGTTCAAGTCTT, | |
| 820, | EPI-104-83, | 877650, | CAGAGTTCAAGTCTTCAGGG, | |
| 821, | EPI-104-84, | 877650, | TTCAAGTCTTCAGGGTGTGA, | |
| 822, | EPI-104-85, | 877650, | GTCTTCAGGGTGTGAGCTTC, | |
| 823, | EPI-104-86, | 877650, | CAGGGTGTGAGCTTCCGGCC, | |
| 824, | EPI-104-87, | 877650, | TGTGAGCTTCCGGCCCAGGT, | |
| 825, | EPI-104-88, | 877650, | GCTTCCGGCCCAGGTGTTTC, | |
| 826, | EPI-104-89, | 877650, | CGGCCCAGGTGTTTCATATA, | |
| 827, | EPI-104-90, | 877650, | GCAGCTCATAGTGGAAGGGA, | |
| 828, | EPI-104-91, | 877650, | GCTTAGAGACAGCAACCTAC, | |
| 829, | EPI-104-92, | 877650, | GAGACAGCAACCTACTTGCT, | |
| 830, | EPI-104-93, | 877650, | AGCAACCTACTTGCTCAAGG, | |
| 831, | EPI-104-94, | 877650, | CCTACTTGCTCAAGGCCTTG, | |
| 832, | EPI-104-95, | 877650, | TTGCTCAAGGCCTTGCTATA, | |
| 833, | EPI-104-96, | 877650, | ACATAGTACATTTTGAATCA, | |
| 834, | EPI-104-97, | 877650, | CCGAATGCATCCTATTTGAA, | |
| 835, | EPI-104-98, | 877650, | AATTATGGTAGAAGTATTCC, | |
| 836, | EPI-104-99, | 877650, | TCAACCCCTGGGAACCGAAT, | |
| 837, | EPI-104-100, | 877650, | TCTCCTTCCTACATTGCGGC, | |
| 838, | EPI-104-101, | 877650, | AATGTCAGCAGTCCTACTAT, | |
| 839, | EPI-104-102, | 877650, | CAGCAGTCCTACTATTGCAT, | |
| 840, | EPI-104-103, | 877650, | GTCCTACTATTGCATTCATC, | |
| 841, | EPI-104-104, | 877650, | ACTATTGCATTCATCTTTCC, | |
| 842, | EPI-104-105, | 877650, | AACATATTTAGCAACACCTC, | |
| 843, | EPI-104-106, | 877650, | ATTTAGCAACACCTCACATT, | |
| 844, | EPI-104-107, | 877650, | GCAACACCTCACATTCACAA, | |
| 845, | EPI-104-108, | 877650, | TGCAAGGAGATCTTCTTACT, | |
| 846, | EPI-104-109, | 877650, | ATGTGAAGCAGCAAGTAGAT, | |
| 847, | EPI-104-110, | 877650, | AAGCAGCAAGTAGATGGGAC, | |
| 848, | EPI-104-111, | 877650, | GCAAGTAGATGGGACGTTGA, | |
| 849, | EPI-104-112, | 877650, | TAGATGGGACGTTGAGTGCA, | |
| 850, | EPI-104-113, | 877650, | GGGACGTTGAGTGCATCTGG, | |
| 851, | EPI-104-114, | 877650, | GTTGAGTGCATCTGGCTGAG, | |
| 852, | EPI-104-115, | 877650, | GTGCATCTGGCTGAGCAAGT, | |
| 853, | EPI-104-116, | 877650, | CACTTCTCCTTTGGGTCAGC, | |
| 854, | EPI-104-117, | 877650, | CTCCTTTGGGTCAGCACAGA, | |
| 855, | EPI-104-118, | 877650, | TTGGGTCAGCACAGATCTCC, | |
| 856, | EPI-104-119, | 877650, | TCAGCACAGATCTCCTTGCC, | |
| 857, | EPI-104-120, | 877650, | ACAGATCTCCTTGCCCAGTT, | |
| 858, | EPI-104-121, | 877650, | TCTCCTTGCCCAGTTTGGTT, | |
| 859, | EPI-104-122, | 877650, | TTGCCCAGTTTGGTTCTGAA, | |
| 860, | EPI-104-123, | 877650, | CAGTTTGGTTCTGAAGATGA, | |
| Concatemer Nucleic Aid Sequences of MCP4gene oligo sequences | | | | |
| (SEQ ID NO: 861) | | | | |
| TCTGGCTGAGCAAGTCCCTGTGCATTCATCTTTCCACAATAGAGCTCTCCTTCCTACATTTTCCTACATTGCGGCATCCCACATTGCGGCATCCCTTCATGCGGCATCCCTTCATGTCCAATCCCTTCATGTCC | | | | |
| ATGACTTTCATGTCCATGACTCCCACGTCCATGACTCCCACAGGCATGACTCCCACAGGCATGCTCCCCACAGGCATGCTCTCAACAGGCATGCTCTCAACCCCTGTGCTCTCAACCCCTGGGAACTGCCAGCA | | | | |
| GCTCATAGTGGAATAGAAGAGGAGGCCAGAGGAGAGGAGGCCAGAGGAGAATAGTCATTTCACGTACTCCAGTTTCACGTACTCCAGCTTGACGTACTCCAGCTTGATTTCAATTCTGGACCCACTTCTCCTGG | | | | |
| ACCCACTTCTCCTTTGGGGGTGATCACATAGCTCTTCATCACATAGCTCTTCAGCCTCTAGCTCTTCAGCCTCTGCAACTTCAGCCTCTGCAAGGAGAGCCTCTGCAAGGAGATCTTCACCTGCTGGTGGTGAT | | | | |
| CACACTGGTGGTGATCACATAGCTTCCAGCTTGATTTCAGTAGGGGTAGCAGAGTTCAAGTCTTCAGAGTTCAAGTCTTCAGGGTTCAAGTCTTCAGGGTGTGAGTCTTCAGGGTGTGAGCTTCCAGGGTGTGA | | | | |
| GCTTCCGGCCTGTGAGCTTCCGGCCCAGGTGCTTCCGGCCCAGGTGTTTCCGGCCCAGGTGTTTCATATAGCAGCTCATAGTGGAAGGGAGCTTAGAGACAGCAACCTACGAGACAGCAACCTACTTGCTAGCA | | | | |
| ACCTACTTGCTCAAGGCCTACTTGCTCAAGGCCTTGTTGCTCAAGGCCTTGCTATAACATAGTACATTTTGAATCACCGAATGCATCCTATTTGAAAATTATGGTAGAAGTATTCCTGGTTCTGAAGATGACAG | | | | |
| CCCTGAAGATGACAGCCTTCTGGGGACACCTGCTGGTGGTGATCAACCCCTGGGAACCGAATTCTCCTTCCTACATTGCGGCAATGTCAGCAGTCCTACTATCAGCAGTCCTACTATTGCATGTCCTACTATTC | | | | |
| CATTCATCACTATTGCATTCATCTTTCCAACATATTTAGCAACACCTCATTTAGCAACACCTCACATTGCAACACCTCACATTCACAATGCAAGGAGATCTTCTTACTATGTGAAGCAGCAAGTAGATAAGCAG | | | | |
| CAAGTAGATGGGACGCAAGTAGATGGGACGTTGATAGATGGGACGTTGAGTGCAGGGACGTTGAGTGCATCTGGGTTGAGTGCATCTGGCTGAGGTGCATCTGGCTGAGCAAGTCACTTCTCCTTTGGGTCAGC | | | | |
| CTCCTTTGGGTCAGCACAGATTGGGTCAGCACAGATCTCCTCAGCACAGATCTCCTTGCCACAGATCTCCTTGCCCAGTTTCTCCTTGCCCAGTTTGGTTTTGCCCAGTTTGGTTCTGAACAGTTTGGTTCTGA | | | | |
| AGATGATGGTTCTGAAGATGACAGCCCTGAAGATGACAGCCTTCTGGGGACACCTGCTGGTGGTGAACCTGCTGGTGGTGATCACACTGGTGGTGATCACATAGCTTCCAGCTTGATTTCAGTAGGGGTAGCAG | | | | |
| AGTTCAAGTCTTCAGAGTTCAAGTCTTCAGGGTTCAAGTCTTCAGGGTGTGAGTCTTCAGGGTGTGAGCTTCCAGGGTGTGAGCTTCCGGCCTGTGAGCTTCCGGCCCAGGTGCTTCCGGCCCAGGTGTTTCCG | | | | |
| GCCCAGGTGTTTCATATAGCAGCTCATAGTGGAAGGGAGCTTAGAGACAGCAACCTACGAGACAGCAACCTACTTGCTAGCAACCTACTTGCTCAAGGCCTACTTGCTCAAGGCCTTGTTGCTCAAGGCCTTGC | | | | |
| TATAACATAGTACATTTTGAATCACCGAATGCATCCTATTTGAAAATTATGGTAGAAGTATTCCTCAACCCCTGGGAACCGAATTCTCCTTCCTACATTGCGGCAATGTCAGCAGTCCTACTATCAGCAGTCCT | | | | |
| ACTATTGCATGTCCTACTATTGCATTCATCACTATTGCATTCATCTTTCCAACATATTTAGCAACACCTCATTTAGCAACACCTCACATTGCAACACCTCACATTCACAATGCAAGGAGATCTTCTTACTATGT | | | | |
| GAAGCAGCAAGTAGATAAGCAGCAAGTAGATGGGACGCAAGTAGATGGGACGTTGATAGATGGGACGTTGAGTGCAGGGACGTTGAGTGCATCTGGGTTGAGTGCATCTGGCTGAGGTGCATCTGGCTGAGCAA | | | | |
| GTCACTTCTCCTTTGGGTCAGCCTCCTTTGGGTCAGCACAGATTGGGTCAGCACAGATCTCCTCAGCACAGATCTCCTTGCCACAGATCTCCTTGCCCAGTTTCTCCTTGCCCAGTTTGGTTTTGCCCAGTTTG | | | | |
| GTTCTGAACAGTTTGGTTCTGAAGATGA | | | | |
| CD23-X04772 Nucleic Acid Sequences (GENBANK ACCESSION NO.X61177) | | | | |
| (SEQ ID NO: 862) | | | | |
| AGTGGCTCTACTTTCAGAAGAAAGTGTCTCTCTTCCTGCTTAAACCTCTGTCTCTGACGGTCCCTGCCAATCGCTCTGGTCGACCCCAACACACTAGGAGGACAGACACAGGCTCCAAACTCCACTAACCAGAG | | | | |
| CTGTGATTGTGCCCGCTGAGTGGACTGCGTTGTCAGGGAGTGAGTGCTCCATCATCGGGAGAATCCAAGCAGGACCGCCATGGAGGAAGGTCAATATTCAGAGATCGAGGAGCTTCCCAGGAGGCGGTGTTGCA | | | | |
| GGCGTGGGACTCAGATCGTGCTGCTGGGGCTGGTGACCGCCGCTCTGTGGGCTGGGCTGCTGACTCTGCTTCTCCTGTGGCACTGGGACACCACACAGAGTCTAAAACAGCTGGAAGAGAGGGCTGCCCGGAAC | | | | |
| GTCTCTCAAGTTTCCAAGAACTTGGAAAGCCACCACGGTGACCAGATGGCGCAGAAATCCCAGTCCACGCAGATTTCACAGGAACTGGAGGAACTTCGAGCTGAACAGCAGAGATTGAAATCTCAGGACTTGGA | | | | |
| GCTGTCCTGGAACCTGAACGGGCTTCAAGCAGATCTGAGCAGCTTCAAGTCCCAGGAATTGAACGAGAGGAACGAAGCTTCAGATTTGCTGGAAAGACTCCGGGAGGAGGTGACAAAGCTAAGGATGGAGTTGC | | | | |
| AGGTGTCCAGCGGCTTTGTGTGCAACACGTGCCCTGAAAAGTGGATCAACTTCCAACGGAAGTGCTACTACTTCGGCAAGGGCACCAAGCAGTGGGTCCACGCCCGGTATGCCTGTGACGACATGGAAGGGCAG | | | | |
| CTGGTCAGCATCCACAGCCCGGAGGAGCAGGACTTCCTGACCAAGCATGCCAGCCACACCGGCTCCTGGATTGGCCTTCGGAACTTGGACCTGAAGGGAGAGTTTATCTGGGTGGATGGGAGCCATGTGGACTA | | | | |
| CAGCAACTGGGCTCCAGGGGAGCCCACCAGCCGGAGCCAGGGCGAGGACTGCGTGATGATGCGGGGCTCCGGTCGCTGGACCGACGCCTTCTGCGACCGTAAGCTGGGCGCCTGGGTGTGCGACCGGCTGGCCA | | | | |
| CATGCACGCCGCCAGCCAGCGAAGGTTCCGCGGAGTCCATGGGACCTGATTCAAGACCAGACCCTGACGGCCGCCTGCCCACCCCCTCTGCCCCTCTCCACTCTTGAGCATGGATACAGCCAGGCCCAGAGCAA | | | | |
| GACCCTGAAGACCCCCAACCACGGCCTAAAAGCCTCTTTGTGGCTGAAAGGTCCCTGTGACATTTTCTGCCACCCAAACGGAGGCAGCTGACACATCTCCCGCTCCTCTATGGCCCCTGCCTTCCCAGGAGTAC | | | | |
| ACCCCAACAGCACCCTCTCCAGATGGGAGTGCCCCCAACAGCACCCTCTCCAGATGAGAGTTACACCCCAACAGCACCCTCTCCAGATGCAGCCCCATCTCCTCAGCACCCCAGGACCTGAGTATCCCCAGCTC | | | | |
| AGGGTGGTGAGTCCTCCTGTCCAGCCTGCATCAATAAAATGGGGCAGTGATGGCC | | | | |
| 863, | CD4, | X04772, | GCC-TGT-GTC-TGT-CCT-CCT, | |
| 864, | CD5, | X04772, | GCT-TCG-TTC-CTC-TCG-TTC, | |
| 865, | CD6, | X04772, | CTG-CTT-GGT-GCC-CTT-GCC, | |
| 866, | CD7, | X04772, | GTC-CTG-CTC-CTC-CGG-GCT-GTG, | |
| 867, | CD8, | X04772, | CCG-GCT-GGT-GGG-CTC-CCC-TGG, | |
| 868, | CD9, | X04772, | GTC-CTC-GCC-CTG-GCTCCG-GCT, | |
| 869, | CD11, | X04772, | CCT-TCG-CTG-GCT-GGC-GGG-GTC, | |
| 870, | CD12, | X04772, | TCT-TGC-TCT-GGG-CCT-GGC-TGT, | |
| 871, | CD13, | X04772, | GCT-GCC-TCC-GTT-TGG-GTG-GC, | |
| 872, | CD14, | X04772, | GAA-GCT-CCT-CGA-TCT-CTG, | |
| 873, | CD15, | X04772, | GGG-AAG-CTC-CTC-GAT-CTC-TG, | |
| 874, | CD16, | X04772, | CGC-CTC-CTG-GGA-AGC-TCC-TC, | |
| 875, | CD17, | X04772, | CCT-GCA-ACA-CCG-CCT-CCT, | |
| 876, | CD18, | X04772, | CCT-GCA-ACA-CCG-CCT-CCT-GG, | |
| 877, | CD19, | X04772, | GAG-TCC-CAC-GCC-TGC-AAC, | |
| 878, | CD20, | X04772, | GAG-TCC-CAC-GCC-TGC-AAC-AC, | |
| 879, | CD21, | X04772, | GAT-CTG-AGT-CCC-ACG-CCT-GC, | |
| 880, | CD22, | X04772, | GCA-CGA-TCT-GAG-TCC-CAC-GC, | |
| 881, | CD23, | X04772, | AGC-ACG-ATC-TGA-GTC-CCA-CGC, | |
| 882, | CD24, | X04772, | CAG-CCC-CAG-CAG-CAC-GAT-CT, | |
| 883, | CD25, | X04772, | CAG-AGT-CAG-CAG-CCC-AGC-CC, | |
| 884, | CD26, | X04772, | GAA-GCA-GAG-TCA-GCA-GCC-CAG, | |
| 885, | CD27, | X04772, | GCC-CAC-AGA-GCG-GCG-GTC, | |
| 886, | CD28, | X04772, | CAG-CAG-CCC-AGC-CCA-CAG, | |
| 887, | CD29, | X04772, | CAG-AGT-CAG-CAG-CCC-AGC-CC, | |
| 888, | CD30, | X04772, | GAA-GCA-GAG-TCA-GCA-GCC-CAG, | |
| 889, | CD31, | X04772, | CAG-GAG-AAG-CAG-AGT-CAG, | |
| 890, | CD32, | X04772, | TGC-CAC-AGG-AGA-AGC-AGA, | |
| 891, | CD33, | X04772, | CCC-AGT-GCC-ACA-GGA-GAA-GC, | |
| 892, | CD13, | X04772, | GGT-GTC-CCA-GTG-CCA-CAG-G, | |
| 893, | CD34, | X04772, | TGT-GTG-GTG-TCC-CAG-TGC-CAC, | |
| 894, | CD35, | X04772, | TGT-GTG-GTG-TCC-CAG-TGC-C, | |
| 895, | CD36, | X04772, | TGT-GTG-GTG-TCC-CAG-TGC, | |
| 896, | CD37, | X04772, | GAC-TCT-GTG-TGG-TGT-CCC-AGT, | |
| 897, | CD38, | X04772, | GAC-TCT-GTG-TGG-TGT-CCC, | |
| 898, | CD39, | X04772, | AGC-TGT-TTT-AGA-CTC-TGT-GT, | |
| 899, | CD40, | X04772, | CCT-CTC-TTC-CAG-CTG-TTT, | |
| 900, | CD41, | X04772, | GCA-GCC-CTC-TCT-TCC-AGC-TG, | |
| 901, | CD42, | X04772, | TCC-GGG-CAG-CCC-TCT-CTT-CC, | |
| 902, | CD43, | X04772, | TCC-GGG-CAG-CCC-TCT-CTT, | |
| 903, | CD44, | X04772, | GAC-GTT-CCG-GGC-AGC-CCT-CTC, | |
| 904, | CD45, | X04772, | GAC-GTT-CCG-GGC-AGC-CCT-CT, | |
| 905, | CD46, | X04772, | GAC-GTT-CCG-GGC-AGC-CCT-C, | |
| 906, | CD47, | X04772, | GAC-GTT-CCG-GGC-AGC-CCT, | |
| 907, | CD48, | X04772, | TGA-GAG-ACG-TTC-CGG-GCA-GCC, | |
| 908, | CD59, | X04772, | CTT-GAG-AGA-CGT-TCC-GGG-C, | |
| 909, | CD50, | X04772, | CTT-GAG-AGA-CGT-TCC-GGG-C, | |
| 910, | CD51, | X04772, | GTT-CTT-GGA-AAC-TTG-AGA-G, | |
| 911, | CD52, | X04772, | TTT-CCA-AGT-TCT-TGG-AAA-CTT, | |
| 912, | CD53, | X04772, | GTG-GCT-TTC-CAA-GTT-CTT-GG, | |
| 913, | CD54, | X04772, | GTG-GCT-TTC-CAA-GTT-CTT, | |
| 914, | CD55, | X04772, | CCG-TGG-TGG-CTT-TCC-AAG-TTC, | |
| 915, | CD56, | X04772, | CCG-TGG-TGG-CTT-TCC-AAG, | |
| 916, | CD57, | X04772, | GGT-CAC-CGT-GGT-GGC-TTT-CC, | |
| 917, | CD58, | X04772, | GGT-CAC-CGT-GGT-GGC-TTT, | |
| 918, | CD59, | X04772, | CAT-CTG-GTC-ACC-GTG-GTG-GCT, | |
| 919, | CD60, | X04772, | CAT-CTG-GTC-ACC-GTG-GTG, | |
| 920, | CD61, | X04772, | TGC-GCC-ATC-TGG-TCA-CCG-TGG, | |
| 921, | CD62, | X04772, | TGC-GCC-ATC-TGG-TCA-CCG, | |
| 922, | CD63, | X04772, | TTT-CTG-CGC-CAT-CTG-GTC-AC, | |
| 923, | CD64, | X04772, | CTG-GGA-TTT-CTG-CGC-CAT-CTG, | |
| 924, | CD65, | X04772, | CTG-GGA-TTT-CTG-CGC-CAT, | |
| 925, | CD66, | X04772, | GTG-GAC-TGG-GAT-TTC-TGC-GCC, | |
| 926, | CD67, | X04772, | GTG-GAC-TGG-GAT-TTC-TGC, | |
| 927, | CD68, | X04772, | TCT-GCG-TGG-ACT-GGG-ATT-TCT, | |
| 928, | CD69, | X04772, | TCT-GCG-TGG-ACT-GGG-ATT, | |
| 930, | CD70, | X04772, | TGA-AAT-CTG-CGT-GGA-CTG-GG, | |
| 931, | CD71, | X04772, | CCA-GTT-CCT-GTG-AAA-TCT-GCG, | |
| 932, | CD72, | X04772, | TTC-CTC-CAG-TTC-CTG-TG, | |
| 933, | CD74, | X04772, | CGA-AGT-TCC-TCC-AGT-TCC, | |
| 934, | CD75, | X04772, | CAG-CTC-GAA-GTT-CCT-CCA-GTT, | |
| 935, | CD76, | X04772, | CTG-TTC-AGC-TCG-AAG-TTC-CTC, | |
| 936, | CD77, | X04772, | CTC-TGC-TGT-TCA-GCT-CGA-AGT, | |
| 937, | CD78, | X04772, | TCA-ATC-TCT-GCT-GTT-CAG-CTC, | |
| 938, | CD79, | X04772, | GAT-TTC-AAT-CTC-TGC-TGT-TC, | |
| 939, | CD80, | X04772, | TCC-TGA-GAT-TTC-AAT-CTC-TGC, | |
| 940, | CD81, | X04772, | CCA-AGT-CCT-GAG-ATT-TCA-ATC, | |
| 941, | CD82, | X04772, | CAG-CTC-CAA-GTC-CTG-AGA-TTT, | |
| 942, | CD83, | X04772, | CAG-GAC-AGC-TCC-AAG-TCC-TG, | |
| 943, | CD84, | X04772, | GGT-TCC-AGG-ACA-GCT-CCA-AGT, | |
| 944, | CD85, | X04772, | GTT-CAG-GTT-CCA-GGA-CAG-CTC, | |
| 945, | CD86, | X04772, | GCC-CGT-TCA-GGT-TCC-AGG, | |
| 946, | CD87, | X04772, | CTT-GAA-GCC-CGT-TCA-GGT-TCC, | |
| 947, | CD88, | X04772, | TCT-GCT-TGA-AGC-CCG-TTC-AGG, | |
| 948, | CD89, | X04772, | CTC-AGA-TCT-GCT-TGA-AGC-CCG, | |
| 949, | CD90, | X04772, | GCT-GCT-CAG-ATC-TGC-TTG, | |
| 950, | CD91, | X04772, | CTT-GAA-GCT-GCT-CAG-ATC-TGC, | |
| 951, | CD92, | X04772, | TGG-GAC-TTG-AAG-CTG-CTC, | |
| 952, | CD93, | X04772, | TTC-CTG-GGA-CTT-GAA-GCT-GCT, | |
| 953, | CD94, | X04772, | GTT-CAA-TTC-CTG-GGA-CTT-G, | |
| 954, | CD95, | X04772, | CTC-CTC-CCG-GAG-TCT-TTC-CAG, | |
| 955, | CD96, | X04772, | GTC-ACC-TCC-TCC-CGG-AGT-CTT, | |
| 956, | CD97, | X04772, | GCT-TTG-TCA-CCT-CCT-CCC-GG, | |
| 957, | CD98, | X04772, | CCT-TAG-CTT-TGT-CAC-CTC-CTC, | |
| 958, | CD99, | X04772, | TCC-ATC-CTT-AGC-TTT-GTC-ACC, | |
| 959, | CD100, | X04772, | GCA-ACT-CCA-TCC-TTA-GCT-TTG, | |
| 960, | CD101, | X04772, | CAC-CTG-CAA-CTC-CAT-CCT-T, | |
| 961, | CD102, | X04772, | CTG-GAC-ACC-TGC-AAC-TCC-ATC, | |
| 962, | CD103, | X04772, | GCC-GCT-GGA-CAC-CTG-CAA-CTC, | |
| 963, | CD104, | X04772, | CAC-AAA-GCC-GCT-GGA-CAC-CTG, | |
| 964, | CD105, | X04772, | TTG-CAC-ACA-AAG-CCG-CTG-G, | |
| 965, | CD106, | X04772, | CGT-GTT-GCA-CAC-AAA-GCC-GCT, | |
| 966, | CD107, | X04772, | CCA-CTT-TTC-AGG-GCA-CGT-GTT, | |
| 967, | CD108, | X04772, | GTT-GAT-CCA-CTT-TTC-AGG-GC, | |
| 968, | CD109, | X04772, | TGG-AAG-TTG-ATC-CAC-TTT-TC, | |
| 969, | CD110, | X04772, | TCC-GTT-GGA-AGT-TGA-TCC, | |
| 970, | CD111, | X04772, | GCA-CTT-CCG-TTG-GAA-GTT-G, | |
| 971, | CD112, | X04772, | TAG-TAG-CAC-TTC-CGT-TGG, | |
| 972, | CD113, | X04772, | CGA-AGT-AGGT-AGC-ACT-TCC-G, | |
| 973, | CD114, | X04772, | CTT-GCC-GAA-GTA-GTA-GCA-CTT, | |
| 974, | CD115, | X04772, | GTG-CCC-TTG-CCG-AAG-TAG-T, | |
| 975, | CD116, | X04772, | TGG-ACC-CAC-TGC-TTG-GTG-CCC, | |
| 976, | CD117, | X04772, | GGG-CGT-GGA-CCC-ACT-GCT, | |
| 977, | CD118, | X04772, | TAC-CGG-GCG-TGG-ACC-CAC, | |
| 978, | CD119, | X04772, | CAG-GCA-TAC-CGG-GCG-TGG, | |
| 979, | CD120, | X04772, | CGT-CAC-AGG-CAT-ACC-GGG, | |
| 980, | CD121, | X04772, | CAT-GTC-GTC-ACA-GGC-ATA-CCG, | |
| 981, | CD122, | X04772, | CCT-TCC-ATG-TCG-TCA-CAG-GC, | |
| 982, | CD123, | X04772, | GCT-GCC-CTT-CCA-TGT-CGT, | |
| 983, | CD124, | X04772, | GAC-CAG-CTG-CCC-TTC-CAT-GT, | |
| 984, | CD125, | X04772, | TGC-TGA-CCA-GCT-GCC-CTT-CC, | |
| 985, | CD126, | X04772, | TGT-GGA-TGC-TGA-CCA-GCT-GC, | |
| 986, | CD127, | X04772, | GCT-TGG-TCA-GGA-AGT-CCT-GC, | |
| 987, | CD128, | X04772, | GGC-ATG-CTT-GGT-CAG-GAA-GTC, | |
| 988, | CD129, | X04772, | TGG-CTG-GCA-TGC-TTG-GTC-AGG, | |
| 989, | CD130, | X04772, | CGG-TGT-GGC-TGG-CAT-GCT-TGG, | |
| 990, | CD131, | X04772, | GGA-GCC-GGT-GTG-GCT-GGC, | |
| 991, | CD132, | X04772, | TCC-AGG-AGC-CGG-TGT-GGC-TGG, | |
| 992, | CD133, | X04772, | GGC-CAA-TCC-AGG-AGC-CGG-TGT, | |
| 993, | CD134, | X04772, | CCG-AAG-GCC-AAT-CCA-GGA-GCC, | |
| 994, | CD135, | X04772, | GGT-CCA-AGT-TCC-GAA-GGC-C, | |
| 995, | CD136, | X04772, | CTT-CAG-GTC-CAA-GTT-CCG, | |
| 996, | CD137, | X04772, | TCT-CCC-TTC-AGG-TCC-AAG-TTC, | |
| 997, | CD138, | X04772, | TAA-ACT-CTC-CCT-TCA-GGT-CC, | |
| 998, | CD139, | X04772, | CCG-AAA-CTT-CCT-CAG, | |
| 999, | CD140, | X04772, | TCC-ACC-CAG-ATA-AAC-TCT-CCC, | |
| 1000, | CD141, | X04772, | TCC-CAT-CCA-CCC-AGA-TAA-ACT, | |
| 1001, | CD142, | X04772, | TGG-CTC-CCA-TCC-ACC-CAG-AT, | |
| 1002, | CD143, | X04772, | TGG-CTC-CCA-TCC-ACC-CAG, | |
| 1003, | CD144, | X04772, | ATG-GCT-CCC-ATC-CAC-CCA-GAT, | |
| 1004, | CD145, | X04772, | TCC-ACA-TGG-CTC-CCA-TCC-ACC, | |
| 1005, | CD146, | X04772, | TCC-ACA-TGG-CTC-CCA-TCC, | |
| 1006, | CD147, | X04772, | TGT-AGT-CCA-CAT-GGC-TCC-CAT, | |
| 1007, | CD148, | X04772, | TGT-CTC-CCA-TCC-ACC-CAG, | |
| 1008, | CD149, | X04772, | GCC-CAG-TTG-CTG-TAG-TCC, | |
| 1009, | CD150, | X04772, | CTG-GAG-CCC-AGT-TGC-TGT, | |
| 1010, | CD151, | X04772, | CTC-CCC-TGG-AGC-CCA-GTT-GCT, | |
| 1011, | CD152, | X04772, | GGT-CCA-GCG-ACC-GGA-GCC, | |
| 1012, | CD153, | X04772, | GTC-GCA-GAA-GGC-GTC-GGT-CC, | |
| 1013, | CD154, | X04772, | TTA-CGG-TCG-CAG-AAG-GCG-TC, | |
| 1014, | CD155, | X04772, | CCA-GCT-TAC-GGT-CGC-AGA-AGG, | |
| 1015, | CD156, | X04772, | GGC-GCC-CAG-CTT-ACG-GTC-GC, | |
| 1016, | CD157, | X04772, | CCC-AGG-CGC-CCA-GCT-TAC-GGT, | |
| 1017, | CD158, | X04772, | CGC-ACA-CCC-AGG-CGC-CC, | |
| 1018, | CD159, | X04772, | CCG-GTC-GCA-CAC-CCA-GGC-GC, | |
| 1019, | CD160, | X04772, | GCC-AGC-CGG-TCG-CAC-ACC, | |
| 1020, | CD161, | X04772, | TGT-GGC-CAG-CCG-GTC-GCA-C, | |
| 1021, | CD162, | X04772, | CGT-GCA-TGT-GGC-CAG-CCG-GTC, | |
| 1022, | CD163, | X04772, | CGT-GCA-TGT-GGC-CAG-CCG, | |
| 1023, | CD164, | X04772, | TGG-CTG-GCG-GCG-TGC-ATG-TG, | |
| 1024, | CD165, | X04772, | GGA-CTC-CGC-GGA-ACC-TTC-GC, | |
| 1025, | CD166, | X04772, | CCC-ATG-GAC-TCC-GCG-GAA-CC, | |
| 1026, | CD167, | X04772, | CAG-GTC-CCA-TGG-ACT-CCG-CG, | |
| 1027, | CD168, | X04772, | TGA-ATC-AGG-TCC-CAT-GGA-CTC, | |
| 1028, | CD169, | X04772, | TGA-ATC-AGG-TCC-CAT-GGA-C, | |
| 1029, | CD170, | X04772, | GGT-CTT-GAA-TCA-GGT-CCC-ATG, | |
| 1030, | CD171, | X04772, | GGT-CTG-GTC-TTG-AAT-CAG-GTC, | |
| 1031, | CD172, | X04772, | GTC-AGG-GTC-TGG-TCT-TGA-ATC, | |
| 1032, | CD173, | X04772, | GCA-GGC-GGC-CGT-CAG-GGT-C, | |
| 1033, | CD174, | X04772, | TCC-ATG-CTC-AAG-AGT-GGA-GAG, | |
| 1034, | CD175, | X04772, | TCC-ATG-CTC-AAG-AGT-GGA-G, | |
| 1035, | CD176, | X04772, | TGG-GCC-TGG-CTG-TAT-CCA-TGC, | |
| 1036, | CD177, | X04772, | GGT-CTT-CAG-GGT-CTT-GCT-CTG, | |
| 1037, | CD178, | X04772, | GGC-TTT-TAG-GCC-GTG-GTT-GG, | |
| 1038, | CD179, | X04772, | GCC-ACA-AAG-AGG-CTT-TTA-GGC, | |
| 1039, | CD180, | X04772, | GGA-CCT-TTC-AGC-CAC-AAA-GAG, | |
| 1040, | CD181, | X04772, | TGT-CAC-AGG-GAC-CTT-TCA-GCC, | |
| 1041, | CD182, | X04772, | CAG-AAA-ATG-TCA-CAG-GGA-CCT, | |
| 1042, | CD183, | X04772, | GGT-GGC-AGA-AAA-TGT-CAC-AGG, | |
| 1043, | CD184, | X04772, | GGT-GGC-AGA-AAA-TGT-CAC, | |
| 1044, | CD185, | X04772, | GTT-TGG-GTG-GCA-GAA-AAT-GTC, | |
| 1045, | CD186, | X04772, | CCT-CCG-TTT-GGG-TGG-CAG, | |
| 1046, | CD187, | X04772, | TGC-CTC-CGT-TTG-GGT-GGC, | |
| 1047, | CD188, | X04772, | GTG-TCA-GCT-GCC-TCC-GTT-TGG, | |
| 1048, | CD189, | X04772, | GTG-TCA-GCT-GCC-TCC-GTT-T, | |
| 1049, | CD190, | X04772, | GTG-TCA-GCT-GCC-TCC-GTT, | |
| 1050, | CD191, | X04772, | GCG-GGA-GAT-GTG-TCA-GCT-GC, | |
| 1051, | CD192, | X04772, | GAG-GAG-CGG-GAG-ATG-TGT-C, | |
| 1052, | CD193, | X04772, | GCC-ATA-GAG-GAG-CGG-GAG, | |
| 1053, | CD194, | X04772, | GAG-GAG-CGG-GAG-ATG-TGT-C, | |
| 1054, | CD195, | X04772, | GGA-GAG-GGT-GCT-GTT-GGG, | |
| 1055, | CD196, | X04772, | TCT-GGA-GAG-GGT-GCT-GTT-GGG, | |
| 1056, | CD197, | X04772, | TCT-GGA-GAG-GGT-GCT-GTT-GG, | |
| 1057, | CD198, | X04772, | TCT-GGA-GAG-GGT-GCT-GTT-G, | |
| 1058, | CD199, | X04772, | TCT-GGA-GAG-GGT-GCT-GTT, | |
| 1059, | CD200, | X04772, | TCT-GGA-GAG-GGT-GCT-GT, | |
| 1060, | CD201, | X04772, | TCT-GGA-GAG-GGT-GCT-G, | |
| 1061, | CD202, | X04772, | CAT-CTG-GAG-AGG-GTG-CTG-TTG, | |
| 1062, | CD203, | X04772, | CAT-CTG-GAG-AGG-GTG-CTG-T, | |
| 1063, | CD204, | X04772, | CAT-CTG-GAG-AGG-GTG-CT, | |
| 1064, | CD205, | X04772, | CAT-CTG-GAG-AGG-GTG, | |
| 1065, | CD206, | X04772, | CTC-CCA-TCT-GGA-GAG-GGT-GCT, | |
| 1066, | CD207, | X04772, | GGG-CAC-TCC-CAT-CTG-GAG-AGG, | |
| 1067, | CD208, | X04772, | GGG-CAC-TCC-CAT-CTG-GAG, | |
| 1068, | CD209, | X04772, | CTC-TCA-TCT-GGA-GAG-GGT-GC, | |
| 1069, | CD210, | X04772, | GAG-GAC-TCA-CCA-CCC-TGA-GC, | |
| 1070, | CD211, | X04772, | GGC-TGG-ACA-GGA-GGA-CTC, | |
| 1071, | CD212, | X04772, | TAT-TGA-TGC-AGG-CTG-GAC-AGG, | |
| 1072, | CD213, | X04772, | GGC-CAT-CAC-TGC-CCC-ATT-T, | |
| Cancatemer Nucleic Acid Sequences of CD23-X04772 gene oligo sequences | | | | |
| (SEQ ID NO: 1073) | | | | |
| GCCTGTGTCTGTCCTCCTGCTTCGTTCCTCTCGTTCCTGCTTGGTGCCCTTGCCGTCCTGCTCCTCCGGGCTGTGCCGGCTGGTGGGCTCCCCTGGGTCCTCGCCCTGGCTCCGGCTCCTTCGCTGGCTGGCGG | | | | |
| GGTCTCTTGCTCTGGGCCTGGCTGTGCTGCCTCCGTTTGGGTGGCGAAGCTCCTCGATCTCTGGGGAAGCTCCTCGATCTCTGCGCCTCCTGGGAAGCTCCTCCCTGCAACACCGCCTCCTCCTGCAACACCGC | | | | |
| CTCCTGGGAGTCCCACGCCTGCAACGAGTCCCACGCCTGCAACACGATCTGAGTCCCACGCCTGCGCACGATCTGAGTCCCACGCAGCACGATCTGAGTCCCACGCCAGCCCCAGCAGCACGATCTGGTCACCA | | | | |
| GCCCCAGCAGCCGGCGGTCACCAGCCCCAGCGCCCACAGAGCGGCGGTCCAGCAGCCCAGCCCACAGCAGAGTCAGCAGCCCAGCCCGAAGCAGAGTCAGCAGCCCAGCAGGAGAAGCAGAGTCAGTGCCACAG | | | | |
| GAGAAGCAGACCCAGTGCCACAGGAGAAGCGGTGTCCCAGTGCCACAGGTGTGTGGTGTCCCAGTGCCACTGTGTGGTGTCCCAGTGCCTGTGTGGTGTCCCAGTGCGACTCTGTGTGGTGTCCCAGTGACTCT | | | | |
| GTGTGGTGTCCCAGCTGTTTTAGACTCTGTGTCCTCTCTTCCAGCTGTTTGCAGCCCTCTCTTCCAGCTGTCCGGGCAGCCCTCTCTTCCTCCGGGCAGCCCTCTCTTGACGTTCCGGGCAGCCCTCTCGACGT | | | | |
| TCCGGGCAGCCCTCTGACGTTCCGGGCAGCCCTCGACGTTCCGGGCAGCCCTTGAGAGACGTTCCGGGCAGCCCTTGAGAGACGTTCCGGGCCTTGGAAACTTGAGAGACGGTTCTTGGAAACTTGAGAGTTTC | | | | |
| CAAGTTCTTGGAAACTTGTGGCTTTCCAAGTTCTTGGGTGGCTTTCCAAGTTCTTCCGTGGTGGCTTTCCAAGTTCCCGTGGTGGCTTTCCAAGGGTCACCGTGGTGGCTTTCCGGTCACCGTGGTGGCTTTCA | | | | |
| TCTGGTCACCGTGGTGGCTCATCTGGTCACCGTGGTGTGCGCCATCTGGTCACCGTGGTGCGCCATCTGGTCACCGTTTCTGCGCCATCTGGTCACCTGGGATTTCTGCGCCATCTGCTGGGATTTCTGCGCCA | | | | |
| TGTGGACTGGGATTTCTGCGCCGTGGACTGGGATTTCTGCTCTGCGTGGACTGGGATTTCTTCTGCGTGGACTGGGATTTGAAATCTGCGTGGACTGGGTCCTGTGAAATCTGCGTGGCCAGTTCCTGTGAAAT | | | | |
| CTGCGTTCCTCCAGTTCCTGTGCGAAGTTCCTCCAGTTCCCAGCTCGAAGTTCCTCCAGTTCTGTTCAGCTCGAAGTTCCTCCTCTGCTGTTCAGCTCGAAGTTCAATCTCTGCTGTTCAGCTCGATTTCAATC | | | | |
| TCTGCTGTTCTCCTGAGATTTCAATCTCTGCCCAAGTCCTGAGATTTCAATCCAGCTCCAAGTCCTGAGATTTCAGGACAGCTCCAAGTCCTGGGTTCCAGGACAGCTCCAAGTGTTCAGGTTCCAGGACAGCT | | | | |
| CGCCCGTTCAGGTTCCAGGCTTGAAGCCCGTTCAGGTTCCTCTGCTTGAAGCCCGTTCAGGCTCAGATCTGCTTGAAGCCCGGCTGCTCAGATCTGCTTGCTTGAAGCTGCTCAGATCTGCTGGGACTTGAAGC | | | | |
| TGCTCTTCCTGGGACTTGAAGCTGCTGTTCAATTCCTGGGACTTGCTCCTCCCGGAGTCTTTCCAGGTCACCTCCTCCCGGAGTCTTGCTTTGTCACCTCCTCCCGGCCTTAGCTTTGTCACCTCCTCTCCATC | | | | |
| CTTAGCTTTGTCACCGCAACTCCATCCTTAGCTTTGCACCTGCAACTCCATCCTTCTGGACACCTGCAACTCCATCGCCGCTGGACACCTGCAACTCCACAAAGCCGCTGGACACCTGTTGCACACAAAGCCGC | | | | |
| TGGCGTGTTGCACACAAAGCCGCTCCACTTTTCAGGGCACGTGTTGTTGATCCACTTTTCAGGGCTGGAAGTTGATCCACTTTTCTCCGTTGGAAGTTGATCCGCACTTCCGTTGGAAGTTGTAGTAGCACTTC | | | | |
| CGTTGGCGAAGTAGTAGCACTTCCGCTTGCCGAAGTAGTAGCACTTGTGCCCTTGCCGAAGTAGTTGGACCCACTGCTTGGTGCCCGGGCGTGGACCCACTGCTTACCGGGCGTGGACCCACCAGGCATACCGG | | | | |
| GCGTGGCGTCACAGGCATACCGGGCATGTCGTCACAGGCATACCGCCTTCCATGTCGTCACAGGCGCTGCCCTTCCATGTCGTGACCAGCTGCCCTTCCATGTTGCTGACCAGCTGCCCTTCCTGTGGATGCTG | | | | |
| ACCAGCTGCGCTTGGTCAGGAAGTCCTGCGGCATGCTTGGTCAGGAAGTCTGGCTGGCATGCTTGGTCAGGCGGTGTGGCTGGCATGCTTGGGGASGCCGGTGTGGCTGGCTCCAGGAGCCGGTGTGGCTGGGC | | | | |
| CAATCCAGGAGCCGGTGTCCGAAGGCCAATCCAGGAGCCGGTCCAAGTTCCGAAGGCCCTTCAGGTCCAAGTTCCGTCTCCCTTCAGGTCCAAGTTCTAAACTCTCCCTTCAGGTCCCCAGATAAACTCTCCCT | | | | |
| TCAGTCCACCCAGATAAACTCTCCCTCCCATCCACCCAGATAAACTTGGCTCCCATCCACCCAGATTGGCTCCCATCCACCCAGATGGCTCCCATCCACCCAGATTCCACATGGCTCCCATCCACCTCCACATG | | | | |
| GCTCCCATCCTGTAGTCCACATGGCTCCCATTGTAGTCCACATGGCTCCCGCCCAGTTGCTGTAGTCCCTGGAGCCCAGTTGCTGTCTCCCCTGGAGCCCAGTTGCTGGTCCAGCGACCGGAGCCGTCGCAGAA | | | | |
| GGCGTCGGTCCTTACGGTCGCAGAAGGCGTCCCAGCTTACGGTCGCAGAAGGGGCGCCCAGCTTACGGTCGCCCCAGGCGCCCAGCTTACGGTCGCACACCCAGGCGCCCCCGGTCGCACACCCAGGCGCGCCA | | | | |
| GCCGGTCGCACACCTGTGGCCAGCCGGTCGCACCGTGCATGTGGCCAGCCGGTCCGTGCATGTGGCCAGCCGTGGCTGGCGGCGTGCATGTGGGACTCCGCGGAACCTTCGCCCCATGGACTCCGCGGAACCCA | | | | |
| GGTCCCATGGACTCCGCGTGAATCAGGTCCCATGGACTCTGAATCAGGTCCCATGGACGGTCTTGAATCAGGTCCCATGGGTCTGGTCTTGAATCAGGTCGTCAGGGTCTGGTCTTGAATCGCAGGCGGCCGTC | | | | |
| AGGGTCTCCATGCTCAAGAGTGGAGAGTCCATGCTCAAGAGTGGAGTGGGCCTGGCTGTATCCATGCGGTCTTCAGGGTCTTGCTCTGGGCTTTTAGGCCGTGGTTGGGCCACAAAGAGGCTTTTAGGCGGACC | | | | |
| TTTCAGCCACAAAGAGTGTCACAGGGACCTTTCAGCCCAGAAAATGTCACAGGGACCTGGTGGCAGAAAATGTCACAGGGGTGGCAGAAAATGTCACGTTTGGGTGGCAGAAAATGTCCCTCCGTTTGGGTGGC | | | | |
| AGTGCCTCCGTTTGGGTGGCGTGTCAGCTGCCTCCGTTTGGGTGTCAGCTGCCTCCGTTTGTGTCAGCTGCCTCCGTTGCGGGAGATGTGTCAGCTGCGAGGAGCGGGAGATGTGTCGCCATAGAGGAGCGGGA | | | | |
| GGTACTCCTGGGAAGGCAGGGGGAGAGGGTGCTGTTGGGTCTGGAGAGGGTGCTGTTGGGTCTGGAGAGGGTGCTGTTGGTCTGGAGAGGGTGCTGTTGTCTGGAGAGGGTGCTGTTTCTGGAGAGGGTGCTGT | | | | |
| TCTGGAGAGGGTGCTGCATCTGGAGAGGGTGCTGTTGCATCTGGAGAGGGTGCTGTCATCTGGAGAGGGTGCTCATCTGGAGAGGGTGCTCCCATCTGGAGAGGGTGCTGGGCACTCCCATCTGGAGAGGGGGC | | | | |
| ACTCCCATCTGGAGCTCTCATCTGGAGAGGGTGCGAGGACTCACCACCCTGAGCGGCTGGACAGGAGGACTCTATTGATGCAGGCTGGACAGGGGCCATCACTGCCCCATTT | | | | |
| CD23-M23562 Nucleic Acid Sequences (GENBANK ACCESSION NO.X61177) | | | | |
| (SEQ ID NO: 1074) | | | | |
| CCATGGAGGAAGGTCAATATTCAGGTAGGAGGACTCTCTGGTTCTAACGTTGGCAGAAGCAATGACCCTTAGCTACTCCTTTCACCCAGAAGAGAAGCGGGGCTTCCCAGTCCCTCTCTGGGAAAGAGGGTGAA | | | | |
| TTTCTAAGAAAGGGACTGGTGTGAGTAAGGAGGTGAGGCCGCACTGACTTTCCTGGCACAGAGCCAGGAAGGAGTGGAAAATTGAGGGCCCCTCCTTTTTCTGATTCAACACCCTCCTGACAAAAAAAGAAAAA | | | | |
| GAAAAAAAAAAACGGCTTCAGCTAGGGAGCGGGGACGCAATAGAGTCAGAGGCCAAATAGAACAGGAACTTGGAACAAGCAGAATTTAGCATAATGAATCCTCCAAGCCAGGGTGAGTGCAGA | | | | |
| 1075, | CD1, | M23562, | TGC-GTC-CCC-GCT-CCC-TAG-CTG, | |
| 1076, | CD2, | M23562, | TCC-TGT-TCT-ATT-TGG-CCT-CTG, | |
| 1077, | CD3, | M23562, | GCT-TGG-AGG-ATT-CAT-TAT-GCT, | |
| Contatemer Nucleic Acid Sequences of CD23-M23562 gene oligo sequences | | | | |
| (SEQ ID NO: 1078) | | | | |
| TGCGTCCCCGCTCCCTAGCTGTCCTGTTCTATTTGGCCTCTGGCTTGGAGGATTCATTATGCT | | | | |
| CD23-M14766 Nucleic Acid Sequences (GENBANK ACCESSION NO.X61177) | | | | |
| (SEQ ID NO: 1079) | | | | |
| CTCCTGCTTAAACCTCTGTCTCTGACGGTCCCTGCCAATCGCTCTGGTCGACCCCAACACACTAGGAGGACAGACACAGGCTCCAAACTCCACTAAGTGACCAGAGCTGTGATTGTGCCCGCTGAGTGGACTGC | | | | |
| GTTGTCAGGGAGTGAGTGCTCCATCATCGGGAGAATCCAAGCAGGACCGCCATGGAGGAAGGTCAATATTCAGAGATCGAGGAGCTTCCCAGGAGGCGGTGTTGCAGGCGTGGGACTCAGATCGTGCTGCTGGG | | | | |
| GCTGGTGACCGCCGCTCTGTGGGCTGGGCTGCTGACTCTGCTTCTCCTGTGGCACTGGGACACCACACAGAGTCTAAAACAGCTGGAAGAGAGGGCTGCCCGGAACGTCTCTCAAGTTTCCAAGAACTTGGAAA | | | | |
| GCCACCACGGTGACCAGATGGCGCAGAAATCCCAGTCCACGCAGATTTCACAGGAACTGGAGGAACTTCGAGCTGAACAGCAGAGATTGAAATCTCAGGACTTGGAGCTGTCCTGGAACCTGAACGGGCTTCAA | | | | |
| GCAGATCTGAGCAGCTTCAAGTCCCAGGAATTGAACGAGAGGAACGAAGCTTCAGATTTGCTGGAAAGACTCCGGGAGGAGGTGACAAAGCTAAGGATGGAGTTGCAGGTGTCCAGCGGCTTTGTGTGCAACAC | | | | |
| GTGCCCTGAAAAGTGGATCAATTTCCAACGGAAGTGCTACTACTTCGGCAAGGGCACCAAGCAGTGGGTCCACGCCCGGTATGCCTGTGACGACATGGAAGGGCAGCTGGTCAGCATCCACAGCCCGGAGGAGC | | | | |
| AGGACTTCCTGACCAAGCATGCCAGCCACACCGGCTCCTGGATTGGCCTTCGGAACTTGGACCTGAAGGGAGAGTTTATCTGGGTGGATGGGAGCCATGTGGACTACAGCAACTGGGCTCCAGGGGAGCCCACC | | | | |
| AGCCGGAGCCAGGGCGAGGACTGCGTGATGATGCGGGGCTCCGGTCGCTGGAACGACGCCTTCTGCGACCGTAAGCTGGGCGCCTGGGTGTGCGACCGGCTGGCCACATGCACGCCGCCAGCCAGCGAAGGTTC | | | | |
| CGCGGAGTCCATGGGACCTGATTCAAGACCAGACCCTGACGGCCGCCTGCCCACCCCCTCTGCCCCTCTCCACTCTTGAGCATGGATACAGCCAGGCCCAGAGCAAGACCCTGAAGACCCCCAACCACGGCCTA | | | | |
| AAAGCCTCTTTGTGGCTGAAAGGTCCCTGTGACATTTTCTGCCACCCAAACGGAGGCAGCTGACACATCTCCCGCTCCTCTATGGCCCCTGCCTTCCCAGGAGTACACCCCAACAGCACCCTCTCCAGATGGGA | | | | |
| GTGCCCCCAACAGCACCCTCTCCAGATGAGAGTACACCCCAACAGCACCCTCTCCAGATGCAGCCCCATCTCCTCAGCACCCCAGGACCTGAGTATCCCCAGCTCAGGTGGTGAGTCCTCCTGTCCAGCCTGCA | | | | |
| TCAATAAAATGGGGCAGTGATGGCCTCCC | | | | |
| 1080, | CD10, | M14766, | GCA-GAA-GGC-GTC-GTT-CC, | |
| Concatemer Nucleic Acid Sequences of CD23-M14766 gene oligo sequences | | | | |
| (SEQ ID NO: 1081) | | | | |
| GCAGAAGGCGTCGTTCC | | | | |
| ICAM Nucleic Acid Sequences (GENBANK ACCESSION NO.X61177) | | | | |
| (SEQ ID NO: 1082) | | | | |
| GCGCCCCAGTCGACGCTGAGCTCCTCTGCTACTCAGAGTTGCAACCTCAGCCTCGCTATGGCTCCCAGCAGCCCCCGGCCCGCGCTGCCCGCACTCCTGGTCCTGCTCGGGGCTCTGTTCCCAGGACCTGGCAA | | | | |
| TGCCCAGACATCTGTGTCCCCCTCAAAAGTCATCCTGCCCCGGGGAGGCTCCGTGCTGGTGACATGCAGCACCTCCTGTGACCAGCCCAAGTTGTTGGGCATAGAGACCCCGTTGCCTAAAAAGGAGTTGCTCC | | | | |
| TGCCTGGGAACAACCGGAAGGTGTATGAACTGAGCAATGTGCAAGAAGATAGCCAACCAATGTGCTATTCAAACTGCCCTGATGGGCAGTCAACAGCTAAAACCTTCCTCACCGTGTACTGGACTCCAGAACGG | | | | |
| GTGGAACTGGCACCCCTCCCCTCTTGGCAGCCAGTGGGCAAGAACCTTACCCTACGCTGCCAGGTGGAGGGTGGGGCACCCCGGGCCAACCTCACCGTGGTGCTGCTCCGTGGGGAGAAGGAGCTGAAACGGGA | | | | |
| GCCAGCTGTGGGGGAGCCCGCTGAGGTCACGACCACGGTGCTGGTGAGGAGAGATCACCATGGAGCCAATTTCTCGTGCCGCACTGAACTGGACCTGCGGCCCCAAGGGCTGGAGCTGTTTGAGAACACCTCGG | | | | |
| CCCCCTACCAGCTCCAGACCTTTGTCCTGCCAGCGACTCCCCCACAACTTGTCAGCCCCCGGGTCCTAGAGGTGGACACGCAGGGGACCGTGGTCTGTTCCCTGGACGGGCTGTTCCCAGTCTCGGAGGCCCAG | | | | |
| GTCCACCTGGCACTGGGGGACCAGAGGTTGAACCCCACAGTCACCTATGGCAACGACTCCTTCTCGGCCAAGGCCTCAGTCAGTGTGACCGCAGAGGACGAGGGCACCCAGCGGCTGACGTGTGCAGTAATACT | | | | |
| GGGGAACCAGAGCCAGGAGACACTGCAGACAGTGACCATCTACAGCTTTCCGGCGCCCAACGTGATTCTGACGAAGCCAGAGGTCTCAGAAGGGACCGAGGTGACAGTGAAGTGTGAGGCCCACCCTAGAGCCA | | | | |
| AGGTGACGCTGAATGGGGTTCCAGCCCAGCCACTGGGCCCGAGGGCCCAGCTCCTGCTGAAGGCCACCCCAGAGGACAACGGGCGCAGCTTCTCCTGCTCTGCAACCCTGGAGGTGGCCGGCCAGCTTATACAC | | | | |
| AAGAACCAGACCCGGGAGCTTCGTGTCCTGTATGGCCCCCGACTGGACGAGAGGGATTGTCCGGGAAACTGGACGTGGCCAGAAAATTCCCAGCAGACTCCAATGTGCCAGGCTTGGGGGAACCCATTGCCCGA | | | | |
| GCTCAAGTGTCTAAAGGATGGCACTTTCCCACTGCCCATCGGGGAATCAGTGACTGTCACTCGAGATCTTGAGGGCACCTACCTCTGTCGGGCCAGGAGCACTCAAGGGGAGGTCACCCGCGAGGTGACCGTGA | | | | |
| ATGTGCTCTCCCCCCGGTATGAGATTGTCATCATCACTGTGGTAGCAGCCGCAGTCATAATGGGCACTGCAGGCCTCAGCACGTACCTCTATAACCGCCAGCGGAAGATCAAGAAATACAGACTACAACAGGCC | | | | |
| CAAAAAGGGACCCCCATGAAACCGAACACACAAGCCACGCCTCCCTGAACCTATCCCGGGACAGGGCCTCTTCCTCGGCCTTCCCATATTGGTGGCAGTGGTGCCACACTGAACAGAGTGGAAGACATATGCCA | | | | |
| TGCAGCTACACCTACCGGCCCTGGGACGCCGGAGGACAGGGCATTGTCCTCAGTCAGATACAACAGCATTTGGGGCCATGGTACCTGCACACCTAAAACACTAGGCCACGCATCTGATCTGTAGTCACATGACT | | | | |
| AAGCCAAGAGGAAGGAGCAAGACTCAAGACATGATTGATGGATGTTAAAGTCTAGCCTGATGAGAGGGGAAGTGGTGGGGGAGACATAGCCCCACCATGAGGACATACAACTGGGAAATACTGAAACTTGCTGC | | | | |
| GCTCAAGTGTCTAAAGGATGGCACTTTCCCACTGCCCATCGGGGAATCAGTGACTGTCACTCGAGATCTTGAGGGCACCTACCTCTGTCGGGCCAGGAGCACTCAAGGGGAGGTCACCCGCGAGGTGACCGTGA | | | | |
| ATGTGCTCTCCCCCCGGTATGAGATTGTCATCATCACTGTGGTAGCAGCCGCAGTCATAATGGGCACTGCAGGCCTCAGCACGTACCTCTATAACCGCCAGCGGAAGATCAAGAAATACAGACTACAACAGGCC | | | | |
| CAAAAAGGGACCCCCATGAAACCGAACACACAAGCCACGCCTCCCTGAACCTATCCCGGGACAGGGCCTCTTCCTCGGCCTTCCCATATTGGTGGCAGTGGTGCCACACTGAACAGAGTGGAAGACATATGCCA | | | | |
| TGCAGCTACACCTACCGGCCCTGGGACGCCGGAGGACAGGGCATTGTCCTCAGTCAGATACAACAGCATTTGGGGCCATGGTACCTGCACACCTAAAACACTAGGCCACGCATCTGATCTGTAGTCACATGACT | | | | |
| AAGCCAAGAGGAAGGAGCAAGACTCAAGACATGATTGATGGATGTTAAAGTCTAGCCTGATGAGAGGGGAAGTGGTGGGGGAGACATAGCCCCACCATGAGGACATACAACTGGGAAATACTGAAACTTGCTGC | | | | |
| CTATTGGGTATGCTGAGGCCCACAGACTTACAGAAGAAGTGGCCCTCCATAGACATGTGTAGCATCAAAACACAAAGGCCCACACTTCCTGACGGATGCCAGCTTGGGCACTGCTGTCTACTGACCCCAACCCT | | | | |
| TGATGATATGTATTTATTCATTTGTTATTTTACCAGCTATTTATTGAGTGTCTTTTATGTAGGCTAAATGAACATAGGTCTCTGGCCTCACGGAGCTCCCAGTCCATGTCACATTCAAGGTCACCAGGTACAGT | | | | |
| TGTACAGGTTGTACACTGCAGGAGAGTGCCTGGCAAAAAGATCAAATGGGGCTGGGACTTCTCATTGGCCAACCTGCCTTTCCCCAGAAGGAGTGATTTTTCTATCGGCACAAAAGCACTATATGGACTGGTAA | | | | |
| TGGTTCACAGGTTCAGAGATTACCCAGTGAGGCCTTATTCCTCCCTTCCCCCCAAAACTGACACCTTTGTTAGCCACCTCCCCACCCACATACATTTCTGCCAGTGTTCACAATGACACTCAGCGGTCATGTCT | | | | |
| GGACATGAGTGCCCAGGGAATATGCCCAAGCTATGCCTTGTCCTCTTGTCCTGTTTGCATTTCACTGGGAGCTTGCACTATTGCAGCTCCAGTTTCCTGCAGTGATCAGGGTCCTGCAAGCAGTGGGGAAGGGG | | | | |
| GCCAAGGTATTGGAGGACTCCCTCCCAGCTTTGGAAGGGTCATCCGCGTGTGTGTGTGTGTGTATGTGTAGACAAGCTCTCGCTCTGTCACCCAGGCTGGAGTGCAGTGGTGCAATCATGGTTCACTGCAGTCT | | | | |
| TGACCTTTTGGGCTCAAGTGATCCTCCCACCTCAGCCTCCTGAGTAGCTGGGACCATAGGCTCACAACACCACACCTGGCAAATTTGATTTTTTTTTTTTTTTTCAGAGACGGGGTCTCGCAACATTGCCCAGA | | | | |
| CTTCCTTTGTGTTAGTTAATAAAGCTTTCTCAACTGCC | | | | |
| 1083, | EPI-37-001, | J03132, | GGAAGTGTGGGCCTTTGTGT, | |
| 1084, | EPI-37-002, | J03132, | GCCTTTGTGTTTTGATGCTA, | |
| 1085, | EPI-37-003, | J03132, | TTTGATGCTACACATGTCTA, | |
| 1086, | EPI-37-004, | J03132, | CACATGTCTATGGAGGGCCA, | |
| 1087, | EPI-37-005, | J03132, | TGGAGGGCCACTTCTTCTGT, | |
| 1088, | EPI-37-006, | J03132, | CTTCTTCTGTAAGTCTGTGG, | |
| 1089, | EPI-37-007, | J03132, | AAGTCTGTGGGGCCTCAGCA, | |
| 1090, | EPI-37-008, | J03132, | GGCCTCAGCATACCCAATAG, | |
| 1091, | EPI-37-009, | J03132, | TACCCAATAGGCAGCAAGTT, | |
| 1092, | EPI-37-010, | J03132, | GCAGCAAGTTTCAGTATTTC, | |
| 1093, | EPI-37-011, | J03132, | TCAGTATTTCCCAGTTGTAT, | |
| 1094, | EPI-37-012, | J03132, | CCAGTTGTATGTCCTCATGG, | |
| 1095, | EPI-37-013, | J03132, | GTCCTCATGGTGGGGCTATG, | |
| 1096, | EPI-37-014, | J03132, | TGGGGCTATGTCTCCCCCAC, | |
| 1097, | EPI-37-015, | J03132, | TCTCCCCCACCACTTCCCCT, | |
| 1098, | EPI-37-016, | J03132, | CACTTCCCCTCTCATCAGGC, | |
| 1099, | EPI-37-017, | J03132, | CTCATCAGGCTAGACTTTAA, | |
| 1100, | EPI-37-018, | J03132, | TAGACTTTAACATCCATCAA, | |
| 1101, | EPI-37-019, | J03132, | CATCCATCAATCATGTCTTG, | |
| 1102, | EPI-37-020, | J03132, | TCATGTCTTGAGTCTTGCTC, | |
| 1103, | EPI-37-021, | J03132, | AGTCTTGCTCCTTCCTCTTG, | |
| 1104, | EPI-37-022, | J03132, | CTTCCTCTTGGCTTAGTCAT, | |
| 1105, | EPI-37-023, | J03132, | GCTTAGTCATGTGACTACAG, | |
| 1106, | EPI-37-024, | J03132, | GTGACTACAGATCAGATGCG, | |
| 1107, | EPI-37-025, | J03132, | ATCAGATGCGTGGCCTAGTG, | |
| 1108, | EPI-37-026, | J03132, | TGGCCTAGTGTTTTAGGTGT, | |
| 1109, | EPI-37-027, | J03132, | TTTTAGGTGTGCAGGTACCA, | |
| 1110, | EPI-37-028, | J03132, | GCAGGTACCATGGCCCCAAA, | |
| 1111, | EPI-37-029, | J03132, | TGGCCCCAAATGCTGTTGTA, | |
| 1112, | EPI-37-030, | J03132, | TGCTGTTGTATCTGACTGAG, | |
| 1113, | EPI-37-031, | J03132, | TCTGACTGAGGACAATGCCC, | |
| 1114, | EPI-37-032, | J03132, | GACAATGCCCTGTCCTCCGG, | |
| 1115, | EPI-37-033, | J03132, | TGTCCTCCGGCGTCCCAGGG, | |
| 1116, | EPI-37-034, | J03132, | CGTCCCAGGGCCGGTAGGTG, | |
| 1117, | EPI-37-035, | J03132, | CCGGTAGGTGTAGCTGCATG, | |
| 1118, | EPI-37-036, | J03132, | TAGCTGCATGGCATATGTCT, | |
| 1119, | EPI-37-037, | J03132, | GCATATGTCTTCCACTCTGT, | |
| 1120, | EPI-37-038, | J03132, | TCCACTCTGTTCAGTGTGGC, | |
| 1121, | EPI-37-039, | J03132, | TCAGTGTGGCACCACTGCCA, | |
| 1122, | EPI-37-040, | J03132, | ACCACTGCCACCAATATGGG, | |
| 1123, | EPI-37-041, | J03132, | CCAATATGGGAAGGCCGAGG, | |
| 1124, | EPI-37-042, | J03132, | AAGGCCGAGGAAGAGGCCCT, | |
| 1125, | EPI-37-043, | J03132, | AAGAGGCCCTGTCCCGGGAT, | |
| 1126, | EPI-37-044, | J03132, | GTCCCGGGATAGGTTCAGGG, | |
| 1127, | EPI-37-045, | J03132, | AGGTTCAGGGAGGCGTGGCT, | |
| 1128, | EPI-37-046, | J03132, | AGGCGTGGCTTGTGTGTTCG, | |
| 1129, | EPI-37-047, | J03132, | TGTGTGTTCGGTTTCATGGG, | |
| 1130, | EPI-37-048, | J03132, | GTTTCATGGGGGTCCCTTTT, | |
| 1131, | EPI-37-049, | J03132, | GGTCCCTTTTTGGGCCTGTT, | |
| 1132, | EPI-37-050, | J03132, | TGGGCCTGTTGTAGTCTGTA, | |
| 1133, | EPI-37-051, | J03132, | GTAGTCTGTATTTCTTGATC, | |
| 1134, | EPI-37-052, | J03132, | TTTCTTGATCTTCCGCTGGC, | |
| 1135, | EPI-37-053, | J03132, | TTCCGCTGGCGGTTATAGAG, | |
| 1136, | EPI-37-054, | J03132, | GGTTATAGAGGTACGTGCTG, | |
| 1137, | EPI-37-055, | J03132, | GTACGTGCTGAGGCCTGCAG, | |
| 1138, | EPI-37-056, | J03132, | AGGCCTGCAGTGCCCATTAT, | |
| 1139, | EPI-37-057, | J03132, | TGCCCATTATGACTGCGGCT, | |
| 1140, | EPI-37-058, | J03132, | GACTGCGGCTGCTACCACAG, | |
| 1141, | EPI-37-059, | J03132, | GCTACCACAGTGATGATGAC, | |
| 1142, | EPI-37-060, | J03132, | TGATGATGACAATCTCATAC, | |
| 1143, | EPI-37-061, | J03132, | AATCTCATACCGGGGGGAGA, | |
| 1144, | EPI-37-062, | J03132, | CGGGGGGAGAGCACATTCAC, | |
| 1145, | EPI-37-063, | J03132, | GCACATTCACGGTCACCTTG, | |
| 1146, | EPI-37-064, | J03132, | GGTCACCTTGCGGGTGACCT, | |
| 1147, | EPI-37-065, | J03132, | CGGGTGACCTCCCCTTGAGT, | |
| 1148, | EPI-37-066, | J03132, | CCCCTTGAGTGCTCCTGGCC, | |
| 1149, | EPI-37-067, | J03132, | GCTCCTGGCCCGACAGAGGT, | |
| 1150, | EPI-37-068, | J03132, | CGACAGAGGTAGGTGCCCTC, | |
| 1151, | EPI-37-069, | J03132, | AGGTGCCCTCAAGATCTCGA, | |
| 1152, | EPI-37-070, | J03132, | AAGATCTCGAGTGACAGTCA, | |
| 1153, | EPI-37-071, | J03132, | GTGACAGTCACTGATTCCCC, | |
| 1154, | EPI-37-072, | J03132, | CTGATTCCCCGATGGGCAGT, | |
| 1155, | EPI-37-073, | J03132, | GATGGGCAGTGGGAAAGTGC, | |
| 1156, | EPI-37-074, | J03132, | GGGAAAGTGCCATCCTTTAG, | |
| 1157, | EPI-37-075, | J03132, | CATCCTTTAGACACTTGAGC, | |
| 1158, | EPI-37-076, | J03132, | ACACTTGAGCTCGGGCAATG, | |
| 1159, | EPI-37-077, | J03132, | TCGGGCAATGGGTTCCCCCA, | |
| 1160, | EPI-37-078, | J03132, | GGTTCCCCCAAGCCTGGCAC, | |
| 1161, | EPI-37-079, | J03132, | AGCCTGGCACATTGGAGTCT, | |
| 1162, | EPI-37-080, | J03132, | ATTGGAGTCTGCTGGGAATT, | |
| 1163, | EPI-37-081, | J03132, | GCTGGGAATTTTCTGGCCAC, | |
| 1164, | EPI-37-082, | J03132, | TTCTGGCCACGTCCAGTTTC, | |
| 1165, | EPI-37-083, | J03132, | GTCCAGTTTCCCGGACAATC, | |
| 1166, | EPI-37-084, | J03132, | CCGGACAATCCCTCTCGTCC, | |
| 1167, | EPI-37-085, | J03132, | CCTCTCGTCCAGTCGGGGGC, | |
| 1168, | EPI-37-086, | J03132, | AGTCGGGGGCCATACAGGAC, | |
| 1169, | EPI-37-087, | J03132, | CATACAGGACACGAAGCTCC, | |
| 1170, | EPI-37-088, | J03132, | ACGAAGCTCCCGGGTCTGGT, | |
| 1171, | EPI-37-089, | J03132, | CGGGTCTGGTTCTTGTGTAT, | |
| 1172, | EPI-37-090, | J03132, | TCTTGTGTATAAGCTGGCCG, | |
| 1173, | EPI-37-091, | J03132, | AAGCTGGCCGGCCACCTCCA, | |
| 1174, | EPI-37-092, | J03132, | GCCACCTCCAGGGTTGCAGA, | |
| 1175, | EPI-37-093, | J03132, | GGGTTGCAGAGCAGGAGAAG, | |
| 1176, | EPI-37-094, | J03132, | GCAGGAGAAGCTGCGCCCGT, | |
| 1177, | EPI-37-095, | J03132, | CTGCGCCCGTTGTCCTCTGG, | |
| 1178, | EPI-37-096, | J03132, | TGTCCTCTGGGGTGGCCTTC, | |
| 1179, | EPI-37-097, | J03132, | GGTGGCCTTCAGCAGGAGCT, | |
| 1180, | EPI-37-098, | J03132, | AGCAGGAGCTGGGCCCTCGG, | |
| 1181, | EPI-37-099, | J03132, | GGGCCCTCGGGCCCAGTGGC, | |
| 1182, | EPI-37-100, | J03132, | GCCCAGTGGCTGGGCTGGAA, | |
| 1183, | EPI-37-101, | J03132, | TGGGCTGGAACCCCATTCAG, | |
| 1184, | EPI-37-102, | J03132, | CCCCATTCAGCGTCACCTTG, | |
| 1185, | EPI-37-103, | J03132, | CGTCACCTTGGCTCTAGGGT, | |
| 1186, | EPI-37-104, | J03132, | GCTCTAGGGTGGGCCTCACA, | |
| 1187, | EPI-37-105, | J03132, | GGGCCTCACACTTCACTGTC, | |
| 1188, | EPI-37-106, | J03132, | CTTCACTGTCACCTCGGTCC, | |
| 1189, | EPI-37-107, | J03132, | ACCTCGGTCCCTTCTGAGAC, | |
| 1190, | EPI-37-108, | J03132, | CTTCTGAGACCTCTGGCTTC, | |
| 1191, | EPI-37-109, | J03132, | CTCTGGCTTCGTCAGAATCA, | |
| 1192, | EPI-37-110, | J03132, | GTCAGAATCACGTTGGGCGC, | |
| 1193, | EPI-37-111, | J03132, | CGTTGGGCGCCGGAAAGCTG, | |
| 1194, | EPI-37-112, | J03132, | CGGAAAGCTGTAGATGGTCA, | |
| 1195, | EPI-37-113, | J03132, | TAGATGGTCACTGTCTGCAG, | |
| 1196, | EPI-37-114, | J03132, | CTGTCTGCAGTGTCTCCTGG, | |
| 1197, | EPI-37-115, | J03132, | TGTCTCCTGGCTCTGGTTCC, | |
| 1198, | EPI-37-116, | J03132, | CTCTGGTTCCCCAGTATTAC, | |
| 1199, | EPI-37-117, | J03132, | CCAGTATTACTGCACACGTC, | |
| 1200, | EPI-37-118, | J03132, | TGCACACGTCAGCCGCTGGG, | |
| 1201, | EPI-37-119, | J03132, | AGCCGCTGGGTGCCCTCGTC, | |
| 1202, | EPI-37-120, | J03132, | TGCCCTCGTCCTCTGCGGTC, | |
| 1203, | EPI-37-121, | J03132, | CTCTGCGGTCACACTGACTG, | |
| 1204, | EPI-37-122, | J03132, | ACACTGACTGAGGCCTTGGC, | |
| 1205, | EPI-37-123, | J03132, | AGGCCTTGGCCGAGAAGGAG, | |
| 1206, | EPI-37-124, | J03132, | CGAGAAGGAGTCGTTGCCAT, | |
| 1207, | EPI-37-125, | J03132, | TCGTTGCCATAGGTGACTGT, | |
| 1208, | EPI-37-126, | J03132, | AGGTGACTGTGGGGTTCAAC, | |
| 1209, | EPI-37-127, | J03132, | GGGGTTCAACCTCTGGTCCC, | |
| 1210, | EPI-37-128, | J03132, | CTCTGGTCCCCCAGTGCCAG, | |
| 1211, | EPI-37-129, | J03132, | CCAGTGCCAGGTGGACCTGG, | |
| 1212, | EPI-37-130, | J03132, | GTGGACCTGGGCCTCCGAGA, | |
| 1213, | EPI-37-131, | J03132, | GCCTCCGAGACTGGGAACAG, | |
| 1214, | EPI-37-132, | J03132, | CTGGGAACAGCCCGTCCAGG, | |
| 1215, | EPI-37-133, | J03132, | CCCGTCCAGGGAACAGACCA, | |
| 1216, | EPI-37-134, | J03132, | GAACAGACCACGGTCCCCTG, | |
| 1217, | EPI-37-135, | J03132, | CGGTCCCCTGCGTGTCCACC, | |
| 1218, | EPI-37-136, | J03132, | CGTGTCCACCTCTAGGACCC, | |
| 1219, | EPI-37-137, | J03132, | TCTAGGACCCGGGGGCTGAC, | |
| 1220, | EPI-37-138, | J03132, | GGGGGCTGACAAGTTGTGGG, | |
| 1221, | EPI-37-139, | J03132, | AAGTTGTGGGGGAGTCGCTG, | |
| 1222, | EPI-37-140, | J03132, | GGAGTCGCTGGCAGGACAAA, | |
| 1223, | EPI-37-141, | J03132, | GCAGGACAAAGGTCTGGAGC, | |
| 1224, | EPI-37-142, | J03132, | GGTCTGGAGCTGGTAGGGGG, | |
| 1225, | EPI-37-143, | J03132, | TGGTAGGGGGCCGAGGTGTT, | |
| 1226, | EPI-37-144, | J03132, | CCGAGGTGTTCTCAAACAGC, | |
| 1227, | EPI-37-145, | J03132, | CTCAAACAGCTCCAGCCCTT, | |
| 1228, | EPI-37-146, | J03132, | TCCAGCCCTTGGGGCCGCAG, | |
| 1229, | EPI-37-147, | J03132, | TCTCTCCTCACCAGCACCGT, | |
| 1230, | EPI-37-148, | J03132, | CCAGCACCGTGGTCGTGACC, | |
| 1231, | EPI-37-149, | J03132, | GTGCGGCACGAGAAATTGGC, | |
| 1232, | EPI-37-150, | J03132, | AGAAATTGGCTCCATGGTGA, | |
| 1233, | EPI-37-151, | J03132, | TCCATGGTGATCTCTCCTCA, | |
| 1234, | EPI-37-152, | J03132, | TCTCTCCTCACCAGCACCGT, | |
| 1235, | EPI-37-153, | J03132, | CCAGCACCGTGGTCGTGACC, | |
| 1236, | EPI-37-154, | J03132, | GGTCGTGACCTCAGCGGGCT, | |
| 1237, | EPI-37-155, | J03132, | TCAGCGGGCTCCCCCACAGC, | |
| 1238, | EPI-37-156, | J03132, | CCCCCACAGCTGGCTCCCGT, | |
| 1239, | EPI-37-157, | J03132, | TGGCTCCCGTTTCAGCTCCT, | |
| 1240, | EPI-37-158, | J03132, | TTCAGCTCCTTCTCCCCACG, | |
| 1241, | EPI-37-159, | J03132, | TCTCCCCACGGAGCAGCACC, | |
| 1242, | EPI-37-160, | J03132, | GAGCAGCACCACGGTGAGGT, | |
| 1243, | EPI-37-161, | J03132, | ACGGTGAGGTTGGCCCGGGG, | |
| 1244, | EPI-37-162, | J03132, | TGGCCCGGGGTGCCCCACCC, | |
| 1245, | EPI-37-163, | J03132, | TGCCCCACCCTCCACCTGGC, | |
| 1246, | EPI-37-164, | J03132, | TCCACCTGGCAGCGTAGGGT, | |
| 1247, | EPI-37-165, | J03132, | AGCGTAGGGTAAGGTTCTTG, | |
| 1248, | EPI-37-166, | J03132, | AAGGTTCTTGCCCACTGGCT, | |
| 1249, | EPI-37-167, | J03132, | CCCACTGGCTGCCAAGAGGG, | |
| 1250, | EPI-37-168, | J03132, | GCCAAGAGGGGAGGGGTGCC, | |
| 1251, | EPI-37-169, | J03132, | GAGGGGTGCCAGTTCCACCC, | |
| 1252, | EPI-37-170, | J03132, | AGTTCCACCCGTTCTGGAGT, | |
| 1253, | EPI-37-171, | J03132, | GTTCTGGAGTCCAGTACACG, | |
| 1254, | EPI-37-172, | J03132, | CCAGTACACGGTGAGGAAGG, | |
| 1255, | EPI-37-173, | J03132, | GTGAGGAAGGTTTTAGCTGT, | |
| 1256, | EPI-37-174, | J03132, | TTTTAGCTGTTGACTGCCCA, | |
| 1257, | EPI-37-175, | J03132, | TGACTGCCCATCAGGGCAGT, | |
| 1258, | EPI-37-176, | J03132, | TCAGGGCAGTTTGAATAGCA, | |
| 1259, | EPI-37-177, | J03132, | TTGAATAGCACATTGGTTGG, | |
| 1260, | EPI-37-178, | J03132, | CATTGGTTGGCTATCTTCTT, | |
| 1261, | EPI-37-179, | J03132, | CTATCTTCTTGCACATTGCT, | |
| 1262, | EPI-37-180, | J03132, | GCACATTGCTCAGTTCATAC, | |
| 1263, | EPI-37-181, | J03132, | CAGTTCATACACCTTCCGGT, | |
| 1264, | EPI-37-182, | J03132, | ACCTTCCGGTTGTTCCCAGG, | |
| 1265, | EPI-37-183, | J03132, | TGTTCCCAGGCAGGAGCAAC, | |
| 1266, | EPI-37-184, | J03132, | CAGGAGCAACTCCTTTTTAG, | |
| 1267, | EPI-37-185, | J03132, | TCCTTTTTAGGCAACGGGGT, | |
| 1268, | EPI-37-186, | J03132, | GCAACGGGGTCTCTATGCCC, | |
| 1269, | EPI-37-187, | J03132, | CTCTATGCCCAACAACTTGG, | |
| 1270, | EPI-37-188, | J03132, | AACAACTTGGGCTGGTCACA, | |
| 1271, | EPI-37-189, | J03132, | GCTGGTCACAGGAGGTGCTG, | |
| 1272, | EPI-37-190, | J03132, | GGAGGTGCTGCATGTCACCA, | |
| 1273, | EPI-37-191, | J03132, | CATGTCACCAGCACGGAGCC, | |
| 1274, | EPI-37-192, | J03132, | GCACGGAGCCTCCCCGGGGC, | |
| 1275, | EPI-37-193, | J03132, | TCCCCGGGGCAGGATGACTT, | |
| 1276, | EPI-37-194, | J03132, | AGGATGACTTTTGAGGGGGA, | |
| 1277, | EPI-37-195, | J03132, | TTGAGGGGGACACAGATGTC, | |
| 1278, | EPI-37-196, | J03132, | CACAGATGTCTGGGCATTGC, | |
| 1279, | EPI-37-197, | J03132, | TGGGCATTGCCAGGTCCTGG, | |
| 1280, | EPI-37-198, | J03132, | CAGGTCCTGGGAACAGAGCC, | |
| 1281, | EPI-37-199, | J03132, | GAACAGAGCCCCGAGCAGGA, | |
| 1282, | EPI-37-200, | J03132, | CCGAGCAGGACCAGGAGTGC, | |
| 1283, | EPI-37-201, | J03132, | CCAGGAGTGCGGGCAGCGCG, | |
| 1284, | EPI-37-202, | J03132, | GGGCAGCGCGGGCCGGGGGC, | |
| 1285, | EPI-37-203, | J03132, | GGCCGGGGGCTGCTGGGAGC, | |
| 1286, | EPI-37-204, | J03132, | TGCTGGGAGCCATAGCGAGG, | |
| 1287, | EPI-37-205, | J03132, | CATAGCGAGGCTGAGGTTGC, | |
| 1288, | EPI-37-206, | J03132, | CTGAGGTTGCAACTCTGAGT, | |
| 1289, | EPI-37-207, | J03132, | AACTCTGAGTAGCAGAGGAG, | |
| 1290, | EPI-37-208, | J03132, | AGCAGAGGAGCTCAGCGTCG, | |
| 1291, | EPI-37-209, | J03132, | CTCAGCGTCGACTGGGGCGC, | |
| Concatemer Nucleic Acid Sequences of ICAM gene oligo sequences | | | | |
| (SEQ ID NO: 1292) | | | | |
| GGAAGTGTGGGCCTTTGTGTGCCTTTGTGTTTTGATGCTATTTGATGCTACACATGTCTACACATGTCTATGGAGGGCCATGGAGGGCCACTTCTTCTGTCTTCTTCTGTAAGTCTGTGGAAGTCTGTGGGGCC | | | | |
| TCAGCAGGCCTCAGCATACCCAATAGTACCCAATAGGCAGCAAGTTGCAGCAAGTTTCAGTATTTCTCAGTATTTCCCAGTTGTATCCAGTTGTATGTCCTCATGGGTCCTCATGGTGGGGCTATGTGGGGCTA | | | | |
| TGTCTCCCCCACTCTCCCCCACCACTTCCCCTCACTTCCCCTCTCATCAGGCCTCATCAGGCTAGACTTTAATAGACTTTAACATCCATCAACATCCATCAATCATGTCTTGTCATGTCTTGAGTCTTGCTCAG | | | | |
| TCTTGCTCCTTCCTCTTGCTTCCTCTTGGCTTAGTCATGCTTAGTCATGTGACTACAGGTGACTACAGATCAGATGCGATCAGATGCGTGGCCTAGTGTGGCCTAGTGTTTTAGGTGTTTTTAGGTGTGCAGGT | | | | |
| ACCAGCAGGTACCATGGCCCCAAATGGCCCCAAATGCTGTTGTATGCTGTTGTATCTGACTGAGTCTGACTGAGGACAATGCCCGACAATGCCCTGTCCTCCGGTGTCCTCCGGCGTCCCAGGGCGTCCCAGGG | | | | |
| CCGGTAGGTGCCGGTAGGTGTAGCTGCATGTAGCTGCATGGCATATGTCTGCATATGTCTTCCACTCTGTTCCACTCTGTTCAGTGTGGCTCAGTGTGGCACCACTGCCAACCACTGCCACCAATATGGGCCAA | | | | |
| TATGGGAAGGCCGAGGAAGGCCGAGGAAGAGGCCCTAAGAGGCCCTGTCCCGGGATGTCCCGGGATAGGTTCAGGGAGGTTCAGGGAGGCGTGGCTAGGCGTGGCTTGTGTGTTCGTGTGTGTTCGGTTTCATG | | | | |
| GGGTTTCATGGGGGTCCCTTTTGGTCCCTTTTTGGGCCTGTTTGGGCCTGTTGTAGTCTGTAGTAGTCTGTATTTCTTGATCTTTCTTGATCTTCCGCTGGCTTCCGCTGGCGGTTATAGAGGGTTATAGAGGT | | | | |
| ACGTGCTGGTACGTGCTGAGGCCTGCAGAGGCCTGCAGTGCCCATTATTGCCCATTATGACTGCGGCTGACTGCGGCTGCTACCACAGGCTACCACAGTGATGATGACTGATGATGACAATCTCATACAATCTC | | | | |
| ATACCGGGGGGAGACGGGGGGAGAGCACATTCACGCACATTCACGGTCACCTTGGGTCACCTTGCGGGTGACCTCGGGTGACCTCCCCTTGAGTCCCCTTGAGTGCTCCTGGCCGCTCCTGGCCCGACAGAGGT | | | | |
| CGACAGAGGTAGGTGCCCTCAGGTGCCCTCAAGATCTCGAAAGATCTCGAGTGACAGTCAGTGACAGTCACTGATTCCCCCTGATTCCCCGATGGGCAGTGATGGGCAGTGGGAAAGTGCGGGAAAGTGCCATC | | | | |
| CTTTAGCATCCTTTAGACACTTGAGCACACTTGAGCTCGGGCAATGTCGGGCAATGGGTTCCCCCAGGTTCCCCCAAGCCTGGCACAGCCTGGCACATTGGAGTCTATTGGAGTCTGCTGGGAATTGCTGGGAA | | | | |
| TTTTCTGGCCACTTCTGGCCACGTCCAGTTTCGTCCAGTTTCCCGGACAATCCCGGACAATCCCTCTCGTCCCCTCTCGTCCAGTCGGGGGCAGTCGGGGGCCATACAGGACCATACAGGACACGAAGCTCCAC | | | | |
| GAAGCTCCCGGGTCTGGTCGGGTCTGGTTCTTGTGTATTCTTGTGTATAAGCTGGCCGAAGCTGGCCGGCCACCTCCAGCCACCTCCAGGGTTGCAGAGGGTTGCAGAGCAGGAGAAGGCAGGAGAAGCTGCGC | | | | |
| CCGTCTGCGCCCGTTGTCCTCTGGTGTCCTCTGGGGTGGCCTTCGGTGGCCTTCAGCAGGAGCTAGCAGGAGCTGGGCCCTCGGGGGCCCTCGGGCCCAGTGGCGCCCAGTGGCTGGGCTGGAATGGGCTGGAA | | | | |
| CCCCATTCAGCCCCATTCAGCGTCACCTTGCGTCACCTTGGCTCTAGGGTGCTCTAGGGTGGGCCTCACAGGGCCTCACACTTCACTGTCCTTCACTGTCACCTCGGTCCACCTCGGTCCCTTCTGAGACCTTC | | | | |
| TGAGACCTCTGGCTTCCTCTGGCTTCGTCAGAATCAGTCAGAATCACGTTGGGCGCCGTTGGGCGCCGGAAAGCTGCGGAAAGCTGTAGATGGTCATAGATGGTCACTGTCTGCAGCTGTCTGCAGTGTCTCCT | | | | |
| GGTGTCTCCTGGCTCTGGTTCCCTCTGGTTCCCCAGTATTACCCAGTATTACTGCACACGTCTGCACACGTCAGCCGCTGGGAGCCGCTGGGTGCCCTCGTCTGCCCTCGTCCTCTGCGGTCCTCTGCGGTCAC | | | | |
| ACTGACTGACACTGACTGAGGCCTTGGCAGGCCTTGGCCGAGAAGGAGCGAGAAGGAGTCGTTGCCATTCGTTGCCATAGGTGACTGTAGGTGACTGTGGGGTTCAACGGGGTTCAACCTCTGGTCCCCTCTGG | | | | |
| TCCCCCAGTGCCAGCCAGTGCCAGGTGGACCTGGGTGGACCTGGGCCTCCGAGAGCCTCCGAGACTGGGAACAGCTGGGAACAGCCCGTCCAGGCCCGTCCAGGGAACAGACCAGAACAGACCACGGTCCCCTG | | | | |
| CGGTCCCCTGCGTGTCCACCCGTGTCCACCTCTAGGACCCTCTAGGACCCGGGGGCTGACGGGGGCTGACAAGTTGTGGGAAGTTGTGGGGGAGTCGCTGGGAGTCGCTGGCAGGACAAAGCAGGACAAAGGTC | | | | |
| TGGAGCGGTCTGGAGCTGGTAGGGGGTGGTAGGGGGCCGAGGTGTTCCGAGGTGTTCTCAAACAGCCTCAAACAGCTCCAGCCCTTTCCAGCCCTTGGGGCCGCAGGGGGCCGCAGGTCCAGTTCAGTCCAGTT | | | | |
| CAGTGCGGCACGGTGCGGCACGAGAAATTGGCAGAAATTGGCTCCATGGTGATCCATGGTGATCTCTCCTCATCTCTCCTCACCAGCACCGTCCAGCACCGTGGTCGTGACCGGTCGTGACCTCAGCGGGCTTC | | | | |
| AGCGGGCTCCCCCACAGCCCCCCACAGCTGGCTCCCGTTGGCTCCCGTTTCAGCTCCTTTCAGCTCCTTCTCCCCACGTCTCCCCACGGAGCAGCACCGAGCAGCACCACGGTGAGGTACGGTGAGGTTGGCCC | | | | |
| GGGGTGGCCCCGGGGTGCCCACCCTGCCCCACCCTCCACCTGGCTCCACCTGGCAGCGTAGGGTAGCGTAGGGTAAGGTTCTTGAAGGTTCTTGCCCACTGGCTCCCACTGGCTGCCAAGAGGGGCCAAGAGGG | | | | |
| GAGGGGTGCCGAGGGGTGCCAGTTCCACCCAGTTCCACCCGTTCTGGAGTGTTCTGGAGTCCAGTACACGCCAGTACACGGTGAGGAAGGGTGAGGAAGGTTTTAGCTGTTTTTAGCTGTTGACTGCCCATGAC | | | | |
| TGCCCATCAGGGCAGTTCAGGGCAGTTTGAATAGCATTGAATAGCACATTGGTTGGCATTGGTTGGCTATCTTCTTCTATCTTCTTGCACATTGCTGCACATTGCTCAGTTCATACCAGTTCATACACCTTCCG | | | | |
| GTACCTTCCGGTTGTTCCCAGGTGTTCCCAGGCAGGAGCAACCAGGAGCAACTCCTTTTTAGTCCTTTTTAGGCAACGGGGTGCAACGGGGTCTCTATGCCCCTCTATGCCCAACAACTTGGAACAACTTGGGC | | | | |
| TGGTCACAGCTGGTCACAGGAGGTGCTGGGAGGTGCTGCATGTCACCACATGTCACCAGCACGGAGCCGCACGGAGCCTCCCCGGGGCTCCCCGGGGCAGGATGACTTAGGATGACTTTTGAGGGGGATTGAGG | | | | |
| GGGACACAGATGTCCACAGATGTCTGGGCATTGCTGGGCATTGCCAGGTCCTGGCAGGTCCTGGGAACAGAGCCGAACAGAGCCCCGAGCAGGACCGAGCAGGACCAGGAGTGCCCAGGAGTGCGGGCAGCGCG | | | | |
| GGGCAGCGCGGGCCGGGGGCGGCCGGGGGCTGCTGGGAGCTGCTGGGAGCCATAGCGAGGCATAGCGAGGCTGAGGTTGCCTGAGGTTGCAACTCTGAGTAACTCTGAGTAGCAGAGGAGAGCAGAGGAGCTCA | | | | |
| GCGTCGCTCAGCGTCGACTGGGGCGC | | | | |
| VCAM Nucleic Acid Sequences (GENBANK ACCESSION NO.X61177) | | | | |
| (SEQ ID NO: 1293) | | | | |
| ATGCCTGGGAAGATGGTCGTGATCCTTGGAGCCTCAAATATACTTTGGATAATGTTTGCAGCTTCTCAAGCTTTTAAAATCGAGACCACCCCAGAATCTAGATATCTTGCTCAGATTGGTGACTCCGTCTCATT | | | | |
| GACTTGCAGCACCACAGGCTGTGAGTCCCCATTTTTCTCTTGGAGAACCCAGATAGATAGTCCACTGAATGGGAAGGTGACGAATGAGGGGACCACATCTACGCTGACAATGAATCCTGTTAGTTTTGGGAACG | | | | |
| AACACTCTTACCTGTGCACAGCAACTTGTGAATCTAGGAAATTGGAAAAAGGAATCCAGGTGGAGATCTACTCTTTTCCTAAGGATCCAGAGATTCATTTGAGTGGCCCTCTGGAGGCTGGGAAGCCGATCACA | | | | |
| GTCAAGTGTTCAGTTGCTGATGTATACCCATTTGACAGGCTGGAGATAGACTTACTGAAAGGAGATCATCTCATGAAGAGTCAGGAATTTCTGGAGGATGCAGACAGGAAGTCCCTGGAAACCAAGAGTTTGGA | | | | |
| AGTAACCTTTACTCCTGTCATTGAGGATATTGGAAAAGTTCTTGTTTGCCGAGCTAAATTACACATTGATGAAATGGATTCTGTGCCCACAGTAAGGCAGGCTGTAAAAGAATTGCAAGTCTACATATCACCCA | | | | |
| AGAATACAGTTATTTCTGTGAATCCATCCACAAAGCTGCAAGAAGGTGGCTCTGTGACCATGACCTGTTCCAGCGAGGGTCTACCAGCTCCAGAGATTTTCTGGAGTAAGAAATTAGATAATGGGAATCTACAG | | | | |
| CACCTTTCTGGAAATGCAACTCTCACCTTAATTGCTATGAGGATGGAAGATTCTGGAATTTATGTGTGTGAAGGAGTTAATTTGATTGGGAAAAACAGAAAAGAGGTGGAATTAATTGTTCAAGAGAAACCATT | | | | |
| TACTGTTGAGATCTCCCCTGGACCCCGGATTGCTGCTCAGATTGGAGACTCAGTCATGTTGACATGTAGTGTCATGGGCTGTGAATCCCCATCTTTCTCCTGGAGAACCCAGATAGACAGCCCTCTGAGCGGGA | | | | |
| AGGTGAGGAGTGAGGGGACCAATTCCACGCTGACCCTGAGCCCTGTGAGTTTTGAGAACGAACACTCTTATCTGTGCACAGTGACTTGTGGACATAAGAAACTGGAAAAGGGAATCCAGGTGGAGCTCTACTCA | | | | |
| TTCCCTAGAGATCCAGAAATCGAGATGAGTGGTGGCCTCGTGAATGGGAGCTCTGTCACTGTAAGCTGCAAGGTTCCTAGCGTGTACCCCCTTGACCGGCTGGAGATTGAATTACTTAAGGGGGAGACTATTCT | | | | |
| GGAGAATATAGAGTTTTTGGAGGATACGGATATGAAATCTCTAGAGAACAAAAGTTTGGAAATGACCTTCATCCCTACCATTGAAGATACTGGAAAAGCTCTTGTTTGTCAGGCTAAGTTACATATTGATGACA | | | | |
| TGGAATTCGAACCCAAACAAAGGCAGAGTACGCAAACACTTTATGTCAATGTTGCCCCCAGAGATACAACCGTCTTGGTCAGCCCTTCCTCCATCCTGGAGGAAGGCAGTTCTGTGAATATGACATGCTTGAGC | | | | |
| CAGGGCTTTCCTGCTCCGAAAATCCTGTGGAGCAGGCAGCTCCCTAACGGGGAGCTACAGCCTCTTTCTGAGAATGCAACTCTCACCTTAATTTCTACAAAAATGGAAGATTCTGGGGTTTATTTATGTGAAGG | | | | |
| AATTAACCAGGCTGGAAGAAGCAGAAAGGAAGTGGAATTAATTATCCAAGTTACTCCAAAAGACATAAAACTTACAGCTTTTCCTTCTGAGAGTGTCAAAGAAGGAGACACTGTCATCATCTCTTGTACATGTG | | | | |
| GAAATGTTCCAGAAACATGGATAATCCTGAAGAAAAAAGCGGAGACAGGAGACACAGTACTAAAATCTATAGATGGCGCCTATACCATCCGAAAGGCCCAGTTGAAGGATGCGGGAGTATATGAATGTGAATCT | | | | |
| AAAAACAAAGTTGGCTCACAATTAAGAAGTTTAACACTTGATGTTCAAGGAAGAGAAAACAACAAAGACTATTTTTCTCCTGAGCTTCTCGTGCTCTATTTTGCATCCTCCTTAATAATACCTGCCATTGGAAT | | | | |
| GATAATTTACTTTGCAAGAAAAGCCAACATGAAGGGGTCATATAGTCTTGTAGAAGCACAGAAATCAAAAGTGTAG | | | | |
| 1294, | EPI-3-029, | X53051, | TTTAGTACTGTGTCTCCTGT, | |
| 1295, | EPI-3-046, | X53051, | CTTTCTGCTTCTTCCAGCCT, | |
| 1296, | EPI-3-047, | X53051, | CTTCCAGCCTGGTTAATTCC, | |
| 1297, | EPI-3-072, | X53051, | TTTGCGTACTCTGCCTTTGT, | |
| 1298, | EPI-3-073, | X53051, | CTGCCTTTGTTTGGGTTCGA, | |
| 1299, | EPI-3-081, | X53051, | TGGTAGGGATGAAGGTCATT, | |
| 1300, | EPI-3-084, | X53051, | TGTTCTCTAGAGATTTCATA, | |
| 1301, | EPI-3-085, | X53051, | AGATTTCATATCCGTATCCT, | |
| 1302, | EPI-3-087, | X53051, | CCAAAAACTCTATATTCTCC, | |
| 1303, | EPI-3-088, | X53051, | TATATTCTCCAGAATAGTCT, | |
| 1304, | EPI-3-091, | X53051, | TAATTCAATCTCCAGCCGGT, | |
| 1305, | EPI-3-094, | X53051, | CACGCTAGGAACCTTGCAGC, | |
| 1306, | EPI-3-098, | X53051, | TTCACGAGGCCACCACTCAT, | |
| 1307, | EPI-3-099, | X53051, | CACCACTCATCTCGATTTCT, | |
| 1308, | EPI-3-116, | X53051, | CCGCTCAGAGGGCTGTCTAT, | |
| 1309, | EPI-3-117, | X53051, | GGCTGTCTATCTGGGTTCTC, | |
| 1310, | EPI-3-118, | X53051, | CTGGGTTCTCCAGGAGAAAG, | |
| 1311, | EPI-3-128, | X53051, | ATCTCAACAGTAAATGGTTT, | |
| 1312, | EPI-3-137, | X53051, | CCAGAATCTTCCATCCTCAT, | |
| 1313, | EPI-3-159, | X53051, | CAGCCTGCCTTACTGTGGGC, | |
| 1314, | EPI-3-160, | X53051, | TACTGTGGGCACAGAATCCA, | |
| 1315, | EPI-3-193, | X53051, | TTCACAAGTTGCTGTGCACA, | |
| 1316, | EPI-3-194, | X53051, | GCTGTGCACAGGTAAGAGTG, | |
| 1317, | EPI-3-196, | X53051, | TTCGTTCCCAAAACTAACAG, | |
| 1318, | EPI-3-213, | X53051, | TAGATTCTGGGGTGGTCTCG, | |
| Concatemer Nucleic Acid Sequences of VCAM gene oligo sequences | | | | |
| (SEQ ID NO: 1319) | | | | |
| TTTAGTACTGTGTCTCCTGTCTTTCTGCTTCTTCCAGCCTCTTCCAGCCTGGTTAATTCCTTTGCGTACTCTGCCTTTGTCTGCCTTTGTTTGGGTTCGATGGTAGGGATGAAGGTCATTTGTTCTCTAGAGAT | | | | |
| TTCATAAGATTTCATATCCGTATCCTCCAAAAACTCTATATTCTCCTATATTCTCCAGAATAGTCTTAATTCAATCTCCAGCCGGTCACGCTAGGAACCTTGCAGCTTCACGAGGCCACCACTCATCACCACTC | | | | |
| ATCTCGATTTCTCCGCTCAGAGGGCTGTCTATGGCTGTCTATCTGGGTTCTCCTGGGTTCTCCAGGAGAAAGATCTCAACAGTAAATGGTTTCCAGAATCTTCCATCCTCATCAGCCTGCCTTACTGTGGGCTA | | | | |
| CTGTGGGCACAGAATCCATTCACAAGTTGCTGTGCACAGCTGTGCACAGGTAAGAGTGTTCGTTCCCAAAACTAACAGTAGATTCTGGGGTGGTCTCG | | | | |
| Tryptase-a Nucleic Acid Sequences (GENBANK ACCESSION NO.X61177) | | | | |
| (SEQ ID NO: 1320) | | | | |
| ACCAGCTGACAGGTGGAGCTGCCAGTCTCCAGTGCTCAGCCCTCAGCGGGGCCTGCCTGGCAGCCCCACACACAGAGGGCATCGGGGTGGCGGGGGCACGTGTTACACGGGGGCCCTGGGTCTGAGTCATCCAC | | | | |
| TTCCTCCGAGTCTGGATGGGAGGACCCAGCGCCCCCTCCTCCGCCCCCTCCTGATCTGGAGGATAAATGGGGAGGGAGAGCCACTGGGTAGAAGGAACAGGGAGTGGCCAGGGTAAGTCCCCCACTCTCAGAGA | | | | |
| CCTGACATCAGCGTCACCTGGAGCAGAGTGGCCCAGCCTCAGACTCAGAGCACCAAGACCCAGGCCCGCAGGCCTGGACCCACCCCGGTCCCCCCCGTCCCAGCTCCATTCTTCACCCCACAATCTGTAGCCCC | | | | |
| CAGCCCTGCCCTGTGAGGCCCGGCCAGGCCCACGATGCTCCTCCTTGCTCCCCAGATGCTGAATCTGCTGCTGCTGGCGCTGCCCGTCCTGGCGAGCCGCGCCTACGCGGCCCCTGGTGAGTCCCCAGCCGGGT | | | | |
| CCACCCTGCCCCTCACCACATTCCACAGGTCAGGGCCTGGGTGGGTTCTGGGGAGGTCGGGCTGGCCCCCCACACAGGGAAGGGCTGGGCCCAGGCCTGGGGCTGCTTCCTGGTCCTGACCTGGCACCTGCCCC | | | | |
| AGCCCCAGGCCAGGCCCTGCAGCGAGTGGGCATCGTCGGGGGTCAGGAGGCCCCCAGGAGCAAGTGGCCCTGGCAGGTGAGCCTGAGAGTCCACGGCCCATACTGGATGCACTTCTGCGGGGGCTCCCTCATCC | | | | |
| ACCCCCAGTGGGTGCTGACCGCAGCGCACTGCGTGGGACCGTGAGTCTCCCGGGGCCTGGAGGGGTGGGGAAGGGCTGGATGTGAGCCCTGGCTCCCGGGTGCTCCTGGGGGCTGCCCAGGGCCCTGAGTGGGA | | | | |
| TCCTCCGCTGCCCAGGGACGTCAAGGATCTGGCCGCCCTCAGGGTGCAACTGCGGGAGCAGCACCTCTACTACCAGGACCAGCTGCTGCCGGTCAGCAGGATCATCGTGCACCCACAGTTCTACACCGCCCAGA | | | | |
| TCGGAGCGGACATCGCCCTGCTGGAGCTGGAGGAGCCGGTGAACGTCTCCAGCCACGTCCACACGGTCACCCTGCCCCCTGCCTCAGAGACCTTCCCCCCGGGGATGCCGTGCTGGGTCACTGGCTGGGGCGAT | | | | |
| GTGGACAATGATGGTGGGTCTGGGGACAGTGGAGGTGGGGCCAGGGTCTTAGCCACAGCCCAGCCCCTGGGTCCCTCTGGGCTCCAGGTGGGGGTTGCCCGGCCCCCTCCTGAGGCTGCACCCTCTTCCCCACC | | | | |
| TGCAGAGCGCCTCCCACCGCCATTTCCTCTGAAGCAGGTGAAGGTCCCCATAATGGAAAACCACATTTGTGACGCAAAATACCACCTTGGCGCCTACACGGGAGACGACGTCCGCATCGTCCGTGACGACATGC | | | | |
| TGTGTGCCGGGAACACCCGGAGGGACTCATGCCAGGTGGGCCCCGCCTGTCCCCCGCCCCCCGCCCCCCAACCCCCACTCCCAGGCCTGTTCGGCGAGCGCTGACCTCTGACCTTCCCAGGGCGACTCCGGAGG | | | | |
| GCCCCTGGTGTGCAAGGTGAATGGCACCTGGCTGCAGGCGGGCGTGGTCAGCTGGGGCGAGGGCTGTGCCCAGCCCAACCGGCCTGGCATCTACACCCGTGTCACCTACTACTTGGACTGGATCCACCACTATG | | | | |
| TCCCCAAAAAGCCGTGAGTCAGGCCTGGGTTGGCCACCTGGGTCACTGGAGGACCAACCCCTGCTGTCCAAAACACCACTGCTTCCTACCCAGGTGGCGACTGCCCCCCACACCTTCCCTGCCCCGTCCTGAGT | | | | |
| GCCCCTTCCTGTCCTAAGCCCCCTGCTCTCTTCTGAGCCCCTTCCCCTGTCCTGAGGACCCTTCCCTATCCTGAGCCCCCTTCCCTGTCCTAAGCCTGACGCCTGCACCGGGCCCTCCAGCCCTCCCCTGCCCA | | | | |
| GATAGCTGGTGGTGGGCGCTAATCCTCCTGAGTGCTGGACCTCATTAAAGTGCATGGAAATCACTGGTGTGCATCGCTGTGTTTCTGGTTGTGGATGTCACTGGGAGAGAAGGGGTCCAGGTGTGCTGAGGACA | | | | |
| CCTGCCACAGTGTGAGGTCCTAGCCCTCAAGGCACAGCCAGTCACCGTGGGAC | | | | |
| 1321, | EPI-15-001, | M33494, | AGGCTCAGCATCCTGGCCAC, | |
| 1322, | EPI-15-002, | M33494, | GCAGCAGGCTCAGCATCCTG, | |
| 1323, | EPI-15-003, | M33494, | CAGCAGCAGCAGGCTCAGCA, | |
| 1324, | EPI-15-004, | M33494, | AGCGCCAGCAGCAGCAGGCT, | |
| 1325, | EPI-15-005, | M33494, | CGGGCAGCGCCAGCAGCAGC, | |
| 1326, | EPI-15-006, | M33494, | CAGGACGGGCAGCGCCAGCA, | |
| 1327, | EPI-15-007, | M33494, | CTCGCCAGGACGGGCAGCGC, | |
| 1328, | EPI-15-008, | M33494, | CGCGGCTCGCCAGGACGGGC, | |
| 1329, | EPI-15-009, | M33494, | GTAGGCGCGGCTCGCCAGGA, | |
| 1330, | EPI-15-010, | M33494, | GCCGCGTAGGCGCGGCTCGC, | |
| 1331, | EPI-15-011, | M33494, | CAGGGGCCGCGTAGGCGCGG, | |
| 1332, | EPI-15-012, | M33494, | TGGGGCAGGGGCCGCGTAGG, | |
| 1333, | EPI-15-013, | M33494, | TGGACTGGGGCAGGGGCCGC, | |
| 1334, | EPI-15-014, | M33494, | GGGCCTGGACTGGGGCAGGG, | |
| 1335, | EPI-15-015, | M33494, | CTGCAGGGCCTGGACTGGGG, | |
| 1336, | EPI-15-016, | M33494, | GCTTGCTGCAGGGCCTGGAC, | |
| 1337, | EPI-15-017, | M33494, | TACCCGCTTGCTGCAGGGCC, | |
| 1338, | EPI-15-018, | M33494, | GACGATACCCGCTTGCTGCA, | |
| 1339, | EPI-15-019, | M33494, | CCCCCGACGATACCCGCTTG, | |
| 1340, | EPI-15-020, | M33494, | CCTGACCCCCGACGATACCC, | |
| 1341, | EPI-15-021, | M33494, | GGCCTCCTGACCCCCGACGA, | |
| 1342, | EPI-15-022, | M33494, | CTGGGGGCCTCCTGACCCCC, | |
| 1343, | EPI-15-023, | M33494, | TGCTCCTGGGGGCCTCCTGA, | |
| 1344, | EPI-15-024, | M33494, | CCACTTGCTCCTGGGGGCCT, | |
| 1345, | EPI-15-025, | M33494, | CAGGGCCACTTGCTCCTGGG, | |
| 1346, | EPI-15-026, | M33494, | CCTGCCAGGGCCACTTGCTC, | |
| 1347, | EPI-15-027, | M33494, | GCTCACCTGCCAGGGCCACT, | |
| 1348, | EPI-15-028, | M33494, | CTCAGGCTCACCTGCCAGGG, | |
| 1349, | EPI-15-029, | M33494, | GGACTCTCAGGCTCACCTGC, | |
| 1350, | EPI-15-030, | M33494, | GTCGCGGACTCTCAGGCTCA, | |
| 1351, | EPI-15-031, | M33494, | TATCGGTCGCGGACTCTCAG, | |
| 1352, | EPI-15-032, | M33494, | TCCAGTATCGGTCGCGGACT, | |
| 1353, | EPI-15-033, | M33494, | GTGCATCCAGTATCGGTCGC, | |
| 1354, | EPI-15-034, | M33494, | CAGAAGTGCATCCAGTATCG, | |
| 1355, | EPI-15-035, | M33494, | CCCCGCAGAAGTGCATCCAG, | |
| 1356, | EPI-15-036, | M33494, | GGAGCCCCCGCAGAAGTGCA, | |
| 1357, | EPI-15-037, | M33494, | ATGAGGGAGCCCCCGCAGAA, | |
| 1358, | EPI-15-038, | M33494, | GGTGGATGAGGGAGCCCCCG, | |
| 1359, | EPI-15-039, | M33494, | CTGGGGGTGGATGAGGGAGC, | |
| 1360, | EPI-15-040, | M33494, | ACCCACTGGGGGTGGATGAG, | |
| 1361, | EPI-15-041, | M33494, | TCAGCACCCACTGGGGGTGG, | |
| 1362, | EPI-15-042, | M33494, | CGCGGTCAGCACCCACTGGG, | |
| 1363, | EPI-15-043, | M33494, | TGCGCCGCGGTCAGCACCCA, | |
| 1364, | EPI-15-044, | M33494, | GGCAGTGCGCCGCGGTCAGC, | |
| 1365, | EPI-15-045, | M33494, | TCCCAGGCAGTGCGCCGCGG, | |
| 1366, | EPI-15-046, | M33494, | TCCGGTCCCAGGCAGTGCGC, | |
| 1367, | EPI-15-047, | M33494, | TGACGTCCGGTCCCAGGCAG, | |
| 1368, | EPI-15-048, | M33494, | ATCCTTGACGTCCGGTCCCA, | |
| 1369, | EPI-15-049, | M33494, | GCCAGATCCTTGACGTCCGG, | |
| 1370, | EPI-15-050, | M33494, | GGGTGGCCAGATCCTTGACG, | |
| 1371, | EPI-15-051, | M33494, | CCTGAGGGTGGCCAGATCCT, | |
| 1372, | EPI-15-052, | M33494, | TGCACCCTGAGGGTGGCCAG, | |
| 1373, | EPI-15-053, | M33494, | GCAGTTGCACCCTGAGGGTG, | |
| 1374, | EPI-15-054, | M33494, | CTCCCGCAGTTGCACCCTGA, | |
| 1375, | EPI-15-055, | M33494, | TGCTGCTCCCGCAGTTGCAC, | |
| 1376, | EPI-15-056, | M33494, | AGAGGTGCTGCTCCCGCAGT, | |
| 1377, | EPI-15-057, | M33494, | GTAGTAGAGGTGCTGCTCCC, | |
| 1378, | EPI-15-058, | M33494, | TCCTGGTAGTAGAGGTGCTG, | |
| 1379, | EPI-15-059, | M33494, | GCTGGTCCTGGTAGTAGAGG, | |
| 1380, | EPI-15-060, | M33494, | CAGCAGCTGGTCCTGGTAGT, | |
| 1381, | EPI-15-061, | M33494, | ACTGGCAGCAGCTGGTCCTG, | |
| 1382, | EPI-15-062, | M33494, | TGCTGACTGGCAGCAGCTGG, | |
| 1383, | EPI-15-063, | M33494, | GATCCTGCTGACTGGCAGCA, | |
| 1384, | EPI-15-064, | M33494, | ACGATGATCCTGCTGACTGG, | |
| 1385, | EPI-15-065, | M33494, | GGTGCACGATGATCCTGCTG, | |
| 1386, | EPI-15-066, | M33494, | CTGTGGGTGCACGATGATCC, | |
| 1387, | EPI-15-067, | M33494, | TAGAACTGTGGGTGCACGAT, | |
| 1388, | EPI-15-068, | M33494, | TGATGTAGAACTGTGGGTGC, | |
| 1389, | EPI-15-069, | M33494, | CTGGATGATGTAGAACTGTG, | |
| 1390, | EPI-15-070, | M33494, | CCAGTCTGGATGATGTAGAA, | |
| 1391, | EPI-15-071, | M33494, | CCGCTCCAGTCTGGATGATG, | |
| 1392, | EPI-15-072, | M33494, | GATATCCGCTCCAGTCTGGA, | |
| 1393, | EPI-15-073, | M33494, | AGGGCGATATCCGCTCCAGT, | |
| 1394, | EPI-15-074, | M33494, | CCAGCAGGGCGATATCCGCT, | |
| 1395, | EPI-15-075, | M33494, | CAGCTCCAGCAGGGCGATAT, | |
| 1396, | EPI-15-076, | M33494, | TCCTCCAGCTCCAGCAGGGC, | |
| 1397, | EPI-15-077, | M33494, | CGGGCTCCTCCAGCTCCAGC, | |
| 1398, | EPI-15-078, | M33494, | GTTCACGGGCTCCTCCAGCT, | |
| 1399, | EPI-15-079, | M33494, | GAGATGTTCACGGGCTCCTC, | |
| 1400, | EPI-15-080, | M33494, | GGCTGGAGATGTTCACGGGC, | |
| 1401, | EPI-15-081, | M33494, | GACGCGGCTGGAGATGTTCA, | |
| 1402, | EPI-15-082, | M33494, | GTGTGGACGCGGCTGGAGAT, | |
| 1403, | EPI-15-083, | M33494, | TGACCGTGTGGACGCGGCTG, | |
| 1404, | EPI-15-084, | M33494, | CAGCATGACCGTGTGGACGC, | |
| 1405, | EPI-15-085, | M33494, | GGGGGCAGCATGACCGTGTG, | |
| 1406, | EPI-15-086, | M33494, | AGGCAGGGGGCAGCATGACC, | |
| 1407, | EPI-15-087, | M33494, | CTCCGAGGCAGGGGGCAGCA, | |
| 1408, | EPI-15-088, | M33494, | AAGGTCTCCGAGGCAGGGGG, | |
| 1409, | EPI-15-089, | M33494, | GGGGGAAGGTCTCCGAGGCA, | |
| 1410, | EPI-15-090, | M33494, | AGGCAGGGGGCAGCATGACC, | |
| 1411, | EPI-15-091, | M33494, | GGCATCCCCGGGGGGAAGGT, | |
| 1412, | EPI-15-092, | M33494, | AGCACGGCATCCCCGGGGGG, | |
| 1413, | EPI-15-093, | M33494, | GACCCAGCACGGCATCCCCG, | |
| 1414, | EPI-15-094, | M33494, | CCAGTGACCCAGCACGGCAT, | |
| 1415, | EPI-15-095, | M33494, | CCCAGCCAGTGACCCAGCAC, | |
| 1416, | EPI-15-096, | M33494, | ATCGCCCCAGCCAGTGACCC, | |
| 1417, | EPI-15-097, | M33494, | TCCACATCGCCCCAGCCAGT, | |
| 1418, | EPI-15-098, | M33494, | CATTGTCCACATCGCCCCAG, | |
| 1419, | EPI-15-099, | M33494, | CTCATCATTGTCCACATCGC, | |
| 1420, | EPI-15-100, | M33494, | AGGGGCTCATCATTGTCCAC, | |
| 1421, | EPI-15-101, | M33494, | GTGGGAGGGGCTCATCATTG, | |
| 1422, | EPI-15-102, | M33494, | TGGCGGTGGGAGGGGCTCAT, | |
| 1423, | EPI-15-103, | M33494, | GGAAATGGCGGTGGGAGGGG, | |
| 1424, | EPI-15-104, | M33494, | TCAGGGGAAATGGCGGTGGG, | |
| 1425, | EPI-15-105, | M33494, | CTGCTTCAGGGGAAATGGCG, | |
| 1426, | EPI-15-106, | M33494, | TTCACCTGCTTCAGGGGAAA, | |
| 1427, | EPI-15-107, | M33494, | GGACCTTCACCTGCTTCAGG, | |
| 1428, | EPI-15-108, | M33494, | TATGGGGACCTTCACCTGCT, | |
| 1429, | EPI-15-109, | M33494, | TCCATTATGGGGACCTTCAC, | |
| 1430, | EPI-15-110, | M33494, | GGTTTTCCATTATGGGGACC, | |
| 1431, | EPI-15-111, | M33494, | AATGTGGTTTTCCATTATGG, | |
| 1432, | EPI-15-112, | M33494, | TCACAAATGTGGTTTTCCAT, | |
| 1433, | EPI-15-113, | M33494, | TTGCGTCACAAATGTGGTTT, | |
| 1434, | EPI-15-114, | M33494, | GTATTTTGCGTCACAAATGT, | |
| 1435, | EPI-15-115, | M33494, | AGGTGGTATTTTGCGTCACA, | |
| 1436, | EPI-15-116, | M33494, | CGCCAAGGTGGTATTTTGCG, | |
| 1437, | EPI-15-117, | M33494, | GTAGGCGCCAAGGTGGTATT, | |
| 1438, | EPI-15-118, | M33494, | CCCGTGTAGGCGCCAAGGTG, | |
| 1439, | EPI-15-119, | M33494, | CGTCTCCCGTGTAGGCGCCA, | |
| 1440, | EPI-15-120, | M33494, | GACGTCGTCTCCCGTGTAGG, | |
| 1441, | EPI-15-121, | M33494, | ATGCGGACGTCGTCTCCCGT, | |
| 1442, | EPI-15-122, | M33494, | GGATGATGCGGACGTCGTCT, | |
| 1443, | EPI-15-123, | M33494, | GTCACGGATGATGCGGACGT, | |
| 1444, | EPI-15-124, | M33494, | ATGTCGTCACGGATGATGCG, | |
| 1445, | EPI-15-125, | M33494, | ACAGCATGTCGTCACGGATG, | |
| 1446, | EPI-15-126, | M33494, | GGCACACAGCATGTCGTCAC, | |
| 1447, | EPI-15-127, | M33494, | TTCCCGGCACACAGCATGTC, | |
| 1448, | EPI-15-128, | M33494, | GGCTGTTCCCGGCACACAGC, | |
| 1449, | EPI-15-129, | M33494, | CCTCTGGCTGTTCCCGGCAC, | |
| 1450, | EPI-15-130, | M33494, | GAGTCCCTCTGGCTGTTCCC, | |
| 1451, | EPI-15-131, | M33494, | TGCAGGAGTCCCTCTGGCTG, | |
| 1452, | EPI-15-132, | M33494, | GCCCTTGCAGGAGTCCCTCT, | |
| 1453, | EPI-15-133, | M33494, | GAGTCGCCCTTGCAGGAGTC, | |
| 1454, | EPI-15-134, | M33494, | CTCCAGAGTCGCCCTTGCAG, | |
| 1455, | EPI-15-135, | M33494, | GGGCCCTCCAGAGTCGCCCT, | |
| 1456, | EPI-15-136, | M33494, | ACCAGGGGCCCTCCAGAGTC, | |
| 1457, | EPI-15-137, | M33494, | TGCACACCAGGGGCCCTCCA, | |
| 1458, | EPI-15-138, | M33494, | CACCTTGCACACCAGGGGCC, | |
| 1459, | EPI-15-139, | M33494, | CCATTCACCTTGCACACCAG, | |
| 1460, | EPI-15-140, | M33494, | AGGTGCCATTCACCTTGCAC, | |
| 1461, | EPI-15-141, | M33494, | TAGCCAGGTGCCATTCACCT, | |
| 1462, | EPI-15-142, | M33494, | GCCTGTAGCCAGGTGCCATT, | |
| 1463, | EPI-15-143, | M33494, | CGCCCGCCTGTAGCCAGGTG, | |
| 1464, | EPI-15-144, | M33494, | CACCACCCCCCCCTCTACCG, | |
| 1465, | EPI-15-145, | M33494, | CAGCTGACCACGCCCGCCTG, | |
| 1466, | EPI-15-146, | M33494, | CGTCCCAGCTGACCACGCCC, | |
| 1467, | EPI-15-147, | M33494, | GCCCTCGTCCCAGCTGACCA, | |
| 1468, | EPI-15-148, | M33494, | GCACAGCCCTCGTCCCAGCT, | |
| 1469, | EPI-15-149, | M33494, | GCTGGGCACAGCCCTCGTCC, | |
| 1470, | EPI-15-150, | M33494, | GTTGGGCTGGGCACAGCCCT, | |
| 1471, | EPI-15-151, | M33494, | GGCCGGTTGGGCTGGGCACA, | |
| 1472, | EPI-15-152, | M33494, | TGCCAGGCCGGTTGGGCTGG, | |
| 1473, | EPI-15-153, | M33494, | GATGATGCCAGGCCGGTTGG, | |
| 1474, | EPI-15-154, | M33494, | CGGGTGTAGATGCCAGGCCG, | |
| 1475, | EPI-15-155, | M33494, | TGACACGGGTGTAGATGCCA, | |
| 1476, | EPI-15-156, | M33494, | GTAGGTGACACGGGTGTAGA, | |
| 1477, | EPI-15-157, | M33494, | AAGTAGTAGGTGACACGGGT, | |
| 1478, | EPI-15-158, | M33494, | AGTCCAAGTAGTAGGTGACA, | |
| 1479, | EPI-15-159, | M33494, | GATCCAGTCCAAGTAGTAGG, | |
| 1480, | EPI-15-160, | M33494, | TGGTGGATCCAGTCCAAGTA, | |
| 1481, | EPI-15-161, | M33494, | CATAGTGGTGGATCCAGTCC, | |
| 1482, | EPI-15-162, | M33494, | GGGGACATAGTGGTGGATCC, | |
| 1483, | EPI-15-163, | M33494, | TTTTTGGGGACATAGTGGTG, | |
| 1484, | EPI-15-164, | M33494, | ACGGCTTTTTGGGGACATAG, | |
| 1366, | EPI-15-165, | M33494, | GACTCACGGCTTTTTGGGGA, | |
| Concatemer Nucleic Acid Sequences of Tryptase-a gene oligo sequences | | | | |
| (SEQ ID NO: 1486) | | | | |
| AGGCTCAGCATCCTGGCCACGCAGCAGGCTCAGCATCCTGCAGCAGCAGCAGGCTCAGCAAGCGCCAGCAGCAGCAGGCTCGGGCAGCGCCAGCAGCAGCCAGGACGGGCAGCGCCAGCACTCGCCAGGACGGG | | | | |
| CAGCGCCGCGGCTCGCCAGGACGGGCGTAGGCGCGGCTCGCCAGGAGCCGCGTAGGCGCGGCTCGCCAGGGGCCGCGTAGGCGCGGTGGGGCAGGGGCCGCGTAGGTGGACTGGGGCAGGGGCCGCGGGCCTGG | | | | |
| ACTGGGGCAGGGCTGCAGGGCCTGGACTGGGGGCTTGCTGCAGGGCCTGGACTACCCGCTTGCTGCAGGGCCGACGATACCCGCTTGCTGCACCCCCGACGATACCCGCTTGCCTGACCCCCGACGATACCCGG | | | | |
| CCTCCTGACCCCCGACGACTGGGGGCCTCCTGACCCCCTGCTCCTGGGGGCCTCCTGACCACTTGCTCCTGGGGGCCTCAGGGCCACTTGCTCCTGGGCCTGCCAGGGCCACTTGCTCGCTCACCTGCCAGGGC | | | | |
| CACTCTCAGGCTCACCTGCCAGGGGGACTCTCAGGCTCACCTGCGTCGCGGACTCTCAGGCTCATATCGGTCGCGGACTCTCAGTCCAGTATCGGTCGCGGACTGTGCATCCAGTATCGGTCGCCAGAAGTGCA | | | | |
| AGATCCTTGACGTCCGGTCCCAGCCAGATCCTTGACGTCCGGGGGTGGCCAGATCCTTGACGCCTGAGGGTGGCCAGATCCTTGCACCCTGAGGGTGGCCAGGCAGTTGCACCCTGAGGGTGCTCCCGCAGTTG | | | | |
| CACCCTGATGCTGCTCCCGCAGTTGCACAGAGGTGCTGCTCCCGCAGTGTAGTAGAGGTGCTGCTCCCTCCTGGTAGTAGAGGTGCTGGCTGGTCCTGGTAGTAGAGGCAGCAGCTGGTCCTGGTAGTACTGGC | | | | |
| AGATCCTTGACGTCCGGTCCCAGCCAGATCCTTGACGTCCGGGGGTGGCCAGATCCTTGACGCCTGAGGGTGGCCAGATCCTTGCACCCTGAGGGTGGCCAGGCAGTTGCACCCTGAGGGTGCTCCCGCAGTTG | | | | |
| CACCCTGATGCTGCTCCCGCAGTTGCACAGAGGTGCTGCTCCCGCAGTGTAGTAGAGGTGCTGCTCCCTCCTGGTAGTAGAGGTGCTGGCTGGTCCTGGTAGTAGAGGCAGCAGCTGGTCCTGGTAGTACTGGC | | | | |
| AGCAGCTGGTCCTGTGCTGACTGGCAGCAGCTGGGATCCTGCTGACTGGCAGCAACGATGATCCTGCTGACTGGGGTGCACGATGATCCTGCTGCTGTGGGTGCACGATGATCCTAGAACTGTGGGTGCACGAT | | | | |
| TGATGTAGAACTGTGGGTGCCTGGATGATGTAGAACTGTGCCAGTCTGGATGATGTAGAACCGCTCCAGTCTGGATGATGGATATCCGCTCCAGTCTGGAAGGGCGATATCCGCTCCAGTCCAGCAGGGCGATA | | | | |
| TCCGCTCAGCTCCAGCAGGGCGATATTCCTCCAGCTCCAGCAGGGCCGGGCTCCTCCAGCTCCAGCGTTCACGGGCTCCTCCAGCTGAGATGTTCACGGGCTCCTCGGCTGGAGATGTTCACGGGCGACGCGGC | | | | |
| TGGAGATGTTCAGTGTGGACGCGGCTGGAGATTGACCGTGTGGACGCGGCTGCAGCATGACCGTGTGGACGCGGGGGCAGCATGACCGTGTGAGGCAGGGGGCAGCATGACCCTCCGAGGCAGGGGGCAGCAAA | | | | |
| GGTCTCCGAGGCAGGGGGGGGGGAAGGTCTCCGAGGCACCCCGGGGGGAAGGTCTCCGGGCATCCCCGGGGGGAAGGTAGCACGGCATCCCCGGGGGGACCCAGCACGGCATCCCCCGCCAGTGACCCAGCACG | | | | |
| GCATCCCAGCCAGTGACCCAGCACATCGCCCCAGCCAGTGACCCTCCACATCGCCCCAGCCAGTCATTGTCCACATCGCCCCAGCTCATCATTGTCCACATCGCAGGGGCTCATCATTGTCCACGTGGGAGGGG | | | | |
| CTCATCATTGTGGCGGTGGGAGGGGCTCATGGAAATGGCGGTGGGAGGGGTCAGGGGAAATGGCGGTGGGCTGCTTCAGGGGAAATGGCGTTCACCTGCTTCAGGGGAAAGGACCTTCACCTGCTTCAGGTATG | | | | |
| GGGACCTTCACCTGCTTCCATTATGGGGACCTTCACGGTTTTCCATTATGGGGACCAATGTGGTTTTCCATTATGGTCACAAATGTGGTTTTCCATTTGCGTCACAAATGTGGTTTGTATTTTGCGTCACAAAT | | | | |
| GTAGGTGGTATTTTGCGTCACACGCCAAGGTGGTATTTTGCGGTAGGCGCCAAGGTGGTATTCCCGTGTAGGCGCCAAGGTGCGTCTCCCGTGTAGGCGCCAGACGTCGTCTCCCGTGTAGGATGCGGACGTCG | | | | |
| TCTCCCGTGGATGATGCGGACGTCGTCTGTCACGGATGATGCGGACGTATGTCGTCACGGATGATGCGACAGCATGTCGTCACGGATGGGCACACAGCATGTCGTCACTTCCCGGCACACAGCATGTCGGCTGT | | | | |
| TCCCGGCACACAGCCCTCTGGCTGTTCCCGGCACGAGTCCCTCTGGCTGTTCCCTGCAGGAGTCCCTCTGGCTGGCCCTTGCAGGAGTCCCTCTGAGTCGCCCTTGCAGGAGTCCTCCAGAGTCGCCCTTGCAG | | | | |
| GGGCCCTCCAGAGTCGCCCTACCAGGGGCCCTCCAGAGTCTGCACACCAGGGGCCCTCCACACCTTGCACACCAGGGGCCCCATTCACCTTGCACACCAGAGGTGCCATTCACCTTGCACTAGCCAGGTGCCAT | | | | |
| TCACCTGCCTGTAGCCAGGTGCCATTCGCCCGCCTGTAGCCAGGTGGACCACGCCCGCCTGTAGCCCAGCTGACCACGCCCGCCTGCGTCCCAGCTGACCACGCCCGCCCTCGTCCCAGCTGACCAGCACAGCC | | | | |
| CTCGTCCCAGCTGCTGGGCACAGCCCTCGTCCGTTGGGCTGGGCACAGCCCTGGCCGGTTGGGCTGGGCACATGCCAGGCCGGTTGGGCTGGGTAGATGCCAGGCCGGTTGGCGGGTGTAGATGCCAGGCCGTG | | | | |
| ACACGGGTGTAGATGCCAGTAGGTGACACGGGTGTAGAAAGTAGTAGGTGACACGGGTAGTCCAAGTAGTAGGTGACAGATCCAGTCCAAGTAGTAGGTGGTGGATCCAGTCCAAGTACATAGTGGTGGATCCA | | | | |
| GTCCGGGGACATAGTGGTGGATCCTTTTTGGGGACATAGTGGTGACGGCTTTTTGGGGACATAGGACTCACGGCTTTTTGGGGA | | | | |
| Tryptase-b Nucleic Acid Sequences (GENBANK ACCESSION NO.X61177) | | | | |
| (SEQ ID NO: 1487) | | | | |
| CCAGGATGCTGAATCTGCTGCTGCTGGCGCTGCCCGTCCTGGCGAGCCGCGCCTACGCGGCCCCTGCCCCAGGCCAGGCCCTGCAGCGAGTGGGCATCGTTGGGGGTCAGGAGGCCCCCAGGAGCAAGTGGCCC | | | | |
| TGGCAGGTGAGCCTGAGAGTCCACGGCCCATACTGGATGCACTTCTGCGGGGGCTCCCTCATCCACCCCCAGTGGGTGCTGACCGCAGCGCACTGCGTGGGACCGGACGTCAAGGATCTGGCCGCCCTCAGGGT | | | | |
| GCAACTGCGGGAGCAGCACCTCTACTACCAGGACCAGCTGCTGCCGGTCAGCAGGATCATCGTGCACCCACAGTTCTACACCGCCCAGATCGGAGCGGACATCGCCCTGCTGGAGCTGGAGGAGCCGGTGAAGG | | | | |
| TCTCCAGCCACGTCCACACGGTCACCCTGCCCCCTGCCTCAGAGACCTTCCCCCCGGGGATGCCGTGCTGGGTCACTGGCTGGGGCGATGTGGACAATGATGAGCGCCTCCCACCGCCATTTCCTCTGAAGCAG | | | | |
| GTGAAGGTCCCCATAATGGAAAACCACATTTGTGACGCAAAATACCACCTTGGCGCCTACACGGGAGACGACGTCCGCATCGTCCGTGACGACATGCTGTGTGCCGGGAACACCCGGAGGGACTCATGCCAGGG | | | | |
| CGACTCCGGAGGGCCCCTGGTGTGCAAGGTGAATGGCACCTGGCTGCAGGCGGGCGTGGTCAGCTGGGGCGAGGGCTGTGCCCAGCCCAACCGGCCTGGCATCTACACCCGTGTCACCTACTACTTGGACTGGA | | | | |
| TCCACCACTATGTCCCCAAAAAGCCGTGAGTCAGGCCTGGGTGTGCCACCTGGGTCACTGGAGGACCAACCCCTGCTGTCCAAAACACCACTGCTTCCTACCCAGGTGGCGACTGCCCCCCACACCTTCCCTGC | | | | |
| CCCGTCCTGAGTGCCCCTTCCTGTCCTAAGCCCCCTGCTCTCTTCTGAGCCCCTTCCCCTGTCCTGAGGACCCTTCCCCATCCTGAGCCCCCTTCCCTGTCCTAAGCCTGACGCCTGCACTGCTCCGGCCCTCC | | | | |
| CCTGCCCAGGCAGCTGGTGGTGGGCGCTAATCCTCCTGAGTGCTGGACCTCATTAAAGTGCATGGAAATCA | | | | |
| 1488, | EPI-15-001B, | M37488, | AGATTCAGCATCCTGGCCAC, | |
| 1489, | EPI-15-002B, | M37488, | GCAGCAGATTCAGCATCCTG, | |
| 1490, | EPI-15-003B, | M37488, | CAGCAGCAGCAGATTCAGCA, | |
| 1491, | EPI-15-004B, | M37488, | AGCGCCAGCAGCAGCAGATT, | |
| 1492, | EPI-15-0013B, | M37488, | TGGCCTGGGGCAGGGGCCGC, | |
| 1493, | EPI-15-0014, | M37488, | GGGCCTGGACTGGGGCAGGG, | |
| 1494, | EPI-15-0015, | M37488, | CTGCAGGGCCTGGCCTGGGG, | |
| 1495, | EPI-15-0016, | M37488, | CCTCGCTGCAGGGCCTGGCC, | |
| 1496, | EPI-15-0017, | M37488, | TGCCCACTCGCTGCAGGGCC, | |
| 1497, | EPI-15-0018, | M37488, | AACGATGCCCACTCGCTGCA, | |
| 1498, | EPI-15-0019B, | M37488, | CCCCCAACGATGCCCACTCG, | |
| 1499, | EPI-15-0020b, | M37488, | CCTGACCCCCAACGATGCCC, | |
| 1500, | EPI-15-0021B, | M37488, | GGCCTCCTGACCCCCAACGA, | |
| 1501, | EPI-15-0030B, | M37488, | GCCGTGGACTCTCAGGCTCA, | |
| 1502, | EPI-15-0031B, | M37488, | TATGGGCCGTGGACTCTCAG, | |
| 1503, | EPI-15-0032B, | M37488, | TCCAGTATGGGCCGTGGACT, | |
| 1504, | EPI-15-0033B, | M37488, | GTGCATCCAGTATGGGCCGT, | |
| 1505, | EPI-15-0034B, | M37488, | CAGAAGTGCATCCAGTATGG, | |
| 1506, | EPI-15-0042B, | M37488, | TGCGGTCAGCACCCACTGGG, | |
| 1507, | EPI-15-0043B, | M37488, | TGCGCTGCGGTCAGCACCCA, | |
| 1508, | EPI-15-0044B, | M37488, | CGCAGTGCGCTGCGGTCAGC, | |
| 1509, | EPI-15-0045B, | M37488, | TCCCACGCAGTGCGCTGCGG, | |
| 1510, | EPI-15-0046B, | M37488, | TCCGGTCCCACGCAGTGCGC, | |
| 1511, | EPI-15-0047B, | M37488, | TGACGTCCGGTCCCACGCAG, | |
| 1512, | EPI-15-0050B, | M37488, | GGGCGGCCAGATCCTTGACG, | |
| 1513, | EPI-15-0051B, | M37488, | CCTGAGGGCGGCCAGATCCT, | |
| 1514, | EPI-15-0052B, | M37488, | TGCACCCTGAGGGCGGCCAG, | |
| 1515, | EPI-15-0053B, | M37488, | GATCCTGCTGACCGGCAGCA, | |
| 1516, | EPI-15-0061B, | M37488, | ACGATGATCCTGCTGACCGG, | |
| 1517, | EPI-15-0062B, | M37488, | TGCTGACCGGCAGCAGCTGG, | |
| 1518, | EPI-15-0063B, | M37488, | GATCCTGCTGACCGGCAGCA, | |
| 1519, | EPI-15-0064B, | M37488, | ACGATGATCCTGCTGACCGG, | |
| 1520, | EPI-15-0068B, | M37488, | CGGTGTAGAACTGTGGGTGC, | |
| 1521, | EPI-15-0069B, | M37488, | CTGGACGGTGTAGAACTGTG, | |
| 1522, | EPI-15-0070B, | M37488, | CCGATCTGGACGGTGTAGAA, | |
| 1523, | EPI-15-0071B, | M37488, | CCGCTCCGATCTGGACGGTG, | |
| 1524, | EPI-15-0072B, | M37488, | GATGTCCGCTCCGATCTGGA, | |
| 1525, | EPI-15-0073B, | M37488, | AGGGCGATGTCCGCTCCGAT, | |
| 1526, | EPI-15-0074B, | M37488, | CCAGCAGGGCGATGTCCGCT, | |
| 1527, | EPI-15-0075B, | M37488, | CAGCTCCAGCAGGGCGATGT, | |
| 1528, | EPI-15-0077B, | M37488, | CCGGCTCCTCCAGCTCCAGC, | |
| 1529, | EPI-15-0078B, | M37488, | CTTCACCGGCTCCTCCAGCT, | |
| 1530, | EPI-15-0079B, | M37488, | GAGACCTTCACCGGCTCCTC, | |
| 1531, | EPI-15-0080B, | M37488, | GGCTGGAGACCTTCACCGGC, | |
| 1532, | EPI-15-0081B, | M37488, | GACGTGGCTGGAGACCTTCA, | |
| 1533, | EPI-15-0082B, | M37488, | GTGTGGACGTGGCTGGAGAC, | |
| 1534, | EPI-15-0083B, | M37488, | TGACCGTGTGGACGTGGCTG, | |
| 1535, | EPI-15-0084B, | M37488, | CAGGGTGACCGTGTGGACGT, | |
| 1536, | EPI-15-0085B, | M37488, | GGGGGCAGGGTGACCGTGTG, | |
| 1537, | EPI-15-0086B, | M37488, | AGGCAGGGGGCAGGGTGACC, | |
| 1538, | EPI-15-0087B, | M37488, | CTCTGAGGCAGGGGGCAGGG, | |
| 1539, | EPI-15-0088B, | M37488, | AAGGTCTCTGAGGCAGGGGG, | |
| 1540, | EPI-15-0089B, | M37488, | GGGGGAAGGTCTCTGAGGCA, | |
| 1541, | EPI-15-0090B, | M37488, | AGGCAGGGGGCAGGGTGACC, | |
| 1542, | EPI-15-00100B, | M37488, | AGGCGCTCATCATTGTCCAC, | |
| 1543, | EPI-15-00101B, | M37488, | GTGGGAGGCGCTCATCATTG, | |
| 1544, | EPI-15-00102B, | M37488, | TGGCGGTGGGAGGCGCTCAT, | |
| 1545, | EPI-15-00103B, | M37488, | GGAAATGGCGGTGGGAGGCG, | |
| 1546, | EPI-15-00104B, | M37488, | TCAGAGGAAATGGCGGTGGG, | |
| 1547, | EPI-15-00105B, | M37488, | CTGCTTCAGAGGAAATGGCG, | |
| 1548, | EPI-15-00106B, | M37488, | TTCACCTGCTTCAGAGGAAA, | |
| 1549, | EPI-15-00107B, | M37488, | GGACCTTCACCTGCTTCAGA, | |
| 1550, | EPI-15-00122B, | M37488, | GGACGATGCGGACGTCGTCT, | |
| 1551, | EPI-15-00123B, | M37488, | GTCACGGACGATGCGGACGT, | |
| 1552, | EPI-15-00124B, | M37488, | ATGTCGTCACGGACGATGCG, | |
| 1553, | EPI-15-00125B, | M37488, | ACAGCATGTCGTCACGGACG, | |
| 1554, | EPI-15-00128B, | M37488, | GGCTGTTCCCGGCACACAGC, | |
| 1555, | EPI-15-00129B, | M37488, | CCTCTGGCTGTTCCCGGCAC, | |
| 1556, | EPI-15-00130B, | M37488, | GAGTCCCTCTGGCTGTTCCC, | |
| 1557, | EPI-15-00131B, | M37488, | TGCAGGAGTCCCTCTGGCTG, | |
| 1558, | EPI-15-00132B, | M37488, | GCCCTTGCAGGAGTCCCTCT, | |
| 1559, | EPI-15-00133B, | M37488, | GAGTCGCCCTTGCAGGAGTC, | |
| 1560, | EPI-15-00134B, | M37488, | CTCCAGAGTCGCCCTTGCAG, | |
| 1561, | EPI-15-00135B, | M37488, | GGGCCCTCCAGAGTCGCCCT, | |
| 1562, | EPI-15-00136B, | M37488, | ACCAGGGGCCCTCCAGAGTC, | |
| 1563, | EPI-15-00137B, | M37488, | TGCACACCAGGGTCCCTCCA, | |
| 1564, | EPI-15-00141B, | M37488, | TAGCCAGGTGCCATTCACCT, | |
| 1565, | EPI-15-00142B, | M37488, | GCCTGTAGCCAGGTGCCATT, | |
| 1566, | EPI-15-00143B, | M37488, | CGCCCGCCTGTAGCCAGGTG, | |
| 1567, | EPI-15-00144B, | M37488, | GACCACGCCCGCCTGTAGCC, | |
| 1568, | EPI-15-00146B, | M37488, | CGTCCCAGCTGACCACGCCC, | |
| 1569, | EPI-15-00147B, | M37488, | GCCCTCGTCCCAGCTGACCA, | |
| 1570, | EPI-15-00148B, | M37488, | GCACAGCCCTCGTCCCAGCT, | |
| 1571, | EPI-15-00149B, | M37488, | GCTGGGCACAGCCCTCGTCC, | |
| Concatemer Nucleic Acid Sequences of Tryptase-b gene oligo sequences | | | | |
| (SEQ ID NO: 1572) | | | | |
| AGATTCAGCATCCTGGCCACGCAGCAGATTCAGCATCCTGCAGCAGCAGCAGATTCAGCAAGCGCCAGCAGCAGCAGATTTGGCCTGGGGCAGGGGCCGCGGGCCTGGACTGGGGCAGGGCTGCAGGGCCTGCC | | | | |
| CTGGGGCCTCGCTGCAGGGCCTGGCCTGCCCACTCGCTGCAGGGCCAACGATGCCCACTCGCTGCACCCCCAACGATGCCCACTCGCCTGACCCCCAACGATGCCCGGCCTCCTGACCCCCAACGAGCCGTGGA | | | | |
| CTCTCAGGCTCATATGGGCCGTGGACTCTCAGTCCAGTATGGGCCGTGGACTGTGCATCCAGTATGGGCCGTCAGAAGTGCATCCAGTATGGTGCGGTCAGCACCCACTGGGTGCGCTGCGGTCAGCACCCACG | | | | |
| CAGTGCGCTGCGGTCAGCTCCCACGCAGTGCGCTGCGGTCCGGTCCCACGCAGTGCGCTGACGTCCGGTCCCACGCAGGGGCGGCCAGATCCTTGACGCCTGAGGGCGGCCAGATCCTTGCACCCTGAGGGCGG | | | | |
| CCAGGCAGTTGCACCCTGAGGGCGACCGGCAGCAGCTGGTCCTGTGCTGACCGGCAGCAGCTGGGATCCTGCTGACCGGCAGCAACGATGATCCTGCTGACCGGCGGTGTAGAACTGTGGGTGCCTGGACGGTG | | | | |
| TAGAACTGTGCCGATCTGGACGGTGTAGAACCGCTCCGATCTGGACGGTGGATGTCCGCTCCGATCTGGAAGGGCGATGTCCGCTCCGATCCAGCAGGGCGATGTCCGCTCAGCTCCAGCAGGGCGATGTCCGG | | | | |
| CTCCTCCAGCTCCAGCCTTCACCGGCTCCTCCAGCTGAGACCTTCACCGGCTCCTCGGCTGGAGACCTTCACCGGCGACGTGGCTGGAGACCTTCAGTGTGGACGTGGCTGGAGACTGACCGTGTGGACGTGGC | | | | |
| TGCAGGGTGACCGTGTGGACGTGGGGGCAGGGTGACCGTGTGAGGCAGGGGGCAGGGTGACCCTCTGAGGCAGGGGGCAGGGAAGGTCTCTGAGGCAGGGGGGGGGGAAGGTCTCTGAGGCACCCCGGGGGGAA | | | | |
| GGTCTCTGAGGCGCTCATCATTGTCCACGTGGGAGGCGCTCATCATTGTGGCGGTGGGAGGCGCTCATGGAAATGGCGGTGGGAGGCGTCAGAGGAAATGGCGGTGGGCTGCTTCAGAGGAAATGGCGTTCACC | | | | |
| TGCTTCAGAGGAAAGGACCTTCACCTGCTTCAGAGGACGATGCGGACGTCGTCTGTCACGGACGATGCGGACGTATGTCGTCACGGACGATGCGACAGCATGTCGTCACGGACGGGCTGTTCCCGGCACACACC | | | | |
| GGTCTCTGAGGCGCTCATCATTGTCCACGTGGGAGGCGCTCATCATTGTGGCGGTGGGAGGCGCTCATGGAAATGGCGGTGGGAGGCGTCAGAGGAAATGGCGGTGGGCTGCTTCAGAGGAAATGGCGTTCACC | | | | |
| TGCTTCAGAGGAAAGGACCTTCACCTGCTTCAGAGGACGATGCGGACGTCGTCTGTCACGGACGATGCGGACGTATGTCGTCACGGACGATGCGACAGCATGTCGTCACGGACGGGCTGTTCCCGGCACACACC | | | | |
| CCTCTGGCTGTTCCCGGCACGAGTCCCTCTGGCTGTTCCCTGCAGGAGTCCCTCTGGCTGGCCCTTGCAGGAGTCCCTCTGAGTCGCCCTTGCAGGAGTCCTCCAGAGTCGCCCTTGCAGGGGCCCTCCAGACT | | | | |
| CGCCCTACCAGGGGCCCTCCAGAGTCTGCACACCAGGGGCCCTCCATAGCCAGGTGCCATTCACCTGCCTGTAGCCAGGTGCCATTCGCCCGCCTGTAGCCAGGTGGACCACGCCCGCCTGTAGCCCGTCCCAG | | | | |
| CTGACCACGCCCGCCCTCGTCCCAGCTGACCAGCACAGCCCTCGTCCCAGCTGCTGGGCACAGCCCTCGTCC | | | | |
| PDE4A Nucleic Acid Sequences (GENBANK ACCESSION NO.X61177) | | | | |
| (SEQ ID NO:1573) | | | | |
| CAGGCGGGCTAAGTCTCCAAGATGCCCTTGGTGGATTTCTTCTGCGAGACCTGCTCTAAGCCTTGGCTGGTGGGCTGGTGGGACCAGTTCAAAAGGATGTTGAACCGTGAGCTCACACACCTGTCAGAAATGAG | | | | |
| CAGGTCCGGAAACCAGGTCTCAGAGTACATTTCCACAACATTCCTGGACAAACAGAATGAAGTGGAGATCCCATCACCCACGATGAAGGAACGAGAAAAACAGCAAGCGCCGCGACCAAGACCCTCCCAGCCGC | | | | |
| CCCCGCCCCCTGTACCACACTTACAGCCCATGTCCCAAATCACAGGGTTGAAAAAGTTGATGCATAGTAACAGCCTGAACAACTCTAACATTCCCCGATTTGGGGTGAAGACCGATCAAGAAGAGCTCCTGGCC | | | | |
| CAAGAACTGGAGAACCTGAACAAGTGGGGCCTGAACATCTTTTGCGTGTCGGATTACGCTGGAGGCCGCTCACTCACCTGCATCATGTACATGATATTCCAGGAGCGGGACCTGCTGAAGAAATTCCGCATCCC | | | | |
| GGTGGACACGATGGTGACATACATGCTGACGCTGGAGGATCACTACCACGCTGACGTGGCCTACCATAACAGCCTGCACGCAGCTGACGTGCTGCAGTCCACCCACGTACTGCTGGCCACGCCTGCACTAGATG | | | | |
| CAGTGTTCACGGACCTGGAGATTCTCGCCGCCCTCTTCGCGGCTGCCATCCACGATGTGGATCACCCTGGGGTCTCCAACCAGTTCCTCATCAACACCAATTCGGAGCTGGCGCTCATGTACAACGATGAGTCG | | | | |
| GTGCTCGAGAATCACCACCTGGCCGTGGGCTTCAAGCTGCTGCAGGAGGACAACTGCGACATCTTCCAGAACCTCAGCAAGCGCCAGCGGCAGAGCCTACGCAAGATGGTCATCGACATGGTGCTGGCCACGGA | | | | |
| CATGTCCAAGCACATGACCCTCCTGGCTGACCTGAAGACCATGGTGGAGACCAAGAAAGTGACCAGCTCAGGGGTCCTCCTGCTAGATAACTACTCCGACCGCATCCAGGTCCTCCGGAACATGGTGCACTGTG | | | | |
| GTGCTCGAGAATCACCACCTGGCCGTGGGCTTCAAGCTGCTGCAGGAGGACAACTGCGACATCTTCCAGAACCTCAGCAAGCGCCAGCGGCAGAGCCTACGCAAGATGGTCATCGACATGGTGCTGGCCACGGA | | | | |
| CATGTCCAAGCACATGACCCTCCTGGCTGACCTGAAGACCATGGTGGAGACCAAGAAAGTGACCAGCTCAGGGGTCCTCCTGCTAGATAACTACTCCGACCGCATCCAGGTCCTCCGGAACATGGTGCACTGTG | | | | |
| CCGACCTCAGCAACCCCACCAAGCCGCTGGAGCTGTACCGCCAGTGGACAGACCGCATCATGGCCGAGTTCTTCCAGCAGGGTGACCGAGAGCGCGAGCGTGGCATGGAAATCAGCCCCATGTGTGACAAGCAC | | | | |
| ACTGCCTCCGTGGAGAAGTCTCAGGTGGGTTTTATTGACTACATTGTGGCACCCATTGTGGGAGACCTGGGCGGACCTTGTCCACCCAGATGCCCAGGAGATCTTGGACACTTTGGAGGACAACCGGGACTGTA | | | | |
| CTACAGCGCCATCCGGCAGAGCCCATCTCCGCCACCCGAGGAGGAGTCAAGGGGGCCAGGCCACCCACCCCTGCCTGACAAGTTCCAGTTTGAGCTGACGCTGGAGGAGGAAGAGGAGGAAGAAATATCAATCG | | | | |
| CCCAGATACCGTGCACAGCCCAAGAGGCATTGACTGCGCAGGGATTGTCAGGAGTCGAGGAAGCTCTGGATGCAACCATAGCCTGGGAGGCATCCCCGGCCCAGGAGTCGTTGGAAGTTATGGCACAGGAAGCA | | | | |
| TCCCTGGAGGCCGAGCTGGAGGCAGTGTATTTGACACAGCAGGCACAGTCCACAGGCAGTGCACCTGTGGCTCCGGATGAGTTCTCGTCCCGGGAGGAATTCGTGGTTGCTGTAAGCCACAGCAGCCCCTCTCC | | | | |
| CCTGGCTCTTCAAAGCCCCCTTCTCCCTGCTTGGAGGACCCTGTCTGTTTCAGAGCATGCCCCGGGCCTCCCGGGCCTCCCCTCCACGGCGGCCGAGGTGGAGGCCCAACGAGAGCACCAGGCTGCCAAGAGGG | | | | |
| CTTGCAGTGCCTGCGCAGGGACATTTGGGGAGGACACATCCGCACTCCCAGCTCCTGGTGGCGGGGGGTCAGGTGGAGACCCTACCTGA | | | | |
| 1574, | U97584, | U97584, | TCAGGTAGGGTCTCCACCTG, | |
| 1575, | U97584, | U97584, | TCTCCACCTGACCCCCCGCC, | |
| 1576, | U97584, | U97584, | ACCCCCCGCCACCAGGAGCT, | |
| 1577, | U97584, | U97584, | ACCAGGAGCTGGGAGTGCGG, | |
| 1578, | U97584, | U97584, | GGGAGTGCGGATGTGTCCTC, | |
| 1579, | U97584, | U97584, | ATGTGTCCTCCCCAAATGTC, | |
| 1580, | U97584, | U97584, | CCCAAATGTCCCTGCGCAGG, | |
| 1581, | U97584, | U97584, | CCTGCGCAGGCACTGCAAGC, | |
| 1582, | U97584, | U97584, | CACTGCAAGCCCTCTTGGCA, | |
| 1583, | U97584, | U97584, | CCTCTTGGCAGCCTGGTGCT, | |
| 1584, | U97584, | U97584, | GCCTGGTGCTCTCGTTGGGC, | |
| 1585, | U97584, | U97584, | CTCGTTGGGCCTCCACCTCG, | |
| 1586, | U97584, | U97584, | CTCCACCTCGGCCGCCGTGG, | |
| 1587, | U97584, | U97584, | GCCGCCGTGGAGGGGAGGCC, | |
| 1588, | U97584, | U97584, | AGGGGAGGCCCGGGAGGCCC, | |
| 1589, | U97584, | U97584, | CGGGAGGCCCGGGGCATGCT, | |
| 1590, | U97584, | U97584, | GGGGCATGCTCTGAAACAGA, | |
| 1591, | U97584, | U97584, | CTGAAACAGACAGGGTCCTC, | |
| 1592, | U97584, | U97584, | CAGGGTCCTCCAAGCAGGGA, | |
| 1593, | U97584, | U97584, | CAAGCAGGGAGAAGGGGGCT, | |
| 1594, | U97584, | U97584, | GAAGGGGGCTTTGAAGAGCC, | |
| 1595, | U97584, | U97584, | TTGAAGAGCCAGGGCAGAGG, | |
| 1596, | U97584, | U97584, | AGGGCAGAGGGGCTGCTGTG, | |
| 1597, | U97584, | U97584, | GGCTGCTGTGGCTTACAGCA, | |
| 1598, | U97584, | U97584, | GCTTACAGCAACCACGAATT, | |
| 1599, | U97584, | U97584, | ACCACGAATTCCTCCCGGGA, | |
| 1600, | U97584, | U97584, | CCTCCCGGGACGAGAACTCA, | |
| 1601, | U97584, | U97584, | CGAGAACTCATCCGGAGCCA, | |
| 1602, | U97584, | U97584, | TCCGGAGCCACAGGTGCACT, | |
| 1603, | U97584, | U97584, | CAGGTGCACTGCCTGTGGAC, | |
| 1604, | U97584, | U97584, | GCCTGTGGACTGTGCCTGCT, | |
| 1605, | U97584, | U97584, | TGTGCCTGCTGTGTCAAATA, | |
| 1606, | U97584, | U97584, | GTGTCAAATACACTGCCTCC, | |
| 1607, | U97584, | U97584, | CACTGCCTCCAGCTCGGCCT, | |
| 1608, | U97584, | U97584, | AGCTCGGCCTCCAGGGATGC, | |
| 1609, | U97584, | U97584, | CCAGGGATGCTTCCTGTGCC, | |
| 1610, | U97584, | U97584, | TTCCTGTGCCATAACTTCCA, | |
| 1611, | U97584, | U97584, | ATAACTTCCAACGACTCCTG, | |
| 1612, | U97584, | U97584, | ACGACTCCTGGGCCGGGGAT, | |
| 1613, | U97584, | U97584, | GGCCGGGGATGCCTCCCAGG, | |
| 1614, | U97584, | U97584, | GCCTCCCAGGCTATGGTTGC, | |
| 1615, | U97584, | U97584, | CTATGGTTGCATCCAGAGCT, | |
| 1616, | U97584, | U97584, | ATCCAGAGCTTCCTCGACTC, | |
| 1617, | U97584, | U97584, | TCCTCGACTCCTGACAATCC, | |
| 1618, | U97584, | U97584, | CTGACAATCCCTGCGCAGTC, | |
| 1619, | U97584, | U97584, | CTGCGCAGTCAATGCCTCTT, | |
| 1620, | U97584, | U97584, | AATGCCTCTTGGGCTGTGCA, | |
| 1621, | U97584, | U97584, | GGGCTGTGCACGGTATCTGG, | |
| 1622, | U97584, | U97584, | CGGTATCTGGGCCATTGATA, | |
| 1623, | U97584, | U97584, | GCCATTGATATTTCTTCCTC, | |
| 1624, | U97584, | U97584, | TTTCTTCCTCCTCTTCCTCC, | |
| 1625, | U97584, | U97584, | CTCTTCCTCCTCCAGCGTCA, | |
| 1626, | U97584, | U97584, | TCCAGCGTCAGCTCAAACTG, | |
| 1627, | U97584, | U97584, | GCTCAAACTGGAACTTGTCA, | |
| 1628, | U97584, | U97584, | GAACTTGTCAGGCAGGGGTG, | |
| 1629, | U97584, | U97584, | GGCAGGGGTGGGTGGCCTGG, | |
| 1630, | U97584, | U97584, | GGTGGCCTGGCCCCCTTGAC, | |
| 1631, | U97584, | U97584, | CCCCCTTGACTCCTCCTCGG, | |
| 1632, | U97584, | U97584, | TCCTCCTCGGGTGGCGGAGA, | |
| 1633, | U97584, | U97584, | GTGGCGGAGATGGGCTCTGC, | |
| 1634, | U97584, | U97584, | TGGGCTCTGCCGGATGGCGC, | |
| 1635, | U97584, | U97584, | CGGATGGCGCTGTAGTACCA, | |
| 1636, | U97584, | U97584, | TGTAGTACCAGTCCCGGTTG, | |
| 1637, | U97584, | U97584, | GTCCCGGTTGTCCTCCAAAG, | |
| 1638, | U97584, | U97584, | TCCTCCAAAGTGTCCAAGAT, | |
| 1639, | U97584, | U97584, | TGTCCAAGATCTCCTGGGCA, | |
| 1640, | U97584, | U97584, | CTCCTGGGCATCTGGGTGGA, | |
| 1641, | U97584, | U97584, | TCTGGGTGGACAAGGTCCGC, | |
| 1642, | U97584, | U97584, | CAAGGTCCGCCCAGGTCTCC, | |
| 1643, | U97584, | U97584, | CCAGGTCTCCCACAATGGGT, | |
| 1644, | U97584, | U97584, | CACAATGGGTGCACAATGTA, | |
| 1645, | U97584, | U97584, | GCACAATGTAGTCAATAAAA, | |
| 1646, | U97584, | U97584, | GTCAATAAAACCCACCTGAG, | |
| 1647, | U97584, | U97584, | CCCACCTGAGACTTCTCCAC, | |
| 1648, | U97584, | U97584, | ACTTCTCCACGGAGGCAGTG, | |
| 1649, | U97584, | U97584, | GGAGGCAGTGTGCTTGTCAC, | |
| 1650, | U97584, | U97584, | TGCTTGTCACACATGGGGCT, | |
| 1651, | U97584, | U97584, | ACATGGGGCTGATTTCCATG, | |
| 1652, | U97584, | U97584, | GATTTCCATGCCACGCTCGC, | |
| 1653, | U97584, | U97584, | CCACGCTCGCGCTCTCGGTC, | |
| 1654, | U97584, | U97584, | GCTCTCGGTCACCCTGCTGG, | |
| 1655, | U97584, | U97584, | ACCCTGCTGGAAGAACTCGG, | |
| 1656, | U97584, | U97584, | AAGAACTCGGCCATGATGCG, | |
| 1657, | U97584, | U97584, | CCATGATGCGGTCTGTCCAC, | |
| 1658, | U97584, | U97584, | GTCTGTCCACTGGCGGTACA, | |
| 1659, | U97584, | U97584, | TGGCGGTACAGCTCCAGCGG, | |
| 1660, | U97584, | U97584, | GCTCCAGCGGCTTGGTGGGG, | |
| 1661, | U97584, | U97584, | CTTGGTGGGGTTGCTGAGGT, | |
| 1662, | U97584, | U97584, | TTGCTGAGGTCGGCACAGTG, | |
| 1663, | U97584, | U97584, | CGGCACAGTGCACCATGTTC, | |
| 1664, | U97584, | U97584, | CACCATGTTCCGGAGGACCT, | |
| 1665, | U97584, | U97584, | CGGAGGACCTGGATGCGGTC, | |
| 1666, | U97584, | U97584, | GGATGCGGTCGGAGTAGTTA, | |
| 1667, | U97584, | U97584, | GGAGTAGTTATCTAGCAGGA, | |
| 1668, | U97584, | U97584, | GCTGGTCACTTTCTTGGTCT, | |
| 1669, | U97584, | U97584, | TTCTTGGTCTCCACCATGGT, | |
| 1670, | U97584, | U97584, | GCTGGTCACTTTCTTGGTCT, | |
| 1671, | U97584, | U97584, | TTCTTGGTCTCCACCATGGT, | |
| 1672, | U97584, | U97584, | CCACCATGGTCTTCAGGTCA, | |
| 1673, | U97584, | U97584, | CTTCAGGTCAGCCAGGAGGG, | |
| 1674, | U97584, | U97584, | GCCAGGAGGGTCATGTGCTT, | |
| 1675, | U97584, | U97584, | TCATGTGCTTGGACATGTCC, | |
| 1676, | U97584, | U97584, | GGACATGTCCGTGGCCAGCA, | |
| 1677, | U97584, | U97584, | GTGGCCAGCACCATGTCGAT, | |
| 1678, | U97584, | U97584, | CCATGTCGATGACCATCTTG, | |
| 1679, | U97584, | U97584, | GACCATCTTGCGTAGGCTCT, | |
| 1680, | U97584, | U97584, | CGTAGGCTCTGCCGCTGGCG, | |
| 1681, | U97584, | U97584, | GCCGCTGGCGCTTGCTGAGG, | |
| 1682, | U97584, | U97584, | CTTGCTGAGGTTCTGGAAGA, | |
| 1683, | U97584, | U97584, | TTCTGGAAGATGTCGCAGTT, | |
| 1684, | U97584, | U97584, | TGTCGCAGTTGTCCTCCTGC, | |
| 1685, | U97584, | U97584, | GTCCTCCTGCAGCAGCTTGA, | |
| 1686, | U97584, | U97584, | AGCAGCTTGAAGCCCACGGC, | |
| 1687, | U97584, | U97584, | AGCCCACGGCCAGGTGGTGA, | |
| 1688, | U97584, | U97584, | CAGGTGGTGATTCTCGAGCA, | |
| 1689, | U97584, | U97584, | TTCTCGAGCACCGACTCATC, | |
| 1690, | U97584, | U97584, | CCGACTCATCGTTGTACATG, | |
| 1691, | U97584, | U97584, | GTTGTACATGAGCGCCAGCT, | |
| 1692, | U97584, | U97584, | AGCGCCAGCTCCGAATTGGT, | |
| 1693, | U97584, | U97584, | CCGAATTGGTGTTGATGAGG, | |
| 1694, | U97584, | U97584, | GTTGATGAGGAACTGGTTGG, | |
| 1695, | U97584, | U97584, | AACTGGTTGGAGACCCCAGG, | |
| 1696, | U97584, | U97584, | AGACCCCAGGGTGATCCACA, | |
| 1697, | U97584, | U97584, | GTGATCCACATCGTGGATGG, | |
| 1698, | U97584, | U97584, | TCGTGGATGGCAGCCGCGAA, | |
| 1699, | U97584, | U97584, | CAGCCGCGAAGAGGGCGGCG, | |
| 1700, | U97584, | U97584, | GAGGGCGGCGAGAATCTCCA, | |
| 1701, | U97584, | U97584, | AGAATCTCCAGGTCCGTGAA, | |
| 1702, | U97584, | U97584, | GGTCCGTGAACACTGCATCT, | |
| 1703, | U97584, | U97584, | CACTGCATCTAGTGCAGGCG, | |
| 1704, | U97584, | U97584, | AGTGCAGGCGTGGCCAGCAG, | |
| 1705, | U97584, | U97584, | TGGCCAGCAGTACGTGGGTG, | |
| 1706, | U97584, | U97584, | TACGTGGGTGGACTGCAGCA, | |
| 1707, | U97584, | U97584, | GACTGCAGCACGTCAGCTGC, | |
| 1708, | U97584, | U97584, | CGTCAGCTGCGTGCAGGCTG, | |
| 1709, | U97584, | U97584, | GTGCAGGCTGTTATGGTAGG, | |
| 1710, | U97584, | U97584, | TTATGGTAGGCCACGTCAGC, | |
| 1711, | U97584, | U97584, | CCACGTCAGCGTGGTAGTGA, | |
| 1712, | U97584, | U97584, | GTGGTAGTGATCCTCCAGCG, | |
| 1713, | U97584, | U97584, | TCCTCCAGCGTCAGCATGTA, | |
| 1714, | U97584, | U97584, | TCAGCATGTATGTCACCATC, | |
| 1715, | U97584, | U97584, | TGTCACCATCGTGTCCACCG, | |
| 1716, | U97584, | U97584, | GTGTCCACCGGGATGCGGAA, | |
| 1717, | U97584, | U97584, | GGATGCGGAATTTCTTCAGC, | |
| 1718, | U97584, | U97584, | TTTCTTCAGCAGGTCCCGCT, | |
| 1719, | U97584, | U97584, | AGGTCCCGCTCCTGGAATAT, | |
| 1720, | U97584, | U97584, | CCTGGAATATCATGTACATG, | |
| 1721, | U97584, | U97584, | CATGTACATGATGCAGGTGA, | |
| 1722, | U97584, | U97584, | ATGCAGGTGAGTGAGCGGCC, | |
| 1723, | U97584, | U97584, | GTGAGCGGCCTCCAGCGTAA, | |
| 1724, | U97584, | U97584, | TCCAGCGTAATCCGACACGC, | |
| 1725, | U97584, | U97584, | TCCGACACGCAAAAGATGTT, | |
| 1726, | U97584, | U97584, | AAAAGATGTTCAGGCCCCAC, | |
| 1727, | U97584, | U97584, | CAGGCCCCACTTGTTCAGGT, | |
| 1728, | U97584, | U97584, | TTGTTCAGGTTCTCCAGTTC, | |
| 1729, | U97584, | U97584, | TCTCCAGTTCTTGGGCCAGG, | |
| 1730, | U97584, | U97584, | TTGGGCCAGGAGCTCTTCTT, | |
| 1731, | U97584, | U97584, | AGCTCTTCTTGATCGGTCTT, | |
| 1732, | U97584, | U97584, | GATCGGTCTTCACCCCAAAT, | |
| 1733, | U97584, | U97584, | CACCCCAAATCGGGGAATGT, | |
| 1734, | U97584, | U97584, | CGGGGAATGTTAGAGTTGTT, | |
| 1735, | U97584, | U97584, | TAGAGTTGTTCAGGCTGTTA, | |
| 1736, | U97584, | U97584, | CAGGCTGTTACTATGCATCA, | |
| 1737, | U97584, | U97584, | CTATGCATCAACTTTTTCAA, | |
| 1738, | U97584, | U97584, | ACTTTTTCAACCCTGTGATT, | |
| 1739, | U97584, | U97584, | CCCTGTGATTTGGGACATGG, | |
| 1740, | U97584, | U97584, | TGGGACATGGGCTGTAAGTG, | |
| 1741, | U97584, | U97584, | GCTGTAAGTGTGGTACAGGG, | |
| 1742, | U97584, | U97584, | TGGTACAGGGGGCGGGGGCG, | |
| 1743, | U97584, | U97584, | GGCGGGGGCGGCTGGGAGGG, | |
| 1744, | U97584, | U97584, | GCTGGGAGGGTCTTGGTCGC, | |
| 1745, | U97584, | U97584, | TCTTGGTCGCGGCGCTTGCT, | |
| 1746, | U97584, | U97584, | GGCGCTTGCTGTTTTTCTCG, | |
| 1747, | U97584, | U97584, | GCTGGGAGGGTCTTGGTCGC, | |
| 1748, | U97584, | U97584, | TCTTGGTCGCGGCGCTTGCT, | |
| 1749, | U97584, | U97584, | GGCGCTTGCTGTTTTTCTCG, | |
| 1750, | U97584, | U97584, | GCTGGGAGGGTCTTGGTCGC, | |
| 1751, | U97584, | U97584, | TCTTGGTCGCGGCGCTTGCT, | |
| 1752, | U97584, | U97584, | GGCGCTTGCTGTTTTTCTCG, | |
| 1753, | U97584, | U97584, | GCTGGGAGGGTCTTGGTCGC, | |
| 1754, | U97584, | U97584, | AATGTACTCTGAGACCTGGT, | |
| 1755, | U97584, | U97584, | GAGACCTGGTTTCCGGACCT, | |
| 1756, | U97584, | U97584, | TTCCGGACCTGCTCATTTCT, | |
| 1757, | U97584, | U97584, | TTGTCCAGGAATGTTGTGGA, | |
| 1758, | U97584, | U97584, | ATGTTGTGGAAATGTACTCT, | |
| 1759, | U97584, | U97584, | AATGTACTCTGAGACCTGGT, | |
| 1760, | U97584, | U97584, | GAGACCTGGTTTCCGGACCT, | |
| 1761, | U97584, | U97584, | TTCCGGACCTGCTCATTTCT, | |
| 1762, | U97584, | U97584, | GCTCATTTCTGACAGGTGTG, | |
| 1763, | U97584, | U97584, | GACAGGTGTGTGAGCTCACG, | |
| 1764, | U97584, | U97584, | TGAGCTCACGGTTCAACATC, | |
| 1765, | U97584, | U97584, | AGCAGGTCTCGCAGAAGAAA, | |
| 1766, | U97584, | U97584, | GCAGAAGAAATCCACCAAGG, | |
| 1767, | U97584, | U97584, | TCCACCAAGGGCATCTTGGA, | |
| 1768, | U97584, | U97584, | GCATCTTGGAGACTTAGCCC, | |
| 1769, | EPI-19-MTA-1, | U97584, | CCATGATGCGGTCTGTCCA, | |
| 1770, | EPI-19-MTA-2, | U97584, | TCTTCAGCAGGTCCCGCTCCTG, | |
| 1771, | EPI-19-MTA-3, | U97584, | AACTGGTTGGAGACCCCAGG, | |
| 1772, | EPI-19-MTA-4, | U97584, | CCTGCAGCAGCTTGAAGCCCAC, | |
| 1773, | EPI-19-MTA-5, | U97584, | GCTGAGGTTCTGGAAGAT, | |
| 1774, | EPI-19-MTA-6, | U97584, | GTGGCCAGCACCATGTC, | |
| 1775, | EPI-19-MTA-7, | U97584, | TTCTTGGTCTCCACCATGGTCTT, | |
| 1776, | EPI-19-MTA-8, | U97584, | CAGCGGCTTGGTGGGGTTGCT, | |
| 1777, | EPI-19-MTA-9, | U97584, | GTCCACTGGCGGTACAG, | |
| 1778, | EPI-19-MTA-10, | U97584, | TGCTTGTCACACATGGG, | |
| 1779, | EPI-19-MTA-11, | U97584, | GTCCACTGGCGGTACAGCT, | |
| 1780, | EPI-19-MTA-12, | U97584, | TGTGCTTGTCACACATGGGGCT, | |
| 1781, | EPI-19-MTA-13, | U97584, | TTGTCCTCCAAAGTGTCCAA, | |
| Concatemer Nucleic Acid Sequences of PDE4A gene oligo sequences | | | | |
| (SEQ ID NO: 1782) | | | | |
| TCAGGTAGGGTCTCCACCTGTCTCCACCTGACCCCCCGCCACCCCCCGCCACCAGGAGCTACCAGGAGCTGGGAGTGCGGGGGAGTGCGGATGTGTCCTCATGTGTCCTCCCCAAATGTCCCCAAATGTCCCTG | | | | |
| CGCAGGCCTGCGCAGGCACTGCAAGCCACTGCAAGCCCTCTTGGCACCTCTTGGCAGCCTGGTGCTGCCTGGTGCTCTCGTTGGGCCTCGTTGGGCCTCCACCTCGCTCCACCTCGGCCGCCGTGGGCCGCCGT | | | | |
| GGAGGGGAGGCCAGGGGAGGCCCGGGAGGCCCCGGGAGGCCCGGGGCATGCTGGGGCATGCTCTGAAACAGACTGAAACAGACAGGGTCCTCCAGGGTCCTCCAAGCAGGGACAAGCAGGGAGAAGGGGGCTGA | | | | |
| AGGGGGCTTTGAAGAGCCTTGAAGAGCCAGGGCAGAGGAGGGCAGAGGGGCTGCTGTGGGCTGCTGTGGCTTACAGCAGCTTACAGCAACCACGAATTACCACGAATTCCTCCCGGGACCTCCCGGGACGAGAA | | | | |
| CTCACGAGAACTCATCCGGAGCCATCCGGAGCCACAGGTGCACTCAGGTGCACTGCCTGTGGACGCCTGTGGACTGTGCCTGCTTGTGCCTGCTGTGTCAAATAGTGTCAAATACACTGCCTCCCACTGCCTCC | | | | |
| AGCTCGGCCTAGCTCGGCCTCCAGGGATGCCCAGGGATGCTTCCTGTGCCTTCCTGTGCCATAACTTCCAATAACTTCCAACGACTCCTGACGACTCCTGGGCCGGGGATGGCCGGGGATGCCTCCCAGGGCCT | | | | |
| CCCAGGCTATGGTTGCCTATGGTTGCATCCAGAGCTATCCAGAGCTTCCTCGACTCTCCTCGACTCCTGACAATCCCTGACAATCCCTGCGCAGTCCTGCGCAGTCAATGCCTCTTAATGCCTCTTGGGCTGTG | | | | |
| CAGGGCTGTGCACGGTATCTGGCGGTATCTGGGCCATTGATAGCCATTGATATTTCTTCCTCTTTCTTCCTCCTCTTCCTCCCTCTTCCTCCTCCAGCGTCATCCAGCGTCAGCTCAAACTGGCTCAAACTGGA | | | | |
| ACTTGTCAGAACTTGTCAGGCAGGGGTGGGCAGGGGTGGGTGGCCTGGGGTGGCCTGGCCCCCTTGACCCCCCTTGACTCCTCCTCGGTCCTCCTCGGGTGGCGGAGAGTGGCGGAGATGGGCTCTGCTGGGCT | | | | |
| CTGCCGGATGGCGCCGGATGGCGCTGTAGTACCATGTAGTACCAGTCCCGGTTGGTCCCGGTTGTCCTCCAAAGTCCTCCAAAGTGTCCAAGATTGTCCAAGATCTCCTGGGCACTCCTGGGCATCTGGGTGGA | | | | |
| TCTGGGTGGACAAGGTCCGCCAAGGTCCGCCCAGGTCTCCCCAGGTCTCCCACAATGGGTCACAATGGGTGCACAATGTAGCACAATGTAGTCAATAAAAGTCAATAAAACCCACCTGAGCCCACCTGAGACTT | | | | |
| CTCCACACTTCTCCACGGAGGCAGTGGGAGGCAGTGTGCTTGTCACTGCTTGTCACACATGGGGCTACATGGGGCTGATTTCCATGGATTTCCATGCCACGCTCGCCCACGCTCGCGCTCTCGGTCGCTCTCGG | | | | |
| TCACCCTGCTGGACCCTGCTGGAAGAACTCGGAAGAACTCGGCCATGATGCGCCATGATGCGGTCTGTCCACGTCTGTCCACTGGCGGTACATGGCGGTACAGCTCCAGCGGGCTCCAGCGGCTTGGTGGGGCT | | | | |
| TGGTGGGGTTGCTGAGGTTTGCTGAGGTCGGCACAGTGCGGCACAGTGCACCATGTTCCACCATGTTCCGGAGGACCTCGGAGGACCTGGATGCGGTCGGATGCGGTCGGAGTAGTTAGGAGTAGTTATCTAGC | | | | |
| AGGATCTAGCAGGAGGACCCCTGAGGACCCCTGAGCTGGTCACTGCTGGTCACTTTCTTGGTCTTTCTTGGTCTCCACCATGGTCCACCATGGTCTTCAGGTCACTTCAGGTCAGCCAGGAGGGGCCAGGAGGG | | | | |
| TCATGTGCTTTCATGTGCTTGGACATGTCCGGACATGTCCGTGGCCAGCAGTGGCCAGCACCATGTCGATCCATGTCGATGACCATCTTGGACCATCTTGCGTAGGCTCTCGTAGGCTCTGCCGCTGGCGGCCG | | | | |
| CTGGCGCTTGCTGAGGCTTGCTGAGGTTCTGGAAGATTCTGGAAGATGTCGCAGTTTGTCGCAGTTGTCCTCCTGCGTCCTCCTGCAGCAGCTTGAAGCAGCTTGAAGCCCACGGCAGCCCACGGCCAGGTGGT | | | | |
| GACAGGTGGTGATTCTCGAGCATTCTCGAGCACCGACTCATCCCGACTCATCGTTGTACATGGTTGTACATGAGCGCCAGCTAGCGCCAGCTCCGAATTGGTCCGAATTGGTGTTGATGAGGGTTGATGAGGAA | | | | |
| CTGGTTGGAACTGGTTGGAGACCCCAGGAGACCCCAGGGTGATCCACAGTGATCCACATCGTGGATGGTCGTGGATGGCAGCCGCGAACAGCCGCGAAGAGGGCGGCGGAGGGCGGCGAGAATCTCCAAGAATC | | | | |
| TCCAGGTCCGTGAAGGTCCGTGAACACTGCATCTCACTGCATCTAGTGCAGGCGAGTGCAGGCGTGGCCAGCAGTGGCCAGCAGTACGTGGGTGTACGTGGGTGGACTGCAGCAGACTGCAGCACGTCAGCTGC | | | | |
| CGTCAGCTGCGTGCAGGCTGGTGCAGGCTGTTATGGTAGGTTATGGTAGGCCACGTCAGCCCACGTCAGCGTGGTAGTGAGTGGTAGTGATCCTCCAGCGTCCTCCAGCGTCAGCATGTATCAGCATGTATGTC | | | | |
| ACCATCTGTCACCATCGTGTCCACCGGTGTCCACCGGGATGCGGAAGGATGCGGAATTTCTTCAGCTTTCTTCAGCAGGTCCCGCTAGGTCCCGCTCCTGGAATATCCTGGAATATCATGTACATGCATGTACA | | | | |
| TGATGCAGGTGAATGCAGGTGAGTGAGCGGCCGTGAGCGGCCTCCAGCGTAATCCAGCGTAATCCGACACGCTCCGACACGCAAAAGATGTTAAAAGATGTTCAGGCCCCACCAGGCCCCACTTGTTCAGGTTT | | | | |
| GTTCAGGTTCTCCAGTTCTCTCCAGTTCTTGGGCCAGGTTGGGCCAGGAGCTCTTCTTAGCTCTTCTTGATCGGTCTTGATCGGTCTTCACCCCAAATCACCCCAAATCGGGGAATGTCGGGGAATGTTAGAGT | | | | |
| TGTTTAGAGTTGTTCAGGCTGTTACAGGCTGTTACTATGCATCACTATGCATCAACTTTTTCAAACTTTTTCAACCCTGTGATTCCCTGTGATTTGGGACATGGTGGGACATGGGCTGTAAGTGGCTGTAAGTG | | | | |
| TGGTACAGGGTGGTACAGGGGGCGGGGGCGGGCGGGGGCGGCTGGGAGGGGCTGGGAGGGTCTTGGTCGCTCTTGGTCGCGGCGCTTGCTGGCGCTTGCTGTTTTTCTCGGTTTTTCTCGTTCCTTCATCTTCC | | | | |
| TTCATCGTGGGTGATGGTGGGTGATGGGATCTCCACGGATCTCCACTTCATTCTGTTTCATTCTGTTTGTCCAGGATTGTCCAGGAATGTTGTGGAATGTTGTGGAAATGTACTCTAATGTACTCTGAGACCTG | | | | |
| GTGAGACCTGGTTTCCGGACCTTTCCGGACCTGCTCATTTCTGCTCATTTCTGACAGGTGTGGACAGGTGTGTGAGCTCACGTGAGCTCACGGTTCAACATCGTTCAACATCCTTTTGAACTCTTTTGAACTGG | | | | |
| TCCCACCAGGTCCCACCAGCCCACCAGCGCCCACCAGCCAAGGCTTAGCAAGGCTTAGAGCAGGTCTCAGCAGGTCTCGCAGAAGAAAGCAGAAGAAATCCACCAAGGTCCACCAAGGGCATCTTGGAGCATCT | | | | |
| TGGAGACTTAGCCCCCATGATGCGGTCTGTCCATCTTCAGCAGGTCCCGCTCCTGAACTGGTTGGAGACCCCAGGCCTGCAGCAGCTTGAAGCCCACGCTGAGGTTCTGGAAGATGTGGCCAGCACCATGTCTT | | | | |
| CTTGGTCTCCACCATGGTCTTCAGCGGCTTGGTGGGGTTGCTGTCCACTGGCGGTACAGTGCTTGTCACACATGGGGTCCACTGGCGGTACAGCTTGTGCTTGTCACACATGGGGCTTTGTCCTCCAAAGTGTC | | | | |
| CAA | | | | |
| PDE4B Nucleic Acid Sequences (GENBANK ACCESSION NO.X61177) | | | | |
| (SEQ ID NO: 1783) | | | | |
| GAATTCCTCCTCTCTTCACCCCGTTAGCTGTTTTCAATGTAATGCTGCCGTCCTTCTCTTGCACTGCCTTCTGCGCTAACACCTCCATTCCTGTTTATAACCGTGTATTTATTACTTAATGTATATAATGTAAT | | | | |
| GTTTTGTAAGTTATTAATTTATATATCTAACATTGCCTGCCAATGGTGGTGTTAAATTTGTGTAGAAAACTCTGCCTAAGAGTTACGACTTTTCTTGTAATGTTTTTGTATTGTGTATTATATAACCCAAACGT | | | | |
| CACTTAGTAGAGACATATGGCCCCCTTGGCAGAGAGGACAGGGGTGGGCTTTTGTTCAAAGGGTCTGCCCTTTCCCTGCCTGAGTTGCTACTTCTGCACAACCCCTTTATGAACCAGTTTTCACCCGAATTTTG | | | | |
| ACTGTTTCATTTAGAAGAAAAGCAAAATGAGAAAAAGCTTTCCTCATTTCTCCTTGAGATGGCAAAGCACTCAGAAATGACATCACATACCCTAAAGAACCCTGGGATGACTAAGGCAGAGAGAGTCTGAGAAA | | | | |
| ACTCTTTGGTGCTTCTGCCTTTAGTTTTAGGACACATTTATGCAGATGAGCTTATAAGAGACCGTTCCCTCCGCCTTCTTCCTCAGAGGAAGTTTCTTGGTAGATCACCGACACCTCATCCAGGCGGGGGGTTG | | | | |
| GGGGGAAACTTGGCACCAGCCATCCCAGGCAGAGCACCACTGTGATTTGTTCTCCTGGTGGAGAGAGCTGGAAGGAAGGAGCCAGCGTGCAAATAATGAAGGAGCACGGGGGCACCTTCAGTAGCACCGGAATC | | | | |
| AGCGGTGGTAGCGGTGACTCTGCTATGGACAGCCTGCAGCCGCTCCAGCCTAACTACATGCCTGTGTGTTTGTTTGCAGAAGAATCTTATCAAAAATTAGCAATGGAAACGCTGGAGGAATTAGACTGGTGTTT | | | | |
| AGACCAGCTAGAGACCATACAGACCTACCGGTCTGTCAGTGAGATGGCTTCTAACAAGTTCAAAAGAATGCTGAACCGGGAGCTGACACACCTCTCAGAGATGAGCCGATCAGGGAACCAGGTGTCTGAATACA | | | | |
| TTTCAAATACTTTCTTAGACAAGCAGAATGATGTGGAGATCCCATCTCCTACCCAGAAAGACAGGGAGAAAAAGAAAAAGCAGCAGCTCATGACCCAGATAAGTGGAGTGAAGAAATTAATGCATAGTTCAAGC | | | | |
| CTAAACAATACAAGCATCTCACGCTTTGGAGTCAACACTGAAAATGAAGATCACCTGGCCAAGGAGCTGGAAGACCTGAACAAATGGGGTCTTAACATCTTTAATGTGGCTGGATATTCTCACAATAGACCCCT | | | | |
| AACATGCATCATGTATGCTATATTCCAGGAAAGAGACCTCCTAAAGACATTCAGAATCTCATCTGACACATTTATAACCTACATGATGACTTTAGAAGACCATTACCATTCTGACGTGGCATATCACAACAGCC | | | | |
| TGCACGCTGCTGATGTAGCCCAGTCGACCCATGTTCTCCTTTCTACACCAGCATTAGACGCTGTCTTCACAGATTTGGAGATCCTGGCTGCCATTTTTGCAGCTGCCATCCATGACGTTGATCATCCTGGAGTC | | | | |
| TCCAATCAGTTTCTCATCAACACAAATTCAGAACTTGCTTTGATGTATAATGATGAATCTGTGTTGGAAAATCATCACCTTGCTGTGGGTTTCAAACTGCTGCAAGAAGAACACTGTGACATCTTCATGAATCT | | | | |
| CACCAAGAAGCAGCGTCAGACACTCAGGAAGATGGTTATTGACATGGTGTTAGCAACTGATATGTCTAAACATATGAGCCTGCTGGCAGACCTGAAGACAATGGTAGAAACGAAGAAAGTTACAAGTTCAGGCG | | | | |
| TTCTTCTCCTAGACAACTATACCGATCGCATTCAGGTCCTTCGCAACATGGTACACTGTGCAGACCTGAGCAACCCCACCAAGTCCTTGGAATTGTATCGGCAATGGACAGACCGCATCATGGAGGAATTTTTC | | | | |
| CAGCAGGGAGACAAAGAGCGGGAGAGGGGAATGGAAATTAGCCCAATGTGTGATAAACACACAGCTTCTGTGGAAAAATCCCAGGTTGGTTTCATCGACTACATTGTCCATCCATTGTGGGAGACATGGGCAGA | | | | |
| TTTGGTACAGCCTGATGCTCAGGACATTCTCGATACCTTAGAAGATAACAGGAACTGGTATCAGAGCATGATACCTCAAAGTCCCTCACCACCACTGGACGAGCAGAACAGGGACTGCCAGGGTCTGATGGAGA | | | | |
| AGTTTCAGTTTGAACTGACTCTCGATGAGGAAGATTCTGAAGGACCTGAGAAGGAGGGAGAGGGACACAGCTATTTCAGCAGCACAAAGACGCTTTGTGTGATTGATCCAGAAAACAGAGATTCCCTGGGAGAG | | | | |
| ACTGACATAGACATTGCAACAGAAGACAAGTCCCCCGTGGATACATAATCCCCCTCTCCCTGTGGAGATGAACATTCTATCCTTGATGAGCATGCCAGCTATGTGGTAGGGCCAGCCCACCATGGGGGCCAAGA | | | | |
| CCTGCACAGGACAAGGGCCACCTGGCCTTTCAGTTACTTGAGTTTGGAGTCAGAAAGCAAGACCAGGAAGCAAATAGCAGCTCAGGAAATCCCACGGTTGACTTGCCTTGATGGCAAGCTTGGTGGAGAGGGCT | | | | |
| GAAGCTGTTGCTGGGGGCCGATTCTGATCAAGACACATGGCTTGAAAATGGAAGACACAAAACTGAGAGATCATTCTGCACTAAGTTTCGGGAACTTATCCCCGACAGTGACTGAACTCACTGACTAATAACTT | | | | |
| CATTTATGAATCTTCTCACTTGTCCCTTTGTCTGCCAACCTGTGTGCCTTTTTTGTAAAACATTTTCATGTCTTTAAAATGCCTGTTGAATACCTGGAGTTTAGTATCAACTTCTACACAGATAAGCTTTCAAA | | | | |
| GTTGACAAACTTTTTTGACTCTTTCTGGAAAAGGGAAAGAAAATAGTCTTCCTTCTTTCTTGGGCAATATCCTTCACTTTACTACAGTTACTTTTGCAAACAGACAGAAAGGATACACTTCTAACCACATTTTA | | | | |
| CTTCCTTCCCCTGTTGTCCAGTCCAACTCCACAGTCACTCTTAAAACTTCTCTCTGTTTGCCTGCCTCCAACAGTACTTTTAACTTTTTGCTGTAAACAGAATAAAATTGAACAAATTAGGGGGTAGAAAGGAG | | | | |
| CAGTGGTGTCGTTCACCGTGAGAGTCTGCATAGAACTCAGCAGTGTGCCCTGCTGTGTCTTGGACCCTGCCCCCCACAGGAGTTGCTACAGTCCCTGGCCCTGCTTCCCATCCTCCTCTCTTCACCCCGTTAGC | | | | |
| TGTTTTCAATGTAATGCTGCCGTCCTTCTCTTGCACTGCCTTCTGCGCTAACACCTCCATTCCTGTTTATAACCGTGTATTTATTACTTAATGTATATAATGTAATGTTTTGTAAGTTATTAATTTATATATCT | | | | |
| AACATTGCCTGCCAATGGTGGTGTTAAATTTGTGTAGAAAACTCTGCCTAAGAGTTACGACTTTTTCTTGTAATGTTTTGTATTGTGTATTATATAACCCAAACGTCACTTAGTAGAGACATATGGCCCCCTTG | | | | |
| GCAGAGAGGACAGGGGTGGGCTTTTGTTCAAAGGGTCTGCCCTTTCCCTGCCTGAGTTGCTACTTCTGCACAACCCCTTTATGAACCAGTTTTGGAAACAATATTCTCACATTAGATACTAAATGGTTTATACT | | | | |
| GAGTCTTTTACTTTTGTATAGCTTGATAGGGGCAGGGGCAATGGGATGTAGTTTTTACCCAGGTTCTATCCAAATCTATGTGGGCATGAGTTGGGTTATAACTGGATCCTACTATCATTGTGGCTTTGGTTCAA | | | | |
| AAGGAAACACTACATTTGCTCACAGATGATTCTTCTGATTCTTCTGAATGCTCCCGAACTACTGACTTTGAAGAGGTAGCCTCCTGCCTGCCATTAAGCAGGAATGTCATGTTCCAGTTCATTACAAAAGAAAA | | | | |
| CAATAAAACAATGTGAATTTTTATAATAAAAAAAAAAAAAAGGAATTC | | | | |
| 1784, | EPI-19-MTA-1, | NM_002600, | CCATGATGCGGTCTGTCCA, | |
| 1785, | EPI-19-MTA-14, | NM_002600, | CTTGGTGGGGTTGCTCAG, | |
| 1786, | EPI-19-MTA-15, | NM_002600, | AGAGTCAGTTCAAACTG, | |
| 1787, | EPI-19-MTA-16, | NM_002600, | AAGACCCCATTTGTTCA, | |
| 1788, | EPI-19-MTA-17, | NM_002600, | TCTGCCCATGTCTCCCA, | |
| Concatemer Nucleic Acid Sequences of PDE4B gene oligo sequences | | | | |
| (SEQ ID NO: 1789) | | | | |
| CCATGATGCGGTCTGTCCACTTGGTGGGGTTGCTCAGAGAGTCAGTTCAAACTGAAGACCCCATTTGTTCATCTGCCCATGTCTCCCA | | | | |
| PDE4C Nucleic Acid Sequences (GENBANK ACCESSION NO.X61177) | | | | |
| (SEQ ID NO:1790) | | | | |
| CCAGTCTGCGGACCGTTCGGAGCAACGTGGCGGCCCTTGCCCGCCAGCAATGCCTAGGAGCAGCCAAGCAGGGACCCGTCGGAAACCCTTCATCCAGCCTTTCTCTGGCGCATGGAGAACCTGGGGGTCGGCGA | | | | |
| AGGGGCAGAGGCTTGCAGCAGGTTGAGTCGCTCTCGCGGCCGCCACAGCATGACCAGAGCCCCGAAGCACCTGTGGCGGCAACCCCGGCGCCCCATCCGCATCCAACAGCGCTTCTATTCGGATCCGGACAAGT | | | | |
| CCGCGGGCTGCCGCGAGAGGGACCTGAGCCCGCGGCCGGAGCTCAGGAAGTCGCGGCTCTCCTGGCCCGTTTCCTCCTGCAGGCGCTTTGACCTGGAAAATGGGCTCTCGTGTGGGAGGAGGGCCCTGGACCCT | | | | |
| CAGTCCAGCCCTGGCCTGGGCCGGATTATGCAGGCTCCAGTCCCGCACAGCCAGCGGCGCGAGTCCTTCCTGTACCGCTCAGATAGCGACTATGAACTCTCGCCCAAGGCCATGTCTCGGAACTCCTCTGTGGC | | | | |
| CAGCGACCTACATGGAGAGGACATGATTGTGACGCCCTTTGCCCAGGTCCTGGCCAGTCTGCGGACCGTTCGGAGCAACGTGGCGGCCCTTGCCCGCCAGCAATGCCTAGGAGCAGCCAAGCAGGGACCCGTCG | | | | |
| GAAACCCTTCATCCAGCAATCAGCTCCCTCCTGCAGAGGACACGGGGCAGAAGCTGGCATTGGAGACGCTAGACGAGCTGGACTGGTGCCTGGATCAGTTGGAGACGCTGCAGACCCGGCACTCGGTGGGGGAG | | | | |
| ATGGCCTCCAACAAGTTCAAGCGGATCCTGAACCGGGAGTTGACCCACCTGTCCGAAACCAGCCGCTCCGGGAACCAGGTGTCCGAGTACATCTCCCGGACCTTCCTGGACCAGCAGACCGAGGTGGAGCTGCC | | | | |
| CAAGGTGACCGCTGAGGAGGCCCCACAGCCCATGTCCCGGATCAGTGGCCTACATGGGCTCTGCCACAGTGCCAGCCTCTCCTCAGCCACTGTCCCACGCTTTGGGGTCCAGACTGACCAGGAGGAGCAACTGG | | | | |
| CCAAGGAGCTAGAAGACACCAACAAGTGGGGACTTGATGTGTTCAAGGTGGCGGACGTAAGTGGGAACCGGCCCCTCACAGCTATCATATTCAGCATTTTTCAGGAGCGGGACCTGCTGAAGACATTCCAGATC | | | | |
| GGCTGTGTTCACAGACTTGGAAATCCTGGCTGCCCTCTTTGCAAGCGCCATCCACGACGTGGACCATCCTGGGGTCTCCAACCAGTTTCTGATTAACACCAACTCAGACGTGGCGCTTATGTACAACGACGCCT | | | | |
| CGGTGCTGGAGAACCATCACCTGGCTGTGGGCTTCAAGCTGCTGCAGGCAGAGAACTGCGATATCTTCCAGAACCTCAGCGCCAAGCAGCGACTGAGTCTGCGCAGGATGGTCATTGACATGGTGCTGGCCACA | | | | |
| GACATGTCCAAACACATGAACCTCCTGGCCGACCTCAAGACCATGGTGGAGACCAAGAAGGTGACAAGCCTCGGTGTCCTCCTCCTGGACAACTATTCCGACCGAATCCAGGTCTTGCAGAACCTGGTGCACTG | | | | |
| TGCTGATCTGAGCAACCCCACCAAGCCGCTGCCCCTGTACCGCCAGTGGACGGACCGCATCATGGCCGAGTTCTTCCAGCAGGGAGACCGCGAGCGTGAGTCGGGCCTGGACATCAGTCCCATGTGTGACAAGC | | | | |
| ATACGGCCTCAGTGGAGAAGTCCCAGGTGGGTTTCATTGACTACATTGCTCACCCACTGTGGGAGACTTGGGCTGACCTGGTCCACCCAGATGCACAGGACCTGCTGGACACGCTGGAGGACAATCGAGAGTGG | | | | |
| TACCAGAGCAAGATCCCCCGAAGTCCCTCAGACCTCACCAACCCCGAGCGGGACGGGCCTGACAGATTCCAGTTTGAACTGACTCTGGAGGAGGCAGAGGAAGAGGATGAGGAGGAAGAAGAGGAGGGGGAAGA | | | | |
| GACAGCTTTAGCCAAAGAGGCCTTGGAGTTGCCTGACACTGAACTCCTGTCCCCTGAAGCCGGCCCAGACCCTGGGGACTTACCCCTCGACAACCAGAGGACTTAGGGCCAGCCCTGCGTGAACTGCAGGGGCA | | | | |
| ATGGATGGTAAAGCCCTTTGGCTCTTGGCAGGCAGACTTTCCAGGAAGAGGCTCCATGTGGCTCCTGCTTCACTTTCCCACCCATTTAGGGAGACAATCAAGCTCTTAGTTATAGGTGGCTCCCAGGGTCTAAT | | | | |
| TGGAGGCACCTGGCTGGGGTCCACTCTGACCCTAGACTTGCCTAAAAGAGCTCTCTAAGGGGCAGCCTCTTACGATGCCCTGGTGTCTTTCTCCTGGGCTTCTATCCCTGTGAGGAGAGGTGCTGTCTGCTGGA | | | | |
| GCCTCTAGTCCACCCTCTCCAGTGGTCACTCTTGAGTCACATCTGTCACTTAATTATTTCCTTCTTTATCAAATATTTATTGCTCATCTACTTCGGGCCAGCTTTCTGCCTCTGTAGTAGCCCTGCACAAAGGG | | | | |
| TGGGGAGTCAGGAGACCATCCCAAAGGCATCTCCCTGTCTTCCTCTACCAAGCGGCTCTCTGCAAGAGCATGGAAATGTGAGTGGGGAAAATTTTCAGCACCAAAGCTTCACTCATACCCAGTTTTGTTTCTGA | | | | |
| AACTACGGTAGGGGGCAGGAAGAGGAGCAGAAAAGAAGGGCTGGGCAAGGCATAGTGGCTTATGCCTGTAATCCCGGTACTTTGGGAGGCTGAGGTGGGAGGACTGCTTAAGCTCAGGAGTTTGAGACCAGCCT | | | | |
| GGGCAACATAGCAAGACCCCCACCATCTCTGAAAAAAAAAATTAGCCAGGCATGGTGGTGTGCACCTGAGAATCCCAGCTACTCAGAAGGTTGAGACAAAGGGGATCGCTTGAGCCCAGGAGTTGGAGGCTGAA | | | | |
| GAGAGCTATGACTGCATCACTGCACTCCAGCCTGGGCAACACAGCAAGATCCTGTCTAAAAATAAAAAGAAAAGAGAAGGAAAGGAAAGAGACGGGGCTCTGAGGCCGAGCACAGTGGCCCATGCCTATAATCC | | | | |
| CAGCACTTTGGGAGGCTGAGGCAGGTGGATCACCTGAGGTTAGGAGTTCGAGACCAGCCTGGCCAACATGGTGAAACCCCATCTCTACTAAAAATACAAAAATTGGCTGGGCATGGTGGCGGGTGCCTGTAATC | | | | |
| CCAGCTACTGGGGAGGCTGAGGCAGGAGAATCACTTGAATTCAGGAGGTGGAGGTTGCAGTGAGCCGACATCATGCCACTGCACTCCAGCCTGGGGCTGACAGAGCAAGACACTGTCTCAAAAAAGAAAAAAAA | | | | |
| AAAAAAAAAAA | | | | |
| 1791, | Z46632, | Z46632, | TTTTTTTTTTTTTTTTTTTC, | |
| 1792, | Z46632, | Z46632, | TTTTTTTTTCTTTTTTGAGA, | |
| 1793, | Z46632, | Z46632, | TTTTTTGAGACAGTGTCTTG, | |
| 1794, | Z46632, | Z46632, | CAGTGTCTTGCTCTGTCAGC, | |
| 1795, | Z46632, | Z46632, | CTCTGTCAGCCCCAGGCTGG, | |
| 1796, | Z46632, | Z46632, | CCCAGGCTGGAGTGCAGTGG, | |
| 1797, | Z46632, | Z46632, | AGTGCAGTGGCATGATGTCG, | |
| 1798, | Z46632, | Z46632, | CATGATGTCGGCTCACTGCA, | |
| 1799, | Z46632, | Z46632, | GCTCACTGCAACCTCCACCT, | |
| 1800, | Z46632, | Z46632, | ACCTCCACCTCCTGAATTCA, | |
| 1801, | Z46632, | Z46632, | CCTGAATTCAAGTGATTCTC, | |
| 1802, | Z46632, | Z46632, | AGTGATTCTCCTGCCTCAGC, | |
| 1803, | Z46632, | Z46632, | CTGCCTCAGCCTCCCCAGTA, | |
| 1804, | Z46632, | Z46632, | CTCCCCAGTAGCTGGGATTA, | |
| 1805, | Z46632, | Z46632, | GCTGGGATTACAGGCACCCG, | |
| 1806, | Z46632, | Z46632, | CAGGCACCCGCCACCATGCC, | |
| 1807, | Z46632, | Z46632, | CCACCATGCCCAGCCAATTT, | |
| 1808, | Z46632, | Z46632, | CAGCCAATTTTTGTATTTTT, | |
| 1809, | Z46632, | Z46632, | TTGTATTTTTAGTAGAGATG, | |
| 1810, | Z46632, | Z46632, | AGTAGAGATGGGGTTTCACC, | |
| 1811, | Z46632, | Z46632, | GGGTTTCACCATGTTGGCCA, | |
| 1812, | Z46632, | Z46632, | ATGTTGGCCAGGCTGGTCTC, | |
| 1813, | Z46632, | Z46632, | GGCTGGTCTCGAACTCCTAA, | |
| 1814, | Z46632, | Z46632, | GAACTCCTAACCTCAGGTGA, | |
| 1815, | Z46632, | Z46632, | CCTCAGGTGATCCACCTGCC, | |
| 1816, | Z46632, | Z46632, | TCCACCTGCCTCAGCCTCCC, | |
| 1817, | Z46632, | Z46632, | TCAGCCTCCCAAAGTGCTGG, | |
| 1818, | Z46632, | Z46632, | AAAGTGCTGGGATTATAGGC, | |
| 1819, | Z46632, | Z46632, | GATTATAGGCATGGGCCACT, | |
| 1820, | Z46632, | Z46632, | ATGGGCCACTGTGCTCGGCC, | |
| 1821, | Z46632, | Z46632, | GTGCTCGGCCTCAGAGCCCC, | |
| 1822, | Z46632, | Z46632, | TCAGAGCCCCGTCTCTTTCC, | |
| 1823, | Z46632, | Z46632, | GTCTCTTTCCTTTCCTTCTC, | |
| 1824, | Z46632, | Z46632, | TTTCCTTCTCTTTTCTTTTT, | |
| 1825, | Z46632, | Z46632, | TTTTCTTTTTATTTTTAGAC, | |
| 1826, | Z46632, | Z46632, | ATTTTTAGACAGGATCTTGC, | |
| 1827, | Z46632, | Z46632, | AGGATCTTGCTGTGTTGCCC, | |
| 1828, | Z46632, | Z46632, | TGTGTTGCCCAGGCTGGAGT, | |
| 1829, | Z46632, | Z46632, | AGGCTGGAGTGCAGTGATGC, | |
| 1830, | Z46632, | Z46632, | GCAGTGATGCAGTCATAGCT, | |
| 1831, | Z46632, | Z46632, | AGTCATAGCTCTCTTCAGCC, | |
| 1832, | Z46632, | Z46632, | CTCTTCAGCCTCCAACTCCT, | |
| 1833, | Z46632, | Z46632, | TCCAACTCCTGGGCTCAAGC, | |
| 1834, | Z46632, | Z46632, | GGGCTCAAGCGATCCCCTTT, | |
| 1835, | Z46632, | Z46632, | GATCCCCTTTGTCTCAACCT, | |
| 1836, | Z46632, | Z46632, | GTCTCAACCTTCTGAGTAGC, | |
| 1837, | Z46632, | Z46632, | TCTGAGTAGCTGGGATTCTC, | |
| 1838, | Z46632, | Z46632, | TGGGATTCTCAGGTGCACAC, | |
| 1839, | Z46632, | Z46632, | AGGTGCACACCACCATGCCT, | |
| 1840, | Z46632, | Z46632, | CACCATGCCTGGCTAATTTT, | |
| 1841, | Z46632, | Z46632, | GGCTAATTTTTTTTTTCAGA, | |
| 1842, | Z46632, | Z46632, | TTTTTTCAGAGATGGTGGGG, | |
| 1843, | Z46632, | Z46632, | GATGGTGGGGGTCTTGCTAT, | |
| 1844, | Z46632, | Z46632, | GTCTTGCTATGTTGCCCAGG, | |
| 1845, | Z46632, | Z46632, | GTTGCCCAGGCTGGTCTCAA, | |
| 1846, | Z46632, | Z46632, | CTGGTCTCAAACTCCTGAGC, | |
| 1847, | Z46632, | Z46632, | ACTCCTGAGCTTAAGCAGTC, | |
| 1848, | Z46632, | Z46632, | TTAAGCAGTCCTCCCACCTC, | |
| 1849, | Z46632, | Z46632, | CTCCCACCTCAGCCTCCCAA, | |
| 1850, | Z46632, | Z46632, | AGCCTCCCAAAGTACCGGGA, | |
| 1851, | Z46632, | Z46632, | AGTACCGGGATTACAGGCAT, | |
| 1852, | Z46632, | Z46632, | TTACAGGCATAAGCCACTAT, | |
| 1853, | Z46632, | Z46632, | AAGCCACTATGCCTTGCCCA, | |
| 1854, | Z46632, | Z46632, | GCCTTGCCCAGCCCTTCTTT, | |
| 1855, | Z46632, | Z46632, | GCCCTTCTTTTCTGCTCCTC, | |
| 1856, | Z46632, | Z46632, | TCTGCTCCTCTTCCTGCCCC, | |
| 1857, | Z46632, | Z46632, | TTCCTGCCCCCTACCGTAGT, | |
| 1858, | Z46632, | Z46632, | CTACCGTAGTTTCAGAAACA, | |
| 1859, | Z46632, | Z46632, | TTCAGAAACAAAACTGGGTA, | |
| 1850, | Z46632, | Z46632, | AAACTGGGTATGAGTGAAGC, | |
| 1851, | Z46632, | Z46632, | AGTACCGGGATTACAGGCAT, | |
| 1852, | Z46632, | Z46632, | TTACAGGCATAAGCCACTAT, | |
| 1853, | Z46632, | Z46632, | AAGCCACTATGCCTTGCCCA, | |
| 1854, | Z46632, | Z46632, | GCCTTGCCCAGCCCTTCTTT, | |
| 1855, | Z46632, | Z46632, | GCCCTTCTTTTCTGCTCCTC, | |
| 1856, | Z46632, | Z46632, | TCTGCTCCTCTTCCTGCCCC, | |
| 1857, | Z46632, | Z46632, | TTCCTGCCCCCTACCGTAGT, | |
| 1858, | Z46632, | Z46632, | CTACCGTAGTTTCAGAAACA, | |
| 1859, | Z46632, | Z46632, | TTCAGAAACAAAACTGGGTA, | |
| 1860, | Z46632, | Z46632, | AAACTGGGTATGAGTGAAGC, | |
| 1861, | Z46632, | Z46632, | TGAGTGAAGCTTTGGTGCTG, | |
| 1862, | Z46632, | Z46632, | TTTGGTGCTGAAAATTTTCC, | |
| 1863, | Z46632, | Z46632, | AAAATTTTCCCCACTCACAT, | |
| 1864, | Z46632, | Z46632, | CCACTCACATTTCCATGCTC, | |
| 1865, | Z46632, | Z46632, | TTCCATGCTCTTGCAGAGAG, | |
| 1866, | Z46632, | Z46632, | TTGCAGAGAGCCGCTTGGTA, | |
| 1867, | Z46632, | Z46632, | CCGCTTGGTAGAGGAAGACA, | |
| 1868, | Z46632, | Z46632, | GAGGAAGACAGGGAGATGCC, | |
| 1869, | Z46632, | Z46632, | GGGAGATGCCTTTGGGATGG, | |
| 1870, | Z46632, | Z46632, | TTTGGGATGGTCTCCTGACT, | |
| 1871, | Z46632, | Z46632, | TCTCCTGACTCCCCACCCTT, | |
| 1872, | Z46632, | Z46632, | CCCCACCCTTTGTGCAGGGC, | |
| 1873, | Z46632, | Z46632, | TGTGCAGGGCTACTACAGAG, | |
| 1874, | Z46632, | Z46632, | TACTACAGAGGCAGAAAGCT, | |
| 1875, | Z46632, | Z46632, | GCAGAAAGCTGGCCCGAAGT, | |
| 1876, | Z46632, | Z46632, | GGCCCGAAGTAGATGAGCAA, | |
| 1877, | Z46632, | Z46632, | AGATGAGCAATAAATATTTG, | |
| 1878, | Z46632, | Z46632, | TAAATATTTGATAAAGAAGG, | |
| 1879, | Z46632, | Z46632, | ATAAAGAAGGAAATAATTAA, | |
| 1880, | Z46632, | Z46632, | AAATAATTAAGTGACAGATG, | |
| 1881, | Z46632, | Z46632, | GTGACAGATGTGACTCAAGA, | |
| 1882, | Z46632, | Z46632, | TGACTCAAGAGTGACCACTG, | |
| 1883, | Z46632, | Z46632, | GTGACCACTGGAGAGGGTGG, | |
| 1884, | Z46632, | Z46632, | GAGAGGGTGGACTAGAGGCT, | |
| 1885, | Z46632, | Z46632, | ACTAGAGGCTCCAGCAGACA, | |
| 1886, | Z46632, | Z46632, | CCAGCAGACAGCACCTCTCC, | |
| 1887, | Z46632, | Z46632, | GCACCTCTCCTCACAGGGAT, | |
| 1888, | Z46632, | Z46632, | TCACAGGGATAGAAGCCCAG, | |
| 1988, | Z46632, | Z46632, | CTGCCTGCAGCAGCTTGAAG, | |
| 1989, | Z46632, | Z46632, | CAGCTTGAAGCCCACAGCCA, | |
| 1990, | Z46632, | Z46632, | CCCACAGCCAGGTGATGGTT, | |
| 1991, | Z46632, | Z46632, | GGTGATGGTTCTCCAGCACC, | |
| 1992, | Z46632, | Z46632, | CTCCAGCACCGAGGCGTCGT, | |
| 1993, | Z46632, | Z46632, | GAGGCGTCGTTGTACATAAG, | |
| 1994, | Z46632, | Z46632, | TGTACATAAGCGCCACGTCT, | |
| 1995, | Z46632, | Z46632, | CGCCACGTCTGAGTTGGTGT, | |
| 1996, | Z46632, | Z46632, | GAGTTGGTGTTAATCAGAAA, | |
| 1997, | Z46632, | Z46632, | TAATCAGAAACTGGTTGGAG, | |
| 1998, | Z46632, | Z46632, | CTGGTTGGAGACCCCAGGAT, | |
| 1999, | Z46632, | Z46632, | ACCCCAGGATGGTCCACGTC, | |
| 2000, | Z46632, | Z46632, | GGTCCACGTCGTGGATGGCG, | |
| 2001, | Z46632, | Z46632, | GTGGATGGCGCTTGCAAAGA, | |
| 2002, | Z46632, | Z46632, | CTTGCAAAGAGGGCAGCCAG, | |
| 2003, | Z46632, | Z46632, | GGGCAGCCAGGATTTCCAAG, | |
| 2004, | Z46632, | Z46632, | GATTTCCAAGTCTGTGAACA, | |
| 2005, | Z46632, | Z46632, | TCTGTGAACACAGCCTCGAG, | |
| 2006, | Z46632, | Z46632, | CAGCCTCGAGGGCGGGCGTA, | |
| 2007, | Z46632, | Z46632, | GGCGGGCGTAGCCAGCAGCA, | |
| 2008, | Z46632, | Z46632, | GCCAGCAGCACATGCGTGGA, | |
| 2009, | Z46632, | Z46632, | CATGCGTGGACTGGGCCACG, | |
| 2010, | Z46632, | Z46632, | CTGGGCCACGTCGGCGGCAT, | |
| 2011, | Z46632, | Z46632, | TCGGCGGCATGTAGGCTGTT, | |
| 2012, | Z46632, | Z46632, | GTAGGCTGTTGTGGTAGGCC, | |
| 2013, | Z46632, | Z46632, | GTGGTAGGCCACATTGGCGT, | |
| 2014, | Z46632, | Z46632, | ACATTGGCGTGGTAGTGACC, | |
| 2015, | Z46632, | Z46632, | GGTAGTGACCCTCCAGCATC, | |
| 2016, | Z46632, | Z46632, | CTCCAGCATCAGCAGGTAGG, | |
| 2017, | Z46632, | Z46632, | AGCAGGTAGGTGGCCAGTGT, | |
| 2018, | Z46632, | Z46632, | TGGCCAGTGTGTCTGCTGGG, | |
| 2019, | Z46632, | Z46632, | GTCTGCTGGGATCTGGAATG, | |
| 2020, | Z46632, | Z46632, | ATCTGGAATGTCTTCAGCAG, | |
| 2021, | Z46632, | Z46632, | TCTTCAGCAGGTCCCGCTCC, | |
| 2022, | Z46632, | Z46632, | GTCCCGCTCCTGAAAAATGC, | |
| 2023, | Z46632, | Z46632, | TGAAAAATGCTGAATATGAT, | |
| 2024, | Z46632, | Z46632, | TGAATATGATAGCTGTGAGG, | |
| 2025, | Z46632, | Z46632, | AGCTGTGAGGGGCCGGTTCC, | |
| 2026, | Z46632, | Z46632, | GGCCGGTTCCCACTTACGTC, | |
| 2027, | Z46632, | Z46632, | CACTTACGTCCGCCACCTTG, | |
| 2028, | Z46632, | Z46632, | CGCCACCTTGAACACATCAA, | |
| 2029, | Z46632, | Z46632, | AACACATCAAGTCCCCACTT, | |
| 2030, | Z46632, | Z46632, | GTCCCCACTTGTTGGTGTCT, | |
| 2031, | Z46632, | Z46632, | GTTGGTGTCTTCTAGCTCCT, | |
| 2032, | Z46632, | Z46632, | TCTAGCTCCTTGGCCAGTTG, | |
| 2033, | Z46632, | Z46632, | TGGCCAGTTGCTCCTCCTGG, | |
| 2034, | Z46632, | Z46632, | CTCCTCCTGGTCAGTCTGGA, | |
| 2035, | Z46632, | Z46632, | TCAGTCTGGACCCCAAAGCG, | |
| 2036, | Z46632, | Z46632, | CCCCAAAGCGTGGGACAGTG, | |
| 2037, | Z46632, | Z46632, | TGGGACAGTGGCTGAGGAGA, | |
| 2038, | Z46632, | Z46632, | GCTGAGGAGAGGCTGGCACT, | |
| 2039, | Z46632, | Z46632, | GGCTGGCACTGTGGCAGAGC, | |
| 2040, | Z46632, | Z46632, | GTGGCAGAGCCCATGTAGGC, | |
| 2041, | Z46632, | Z46632, | CCATGTAGGCCACTGATCCG, | |
| 2042, | Z46632, | Z46632, | CACTGATCCGGGACATGGGC, | |
| 2043, | Z46632, | Z46632, | GGACATGGGCTGTGGGGCCT, | |
| 2044, | Z46632, | Z46632, | TGTGGGGCCTCCTCAGCGGT, | |
| 2045, | Z46632, | Z46632, | CCTCAGCGGTCACCTTGGGC, | |
| 2046, | Z46632, | Z46632, | CACCTTGGGCAGCTCCACCT, | |
| 2047, | Z46632, | Z46632, | AGCTCCACCTCGGTCTGCTG, | |
| 2048, | Z46632, | Z46632, | CGGTCTGCTGGTCCAGGAAG, | |
| 2049, | Z46632, | Z46632, | GTCCAGGAAGGTCCGGGAGA, | |
| 2050, | Z46632, | Z46632, | GTCCGGGAGATGTACTCGGA, | |
| 2051, | Z46632, | Z46632, | TGTACTCGGACACCTGGTTC, | |
| 2052, | Z46632, | Z46632, | CACCTGGTTCCCGGAGCGGC, | |
| 2053, | Z46632, | Z46632, | CCGGAGCGGCTGGTTTCGGA, | |
| 2054, | Z46632, | Z46632, | TGGTTTCGGACAGGTGGGTC, | |
| 2055, | Z46632, | Z46632, | CAGGTGGGTCAACTCCCGGT, | |
| 2056, | Z46632, | Z46632, | AACTCCCGGTTCAGGATCCG, | |
| 2057, | Z46632, | Z46632, | TCAGGATCCGCTTGAACTTG, | |
| 2058, | Z46632, | Z46632, | CTTGAACTTGTTGGAGGCCA, | |
| 2059, | Z46632, | Z46632, | TTGGAGGCCATCTCCCCCAC, | |
| 2060, | Z46632, | Z46632, | TCTCCCCCACCGAGTGCCGG, | |
| 2061, | Z46632, | Z46632, | CGAGTGCCGGGTCTGCAGCG, | |
| 2062, | Z46632, | Z46632, | GTCTGCAGCGTCTCCAACTG, | |
| 2063, | Z46632, | Z46632, | TCTCCAACTGATCCAGGCAC, | |
| 2064, | Z46632, | Z46632, | ATCCAGGCACCAGTCCAGCT, | |
| 2065, | Z46632, | Z46632, | CAGTCCAGCTCGTCTAGCGT, | |
| 2066, | Z46632, | Z46632, | CGTCTAGCGTCTCCAATGCC, | |
| 2067, | Z46632, | Z46632, | CTCCAATGCCAGCTTCTGCC, | |
| 2068, | Z46632, | Z46632, | AGCTTCTGCCCCGTGTCCTC, | |
| 2069, | Z46632, | Z46632, | CCGTGTCCTCTGCAGGAGGG, | |
| 2070, | Z46632, | Z46632, | TGCAGGAGGGAGCTGATTGC, | |
| 2071, | Z46632, | Z46632, | AGCTGATTGCTGGATGAAGG, | |
| 2072, | Z46632, | Z46632, | TGGATGAAGGGTTTCCGACG, | |
| 2073, | Z46632, | Z46632, | GTTTCCGACGGGTCCCTGCT, | |
| 2074, | Z46632, | Z46632, | GGTCCCTGCTTGGCTGCTCC, | |
| 2075, | Z46632, | Z46632, | TGGCTGCTCCTAGGCATTGC, | |
| 2076, | Z46632, | Z46632, | TAGGCATTGCTGGCGGGCAA, | |
| 2077, | Z46632, | Z46632, | TGGCGGGCAAGGGCCGCCAC, | |
| 2078, | Z46632, | Z46632, | GGGCCGCCACGTTGCTCCGA, | |
| 2079, | Z46632, | Z46632, | GTTGCTCCGAACGGTCCGCA, | |
| 2080, | Z46632, | Z46632, | ACGGTCCGCAGACTGGCCAG, | |
| 2081, | Z46632, | Z46632, | GACTGGCCAGGACCTGGGCA, | |
| 2082, | Z46632, | Z46632, | GACCTGGGCAAAGGGCGTCA, | |
| 2083, | Z46632, | Z46632, | AAGGGCGTCACAATCATGTC, | |
| 2084, | Z46632, | Z46632, | CAATCATGTCCTCTCCATGT, | |
| 2085, | Z46632, | Z46632, | CTCTCCATGTAGGTCGCTGG, | |
| 2086, | Z46632, | Z46632, | AGGTCGCTGGCCACAGAGGA, | |
| 2087, | Z46632, | Z46632, | CCACAGAGGAGTTCCGAGAC, | |
| 2088, | Z46632, | Z46632, | GTTCCGAGACATGGCCTTGG, | |
| 2089, | Z46632, | Z46632, | ATGGCCTTGGGCGAGAGTTC, | |
| 2090, | Z46632, | Z46632, | GCGAGAGTTCATAGTCGCTA, | |
| 2091, | Z46632, | Z46632, | ATAGTCGCTATCTGAGCGGT, | |
| 2092, | Z46632, | Z46632, | TCTGAGCGGTACAGGAAGGA, | |
| 2093, | Z46632, | Z46632, | ACAGGAAGGACTCGCGCCGC, | |
| 2094, | Z46632, | Z46632, | CTCGCGCCGCTGGCTGTGCG, | |
| 2095, | Z46632, | Z46632, | TGGCTGTGCGGGACTGGAGC, | |
| 2096, | Z46632, | Z46632, | GGACTGGAGCCTGCATAATC, | |
| 2097, | Z46632, | Z46632, | CTGCATAATCCGGCCCAGGC, | |
| 2098, | Z46632, | Z46632, | CGGCCCAGGCCAGGGCTGGA, | |
| 2099, | Z46632, | Z46632, | CAGGGCTGGACTGAGGGTCC, | |
| 2100, | Z46632, | Z46632, | CTGAGGGTCCAGGGCCCTCC, | |
| 2101, | Z46632, | Z46632, | AGGGCCCTCCTCCCACACGA, | |
| 2102, | Z46632, | Z46632, | TCCCACACGAGAGCCCATTT, | |
| 2103, | Z46632, | Z46632, | AGCGCCTGCAGGAGGAAACG, | |
| 2104, | Z46632, | Z46632, | GGAGGAAACGGGCCAGGAGA, | |
| 2105, | Z46632, | Z46632, | GGCCAGGAGAGCCGCGACTT, | |
| 2106, | Z46632, | Z46632, | GGAGGAAACGGGCCAGGAGA, | |
| 2107, | Z46632, | Z46632, | GGCCAGGAGAGCCGCGACTT, | |
| 2108, | Z46632, | Z46632, | GCCGCGACTTCCTGAGCTCC, | |
| 2109, | Z46632, | Z46632, | CCTGAGCTCCGGCCGCGGGC, | |
| 2110, | Z46632, | Z46632, | GGCCGCGGGCTCAGGTCCCT, | |
| 2111, | Z46632, | Z46632, | TCAGGTCCCTCTCGCGGCAG, | |
| 2112, | Z46632, | Z46632, | CTCGCGGCAGCCCGCGGACT, | |
| 2113, | Z46632, | Z46632, | CCCGCGGACTTGTCCGGATC, | |
| 2114, | Z46632, | Z46632, | TGTCCGGATCCGAATAGAAG, | |
| 2115, | Z46632, | Z46632, | CGAATAGAAGCGCTGTTGGA, | |
| 2116, | Z46632, | Z46632, | CGCTGTTGGATGCGGATGGG, | |
| 2117, | Z46632, | Z46632, | TGCGGATGGGGCGCCGGGGT, | |
| 2118, | Z46632, | Z46632, | GCGCCGGGGTTGCCGCCACA, | |
| 2119, | Z46632, | Z46632, | TGCCGCCACAGGTGCTTCGG, | |
| 2120, | Z46632, | Z46632, | GGTGCTTCGGGGCTCTGGTC, | |
| 2121, | Z46632, | Z46632, | GGCTCTGGTCATGCTGTGGC, | |
| 2122, | Z46632, | Z46632, | ATGCTGTGGCGGCCGCGAGA, | |
| 2123, | Z46632, | Z46632, | GGCCGCGAGAGCGACTCAAC, | |
| 2124, | Z46632, | Z46632, | GCGACTCAACCTGCTGCAAG, | |
| 2125, | Z46632, | Z46632, | CTGCTGCAAGCCTCTGCCCC, | |
| 2126, | Z46632, | Z46632, | CCTCTGCCCCTTCGCCGACC, | |
| 2127, | Z46632, | Z46632, | TTCGCCGACCCCCAGGTTCT, | |
| 2128, | Z46632, | Z46632, | CCCAGGTTCTCCATGCGCCA, | |
| 2129, | Z46632, | Z46632, | CCATGCGCCAGAGAAAGGCT, | |
| 2130, | Z46632, | Z46632, | GAGAAAGGCTGGATGAAGGG, | |
| 2131, | Z46632, | Z46632, | GGATGAAGGGTTTCCGACGG, | |
| 2132, | Z46632, | Z46632, | TTTCCGACGGGTCCCTGCTT, | |
| 2133, | Z46632, | Z46632, | GTCCCTGCTTGGCTGCTCCT, | |
| 2134, | Z46632, | Z46632, | GGCTGCTCCTAGGCATTGCT, | |
| 2135, | Z46632, | Z46632, | AGGCATTGCTGGCGGGCAAG, | |
| 2136, | Z46632, | Z46632, | GGCGGGCAAGGGCCGCCACG, | |
| 2137, | Z46632, | Z46632, | GGCCGCCACGTTGCTCCGAA, | |
| 2138, | Z46632, | Z46632, | TTGCTCCGAACGGTCCGCAG, | |
| 2139, | EPI-19-MTA-2, | Z46632, | TCTTCAGCAGGTCCCGCTCCTG, | |
| 2140, | EPI-19-MTA-3, | Z46632, | AACTGGTTGGAGACCCCAGG, | |
| 2141, | EPI-19-MTA-4, | Z46632, | CCTGCAGCAGCTTGAAGCCCAC, | |
| 2142, | EPI-19-MTA-5, | Z46632, | GCTGAGGTTCTGGAAGAT, | |
| 2143, | EPI-19-MTA-6, | Z46632, | GTGGCCAGCACCATGTC, | |
| 2144, | EPI-19-MTA-7, | Z46632, | TTCTTGGTCTCCACCATGGTCTT, | |
| 2145, | EPI-19-MTA-8, | Z46632, | CAGCGGCTTGGTGGGGTTGCT, | |
| 2146, | EPI-19-MTA-9, | Z46632, | GTCCACTGGCGGTACAG, | |
| 2147, | EPI-19-MTA-10, | Z46632, | TGCTTGTCACACATGGG, | |
| 2148, | EPI-19-MTA-14, | Z46632, | CTTGGTGGGGTTGCTCAG, | |
| 2149, | EPI-19-MTA-2, | Z46632, | AGAGTCAGTTCAAACTG, | |
| 2150, | EPI-19-MTA-2, | Z46632, | AACTTGTTGGAGGCCATCTC, | |
| 2151, | EPI-19-MTA-2, | Z46632, | CGGTCCGTCCACTGGCGGTACAG, | |
| 2152, | EPI-19-MTA-2, | Z46632, | TGCTTGTCACACATGGG, | |
| Concatemer Nucleic Acid Sequences of PDE4C gene oligo sequences | | | | |
| (SEQ ID NO: 2153) | | | | |
| TTTTTTTTTTTTTTTTTTTCTTTTTTTTTCTTTTTTGAGATTTTTTGAGACAGTGTCTTGCAGTGTCTTGCTCTGTCAGCCTCTGTCAGCCCCAGGCTGGCCCAGGCTGGAGTGCAGTGGAGTGCAGTGGCATG | | | | |
| ATGTCGCATGATGTCGGCTCACTGCAGCTCACTGCAACCTCCACCTACCTCCACCTCCTGAATTCACCTGAATTCAAGTGATTCTCAGTGATTCTCCTGCCTCAGCCTGCCTCAGCCTCCCCAGTACTCCCCAG | | | | |
| TAGCTGGGATTAGCTGGGATTACAGGCACCCGCAGGCACCCGCCACCATGCCCCACCATGCCCAGCCAATTTCAGCCAATTTTTGTATTTTTTTGTATTTTTAGTAGAGATGAGTAGAGATGGGGTTTCACCGG | | | | |
| GTTTCACCATGTTGGCCAATGTTGGCCAGGCTGGTCTCGGCTGGTCTCGAACTCCTAAGAACTCCTAACCTCAGGTGACCTCAGGTGATCCACCTGCCTCCACCTGCCTCAGCCTCCCTCAGCCTCCCAAAGTG | | | | |
| CTGGAAAGTGCTGGGATTATAGGCGATTATAGGCATGGGCCACTATGGGCCACTGTGCTCGGCCGTGCTCGGCCTCAGAGCCCCTCAGAGCCCCGTCTCTTTCCGTCTCTTTCCTTTCCTTCTCTTTCCTTCTC | | | | |
| TTTTCTTTTTTTTTCTTTTTATTTTTAGACATTTTTAGACAGGATCTTGCAGGATCTTGCTGTGTTGCCCTGTGTTGCCCAGGCTGGAGTAGGCTGGAGTGCAGTGATGCGCAGTGATGCAGTCATAGCTAGTC | | | | |
| ATAGCTCTCTTCAGCCCTCTTCAGCCTCCAACTCCTTCCAACTCCTGGGCTCAAGCGGGCTCAAGCGATCCCCTTTGATCCCCTTTGTCTCAACCTGTCTCAACCTTCTGAGTAGCTCTGAGTAGCTGGGATTC | | | | |
| TCTGGGATTCTCAGGTGCACACAGGTGCACACCACCATGCCTCACCATGCCTGGCTAATTTTGGCTAATTTTTTTTTTCAGATTTTTTCAGAGATGGTGGGGGATGGTGGGGGTCTTGCTATGTCTTGCTATGT | | | | |
| TGCCCAGGGTTGCCCAGGCTGGTCTCAACTGGTCTCAAACTCCTGAGCACTCCTGAGCTTAAGCAGTCTTAAGCAGTCCTCCCACCTCCTCCCACCTCAGCCTCCCAAAGCCTCCCAAAGTACCGGGAAGTACC | | | | |
| GGGATTACAGGCATTTACAGGCATAAGCCACTATAAGCCACTATGCCTTGCCCAGCCTTGCCCAGCCCTTCTTTGCCCTTCTTTTCTGCTCCTCTCTGCTCCTCTTCCTGCCCCTTCCTGCCCCCTACCGTAGT | | | | |
| CTACCGTAGTTTCAGAAACATTCAGAAACAAAACTGGGTAAAACTGGGTATGAGTGAAGCTGAGTGAAGCTTTGGTGCTGTTTGGTGCTGAAAATTTTCCAAAATTTTCCCCACTCACATCCACTCACATTTCC | | | | |
| ATGCTCTTCCATGCTCTTGCAGAGAGTTGCAGAGAGCCGCTTGGTACCGCTTGGTAGAGGAAGACAGAGGAAGACAGGGAGATGCCGGGAGATGCCTTTGGGATGGTTTGGGATGGTCTCCTGACTTCTCCTGA | | | | |
| CTCCCCACCCTTCCCCACCCTTTGTGCAGGGCTGTGCAGGGCTACTACAGAGTACTACAGAGGCAGAAAGCTGCAGAAAGCTGGCCCGAAGTGGCCCGAAGTAGATGAGCAAAGATGAGCAATAAATATTTGTA | | | | |
| AATATTTGATAAAGAAGGATAAAGAAGGAAATAATTAAAAATAATTAAGTGACAGATGGTGACAGATGTGACTCAAGATGACTCAAGAGTGACCACTGGTGACCACTGGAGAGGGTGGGAGAGGGTGGACTAGA | | | | |
| GGCTACTAGAGGCTCCAGCAGACACCAGCAGACAGCACCTCTCCGCACCTCTCCTCACAGGGATTCASCAGGGATAGAAGCCCAGAGAAGCCCAGGAGAAAGACAGAGAAGACACCAGGGCATCCCAGGGCATC | | | | |
| GTAAGAGGCTGTAAGAGGCTGCCCCTTAGAGCCCCTTAGAGAGCTCTTTTGAGCTCTTTTAGGCAAGTCTAGGCAAGTCTAGGGTCAGAGAGGGTCAGAGTGGACCCCAGTGGACCCCAGCCAGGTGCCTCCAG | | | | |
| GTGCCTCCAATTAGACCCAATTAGACCCTGGGAGCCCCTGGGAGCCACCTATAACTACCTATAACTAAGAGCTTGAAAGAGCTTGATTGTCTCCCTTTGTCTCCCTAAATGGGTGGAAATGGGTGGGAAAGTGA | | | | |
| AGGAAAGTGAAGCAGGAGCCACCAGGAGCCACATGGAGCCTCATGGAGCCTCTTCCTGGAAATTCCTGGAAAGTCTGCCTGCGTCTGCCTGCCAAGAGCCAACAAGAGCCAAAGGGCTTTACAGGGCTTTACCA | | | | |
| TCCATTGCCATCCATTGCCCCTGCAGTTCCCTGCAGTTCACGCAGGGCCACGCAGGGCTGGCCCTAAGTGGCCCTAAGTCCTCTGGTTTCCTCTGGTTGTCGAGGGGTGTCGAGGGGTAAGTCCCCAGAAGTCC | | | | |
| CCAGGGTCTGGGCCGGTCTGGGCCGGCTTCAGGGGGCTTCAGGGGACAGGAGTTGACAGGAGTTCAGTGTCAGGCAGTGTCAGGCAACTCCAAGCAACTCCAAGGCCTCTTTGGGCCTCTTTGGCTAAAGCTGT | | | | |
| CTAAAGCTGTCTCTTCCCCCCTCTTCCCCCTCCTCTTCTTTCCTCTTCTTCCTCCTCATCCCTCCTCATCCTCTTCCTCTCTCTTCCTCTGCCTCCTCCAGCCTCCTCCAGAGTCAGTTCGAGTCAGTTCAAAC | | | | |
| TGGAATAAACTGGAATCTGTCAGGCCCTGTCAGGCCCGTCCCGCTCCGTCCCGCTCGGGGTTGGTGGGGGTTGGTGAGGTCTGAGGAGGTCTGAGGGACTTCGGGGGACTTCGGGGGATCTTGCTCGATCTTGC | | | | |
| TCTGGTACCACTTGGTACCACTCTCGATTGTCCTCGATTGTCCTCCAGCGTGCTCCAGCGTGTCCAGCAGGTTCCAGCAGGTCCTGTGCATCCCTGTGCATCTGGGTGGACCTGGGTGGACCAGGTCAGCCCAG | | | | |
| GTCAGCCCAAGTCTCCCAAAGTCTCCCACAGTGGGTGACAGTGGGTGAGCAATGTAGTGCAATGTAGTCAATGAAACCCAATGAAACCCACCTGGGACCACCTGGGACTTCTCCACTGTTCTCCACTGAGGCCG | | | | |
| TATGAGGCCGTATGCTTGTCACACCTTGTCACACATGGGACTGAATGGGACTGATGTCCAGGCCTGTCCAGGCCCGACTCACGCCGACTCACGCTCGCGGTCTCTCGCGGTCTCCCTGCTGGAACCTGCTGGAA | | | | |
| GAACTCGGCCGAACTCGGCCATGATGCGGTATGATGCGGTCCGTCCACTGCCGTCCACTGGCGGTACAGGGCGGTACAGGGGCAGCGGCTGGCAGCGGCTTGGTGGGGTTTGGTGGGGTTGCTCAGATCAGCTC | | | | |
| AGATCAGCACAGTGCAGCACAGTGCACCAGGTTCTGCCAGGTTCTGCAAGACCTGGCAAGACCTGGATTCGGTCGGATTCGGTCGGAATAGTTGTCAATAGTTGTCCAGGAGGAGGCAGGAGGAGGACACCGAC | | | | |
| GCACACCGAGGCTTGTCACCTTTTGTCACCTTCTTGGTCTCCCTTGGTCTCCACCATGGTCTACCATGGTCTTGAGGTCGGCTGAGGTCGGCCAGGAGGTTCCAGGAGGTTCATGTGTTTGGATGTGTTTGGAC | | | | |
| ATGTCTGTACATGTCTGTGGCCAGCACCGGCCAGCACCATGTCAATGAATGTCAATGACCATCCTGCGCCATCCTGCGCAGACTCAGTCAGACTCAGTCGCTGCTTGGCGCTGCTTGGCGCTGAGGTTCGCTGA | | | | |
| GGTTCTGGAAGATACTGGAAGATATCGCAGTTCTTCGCAGTTCTCTGCCTGCAGCTGCCTGCAGCAGCTTGAAGCAGCTTGAAGCCCACAGCCACCCACAGCCAGGTGATGGTTGGTGATGGTTCTCCAGCACC | | | | |
| CTCCAGCACCGAGGCGTCGTGAGGCGTCGTTGTACATAAGTGTACATAAGCGCCACGTCTCGCCACGTCTGAGTTGGTGTGAGTTGGTGTTAATCAGAAATAATCAGAAACTGGTTGGAGCTGGTTGGAGACCC | | | | |
| CAGGATACCCCAGGATGGTCCACGTCGGTCCACGTCGTGGATGGCGGTGGATGGCGCTTGCAAAGACTTGCAAAGAGGGCAGCCAGGGGCAGCCAGGATTTCCAAGGATTTCCAAGTCTGTGAACATCTGTGAA | | | | |
| CACAGCCTCGAGCAGCCTCGAGGGCGGGCGTAGGCGGGCGTAGCCAGCAGCAGCCAGCAGCACATGCGTGGACATGCGTGGACTGGGCCACGCTGGGCCACGTCGGCGGCATTCGGCGGCATGTAGGCTGTTGT | | | | |
| AGGCTGTTGTGGTAGGCCGTGGTAGGCCACATTGGCGTACATTGGCGTGGTAGTGACCGGTAGTGACCCTCCAGCATCCTCCAGCATCAGCAGGTAGGAGCAGGTAGGTGGCCAGTGTTGGCCAGTGTGTCTGC | | | | |
| TGGGGTCTGCTGGGATCTGGAATGATCTGGAATGTCTTCAGCAGTCTTCAGCAGGTCCCGCTCCGTCCCGCTCCTGAAAAATGCTGAAAAATGCTGAATATGATTGAATATGATAGCTGTGAGGAGCTGTGAGG | | | | |
| GGCCGGTTCCGGCCGGTTCCCACTTACGTCCACTTACGTCCGCCACCTTGCGCCACCTTGAACACATCAAAACACATCAAGTCCCCACTTGTCCCCACTTGTTGGTGTCTGTTGGTGTCTTCTAGCTCCTTCTA | | | | |
| GCTCCTTGGCCAGTTGTGGCCAGTTGCTCCTCCTGGCTCCTCCTGGTCAGTCTGGATCAGTCTGGACCCCAAAGCGCCCCAAAGCGTGGGACAGTGTGGGACAGTGGCTGAGGAGAGCTGAGGAGAGGCTGGCA | | | | |
| CTGGCTGGCACTGTGGCAGAGCGTGGCAGAGCCCATGTAGGCCCATGTAGGCCACTGATCCGCACTGATCCGGGACATGGGCGGACATGGGCTGTGGGGCCTTGTGGGGCCTCCTCAGCGGTCCTCAGCGGTCA | | | | |
| CCTTGGGCCACCTTGGGCAGCTCCACCTAGCTCCACCTCGGTCTGCTGCGGTCTGCTGGTCCAGGAAGGTCCAGGAAGGTCCGGGAGAGTCCGGGAGATGTACTCGGATGTACTCGGACACCTGGTTCCACCTG | | | | |
| GTTCCCGGAGCGGCCCGGAGCGGCTGGTTTCGGATGGTTTCGGACAGGTGGGTCCAGGTGGGTCAACTCCCGGTAACTCCCGGTTCAGGATCCGTCAGGATCCGCTTGAACTTGCTTGAACTTGTTGGAGGCCA | | | | |
| TTGGAGGCCATCTCCCCCACTCTCCCCCACCGAGTGCCGGCGAGTGCCGGGTCTGCAGCGGTCTGCAGCGTCTCCAACTGTCTCCAACTGATCCAGGCACATCCAGGCACCAGTCCAGCTCAGTCCAGCTCGTC | | | | |
| TAGCGTCGTCTAGCGTCTCCAATGCCCTCCAATGCCAGCTTCTGCCAGCTTCTGCCCCGTGTCCTCCCGTGTCCTCTGCAGGAGGGTGCAGGAGGGAGCTGATTGCAGCTGATTGCTGGATGAAGGTGGATGAA | | | | |
| GGGTTTCCGACGGTTTCCGACGGGTCCCTGCTGGTCCCTGCTTGGCTGCTCCTGGCTGCTCCTAGGCATTGCTAGGCATTGCTGGCGGGCAATGGCGGGCAAGGGCCGCCACGGGCCGCCACGTTGCTCCGAGT | | | | |
| TGCTCCGAACGGTCCGCAACGGTCCGCAGACTGGCCAGGACTGGCCAGGACCTGGGCAGACCTGGGCAAAGGGCGTCAAAGGGCGTCACAATCATGTCCAATCATGTCCTCTCCATGTCTCTCCATGTAGGTCG | | | | |
| CTGGAGGTCGCTGGCCACAGAGGACCACAGAGGAGTTCCGAGACGTTCCGAGACATGGCCTTGGATGGCCTTGGGCGAGAGTTCGCGAGAGTTCATAGTCGCTAATAGTCGCTATCTGAGCGGTTCTGAGCGGT | | | | |
| ACAGGAAGGAACAGGAAGGACTCGCGCCGCCTCGCGCCGCTGGCTGTGCGTGGCTGTGCGGGACTGGAGCGGACTGGAGCCTGCATAATCCTGCATAATCCGGCCCAGGCCGGCCCAGGCCAGGGCTGGACAGG | | | | |
| GCTGGACTGAGGGTCCCTGAGGGTCCAGGGCCCTCCAGGGCCCTCCTCCCACACGATCCCACACGAGAGCCCATTTGAGCCCATTTTCCAGGTCAATCCAGGTCAAAGCGCCTGCAAGCGCCTGCAGGAGGAAA | | | | |
| CGGGAGGAAACGGGCCAGGAGAGGCCAGGAGAGCCGCGACTTGCCGCGACTTCCTGAGCTCCCCTGAGCTCCGGCCGCGGGCGGCCGCGGGCTCAGGTCCCTTCAGGTCCCTCTCGCGGCAGCTCGCGGCAGCC | | | | |
| CGCGGACTCCCGCGGACTTGTCCGGATCTGTCCGGATCCGAATAGAAGCGAATAGAAGCGCTGTTGGACGCTGTTGGATGCGGATGGGTGCGGATGGGGCGCCGGGGTGCGCCGGGGTTGCCGCCACATGCCGC | | | | |
| CACAGGTGCTTCGGGGTGCTTCGGGGCTCTGGTCGGCTCTGGTCATGCTGTGGCATGCTGTGGCGGCCGCGAGAGGCCGCGAGAGCGACTCAACGCGACTCAACCTGCTGCAAGCTGCTGCAAGCCTCTGCCCC | | | | |
| CCTCTGCCCCTTCGCCGACCTTCGCCGACCCCCAGGTTCTCCCAGGTTCTCCATGCGCCACCATGCGCCAGAGAAAGGCTGAGAAAGGCTGGATGAAGGGGGATGAAGGGTTTCCGACGGTTTCCGACGGGTCC | | | | |
| CTGCTTGTCCCTGCTTGGCTGCTCCTGGCTGCTCCTAGGCATTGCTAGGCATTGCTGGCGGGCAAGGGCGGGCAAGGGCCGCCACGGGCCGCCACGTTGCTCCGAATTGCTCCGAACGGTCCGCAGTCTTCAGC | | | | |
| AGGTCCCGCTCCTGAACTGGTTGGAGACCCCAGGCCTGCAGCAGCTTGAAGCCCACGCTGAGGTTCTGGAAGATGTGGCCAGCACCATGTCTTCTTGGTCTCCACCATGGTCTTCAGCGGCTTGGTGGGGTTGC | | | | |
| TGTCCACTGGCGGTACAGTGCTTGTCACACATGGGCTTGGTGGGGTTGCTCAGAGAGTCAGTTCAAACTGAACTTGTTGGAGGCCATCTCCGGTCCGTCCACTGGCGGTACAGTGCTTGTCACACATGGG | | | | |
| PDE4D Nucleic Acid Sequences (GENBANK ACCESSION NO.X61177) | | | | |
| (SEQ ID NO: 2154) | | | | |
| GAATTCCCTGGATACAGCCTTTTTATGACTTTTACTTCCTTTATANNCAAATTCCAACGTCTTCTCATTCCTCCACCAGGSCTGTGCCAACCTGGGCCCAACCCAAGGSCCTCACTAAACCATCCAATCAGTAG | | | | |
| GAGCCATAGACTACTTTATTTAGCCAAAGCAAAAATGAGTCAACTGAATTCTGTTTTTCCATTTACTTCTGTCTGTTTTTCCTTCCTCTTGCCACCCTCAGTGCCACAAGAGGGGACCCCTCTCGGTAGCCCTG | | | | |
| AGGCTCTGGCGCCTTCAAGTGAGAAGCTAAGCACCAGCCTCTGCTGGGCTGCAGAAGCGGCGGCGGCGGCAGCAGCAGCAGCAGCATCAGGAAGGCTCTCGGGCCAGCGCGGTGAACCCGGGCTGGGCAGCAGG | | | | |
| TCGCGGAGCCGCGAGCCAGGATGGAGGCAGAGGGCAGCAGCGCGCCGGCCCGGGCGGGCAGCGGAGAGGGCAGCGACAGCGCCGGCGGGGCCACGCTCAAAGCCCCCAAGCATCTCTGGAGGCACGAGCAGCAC | | | | |
| CACCAGTACCCGCTCCGGCAGCCCCAGTTCCGCCTCCTGCATCCCCATCACCACCTGCCCCCGCCGCCGCCACCCTCGCCCCAGCCCCAGCCCCAGTGTCCGCTACAGCCGCCGCCGCCGCCCCCCCTGCCGCC | | | | |
| GCCCCCGCCGCCGCCCGGGGCTGCCCGCGGCCGCTACGCCTCGAGCGGGGCCACCGGCCGCGTCCGGCATCGCGGCTACTCGGACACCGAGCGCTACCTGTACTGTCGCGCCATGGACCGCACCTCCTACGCGG | | | | |
| TGGAGACCGGCCACCGGCCCGGCCTGAAGAAATCCAGGATGTCCTGGCCCTCCTCGTTCCAGGGACTCAGGCGTTTTGATGTGGACAATGGCACATCTGCGGGACGGAGTCCCTTGGATCCCATGACCAGCCCA | | | | |
| GGATCCGGGCTAATTCTCCAAGCAAATTTTGTCCACAGTCAACGACGGGAGTCCTTCCTGTATCGATCCGACAGCGATTATGACCTCTCTCCAAAGTCTATGTCCCGGAACTCCTCCATTGCCAGTGATATACA | | | | |
| CGGAGATGACTTGATTGTGACTCCATTTGCTCAGGTCTTGGCCAGTCTGCGAACTGTACGAAACAACTTTGCTGCATTAACTAATTTGCAAGATCGAGCACCTAGCAAAAGATCACCCATGTGCAACCAACCAT | | | | |
| CCATCAACAAAGCCACCATAACAGAGGAGGCCTACCAGAAACTGGCCAGCGAGACCCTGGAGGAGCTGGACTGGTGTCTGGACCAGCTAGAGACCCTACAGACCAGGCACTCCGTCAGTGAGATGGCCTCCAAC | | | | |
| AAGTTTAAAAGGATGCTTAATCGGGAGCTCACCCATCTCTCTGAAATGAGTCGGTCTGGAAATCAAGTGTCAGAGTTTATATCAAACACATTCTTAGATAAGCAACATGAAGTGGAAATTCCTTCTCCAACTCA | | | | |
| GAAGGAAAAGGAGAAAAAGAAAAGACCAATGTCTCAGATCAGTGGAGTCAAGAAATTGATGCACAGCTCTAGTCTGACTAATTCAAGTATCCCAAGGTTTGGAGTTAAAACTGAACAAGAAGATGTCCTTGCCA | | | | |
| AGGAACTAGAAGATGTGAACAAATGGGGTCTTCATGTTTTCAGAATAGCAGAGTTGTCTGGTAACCGGCCCTTGACTGTTATCATGCACACCATTTTTCAGGAACGGGATTTATTAAAAACATTTAAAATTCCA | | | | |
| GTAGATACTTTAATTACATATCTTATGACTCTCGAAGACCATTACCATGCTGATGTGGCCTATCACAACAATATCCATGCTGCAGATGTTGTCCAGTCTACTCATGTGCTATTATCTACACCTGCTTTGGAGGC | | | | |
| TGTGTTTACAGATTTGGAGATTCTTGCAGCAATTTTTGCCAGTGCAATACATGATGTAGATCATCCTGGTGTGTCCAATCAATTTCTGATCAATACAAACTCTGAACTTGCCTTGATGTACAATGATTCCTCAG | | | | |
| TCTTAGAGAACCATCATTTGGCTGTGGGCTTTAAATTGCTTCAGGAAGAAAACTGTGACATTTTCCAGAATTTGACCAAAAAACAAAGACAATCTTTAAGGAAAATGGTCATTGACATCGTACTTGCAACAGAT | | | | |
| ATGTCAAAACACATGAATCTACTGGCTGATTTGAAGACTATGGTTGAAACTAAGAAAGTGACAAGCTCTGGAGTTCTTCTTCTTGATAATTATTCCGATAGGATTCAGGTTCTTCAGAATATGGTGCACTGTGC | | | | |
| AGATCTGAGCAACCCAACAAAGCCTCTCCAGCTGTACCGCCAGTGGACGGACCGGATAATGGAGGAGTTCTTCCGCCAAGGAGACCGAGAGAGGGAACGTGGCATGGAGATAAGCCCCATGTGTGACAAGCACA | | | | |
| ATGCTTCCGTGGAAAAATCACAGGTGGGCTTCATAGACTATATTGTTCATCCCCTCTGGGAGACATGGGCAGACCTCGTCCACCCTGACGCCCAGGATATTTTGGACACTTTGGAGGACAATCGTGAATGGTAC | | | | |
| CAGAGCACAATCCCTCAGAGCCCCTCTCCTGCACCTGATGACCCAGAGGAGGGCCGGCAGGGTCAAACTGAGAAATTCCAGTTTGAACTAACTTTAGAGGAAGATGGTGAGTCAGACACGGAAAAGGACAGTGG | | | | |
| CAGTCAAGTGGAAGAAGACACTAGCTGCAGTGACTCCAAGACTCTTTGTACTCAAGACTCAGAGTCTACTGAAATTCCCCTTGATGAACAGGTTGAAGAGGAGGCAGTAGGGGAAGAAGAGGAAAGCCAGCCTG | | | | |
| AAGCCTGTGTCATAGATGATCGTTCTCCTGACACGTAACAGTGCAAAAACTTTCATGCCTTTTTTTTTTTTAAGTAGAAAAATTGTTTCCAAAGTGCATGTCACATGCCACAACCACGGTCACACCTCACTGTC | | | | |
| ATCTGCCAGGACGTTTGTTGAACAAAACTGACCTTGACTACTCAGTCCAGCGCTCAGGAATATCGTAACCAGTTTTTTCACCTCCATGTTCATCCGAGCAAGGTGGACATCTTCACGAACAGCGTTTTTAACAA | | | | |
| GATTTCAGCTTGGTAGAGCTGACAAAGCAGATAAAATCTACTCCAAATTATTTTCAAGAGAGTGTGACTCATCAGGCAGCCCAAAAGTTTATTGGACTTGGGGTTTCTATTCCTTTTTATTTGTTTGCAATATT | | | | |
| TTCAGAAGAAAGGCATTGCACAGAGTGAACTTAATGGACGAAGCAACAAATATGTCAAGAACAGGACATAGCACGAATCTGTTACCAGTAGGAGGAGGATGAGCCACAGAAATTGCATAATTTTCTAATTTCAA | | | | |
| GTCTTCCTGATACATGACTGAATAGTGTGGTTCAGTGAGCTGCACTGACCTCTACATTTTGTATGATATGTAAAACAGATTTTTTGTAGAGCTTACTTTTATTATTAAATGTATTGAGGTATTATATTTAAAAA | | | | |
| AAACTATGTTCAGAACTTCATCTGCCACTGGTTATTTTTTTCTAAGGAGTAACTTGCAAGTTTTCAGTACAAATCTGTGCTACACTGGATAAAAATCTAATTTATGAATTTTACTTGCACCTTATAGTTCATAG | | | | |
| CAATTAACTGATTTGTAGTGATTCATTGTTTGTTTTATATACCAATGACTTCCATATTTTAAAAGAGAAAAACAACTTTATGTTGCAGGAAACCCTTTTTGTAAGTCTTTATTATTTACTTTGCATTTTGTTTC | | | | |
| ACTCTTTCCAGATAAGCAGAGTTGCTCTTCACCAGTGTTTTTCTTTCATGTGCAAAGTGACTATTTGTTCTATAATAC | | | | |
| 2155, | EPI-19-MTA-11, | NM_006203, | GTCCACTGGCGGTACAGCT, | |
| 2156, | EPI-19-MTA-12, | NM_006203, | TGTGCTTGTCACACATGGGGCT, | |
| 2157, | EPI-19-MTA-13, | NM_006203, | TTGTCCTCCAAAGTGTCCAA, | |
| 2158, | EPI-19-MTA-16, | NM_006203, | AAGACCCCATTTGTTCA, | |
| 2159, | EPI-19-MTA-17, | NM_006203, | TCTGCCCATGTCTCCCA, | |
| 2160, | EPI-19-MTA-18, | NM_006203, | AACTTGTTGGAGGCCATCTC, | |
| 2161, | EPI-19-MTA-19, | NM_006203, | CGGTCCGTCCACTGGCGGTACAG, | |
| 2162, | EPI-19-MTA-20, | NM_006203, | TGCTTGTCACACATGGG, | |
| Concatemer Nucleic Acid Sequences of PDE4D gene oligo sequences | | | | |
| (SEQ ID NO: 2163) | | | | |
| GTCCACTGGCGGTACAGCTTGTGCTTGTCACACATGGGGCTTTGTCCTCCAAAGTGTCCAAAAGACCCCATTTGTTCATCTGCCCATGTCTCCCAAACTTGTTGGAGGCCATCTCCGGTCCGTCCACTGGCGGT | | | | |
| ACAGTGCTTGTCACACATGGG | | | | |
| IL5R-X61176 Nucleic Acid Sequences (GENBANK ACCESSION NO.X61177) | | | | |
| (SEQ ID NO: 2164) | | | | |
| CGGTCCTCGCCATCTTCTGTTGAGTACTGGTCGGAACAAGAGGATCGTCTGTAGACAGGATATGATCATCGTGGCGCATGTATTACTCATCCTTTTGGGGGCCACTGAGATACTGCAAGCTGACTTACTTCCTG | | | | |
| ATGAAAAGATTTCACTTCTCCCACCTGTCAATTTCACCATTAAAGTTACTGGTTTGGCTCAAGTTCTTTTACAATGGAAACCAAATCCTGATCAAGAGCAAAGGAATGTTAATCTAGAATATCAAGTGAAAATA | | | | |
| AACGCTCCAAAAGAAGATGACTATGAAACCAGAATCACTGAAAGCAAATGTGTAACCATCCTCCACAAAGGCTTTTCAGCAAGTGTGCGGACCATCCTGCAGAACGACCACTCACTACTGGCCAGCAGCTGGGC | | | | |
| TTCTGCTGAACTTCATGCCCCACCAGGGTCTCCTGGAACCTCAGTTGTGAATTTAACTTGCACCACAAACACTACAGAAGACAATTATTCACGTTTAAGGTCATACCAAGTTTCCCTTCACTGCACCTGGCTTG | | | | |
| TTGGCACAGATGCCCCTGAGGACACGCAGTATTTTCTCTACTATAGGTATGGCTCTTGGACTGAAGAATGCCAAGAATACAGCAAAGACACACTGGGGAGAAATATCGCATGCTGGTTTCCCAGGACTTTTATC | | | | |
| CTCAGCAAAGGGCGTGACTGGCTTGCGGTGCTTGTTAACGGCTCCAGCAAGCACTCTGCTATCAGGCCCTTTGATCAGCTGTTTGCCCTTCACGCCATTGATCAAATAAATCCTCCACTGAATGTCACAGCAGA | | | | |
| GATTGAAGGAACTCGTCTCTCTATCCAATGGGAGAAACCAGTGTCTGCTTTTCCAATCCATTGCTTTGATTATGAAGTAAAAATACACAATACAAGGAATGGATATTTGCAGATAGAAAAATTGATGACCAATG | | | | |
| CATTCATCTCAATAATTGATGATCTTTCTAAGTACGATGTTCAAGTGAGAGCAGCAGTGAGCTCCATGTGCAGAGAGGCAGGGCTCTGGAGTGAGTGGAGCCAACCTATTTATGTGGGAAATGATGAACACAAG | | | | |
| CCCTTGAGAGAGTGGTTTGTCATTGTGATTATGGCAACCATCTGCTTCATCTTGTTAATTCTCTCGCTTATCTGTAAAATATGTCATTTATGGATCAAGTTGTTTCCACCAATTCCAGCACCAAAAAGTAATAT | | | | |
| CAAAGATCTCTTTGTAACCACTAACTATGAGAAAGCTGGGTCCAGTGAGACGGAAATTGAAGTCATCTGTTATATAGAGAAGCCTGGAGTTGAGACCCTGGAGGATTCTGTGTTTTGACTGTCACTTTGGCATC | | | | |
| CTCTGATGAACTCACACATGCCTCAGTGCCTCAGTGAAAAGAACAGGGATGCTGGCTCTTGGCTAAGAGGTGTTCAGAATTTAGGCAACACTCAATTTACCTGCGAAGCAATACACCCAGACACACCAGTCTTG | | | | |
| TATCTCTTAAAAGTATGGATGCTTCATCCAAATCGCCTCACCTACAGCAGGGAAGTTGACTCATCCAAGCATTTTGCCATGTTTTTTCTCCCCATGCCGTACAGGGTAGCACCTCCTCACCTGCCAATCTTTGC | | | | |
| AATTTGCTTGACTCACCTCAGACTTTTCATTCACAACAGACAGCTTTTAAGGCTAACGTCCAGCTGTATTTACTTCTGGCTGTGCCCGTTTGGCTGTTTAAGCTGCCAATTGTAGCACTCAGCTACCATCTGAG | | | | |
| GAAGAAAGCATTTTGCATCAGCCTGGAGTGAATCATGAACTTGGATTCAAGACTGTCTTTTCTATAGCAAGTGAGAGCCACAAATTCCTCACCCCCCTACATTCTAGAATGATCTTTTTCTAGGTAGATTGTGT | | | | |
| ATGTGTGTGTATGAGAGAGAGAGAGAGAGAGAGAGAGAGAGAGAGAGAAATTATCTCAAGCTCCAGAGGCCTGATCCAGGATACATCATTTGAAACCAACTAATTTAAAAGCATAATAGAGCTAATATAT | | | | |
| 2165, | X61176, | X61176, | ATATATTAGCTCTATTATGC, | |
| 2166, | X61176, | X61176, | TAGCTCTATTATGCTTTTAA, | |
| 2167, | X61176, | X61176, | TATTATGCTTTTAAATTAGT, | |
| 2168, | X61176, | X61176, | GCTTTTAAATTAGTTGGTTT, | |
| 2169, | X61176, | X61176, | AAATTAGTTGGTTTCAAATG, | |
| 2170, | X61176, | X61176, | GTTGGTTTCAAATGATGTAT, | |
| 2171, | X61176, | X61176, | TTCAAATGATGTATCCTGGA, | |
| 2172, | X61176, | X61176, | TGATGTATCCTGGATCAGGC, | |
| 2173, | X61176, | X61176, | ATCCTGGATCAGGCCTCTGG, | |
| 2174, | X61176, | X61176, | GATCAGGCCTCTGGAGCTTG, | |
| 2175, | X61176, | X61176, | GCCTCTGGAGCTTGAGATAA, | |
| 2176, | X61176, | X61176, | GGAGCTTGAGATAATTTCTC, | |
| 2177, | X61176, | X61176, | TGAGATAATTTCTCTCTCTC, | |
| 2178, | X61176, | X61176, | AATTTCTCTCTCTCTCTCTC, | |
| 2179, | X61176, | X61176, | TCTCTCTCTCTCTCTCTCTC, | |
| 2180, | X61176, | X61176, | TCTCTCTCTCTCTCTCTCTC, | |
| 2181, | X61176, | X61176, | TCTCTCTCTCTCTCTCTCTC, | |
| 2182, | X61176, | X61176, | TCTCTCTCTCTCTCTCTCTC, | |
| 2183, | X61176, | X61176, | TCTCTCTCTCATACACACAC, | |
| 2184, | X61176, | X61176, | TCTCATACACACACATACAC, | |
| 2185, | X61176, | X61176, | ACACACACATACACAATCTA, | |
| 2186, | X61176, | X61176, | ACATACACAATCTACCTAGA, | |
| 2187, | X61176, | X61176, | ACAATCTACCTAGAAAAAGA, | |
| 2188, | X61176, | X61176, | TACCTAGAAAAAGATCATTC, | |
| 2189, | X61176, | X61176, | GAAAAAGATCATTCTAGAAT, | |
| 2190, | X61176, | X61176, | GATCATTCTAGAATGTAGGG, | |
| 2191, | X61176, | X61176, | TCTAGAATGTAGGGGGGTGA, | |
| 2192, | X61176, | X61176, | ATGTAGGGGGGTGAGGAATT, | |
| 2193, | X61176, | X61176, | GGGGGTGAGGAATTTGTGGC, | |
| 2194, | X61176, | X61176, | GAGGAATTTGTGGCTCTCAC, | |
| 2195, | X61176, | X61176, | TTTGTGGCTCTCACTTGCTA, | |
| 2196, | X61176, | X61176, | GCTCTCACTTGCTATAGAAA, | |
| 2197, | X61176, | X61176, | ACTTGCTATAGAAAAGACAG, | |
| 2198, | X61176, | X61176, | TATAGAAAAGACAGTCTTGA, | |
| 2199, | X61176, | X61176, | AAAGACAGTCTTGAATCCAA, | |
| 2200, | X61176, | X61176, | AGTCTTGAATCCAAGTTCAT, | |
| 2201, | X61176, | X61176, | GAATCCAAGTTCATGATTCA, | |
| 2202, | X61176, | X61176, | AAGTTCATGATTCACTCCAG, | |
| 2203, | X61176, | X61176, | ATGATTCACTCCAGGCTGAT, | |
| 2204, | X61176, | X61176, | CACTCCAGGCTGATGCAAAA, | |
| 2205, | X61176, | X61176, | AGGCTGATGCAAAATGCTTT, | |
| 2206, | X61176, | X61176, | ATGCAAAATGCTTTCTTCCT, | |
| 2207, | X61176, | X61176, | AATGCTTTCTTCCTCAGATG, | |
| 2208, | X61176, | X61176, | TTCTTCCTCAGATGGTAGCT, | |
| 2209, | X61176, | X61176, | CTCAGATGGTAGCTGAGTGC, | |
| 2210, | X61176, | X61176, | TGGTAGCTGAGTGCTACAAT. | |
| 2211, | X61176, | X61176, | CTGAGTGCTACAATTGGCAG, | |
| 2212, | X61176, | X61176, | GCTACAATTGGCAGCTTAAA, | |
| 2213, | X61176, | X61176, | ATTGGCAGCTTAAACAGCCA, | |
| 2214, | X61176, | X61176, | AGCTTAAACAGCCAAACGGG, | |
| 2215, | X61176, | X61176, | AACAGCCAAACGGGCACAGC, | |
| 2216, | X61176, | X61176, | CAAACGGGCACAGCCAGAAG, | |
| 2217, | X61176, | X61176, | GGCACAGCCAGAAGTAAATA, | |
| 2218, | X61176, | X61176, | GCCAGAAGTAAATACAGCTG, | |
| 2219, | X61176, | X61176, | AGTAAATACAGCTGGACGTT, | |
| 2220, | X61176, | X61176, | TACAGCTGGACGTTAGCCTT, | |
| 2221, | X61176, | X61176, | TGGACGTTAGCCTTAAAAGC, | |
| 2222, | X61176, | X61176, | TTAGCCTTAAAAGCTGTCTG, | |
| 2223, | X61176, | X61176, | TTAAAAGCTGTCTGTTGTGA, | |
| 2224, | X61176, | X61176, | GCTGTCTGTTGTGAATGAAA, | |
| 2225, | X61176, | X61176, | TGTTGTGAATGAAAAGTCTG, | |
| 2226, | X61176, | X61176, | GAATGAAAAGTCTGAGGTGA, | |
| 2227, | X61176, | X61176, | AAAGTCTGAGGTGAGTCAAG, | |
| 2228, | X61176, | X61176, | TGAGGTGAGTCAAGCAAATT, | |
| 2229, | X61176, | X61176, | GAGTCAAGCAAATTGCAAAG, | |
| 2230, | X61176, | X61176, | AGCAAATTGCAAAGATTGGC, | |
| 2231, | X61176, | X61176, | TTGCAAAGATTGGCAGGTGA, | |
| 2232, | X61176, | X61176, | AGATTGGCAGGTGAGGAGGT, | |
| 2233, | X61176, | X61176, | GCAGGTGAGGAGGTGCTACC, | |
| 2234, | X61176, | X61176, | GAGGAGGTGCTACCCTGTAC, | |
| 2235, | X61176, | X61176, | GTGCTACCCTGTACGGCATG, | |
| 2236, | X61176, | X61176, | CCCTGTACGGCATGGGGAGA, | |
| 2237, | X61176, | X61176, | ACGGCATGGGGAGAAAAAAC, | |
| 2238, | X61176, | X61176, | TGGGGAGAAAAAACATGGCA, | |
| 2239, | X61176, | X61176, | GAAAAAACATGGCAAAATGC, | |
| 2240, | X61176, | X61176, | ACATGGCAAAATGCTTGGAT, | |
| 2241, | X61176, | X61176, | CAAAATGCTTGGATGAGTCA, | |
| 2242, | X61176, | X61176, | GCTTGGATGAGTCAACTTCC, | |
| 2243, | X61176, | X61176, | ATGAGTCAACTTCCCTGCTG, | |
| 2244, | X61176, | X61176, | CAACTTCCCTGCTGTAGGTG, | |
| 2245, | X61176, | X61176, | CCCTGCTGTAGGTGAGGCGA, | |
| 2246, | X61176, | X61176, | TGTAGGTGAGGCGATTTGGA, | |
| 2247, | X61176, | X61176, | TGAGGCGATTTGGATGAAGC, | |
| 2248, | X61176, | X61176, | GATTTGGATGAAGCATCCAT, | |
| 2249, | X61176, | X61176, | GATGAAGCATCCATACTTTT, | |
| 2250, | X61176, | X61176, | GCATCCATACTTTTAAGAGA, | |
| 2251, | X61176, | X61176, | ATACTTTTAAGAGATACAAG, | |
| 2252, | X61176, | X61176, | TTAAGAGATACAAGACTGGT, | |
| 2253, | X61176, | X61176, | GATACAAGACTGGTGTGTCT, | |
| 2254, | X61176, | X61176, | AGACTGGTGTGTCTGGGTGT, | |
| 2255, | X61176, | X61176, | GTGTGTCTGGGTGTATTGCT, | |
| 2256, | X61176, | X61176, | CTGGGTGTATTGCTTCGCAG, | |
| 2257, | X61176, | X61176, | GTATTGCTTCGCAGGTAAAT, | |
| 2258, | X61176, | X61176, | CTTCGCAGGTAAATTGAGTG, | |
| 2259, | X61176, | X61176, | AGGTAAATTGAGTGTTGCCT, | |
| 2260, | X61176, | X61176, | ATTGAGTGTTGCCTAAATTC, | |
| 2261, | X61176, | X61176, | TGTTGCCTAAATTCTGAACA, | |
| 2262, | X61176, | X61176, | CTAAATTCTGAACACCTCTT, | |
| 2263, | X61176, | X61176, | TCTGAACACCTCTTAGCCAA, | |
| 2264, | X61176, | X61176, | CACCTCTTAGCCAAGAGCCA, | |
| 2265, | X61176, | X61176, | TTAGCCAAGAGCCAGCATCC, | |
| 2266, | X61176, | X61176, | AAGAGCCAGCATCCCTGTTC, | |
| 2267, | X61176, | X61176, | CAGCATCCCTGTTCTTTTCA, | |
| 2268, | X61176, | X61176, | CCCTGTTCTTTTCACTGAGG, | |
| 2269, | X61176, | X61176, | TCTTTTCACTGAGGCACTGA, | |
| 2270, | X61176, | X61176, | CACTGAGGCACTGAGGCATG, | |
| 2271, | X61176, | X61176, | GGCACTGAGGCATGTGTGAG, | |
| 2272, | X61176, | X61176, | GAGGCATGTGTGAGTTCATC, | |
| 2273, | X61176, | X61176, | TGTGTGAGTTCATCAGAGGA, | |
| 2274, | X61176, | X61176, | AGTTCATCAGAGGATGCCAA, | |
| 2275, | X61176, | X61176, | TCAGAGGATGCCAAAGTGAC, | |
| 2276, | X61176, | X61176, | GATGCCAAAGTGACAGTCAA, | |
| 2277, | X61176, | X61176, | AAAGTGACAGTCAAAACACA, | |
| 2278, | X61176, | X61176, | ACAGTCAAAACACAGAATCC, | |
| 2279, | X61176, | X61176, | AAAACACAGAATCCTCCAGG, | |
| 2280, | X61176, | X61176, | CAGAATCCTCCAGGGTCTCA, | |
| 2281, | X61176, | X61176, | CCTCCAGGGTCTCAACTCCA, | |
| 2282, | X61176, | X61176, | GGGTCTCAACTCCAGGCTTC, | |
| 2283, | X61176, | X61176, | CAACTCCAGGCTTCTCTATA, | |
| 2284, | X61176, | X61176, | CAGGCTTCTCTATATAACAG, | |
| 2285, | X61176, | X61176, | TCTCTATATAACAGATGACT, | |
| 2286, | X61176, | X61176, | TATAACAGATGACTTCAATT, | |
| 2287, | X61176, | X61176, | AGATGACTTCAATTTCCGTC, | |
| 2288, | X61176, | X61176, | CTTCAATTTCCGTCTCACTG, | |
| 2289, | X61176, | X61176, | TTTCCGTCTCACTGGACCCA, | |
| 2290, | X61176, | X61176, | TCTCACTGGACCCAGCTTTC, | |
| 2291, | X61176, | X61176, | TGGACCCAGCTTTCTCATAG, | |
| 2292, | X61176, | X61176, | CAGCTTTCTCATAGTTAGTG, | |
| 2293, | X61176, | X61176, | TCTCATAGTTAGTGGTTACA, | |
| 2294, | X61176, | X61176, | AGTTAGTGGTTACAAAGAGA, | |
| 2295, | X61176, | X61176, | TGGTTACAAAGAGATCTTTG, | |
| 2296, | X61176, | X61176, | CAAAGAGATCTTTGATATTA, | |
| 2297, | X61176, | X61176, | GATCTTTGATATTACTTTTT, | |
| 2298, | X61176, | X61176, | TGATATTACTTTTTGGTGCT, | |
| 2299, | X61176, | X61176, | TACTTTTTGGTGCTGGAATT, | |
| 2300, | X61176, | X61176, | TTGGTGCTGGAATTGGTGGA, | |
| 2301, | X61176, | X61176, | GAAACAACTTGATCCATAAA, | |
| 2302, | X61176, | X61176, | ACTTGATCCATAAATGACAT, | |
| 2303, | X61176, | X61176, | TCCATAAATGACATATTTTA, | |
| 2304, | X61176, | X61176, | AATGACATATTTTACAGATA, | |
| 2305, | X61176, | X61176, | ATATTTTACAGATAAGCGAG, | |
| 2306, | X61176, | X61176, | TACAGATAAGCGAGAGAATT, | |
| 2307, | X61176, | X61176, | TAAGCGAGAGAATTAACAAG, | |
| 2308, | X61176, | X61176, | AGAGAATTAACAAGATGAAG, | |
| 2309, | X61176, | X61176, | TTAACAAGATGAAGCAGATG, | |
| 2310, | X61176, | X61176, | AGATGAAGCAGATGGTTGCC, | |
| 2311, | X61176, | X61176, | TTAACAAGATGAAGCAGATG, | |
| 2312, | X61176, | X61176, | AGATGAAGCAGATGGTTGCC, | |
| 2313, | X61176, | X61176, | AGCAGATGGTTGCCATAATC, | |
| 2314, | X61176, | X61176, | TGGTTGCCATAATCACAATG, | |
| 2315, | X61176, | X61176, | CCATAATCACAATGACAAAC, | |
| 2316, | X61176, | X61176, | TCACAATGACAAACCACTCT, | |
| 2317, | X61176, | X61176, | TGACAAACCACTCTCTCAAG, | |
| 2318, | X61176, | X61176, | ACCACTCTCTCAAGGGCTTG, | |
| 2319, | X61176, | X61176, | CTCTCAAGGGCTTGTGTTCA, | |
| 2320, | X61176, | X61176, | AGGGCTTGTGTTCATCATTT, | |
| 2321, | X61176, | X61176, | TGTGTTCATCATTTCCCACA, | |
| 2322, | X61176, | X61176, | CATCATTTCCCACATAAATA, | |
| 2323, | X61176, | X61176, | TTCCCACATAAATAGGTTGG, | |
| 2324, | X61176, | X61176, | CATAAATAGGTTGGCTCCAC, | |
| 2325, | X61176, | X61176, | TAGGTTGGCTCCACTCACTC, | |
| 2326, | X61176, | X61176, | GGCTCCACTCACTCCAGAGC, | |
| 2327, | X61176, | X61176, | ACTCACTCCAGAGCCCTGCC, | |
| 2328, | X61176, | X61176, | TCCAGAGCCCTGCCTCTCTG, | |
| 2329, | X61176, | X61176, | GCCCTGCCTCTCTGCACATG, | |
| 2330, | X61176, | X61176, | CCTCTCTGCACATGGAGCTC, | |
| 2331, | X61176, | X61176, | TGCACATGGAGCTCACTGCT, | |
| 2332, | X61176, | X61176, | TGGAGCTCACTGCTGCTCTC, | |
| 2333, | X61176, | X61176, | TCACTGCTGCTCTCACTTGA, | |
| 2334, | X61176, | X61176, | CTGCTCTCACTTGAACATCG, | |
| 2335, | X61176, | X61176, | TCACTTGAACATCGTACTTA, | |
| 2336, | X61176, | X61176, | GAACATCGTACTTAGAAAGA, | |
| 2337, | X61176, | X61176, | CGTACTTAGAAAGATCATCA, | |
| 2338, | X61176, | X61176, | TAGAAAGATCATCAATTATT, | |
| 2339, | X61176, | X61176, | GATCATCAATTATTGAGATG, | |
| 2340, | X61176, | X61176, | CAATTATTGAGATGAATGCA, | |
| 2341, | X61176, | X61176, | TTGAGATGAATGCATTGGTC, | |
| 2342, | X61176, | X61176, | TGAATGCATTGGTCATCAAT, | |
| 2343, | X61176, | X61176, | CATTGGTCATCAATTTTTCT, | |
| 2344, | X61176, | X61176, | TCATCAATTTTTCTATCTGC, | |
| 2345, | X61176, | X61176, | ATTTTTCTATCTGCAAATAT, | |
| 2346, | X61176, | X61176, | CTATCTGCAAATATCCATTC, | |
| 2347, | X61176, | X61176, | GCAAATATCCATTCCTTGTA, | |
| 2348, | X61176, | X61176, | ATCCATTCCTTGTATTGTGT, | |
| 2349, | X61176, | X61176, | TCCTTGTATTGTGTATTTTT, | |
| 2350, | X61176, | X61176, | TATTGTGTATTTTTACTTCA, | |
| 2351, | X61176, | X61176, | GTATTTTTACTTCATAATCA, | |
| 2352, | X61176, | X61176, | TTACTTCATAATCAAAGCAA, | |
| 2353, | X61176, | X61176, | CATAATCAAAGCAATGGATT, | |
| 2354, | X61176, | X61176, | CAAAGCAATGGATTGGAAAA, | |
| 2355, | X61176, | X61176, | AATGGATTGGAAAAGCAGAC, | |
| 2356, | X61176, | X61176, | TTGGAAAAGCAGACACTGGT, | |
| 2357, | X61176, | X61176, | AAGCAGACACTGGTTTCTCC, | |
| 2358, | X61176, | X61176, | ACACTGGTTTCTCCCATTGG, | |
| 2359, | X61176, | X61176, | GTTTCTCCCATTGGATAGAG, | |
| 2360, | X61176, | X61176, | CCCATTGGATAGAGAGACGA, | |
| 2361, | X61176, | X61176, | GGATAGAGAGACGAGTTCCT, | |
| 2362, | X61176, | X61176, | AGAGACGAGTTCCTTCAATC, | |
| 2363, | X61176, | X61176, | GAGTTCCTTCAATCTCTGCT, | |
| 2364, | X61176, | X61176, | CTTCAATCTCTGCTGTGACA, | |
| 2365, | X61176, | X61176, | TCTCTGCTGTGACATTCAGT, | |
| 2366, | X61176, | X61176, | CTGTGACATTCAGTGGAGGA, | |
| 2367, | X61176, | X61176, | CATTCAGTGGAGGATTTATT, | |
| 2368, | X61176, | X61176, | GTGGAGGATTTATTTGATCA, | |
| 2369, | X61176, | X61176, | GATTTATTTGATCAATGGCG, | |
| 2370, | X61176, | X61176, | TTTGATCAATGGCGTGAAGG, | |
| 2371, | X61176, | X61176, | CAATGGCGTGAAGGGCAAAC, | |
| 2372, | X61176, | X61176, | CGTGAAGGGCAAACAGCTGA, | |
| 2373, | X61176, | X61176, | GGGCAAACAGCTGATCAAAG, | |
| 2374, | X61176, | X61176, | ACAGCTGATCAAAGGGCCTG, | |
| 2375, | X61176, | X61176, | GATCAAAGGGCCTGATAGCA, | |
| 2376, | X61176, | X61176, | AGGGCCTGATAGCAGAGTGC, | |
| 2377, | X61176, | X61176, | TGATAGCAGAGTGCTTGCTG, | |
| 2378, | X61176, | X61176, | CAGAGTGCTTGCTGGAGCCG, | |
| 2379, | X61176, | X61176, | GCTTGCTGGAGCCGTTAACA, | |
| 2380, | X61176, | X61176, | TGGAGCCGTTAACAAGCACC, | |
| 2381, | X61176, | X61176, | CGTTAACAAGCACCGCAAGC, | |
| 2382, | X61176, | X61176, | CAAGCACCGCAAGCCAGTCA, | |
| 2383, | X61176, | X61176, | CCGCAAGCCAGTCACGCCCT, | |
| 2384, | X61176, | X61176, | GCCAGTCACGCCCTTTGCTG, | |
| 2385, | X61176, | X61176, | CACGCCCTTTGCTGAGGATA, | |
| 2386, | X61176, | X61176, | CTTTGCTGAGGATAAAAGTC, | |
| 2387, | X61176, | X61176, | TGAGGATAAAAGTCCTGGGA, | |
| 2388, | X61176, | X61176, | TAAAAGTCCTGGGAAACCAG, | |
| 2389, | X61176, | X61176, | TCCTGGGAAACCAGCATGCG, | |
| 2390, | X61176, | X61176, | GAAACCAGCATGCGATATTT, | |
| 2391, | X61176, | X61176, | AGCATGCGATATTTCTCCCC, | |
| 2392, | X61176, | X61176, | CGATATTTCTCCCCAGTGTG, | |
| 2393, | X61176, | X61176, | TTCTCCCCAGTGTGTCTTTG, | |
| 2394, | X61176, | X61176, | CCAGTGTGTCTTTGCTGTAT, | |
| 2395, | X61176, | X61176, | TGTCTTTGCTGTATTCTTGG, | |
| 2396, | X61176, | X61176, | TGCTGTATTCTTGGCATTCT, | |
| 2397, | X61176, | X61176, | ATTCTTGGCATTCTTCAGTC, | |
| 2398, | X61176, | X61176, | GGCATTCTTCAGTCCAAGAG, | |
| 2399, | X61176, | X61176, | CTTCAGTCCAAGAGCCATAC, | |
| 2400, | X61176, | X61176, | TCCAAGAGCCATACCTATAG, | |
| 2401, | X61176, | X61176, | AGCCATACCTATAGTAGAGA, | |
| 2402, | X61176, | X61176, | ACCTATAGTAGAGAAAATAC, | |
| 2403, | X61176, | X61176, | AGTAGAGAAAATACTGCGTG, | |
| 2404, | X61176, | X61176, | GAAAATACTGCGTGTCCTCA, | |
| 2405, | X61176, | X61176, | ACTGCGTGTCCTCAGGGGCA, | |
| 2406, | X61176, | X61176, | TGTCCTCAGGGGCATCTGTG, | |
| 2407, | X61176, | X61176, | CAGGGGCATCTGTGCCAACA, | |
| 2408, | X61176, | X61176, | CATCTGTGCCAACAAGCCAG, | |
| 2409, | X61176, | X61176, | TGCCAACAAGCCAGGTGCAG, | |
| 2410, | X61176, | X61176, | CAAGCCAGGTGCAGTGAAGG, | |
| 2411, | X61176, | X61176, | AGGTGCAGTGAAGGGAAACT, | |
| 2412, | X61176, | X61176, | AGTGAAGGGAAACTTGGTAT, | |
| 2413, | X61176, | X61176, | GGGAAACTTGGTATGACCTT, | |
| 2414, | X61176, | X61176, | CTTGGTATGACCTTAAACGT, | |
| 2415, | X61176, | X61176, | ATGACCTTAAACGTGAATAA, | |
| 2416, | X61176, | X61176, | TTAAACGTGAATAATTGTCT, | |
| 2417, | X61176, | X61176, | GTGAATAATTGTCTTCTGTA, | |
| 2418, | X61176, | X61176, | AATTGTCTTCTGTAGTGTTT, | |
| 2419, | X61176, | X61176, | CTTCTGTAGTGTTTGTGGTG, | |
| 2420, | X61176, | X61176, | TAGTGTTTGTGGTGCAAGTT, | |
| 2421, | X61176, | X61176, | TTGTGGTGCAAGTTAAATTC, | |
| 2422, | X61176, | X61176, | TGCAAGTTAAATTCACAACT, | |
| 2423, | X61176, | X61176, | TTAAATTCACAACTGAGGTT, | |
| 2424, | X61176, | X61176, | TCACAACTGAGGTTCCAGGA, | |
| 2425, | X61176, | X61176, | CTGAGGTTCCAGGAGACCCT, | |
| 2426, | X61176, | X61176, | TTCCAGGAGACCCTGGTGGG, | |
| 2427, | X61176, | X61176, | GAGACCCTGGTGGGGCATGA, | |
| 2428, | X61176, | X61176, | CTGGTGGGGCATGAAGTTCA, | |
| 2429, | X61176, | X61176, | GGGCATGAAGTTCAGCAGAA, | |
| 2430, | X61176, | X61176, | GAAGTTCAGCAGAAGCCCAG, | |
| 2431, | X61176, | X61176, | CAGCAGAAGCCCAGCTGCTG, | |
| 2432, | X61176, | X61176, | AAGCCCAGCTGCTGGCCAGT, | |
| 2433, | X61176, | X61176, | AGCTGCTGGCCAGTAGTGAG, | |
| 2434, | X61176, | X61176, | TGGCCAGTAGTGAGTGGTCG, | |
| 2435, | X61176, | X61176, | GTAGTGAGTGGTCGTTCTGC, | |
| 2436, | X61176, | X61176, | AGTGGTCGTTCTGCAGGATG, | |
| 2437, | X61176, | X61176, | CGTTCTGCAGGATGGTCCGC, | |
| 2438, | X61176, | X61176, | GCAGGATGGTCCGCACACTT, | |
| 2439, | X61176, | X61176, | TGGTCCGCACACTTGCTGAA, | |
| 2440, | X61176, | X61176, | GCACACTTGCTGAAAAGCCT, | |
| 2441, | X61176, | X61176, | TTGCTGAAAAGCCTTTGTGG, | |
| 2442, | X61176, | X61176, | AAAAGCCTTTGTGGAGGATG, | |
| 2443, | X61176, | X61176, | CTTTGTGGAGGATGGTTACA, | |
| 2444, | X61176, | X61176, | GGAGGATGGTTACACATTTG, | |
| 2445, | X61176, | X61176, | TGGTTACACATTTGCTTTCA, | |
| 2446, | X61176, | X61176, | CACATTTGCTTTCAGTGATT, | |
| 2447, | X61176, | X61176, | TGCTTTCAGTGATTCTGGTT, | |
| 2448, | X61176, | X61176, | CAGTGATTCTGGTTTCATAG, | |
| 2449, | X61176, | X61176, | TTCTGGTTTCATAGTCATCT, | |
| 2450, | X61176, | X61176, | TTTCATAGTCATCTTCTTTT, | |
| 2451, | X61176, | X61176, | AGTCATCTTCTTTTGGAGCG, | |
| 2452, | X61176, | X61176, | CTTCTTTTGGAGCGTTTATT, | |
| 2453, | X61176, | X61176, | TTGGAGCGTTTATTTTCACT, | |
| 2454, | X61176, | X61176, | CGTTTATTTTCACTTGATAT, | |
| 2455, | X61176, | X61176, | TTTTCACTTGATATTCTAGA, | |
| 2456, | X61176, | X61176, | CTTGATATTCTAGATTAACA, | |
| 2457, | X61176, | X61176, | ATTCTAGATTAACATTCCTT, | |
| 2458, | X61176, | X61176, | GATTAACATTCCTTTGCTCT, | |
| 2459, | X61176, | X61176, | CATTCCTTTGCTCTTGATCA, | |
| 2460, | X61176, | X61176, | TTTGCTCTTGATCAGGATTT, | |
| 2461, | X61176, | X61176, | CTTGATCAGGATTTGGTTTC, | |
| 2462, | X61176, | X61176, | CAGGATTTGGTTTCCATTGT, | |
| 2463, | X61176, | X61176, | TTGGTTTCCATTGTAAAAGA, | |
| 2464, | X61176, | X61176, | TCCATTGTAAAAGAACTTGA, | |
| 2465, | X61176, | X61176, | GTAAAAGAACTTGAGCCAAA, | |
| 2466, | X61176, | X61176, | GAACTTGAGCCAAACCAGTA, | |
| 2467, | X61176, | X61176, | GAGCCAAACCAGTAACTTTA, | |
| 2468, | X61176, | X61176, | AACCAGTAACTTTAATGGTG, | |
| 2469, | X61176, | X61176, | TAACTTTAATGGTGAAATTG, | |
| 2470, | X61176, | X61176, | TAATGGTGAAATTGACAGGT, | |
| 2471, | X61176, | X61176, | TGAAATTGACAGGTGGGAGA, | |
| 2472, | X61176, | X61176, | TGACAGGTGGGAGAAGTGAA, | |
| 2473, | X61176, | X61176, | GTGGGAGAAGTGAAATCTTT, | |
| 2474, | X61176, | X61176, | GAAGTGAAATCTTTTCATCA, | |
| 2475, | X61176, | X61176, | AAATCTTTTCATCAGGAAGT, | |
| 2476, | X61176, | X61176, | TTTCATCAGGAAGTAAGTCA, | |
| 2477, | X61176, | X61176, | CAGGAAGTAAGTCAGCTTGC, | |
| 2478, | X61176, | X61176, | GTAAGTCAGCTTGCAGTATC, | |
| 2479, | X61176, | X61176, | CAGCTTGCAGTATCTCAGTG, | |
| 2480, | X61176, | X61176, | GCAGTATCTCAGTGGCCCCC, | |
| 2481, | X61176, | X61176, | TCTCAGTGGCCCCCAAAAGG, | |
| 2482, | X61176, | X61176, | TGGCCCCCAAAAGGATGAGT, | |
| 2483, | X61176, | X61176, | CCAAAAGGATGAGTAATACA, | |
| 2484, | X61176, | X61176, | GGATGAGTAATACATGCGCC, | |
| 2485, | X61176, | X61176, | GTAATACATGCGCCACGATG, | |
| 2486, | X61176, | X61176, | CATGCGCCACGATGATCATA, | |
| 2487, | X61176, | X61176, | CCACGATGATCATATCCTGT, | |
| 2488, | X61176, | X61176, | TGATCATATCCTGTCTACAG, | |
| 2489, | X61176, | X61176, | TATCCTGTCTACAGACGATC, | |
| 2490, | X61176, | X61176, | GTCTACAGACGATCCTCTTG, | |
| 2491, | X61176, | X61176, | AGACGATCCTCTTGTTCCGA, | |
| 2492, | X61176, | X61176, | TCCTCTTGTTCCGACCAGTA, | |
| 2493, | X61176, | X61176, | TGTTCCGACCAGTACTCAAC, | |
| 2494, | X61176, | X61176, | GACCAGTACTCAACAGAAGA, | |
| 2495, | X61176, | X61176, | TACTCAACAGAAGATGGCGA, | |
| 2496, | X61176, | X61176, | ACAGAAGATGGCGAGGACCG, | |

| Concatemer Nucleic Acid Sequences of IL4R-X61176 gene oligo sequences | |
|---|---|
| (SEQ ID NO: 2497) | |
| ATATATTAGCTCTATTATGCTAGCTCTATTATGCTTTTAATATTATGCTTTTAAATTAGTGCTTTTAAATTAGTTGGTTTAAATTAGTTGGTTTCAAATGGTTGGTTTCAAATGATGTATTTCAAATGATGTAT | |
| CCTGGATGATGTATCCTGGATCAGGCATCCTGGATCAGGCCTCTGGGATCAGGCCTCTGGAGCTTGGCCTCTGGAGCTTGAGATAAGGAGCTTGAGATAATTTCTCTGAGATAATTTCTCTCTCTCAATTTCTC | |
| TCTCTCTCTCTCTCTCTCTCTCTCTCTCTCTCTCTCTCTCTCTCTCTCTCTCTCTCTCTCTCTCTCTCTCTCTCTCTCTCTCTCTCTCATACTCTCTCTCTCATACACACACTCTCATACACACACATACACAC | |
| ACACACATACACAATCTAACATACACAATCTACCTAGAACAATCTACCTAGAAAAAGATACCTAGAAAAAGATCATTCGAAAAAGATCATTCTAGAATGATCATTCTAGAATGTAGGGTCTAGAATGTAGGGGG | |
| GTGAATGTAGGGGGGTGAGGAATTGGGGGTGAGGAATTTGTGGCGAGGAATTTGTGGCTCTCACTTTGTGGCTCTCACTTGCTAGCTCTCACTTGCTATAGAAAACTTGCTATAGAAAAGACAGTATAGAAAAG | |
| ACAGTCTTGAAAAGACAGTCTTGAATCCAAAGTCTTGAATCCAAGTTCATGAATCCAAGTTCATGATTCAAAGTTCATGATTCACTCCAGATGATTCACTCCAGGCTGATCACTCCAGGCTGATGCAAAAAGGC | |
| TGATGCAAAATGCTTTATGCAAAATGCTTTCTTCCTAATGCTTTCTTCCTCAGATGTTCTTCCTCAGATGGTAGCTCTCAGATGGTAGCTGAGTGCTGGTAGCTGAGTGCTACAATCTGAGTGCTACAATTGGC | |
| AGGCTACAATTGGCAGCTTAAAATTGGCAGCTTAAACAGCCAAGCTTAAACAGCCAAACGGGAACAGCCAAACGGGCACAGCCAAACGGGCACAGCCAGAAGGGCACAGCCAGAAGTAAATAGCCAGAAGTAAA | |
| TACAGCTGAGTAAATACAGCTGGACGTTTACAGCTGGACGTTAGCCTTTGGACGTTAGCCTTAAAAGCTTAGCCTTAAAAGCTGTCTGTTAAAAGCTGTCTGTTGTGAGCTGTCTGTTGTGAATGAAATGTTGT | |
| GAATGAAAAGTCTGGAATGAAAAGTCTGAGGTGAAAAGTCTGAGGTGAGTCAAGTGAGGTGAGTCAAGCAAATTGAGTCAAGCAAATTGCAAAGAGCAAATTGCAAAGATTGGCTTGCAAAGATTGGCAGGTGA | |
| AGATTGGCAGGTGAGGAGGTGCAGGTGAGGAGGTGCTACCGAGGAGGTGCTACCCTGTACGTGCTACCCTGTACGGCATGCCCTGTACGGCATGGGGAGAACGGCATGGGGAGAAAAAACTGGGGAGAAAAAAC | |
| ATGGCAGAAAAAACATGGCAAAATGCACATGGCAAAATGCTTGGATCAAAATGCTTGGATGAGTCAGCTTGGATGAGTCAACTTCCATGAGTCAACTTCCCTGCTGCAACTTCCCTGCTGTAGGTGCCCTGCTG | |
| TAGGTGAGGCGATGTAGGTGAGGCGATTTGGATGAGGCGATTTGGATGAAGCGATTTGGATGAAGCATCCATGATGAAGCATCCATACTTTTGCATCCATACTTTTAAGAGAATACTTTTAAGAGATACAAGTT | |
| AAGAGATACAAGACTGGTGATACAAGACTGGTGTGTCTAGACTGGTGTGTCTGGGTGTGTGTGTCTGGGTGTATTGCTCTGGGTGTATTGCTTCGCAGGTATTGCTTCGCAGGTAAATCTTCGCAGGTAAATTG | |
| AGTGAGGTAAATTGAGTGTTGCCTATTGAGTGTTGCCTAAATTCTGTTGCCTAAATTCTGAACACTAAATTCTGAACACCTCTTTCTGAACACCTCTTAGCCAACACCTCTTAGCCAAGAGCCATTAGCCAAGA | |
| GCCAGCATCCAAGAGCCAGCATCCCTGTTCCAGCATCCCTGTTCTTTTCACCCTGTTCTTTTCACTGAGGTCTTTTCACTGAGGCACTGACACTGAGGCACTGAGGCATGGGCACTGAGGCATGTGTGAGGAGG | |
| CATGTGTGAGTTCATCTGTGTGAGTTCATCAGAGGAAGTTCATCAGAGGATGCCAATCAGAGGATGCCAAAGTGACGATGCCAAAGTGACAGTCAAAAAGTGACAGTCAAAACACAACAGTCAAAACACAGAAT | |
| CCAAAACACAGAATCCTCCAGGCAGAATCCTCCAGGGTCTCACCTCCAGGGTCTCAACTCCAGGGTCTCAACTCCAGGCTTCCAACTCCAGGCTTCTCTATACAGGCTTCTCTATATAACAGTCTCTATATAAC | |
| AGATGACTTATAACAGATGACTTCAATTAGATGACTTCAATTTCCGTCCTTCAATTTCCGTCTCACTGTTTCCGTCTCACTGGACCCATCTCACTGGACCCAGCTTTCTGGACCCAGCTTTCTCATAGCAGCTT | |
| TCTCATAGTTAGTGTCTCATAGTTAGTGGTTACAAGTTAGTGGTTACAAAGAGATGGTTACAAAGAGATCTTTGCAAAGAGATCTTTGATATTAGATCTTTGATATTACTTTTTTGATATTACTTTTTGGTGCT | |
| TACTTTTTGGTGCTGGAATTTTGGTGCTGGAATTGGTGGACTGGAATTGGTGGAAACAACTTGGTGGAAACAACTTGATCGAAACAACTTGATCCATAAAACTTGATCCATAAATGACATTCCATAAATGACAT | |
| ATTTTAAATGACATATTTTACAGATAATATTTTACAGATAAGCGAGTACAGATAAGCGAGAGAATTTAAGCGAGAGAATTAACAAGAGAGAATTAACAAGATGAAGTTAACAAGATGAAGCAGATGAGATGAAG | |
| CAGATGGTTGCCAGCAGATGGTTGCCATAATCTGGTTGCCATAATCACAATGCCATAATCACAATGACAAACTCACAATGACAAACCACTCTTGACAAACCACTCTCTCAAGACCACTCTCTCAAGGGCTTGCT | |
| CTCAAGGGCTTGTGTTCAAGGGCTTGTGTTCATCATTTTGTGTTCATCATTTCCCACACATCATTTCCCACATAAATATTCCCACATAAATAGGTTGGCATAAATAGGTTGGCTCCACTAGGTTGGCTCCACTC | |
| ACTCGGCTCCACTCACTCCAGAGCACTCACTCCAGAGCCCTGCCTCCAGAGCCCTGCCTCTCTGGCCCTGCCTCTCTGCACATGCCTCTCTGCACATGGAGCTCTGCACATGGAGCTCACTGCTTGGAGCTCAC | |
| TGCTGCTCTCTCACTGCTGCTCTCACTTGACTGCTCTCACTTGAACATCGTCACTTGAACATCGTACTTAGAACATCGTACTTAGAAAGACGTACTTAGAAAGATCATCATAGAAAGATCATCAATTATTGATC | |
| ATCAATTATTGAGATGCAATTATTGAGATGAATGCATTGAGATGAATGCATTGGTCTGAATGCATTGGTCATCAATCATTGGTCATCAATTTTTCTTCATCAATTTTTCTATCTGCATTTTTCTATCTGCAAAT | |
| ATCTATCTGCAAATATCCATTCGCAAATATCCATTCCTTGTAATCCATTCCTTGTATTGTGTTCCTTGTATTGTGTATTTTTTATTGTGTATTTTTACTTCAGTATTTTTACTTCATAATCATTACTTCATAAT | |
| CAAAGCAACATAATCAAAGCAATGGATTCAAAGCAATGGATTGGAAAAAATGGATTGGAAAAGCAGACTTGGAAAAGCAGACACTGGTAAGCAGACACTGGTTTCTCCACACTGGTTTCTCCCATTGGGTTTCT | |
| CCCATTGGATAGAGCCCATTGGATAGAGAGACGAGGATAGAGAGACGAGTTCCTAGAGACGAGTTCCTTCAATCGAGTTCCTTCAATCTCTGCTCTTCAATCTCTGCTGTGACATCTCTGCTGTGACATTCAGT | |
| CTGTGACATTCAGTGGAGGACATTCAGTGGAGGATTTATTGTGGAGGATTTATTTGATCAGATTTATTTGATCAATGGCGTTTGATCAATGGCGTGAAGGCAATGGCGTGAAGGGCAAACCGTGAAGGGCAAAC | |
| AGCTGAGGGCAAACAGCTGATCAAAGACAGCTGATCAAAGGGCCTGGATCAAAGGGCCTGATAGCAAGGGCCTGATAGCAGAGTGCTGATAGCAGAGTGCTTGCTGCAGAGTGCTTGCTGGAGCCGGCTTGCTG | |
| GAGCCGTTAACATGGAGCCGTTAACAAGCACCCGTTAACAAGCACCGCAAGCCAAGCACCGCAAGCCAGTCACCGCAAGCCAGTCACGCCCTGCCAGTCACGCCCTTTGCTGCACGCCCTTTGCTGAGGATACT | |
| TTGCTGAGGATAAAAGTCTGAGGATAAAAGTCCTGGGATAAAAGTCCTGGGAAACCAGTCCTGGGAAACCAGCATGCGGAAACCAGCATGCGATATTTAGCATGCGATATTTCTCCCCCGATATTTCTCCCCAG | |
| TGTGTTCTCCCCAGTGTGTCTTTGCCAGTGTGTCTTTGCTGTATTGTCTTTGCTGTATTCTTGGTGCTGTATTCTTGGCATTCTATTCTTGGCATTCTTCAGTCGGCATTCTTCAGTCCAAGAGCTTCAGTCCA | |
| AGAGCCATACTCCAAGAGCCATACCTATAGAGCCATACCTATAGTAGAGAACCTATAGTAGAGAAAATACAGTAGAGAAAATACTGCGTGGAAAATACTGCGTGTCCTCAACTGCGTGTCCTCAGGGGCATGTC | |
| CTCAGGGGCATCTGTGCAGGGGCATCTGTGCCAACACATCTGTGCCAACAAGCCAGTGCCAACAAGCCAGGTGCAGCAAGCCAGGTGCAGTGAAGGAGGTGCAGTGAAGGGAAACTAGTGAAGGGAAACTTGGT | |
| ATGGGAAACTTGGTATGACCTTCTTGGTATGACCTTAAACGTATGACCTTAAACGTGAATAATTAAACGTGAATAATTGTCTGTGAATAATTGTCTTCTGTAAATTGTCTTCTGTAGTGTTTCTTCTGTAGTGT | |
| TTGTGGTGTAGTGTTTGTGGTGCAAGTTTTGTGGTGCAAGTTAAATTCTGCAAGTTAAATTCACAACTTTAAATTCACAACTGAGGTTTCACAACTGAGGTTCCAGGACTGAGGTTCCAGGAGACCCTTTCCAG | |
| GAGACCCTGGTGGGGAGACCCTGGTGGGGCATGACTGGTGGGGCATGAAGTTCAGGGCATGAAGTTCAGCAGAAGAAGTTCAGCAGAAGCCCAGCAGCAGAAGCCCAGCTGCTGAAGCCCAGCTGCTGGCCAGT | |
| AGCTGCTGGCCAGTAGTGAGTGGCCAGTAGTGAGTGGTCGGTAGTGAGTGGTCGTTCTGCAGTGGTCGTTCTGCAGGATGCGTTCTGCAGGATGGTCCGCGCAGGATGGTCCGCACACTTTGGTCCGCACACTT | |
| GCTGAAGCACACTTGCTGAAAAGCCTTTGCTGAAAAGCCTTTGTGGAAAAGCCTTTGTGGAGGATGCTTTGTGGAGGATGGTTACAGGAGGATGGTTACACATTTGTGGTTACACATTTGCTTTCACACATTTG | |
| CTTTCAGTGATTTGCTTTCAGTGATTCTGGTTCAGTGATTCTGGTTTCATAGTTCTGGTTTCATAGTCATCTTTTCATAGTCATCTTCTTTTAGTCATCTTCTTTTGGAGCGCTTCTTTTGGAGCGTTTATTTT | |
| GGAGCGTTTATTTTCACTCGTTTATTTTCACTTGATATTTTTCACTTGATATTCTAGACTTGATATTCTAGATTAACAATTCTAGATTAACATTCCTTGATTAACATTCCTTTGCTCTCATTCCTTTGCTCTTG | |
| ATCATTTGCTCTTGATCAGGATTTCTTGATCAGGATTTGGTTTCCAGGATTTGGTTTCCATTGTTTGGTTTCCATTGTAAAAGATCCATTGTAAAAGAACTTGAGTAAAAGAACTTGAGCCAAAGAACTTGAGC | |
| CAAACCAGTAGAGCCAAACCAGTAACTTTAAACCAGTAACTTTAATGGTGTAACTTTAATGGTGAAATTGTAATGGTGAAATTGACAGGTTGAAATTGACAGGTGGGAGATGACAGGTGGGAGAAGTGAAGTGG | |
| GAGAAGTGAAATCTTTGAAGTGAAATCTTTTCATCAAAATCTTTTCATCAGGAAGTTTTCATCAGGAAGTAAGTCACAGGAAGTAAGTCAGCTTGCGTAAGTCAGCTTGCAGTATCCAGCTTGCAGTATCTCAG | |
| TGGCAGTATCTCAGTGGCCCCCTCTCAGTGGCCCCCAAAAGGTGGCCCCCAAAAGGATGAGTCCAAAAGGATGAGTAATACAGGATGAGTAATACATGCGCCGTAATACATGCGCCACGATGCATGCGCCACGA | |
| TGATCATACCACGATGATCATATCCTGTTGATCATATCCTGTCTACAGTATCCTGTCTACAGACGATCGTCTACAGACGATCCTCTTGAGACGATCCTCTTGTTCCGATCCTCTTGTTCCGACCAGTATGTTCC | |
| GACCAGTACTCAACGACCAGTACTCAACAGAAGATACTCAACAGAAGATGGCGAACAGAAGATGGCGAGGACCG | |
| Concatemer Nucleic Acid Sequences of all gene sequences | |
| | (SEQ ID NO: 2498) |
| GGCGAATGGAGCAGGGGCGCGCAGATAATTAAAGATTTACACACAGCTGGAAGAAATCATAGAGAAGCCGGGCGTGGTGGCTCATGCCTATAATCCCAGCACTTTTGGAGGCTGAGGCGGGCAGATCACTTGAG | |
| ATCAGGAGTTCGAGACCAGCCTGGTGCCTTGGCATCTCCCAATGGGGTGGCTTTGCTCTGGGCTCCTGTTCCCTGTGAGCTGCCTGGTCCTGCTGCAGGTGGCAAGCTCTGGGAACATGAAGGTCTTGCAGGAG | |
| CCCACCTGCGTCTCCGACTACATGAGCATCTCTACTTGCGAGTGGAAGATGAATGGTCCCACCAATTGCAGCACCGAGCTCCGCCTGTTGTACCAGCTGGTTTTTCTGCTCTCCGAAGCCCACACGTGTATCCC | |
| TGAGAACAACGGAGGCGCGGGGTGCGTGTGCCACCTGCTCATGGATGACGTGGTCAGTGCGGATAACTATACACTGGACCTGTGGGCTGGGCAGCAGCTGCTGTGGAAGGGCTCCTTCAAGCCCAGCGAGCATG | |
| TGAAACCCAGGGCCCCAGGAAACCTGACAGTTCACACCAATGTCTCCGACACTCTGCTGCTGACCTGGAGCAACCCGTATCCCCCTGACAATTACCTGTATAATCATCTCACCTATGCAGTCAACATTTGGAGT | |
| GAAAACGACCCGGCAGATTTCAGAATCTATAACGTGACCTACCTAGAACCCTCCCTCCGCATCGCAGCCAGCACCCTGAAGTCTGGGATTTCCTACAGGGCACGGGTGAGGGCCTGGGCTCAGTGCTATAACAC | |
| CACCTGGAGTGAGTGGAGCCCCAGCACCAAGTGGCACAACTCCTACAGGGAGCCCTTCGAGCAGCACCTCCTGCTGGGCGTCAGCGTTTCCTGCATTGTCATCCTGGCCGTCTGCCTGTTGTGCTATGTCAGCA | |
| TCACCAAGATTAAGAAAGAATGGTGGGATCAGATTCCCAACCCAGCCCGCAGCCGCCTCGTGGCTATAATAATCCAGGATGCTCAGGGGTCACAGTGGGAGAAGCGGTCCCGAGGCCAGGAACCAGCCAAGTGC | |
| CCACACTGGAAGAATTGTCTTACCAAGCTCTTGCCCTGTTTTCTGGAGCACAACATGAAAAGGGATGAAGATCCTCACAAGGCTGCCAAAGAGATGCCTTTCCAGGGCTCTGGAAAATCAGCATGGTGCCCAGT | |
| GGAGATCAGCAAGACAGTCCTCTGGCCAGAGAGCATCAGCGTGGTGCGATGTGTGGAGTTGTTTGAGGCCCCGGTGGAGTGTGAGGAGGAGGAGGAGGTAGAGGAAGAAAAAGGGAGCTTCTGTGCATCGCCTG | |
| AGAGCAGCAGGGATGACTTCCAGGAGGGAAGGGAGGGCATTGTGGCCGGCTAACAGAGAGCCTGTTCCTGGACCTGCTCGGAGAGGAGAATGGGGGCTTTTGCCAGCAGGACATGGGGGGAGTCATGCCTTCTT | |
| CCACCTTCGGGAAGTACGAGTGCTCACATGCCCTGGGATGAGTTCCCAAGTGCAGGGCCCAAGGAGGCACCTCCCTGGGGCAAGGAGCAGCCTCTCCACCTGGAGCCAAGTCCTCCTGCCAGCCCGACCCAGAG | |
| TCCAGACAACCTGACTTGCACAGAGACGCCCCTCGTCATCGCAGGCAACCCTGCTTACCGCAGCTTCAGCAACTCCCTGAGCCAGTCACCGTGTCCCAGAGAGCTGGGTCCAGACCCACTGCTGGCCAGACACC | |
| TGGAGGAAGTAGAACCCGAGATGCCCTGTGTCCCCCAGCTCTCTGAGCCAACCACTGTGCCCCAACCTGAGCCAGAAACCTGGGAGCAGATCCTCCGCCGAAATGTCCTCCAGCATGGGGCAGCTGCAGCCCCC | |
| GTCTCGGCCCCCACCAGTGGCTATCAGGAGTTTGTACATGCGGTGGAGCAGGGTGGCACCCAGGCCAGTGCGGTGGTGGGCTTGGGTCCCCCAGGAGAGGCTGGTTACAAGGCCTTCTCAAGCCTGCTTGCCAG | |
| CAGTGCTGTGTCCCCAGAGAAATGTGGGTTTGGGGCTAGCAGTGGGGAAGAGGGGTATAAGCCTTTCCAAGACCTCATTCCTGGCTGCCCTGGGGACCCTGCCCCAGTCCCTGTCCCCTTGTTCACCTTTGGAC | |
| TGGACAGGGAGCCACCTCGCAGTCCGCAGAGCTCACATCTCCCAAGCAGCTCCCCAGAGCACCTGGGTCTGGAGCCGGGGGAAAAGGTAGAGGACATGCCAAAGCCCCCACTTCCCCAGGAGCAGGCCACAGAC | |
| CCCCTTGTGGACAGCCTGGGCAGTGGCATTGTCTACTCAGCCCTTACCTGCCACCTGTGCGGCCACCTGAAACAGTGTCATGGCCAGGAGGATGGTGGCCAGACCCCTGTCATGGCCAGTCCTTGCTGTGGCTG | |
| CTGCTGTGGAGACAGGTCCTCGCCCCCTACAACCCCCCTGAGGGCCCCAGACCCCTCTCCAGGTGGGGTTCCACTGGAGGCCAGTCTGTGTCCGGCCTCCCTGGCACCCTCGGGCATCTCAGAGAAGAGTAAAT | |
| CCTCATCATCCTTCCATCCTGCCCCTGGCAATGCTCAGAGCTCAAGCCAGACCCCCAAAATCGTGAACTTTGTCTCCGTGGGACCCACATACATGAGGGTCTCTTAGGTGCATGTCCTCTTGTTGCTGAGTCTG | |
| CAGATGAGGACTAGGGCTTATCCATGCCTGGGAAATGCCACCTCCTGGAAGGCAGCCAGGCTGGCAGATTTCCAAAAGACTTGAAGAACCATGGTATGAAGGTGATTGGCCCCACTGACGTTGGCCTAACACTG | |
| GGCTGCAGAGACTGGACCCCGCCCAGCATTGGGCTGGGCTCGCCACATCCCATGAGAGTAGAGGGCACTGGGTCGCCGTGCCCCACGGCAGGCCCCTGCAGGAAAACTGAGGCCCTTGGGCACCTCGACTTGTG | |
| AACGAGTTGTTGGCTGCTCCCTCCACAGCTTCTGCAGCAGACTGTCCCTGTTGTAACTGCCCAAGGCATGTTTTGCCCACCAGATCATGGCCCACGTGGAGGCCCACCTGCCTCTGTCTCACTGAACTAGAAGC | |
| CGAGCCTAGAAACTAACACAGCCATCAAGGGAATGACTTGGGCGGCCTTGGGAAATCGATGAGAAATTGAACTTCAGGGAGGGTGGTCATTGCCTAGAGGTGCTCATTCATTTAACAGAGCTTCCTTAGGTTGA | |
| TGCTGGAGGCAGAATCCCGGCTGTCAAGGGGTGTTCAGTTAAGGGGAGCAACAGAGGACATGAAAAATTGCTATGACTAAAGCAGGGACAATTTGCTGCCAAACACCCATGCCCAGCTGTATGGCTGGGGGCTC | |
| CTCGTATGCATGGAACCCCCAGAATAAATATGCTCAGCCACCCTGTGGGCCGGGCAATCCAGACAGCAGGCATAAGGCACCAGTTACCCTGCATGTTGGCCCAGACCTCAGGTGCTAGGGAAGGCGGGAACCTT | |
| GGGTTGAGTAATGCTCGTCTGTGTGTTTTAGTTTCATCACCTGTTATCTGTGTTTGCTGAGGAGAGTGGAACAGAAGGGGTGGAGTTTTGTATAAATAAAGTTTCTTTGTCTCTAGATGCTGGGGTTGCAGCCA | |
| CGAGCATAGACACGACAGACACGGTCCTCGCCATCTTCTGTTGAGTACTGGTCGGAACAAGAGGATCGTCGTAGACAGGATATGATCATCGTGGCGCATGTATTACTCATCCTTTTGGGGGGCCACTGAGATAC | |
| TGCAAGCTGACTTACTTCCTGATGAAAAGATTTCACTTCTCCCACCTGTCAATTTCACCATTAAAGTTACTGGTTTGGCTCAAGTTCTTTTACAATGGAAACCAAATCCTGATCAAGAGCAAAGGAATGTTAAT | |
| CTAGAATATCAAGTGAAAATAAACGCTCCAAAAGAAGATGACTATGAAACCAGAATCACTGAAAGCAAATGTGTAACCATCCTCCACAAAGGCTTTTCAGCAAGTGTGCGGACCATCCTGCAGAACGACCACTC | |
| ACTACTGGCCAGCAGCTGGGCTTCTGCTGAACTTCATGCCCCACCAGGGTCTCCTGGAACCTCAATTGTGAATTTAACTTGCACCACAAACACTACAGAAGACAATTATTCACGTTTAAGGTCATACCAAGTTT | |
| CCCTTCACTGCACCTGGCTTGTTGGCACAGATGCCCCTGAGGACACGCAGTATTTTCTCTACTATAGGTATGGCTCTTGGACTGAAGAATGCCAAGAATACAGCAAAGACACACTGGGGAGAAATATCGCATGC | |
| TGGTTTCCCAGGACTTTTATCCTCAGCAAAGGGCGTGACTGGCTTGCGGTGCTTGTTAACGGCTCCAGCAAGCACTCTGCTATCAGGCCCTTTGATCAGCTGTTTGCCCTTCACGCCATTGATCAAATAAATCC | |
| TCCACTGAATGTCACAGCAGAGATTGAAGGAACTCGTCTCTCTATCCAATGGGAGAAACCAGTGTCTGCTTTTCCAATCCATTGCTTTGATTATGAAGTAAAAATACACAATACAAGGAATGGATATTTGCAGA | |
| TAGAAAAATTGATGACCAATGCATTCATCTCAATAATTGATGATCTTTCTAAGTACGATGTTCAAGTGAGAGCAGCAGTGAGCTCCATGTGCAGAGAGGCAGGGCTCTGGAGTGAGTGGAGCCAACCTATTTAT | |
| GTGGGAAATGATGAACACAAGCCCTTGAGAGAGTGGTTTGTCATTGTGATTATGGCAACCATCTGCTTCATCTTGTTAATTCTCTCGCTTATCTGTAAAATATGTCATTTATGGATCAAGTTGTTTCCACCAAT | |
| TCCAGCACCAAAAAGTAATATCAAAGATCTCTTTGTAACCACTAACTATGAGAAAGCTGGAATTTAAATTCAAGCATGTTTTAACTTTTGGTTTAAGGTACTTGGGTGTACCTGGCAGTGTTGTAAGCTCTTTA | |
| CATTAATTAATTAACTCTCTAGGTACTGTTATCTTCATTTTATAAACAAGGCAGCTGAAGTTGAGAGAAATAAGTAACCTGTCCTAGGTCACACAATTAGGAAATGACAGATCTGGCAGTCTATTTCCAGGCAG | |
| TCTATTTCCACGAGGTCATGAGTGCGAAAGAGGGACTAGGGGAAGAATGATTAACTCCAGGGAGCTGACTTTTCTAGTGTGCTTACCTGTTTTGCATCTCTCAAGGATGTGCCATGAAGCTGTAGCCAGGTGGA | |
| ATTGTACCACAGCCCTGACATGAACACCTGATGGCAGCTGCTGGGTTGGAGCCTAGACAAAAACATGAAGAACCATGGCTGCTGCCTGAGCCCATCGTGCTGTAATTATAGAAAACCTTCTAAGGGAAGAATAT | |
| GCTGATATTTTTCAGATAAGTACCCCTTTTATAAAAATCCTCCAAGTTAGCCCTCGATTTTCCATGTAAGGAAACAGAGGCTTTGAGATAATGTCTGTCTCCTAAGGGACAAAGCCAGGACTTGATCCTGTCTT | |
| AAAAATGCAAAATGTAGTACTTCTTCCATCAAAGGTAGACATGCACTAAGGGACAGGTTTTGGCTTGGTATCAGAATAGCATTTTTAAAAGCTGTGTAAGAATTGAACGGGCTGTACTAGGGGTATAGAGAAGC | |
| CGGGATGGAAACTCCAAACACCACAGAGGACTATGACACGACCACAGAGTTTGACTATGGGGATGCAACTCCGTGCCAGAAGGTGAACGAGAGGGCCTTTGGGGCCCAACTGCTGCCCCCTCTGTACTCCTTGG | |
| TATTTGTCATTGGCCTGGTTGGAAACATCCTGGTGGTCCTGGTCCTTGTGCAATACAAGAGGCTAAAAAACATGACCAGCATCTACCTCCTGAACCTGGCCATTTCTGACCTGCTCTTCCTGTTCACGCTTCCC | |
| TTCTGGATCGACTACAAGTTGAAGGATGACTGGGTTTTGGTGATGCCATGTGTAAGATCCTCTCTGGGTTTTATTACACAGGCTTGTACAGCGAGATCTTTTTCTATCATCCTGCTGACGATTGACAGGTACCT | |
| GGCCATCGTCCACGCCGTGTTTGCCTTGCGGGCACGGACCGTCACTTTTGGTGTCATCACCAGCATCATCATTTGGGCCCTGGCCATCTTGGCTTCCATGCCAGGCTTATACTTTTCCAAGACCCAATGGGAAT | |
| TCACTCACCACACCTGCAGCCTTCACTTTCCTCACGAAAGCCTACGAGAGTGGAAGCTGTTTCAGGCTCTGAAACTGAACCTCTTTGGGCTGGTATTGCCTTTGTTGGTCATGATCATCTGCTACACAGGGATT | |
| ATAAAGATTCTGCTAAGACGACCAAATGAGAAGAAATCCAAAGCTGTCCGTTTGATTTTTGTCATCATGATCATCTTTTTTCTCTTTTGGACCCCCTACAATTTGACTATACTTATTTCTGTTTTCCAAGACTT | |
| CCTGTTCACCCATGAGTGTGAGCAGAGCAGACATTTGGACCTGGCTGTGCAAGTGACGGAGGTGATCGCCTACACGCACTGCTGTGTCAACCCAGTGATCTACGCCTTCGTTGGTGAGAGGTTCCGGAAGTACC | |
| TGCGGCAGTTGTTCCACAGGCGTGTGGCTGTGCACCTGGTTAAATGGCTCCCCTTCCTCTCCGTGGACAGGCTGGAGAGGGTCAGCTCCACATCTCCCTCCACAGGGGAGCATGAACTCTCTGCTGGGTTCTGA | |
| CTCAGACCATAGGAGGCCAACCCAAAATAAGCAGGCGTGACCTGCCAGGCACACTGAGCCAGCAGCCTGGCTCTCCCAGCCAGGTTCTGACTCTTGGCACAGCATGGAGTCACAGCCACTTGGGATAGAGAGGG | |
| AATGTAATGGTGGCCTGGGGCTTCTGAGGCTTCTGGGGCTTCAGTCTTTTCCATGAACTTCTCCCCTGGTAGAAAGAAGATGAATGAGCAAAACCAAATATTCCAGAGACTGGGACTAAGTGTACCAGAGAAGG | |
| GCTTGGACTCAAGCAAGATTTCAGATTTGTGACCATTAGCATTTGTCAACAAAGTCACCCACTTCCCACTATTGCTTGCACAAACCAATTAAACCCAGTAGTGGTGACTGTGGGCTCCATTCAAAGTGAGCTCC | |
| TAAGCCATGGGAGACACTGATGTATGAGGAATTTCTGTTCTTCCATCACCTCCCCCCCCCCGCCACCCTCCCACTGCCAAAGAACTTGGAAATAGTGATTTCCACAGTGACTCCACTCTGAGTCCCAGAGCCAA | |
| TCAGTAGCCAGCATCTGCCTCCCCTTCACTCCCACCGCAGGATTTGGGCTCTTGGAATCCTGGGGAACATAGAACTCATGACGGAAGAGTTGAGACCTAAGCGAGAATAGAAATGGGGAACTACTGCTGGCAGT | |
| GGAACTAAGAAAGCCCTTAGGAAGAATTTTTATATCCACTAAAATCAAACAATTCAGGGAGTGGGCTAAGCACGGGCCATATGAATAACATGGTGTGCTTCTTAAAATAGCCATAAAGGGGAGGGACTCATCAT | |
| TTCCATTTACCCTTCTTTTCTGACTATTTTTCAGAATCTCTCTTCTTTTCAAGTTGGGTGATATGTTGGTAGATTCTAATGGCTTTATTGCAGCGATTAATAACAGGCAAAAGGAAGCAGGGTTGGTTTCCCTT | |
| CTTTTTGTTCTTCATCTAAGCCTTCTGGTTTTATGGGTCAGAGTTCCGACTGCCATCTTGGACTTGTCAGCAAAAAAAAAAAATAATAATAATAATAAGGCCTGCTGTGTAAGCTGACAGTATTTGTAGCTGAT | |
| AGGGGGTTGGGAGGAAAGTGTCTACTAGGAGGGTGGGGTGAGATTCTGTGTTGATGTTTTTTCTTCTTCTATCACAGGGAGAAGTGAAATGACAACCTCACTAGATACAGTTGAGACCTTTGGTACCACATCCT | |
| ACTATGATGACGTGGGCCTGCTCTGTGAAAAAGCTGATACCAGAGCACTGATGGCCCAGTTTGTGCCCCCGCTGTACTCCCTGGTGTTCACTGTGGGCCTCTTGGGCAATGTGGTGGTGGTGATGATCCTCATA | |
| AAATACAGGAGGCTCCGAATTATGACCAACATCTACCTGCTCAACCTGGCCATTTCGGACCTGCTCTTCCTCGTCACCCTTCCATTCTGGATCCACTATGTCAGGGGGCATAACTGGGTTTTTGGCCATGGCAT | |
| GTGTAAGCTCCTCTCAGGGTTTTATCACACAGGCTTGTACAGCGAGATCTTTTTCATAATCCTGCTGACAATCGACAGGTACCTGGCCATTGTCCATGCTGTGTTTGCCCTTCGAGCCCGGACTGTCACTTTTG | |
| GTGTCATCACCAGCATCGTCACCTGGGGCCTGGCAGTGCTAGCAGCTCTTCCTGAATTTATCTTCTATGAGACTGAAGAGTTGTTTGAAGAGACTCTTTGCAGTGCTCTTTACCCAGAGGATACAGTATATAGC | |
| TGGAGGCATTTCCACACTCTGAGAATGACCATCTTCTGTCTCGTTCTCCCTCTGCTCGTTATGGCCATCTGCTACACAGGAATCATCAAAACGCTGCTGAGGTGCCCCAGTAAAAAAAAGTACAAGGCCATCCG | |
| GCTCATTTTTGTCATCATGGCGGTGTTTTTCATTTTCTGGACACCCTACAATGTGGCTATCCTTCTCTCTTCCTATCAATCCATCTTATTTGGAAATGACTGTGAGCGGAGCAAGCATCTGGACCTGGTCATGC | |
| TGGTGACAGAGGTGATCGCCTACTCCCACTGCTGCATGAACCCGGTGATCTACGCCTTTGTTGGAGAGAGGTTCCGGAAGTACCTGCGCCACTTCTTCCACAGGCACTTGCTCATGCACCTGGGCAGATACATC | |
| CCATTCCTTCCTAGTGAGAAGCTGGAAAGAACCAGCTCTGTCTCTCCATCCACAGCAGAGCCGGAACTCTCTATTGTGTTTTAGGTCAGATGCAGAAAATTGCCTAAAGAGGAAGGACCAAGGAGATGAAGCAA | |
| ACACATTAAGCCTTCCACACTCACCTCTAAAACAGTCCTTCAAACTTCCAGTGCATTTTTTCAAGTTTTATGATTTATTTAACTTGTGGAACAAAAATAAACCAGAAACCACCACCTCTCACGCCAAAGCTCAC | |
| ACCTTCAGCCTCCAACATGAAGGTCTCCGCAGCACTTCTGTGGCTGCTGCTCATAGCAGCTGCCTTCAGCCCCCAGGGGCTCGCTGGGCCAGCTTCTGTCCCAACCACCTGCTGCTTTAACCTGGCCAATAGGA | |
| AGATACCCCTTCAGCGACTAGAGAGCTACAGGAGAATCACCAGTGGCAAATGTCCCCAGAAAGCTGTGATCTTCAAGACCAAACTGGCCAAGGATATCTGTGCCGACCCCAAGAAGAAGTGGGTGCAGGATTCC | |
| ATGAAGTATCTGGACCAAAAATCTCCAACTCCAAAGCCATAAATAATCACCATTTTTGAAACCAAACCAGAGCCTGAGTGTTGCCTAATTTGTTTTCCCTTCTTACAATGCATTCTGAGGTAACCTCATTATCA | |
| GTCCAAAGGGCATGGGTTTTATTATATATATATATATTTTTTTTTTAAAAAAAAACGTATTGCATTTAATTTATTGAGGCTTTAAAACTTATCCTCCATGAATATCAGTTATTTTTAAACTGTAAAGCTTTGTG | |
| CAGATTCTTTACCCCCTGGGAGCCCCAATTCGATCCCCTGTCACGTGTGGGCAATGTTCCCCCTCTCCTCTCTTCCTCCCTGGAATCTTGTAAAGGTCCTGGCAAAGATGATCAGTATGAAAATGTCATTGTTC | |
| TTGTGAACCCAAAGTGTGACTCATTAAATGGAAGTAAATGTTGTTTTAGGAATACCAACCCAGAAACCACCACCTCTCACGCCAAAGCTCACACCTTCAGCCTCCAACATGAAGGTCTCCGCAGCACTTCTGTG | |
| GCTGCTGCTCATAGCAGCTGCCTTCAGCCCCCAGGGGCTCGCTGGGCCAGCTTCTGTCCCAACCACCTGCTGCTTTAACCTGGCCAATAGGAAGATACCCCTTCAGCGACTAGAGAGCTACAGGAGAATCACCA | |
| GTGGCAAATGTCCCCAGAAAGCTGTGATCTTCAAGACCAAACTGGCCAAGGATATCTGTGCCGACCCCAAGAAGAAGTGGGTGCAGGATTCCATGAAGTATCTGGACCAAAAATCTCCAACTCCAAAGCCATAA | |
| ATAATCACCATTTTTGAAACCAAACCAGAGCCTGAGTGTTGCCTAATTTGTTTTCCCTTCTTACAATGCATTCTGAGGTAACCTCATTATCAGTCCAAAGGGCATGGGTTTTATTATATATATATATATATATT | |
| TTTTTTTAAAAAAAAACGTATTGCATTTAATTTATTGAGGCTTTAAAACTTATCCTCCATGAATATCAGTTATTTTTAAACTGTAAAGCTTTGTGCAGATTCTTTACCCCCTGGGAGCCCCAATTCGATCCCCT | |
| GTCACGTGTGGGCAATGTTCCCCCTCTCCTCTCTTCCTCCCTGGAATCTTGTAAAGGTCCTGGCAAAGATGATCAGTATGAAAATGTCATTGTTCTTGTGAACCCAAAGTGTGACTCATTAAATGGAAGTAATG | |
| TTGTTTTAGGAATACATAAAGTATGTGCATATTTTATTATAGTCACTAGTTGTAATTTTTTTGTGGGAAATCCACACTGAGCTGAGGGGGCCACATATTCCCCTCCTTTTCCAAGGCAAGATCCAGATGGATTA | |
| AAAAATGTACCAAGTCCCTCCTACTAGCTTGCCTCTCTTCTGTTCTGCTTGACTTCCTAGGATCTGGAATCTGGTCAGCAATCAGGAATCCCTTCATCGTGACCCCCGCATGGGCAAAGGCTTCCCTGGAATCT | |
| CCCACACTGTCTGCTCCCTATAAAAGGCAGGCAGATGGGCCAGAGGAGCAGAGAGGCTGAGACCAACCCAGAAACCACCACCTCTCACGCCAAAGCTCACACCTTCAGCCTCCAACATGAAGGTCTCCGCAGCA | |
| CTTCTGTGGCTGCTGCTCATAGCAGCTGCCTTCAGCCCCCAGGGGCTCGCTGGGCCAGGTAAGCCCCCCAACTCCTTACAGGAAAGGTAAGGTAACCACCTCCAGGCTACTAGGTCAGCAAGAATCTTTACAGA | |
| CTCACTGCAAATTCTCCATTTGAAAAATAGGGAAACAGGTTTTGTGGGTGGACAAGAAATGCCTCAACCGTCACATCCAGTCACTGGAAGAGCCAGAACTAGAAAGCTCCCGAGTCTTTTCCCCACATTCAAGA | |
| GGGCCGCTGGGTGCATCCTTACCCAGCTATCCTTACAGTGTTTGGGAATGGGGAATGGCTCTGTCTTACTGTGGGCATGGTGGGCATTTTTGGCAGTGGGAGAGAAGGAAAATCTGTTGATTAGAAGCTCAGTA | |
| TGTTAATTCGACTCCAGGACAGCTTTCAGAGACAGTGGCTAAGAGAAGAACGAGGTCCCAGGGGATCTCTTGAGGTGACTTATTTTGACACTCTTTGGGAAAGTTATCTAGGAGATTTGTTCCATAACTCATTT | |
| AAAAATGTACCAAGTCCCTCCTACTAGCTTGCCTCTCTTCTGTTCTGCTTGACTTCCTAGGATCTGGAATCTGGTCAGCAATCAGGAATCCCTTCATCGTGACCCCCGCATGGGCAAAGGCTTCCCTGGAATCT | |
| CCCACACTGTCTGCTCCCTATAAAAGGCAGGCAGATGGGCCAGAGGAGCAGAGAGGCTGAGACCAACCCAGAAACCACCACCTCTCACGCCAAAGCTCACACCTTCAGCCTCCAACATGAAGGTCTCCGCAGCA | |
| CTTCTGTGGCTGCTGCTCATAGCAGCTGCCTTCAGCCCCCAGGGGCTCGCTGGGCCAGGTAAGCCCCCCAACTCCTTACAGGAAAGGTAAGGTAACCACCTCCAGGCTACTAGGTCAGCAAGAATCTTTACAGA | |
| CTCACTGCAAATTCTCCATTTGAAAAATAGGGAAACAGGTTTTGTGGGTGGACAAGAAATGCCTCAACCGTCACATCCAGTCACTGGAAGAGCCAGAACTAGAAAGCTCCCGAGTCTTTTCCCCACATTCAAGA | |
| GGGCCGCTGGGTGCATCCTTACCCAGCTATCCTTACAGTGTTTGGGAATGGGGAATGGCTCTGTCTTACTGTGGGCATGGTGGGCATTTTTGGCAGTGGGAGAGAAGGAAAATCTGTTGATTAGAAGCTCAGTA | |
| TGTTAATTCGACTCCAGGACAGCTTTCAGAGACAGTGGCTAAGAGAAGAACGAGGTCCCAGGGGATCTCTTGAGGTGACTTATTTTGACACTCTTTGGGAAAGTTATCTAGGAGATTTGTTCCATAACTCATTT | |
| TCCCATACTCTGGTGACAAATTTACTGAGTGTATCGGTCCCACTGAGCCAGTGCATAGCATGGTAACAAACAGTTCTAAATTATCAATGACTTAACAGAATTAACTAAATTAACAAAAGTTACTTTCTCACTTG | |
| TACTAAATATCTATAATGTATGGGCTCAGGCTTCTGCATTTTATACTCAGGATTCTAGACTGATGGAGAAGTTGCCATGTGGGGGAACATTGATGGATACTGTGATAAAGCAGAAGAAAGCTCTCAGGAGTCTT | |
| GCATAGGCAATGCACTGTGGCTCAAAAATGACACCCATCACTTTGTCTCCTTCTTTATTGATCAAAACTAATTAATGCCTCCAACCAAACAAAAGTGGCCAAGAAATGCAAGTCTACCTTGTGTCTCAAAACAG | |
| AGGATGGAGAATATTTGGTGAAAATTACCATGACCATCACATGGCCACGTAGGTCTTTATAATGACAGAGCTAGCATTTGTCACATTGACCAAGCTTTGTCCATACACTCTACAGTAATGATGAGTCCTCAGTG | |
| CACAGGGGAGGATGCTGAAGACACAGGACAGCATCCTCCAGACACATAAGACTTCAGAGCAGAGGGATTCTCCCTCCACCTCTCGCAATTCCTTGCTTTCTCCTAACTTCCTTTACAAAGTCATGCTTGGAAAT | |
| GTCTATGTATCATCATGTGGCTCATTTTTTTCTCTGTTCATTTTTTTTCCCCAAAATTCAGCTTCTGTCCCAACCACCTGCTGCTTTAACCTGGCCAATAGGAAGATACCCCTTCAGCGACTAGAGAGCTACAG | |
| GAGAATCACCAGTGGCAAATGTCCCCAGAAAGCTGTGATGTAAGTAAATAAAGTTCACCCTCCCCTAGACAAAAAAATAATGTCTAGGGCACAGAGTCAAGAACTGTGGGAGTCATAGACTCTGATAGTTTGAC | |
| CTCTATGGTCCAATTCATTAATTTTCACAAGTGAGTGTTCACTCCCAGCTCCCTGCCTGGGAGATTGCTGTAGTCATATCAATTTCTTCAAGTCAAGAGCAAAGATGGTTTTACTGGGCCTTTAAGAGCAGCAA | |
| CTAACCCAAGAGTCTCATCCTTCCTCCTCTCCGTAGCAACCCTTTGTCCAGGGGCAGATGGTCCTTAAATATTTAGGGTCAAATGGGCAGAATTTTCAAAAACAATCCTTCCAATTGCATCCTGATTCTCCCCA | |
| CAGCTTCAAGACCAAACTGGCCAAGGATATCTGTGCCGACCCCAAGAAGAAGTGGGTGCAGGATTCCATGAAGTATCTGGACCAAAAATCTCCAACTCCAAAGCCATAAATAATCACCATTTTTGAAACCAAAC | |
| CAGAGCCTGAGTGTTGCCTAATTTGTTTTCCCTTCTTACAATGCATTCTGAGGTAACCTCATTATCAGTCCAAAGGGCATGGGTTTTATTATATATATATATATATATTTTTTTTTAAAAAAAAACGTATTGCA | |
| TTTAATTTATTGAGGCTTTAAAACTTATCCTCCATGAATATCAGTTATTTTTAAACTGTAAAGCTTTGTGCAGATTCTTTACCCCCTGGGAGCCCCAATTCGATCCCCTGTCACGTGTGGGCAATGTTCCCCCT | |
| CTCCTCTCTTCCTCCCTGGAATCTTGTAAAGGTCCTGGCAAAGATGATCAGTATGAAAATGTCATTGTTCTTGTGAACCCAAAGTGTGACTCATTAAATGGAAGTAATGTTGTTTTAGGAATACATAAAGTATG | |
| TGCATATTTTATTATAGTCACTAGTTGTAATTTTTTTGTGGGAAATCCACACTGAGCTGAGGGGGCCTCCGACAGCCTCTCCACAGGTACCATGAAGGTCTCCGCGGCACGCCTCGCTGTCATCCTCATTGCTA | |
| TGGCTCAGCCTTGTAATCCCAGCACTTTGGGAGGCCAAGGTGGGTGGATCACTTGAGGTCAGGAGTTCGAGACAGCCTGGCCAACATGATGAAACCCCATGTGTACTAAAAATACAAAAAATTAGCCGGGCGTG | |
| GTAGCGGGCGCCTGTAGTCCCAGCTACTCGGGAGGCTGAGGCAGGAGAATGGCGTGAACCCGGGAGCGGAGCTTGCAGTGAGCCGAGATCGCGCCACTGCACTCCAGCCTGGGCGACAGAGCGAGACTCCGTCT | |
| CAAAAAAAAAAAAAAAAAAAAAAAAAATACAAAAATTAGCCGCGTGGTGGCCCACGCCTGTAATCCCAGCTACTCGGGAGGCTAAGGCAGGAAAATTGTTTGAACCCAGGAGGTGGAGGCTGCAGTGAGCTGAG | |
| ATTGTGCCACTTCACTCCAGCCTGGGTGACAAAGTGAGACTCCGTCACAACAACAACAACAAAAAGCTTCCCCAACTAAAGCCTAGAAGAGCTTCTGAGGCGCTGCTTTGTCAAAAGGAAGTCTCTAGGTTCTG | |
| AGCTCTGGCTTTGCCTTGGCTTTGCAAGGGCTCTGTGACAAGGAAGGAAGTCAGCATGCCTCTAGAGGCAAGGAAGGGAGGAACACTGCACTCTTAAGCTTCCGCCGTCTCAACCCCTCACAGGAGCTTACTGG | |
| CAAACATGAAAAATCGGGGTTCAACCCCCAGGGACTTGCTCAGCCAGATGCACTCAACGTCCCATCTACTTGCTGCTTCACATTTAGCAGTAAGAAGATCTCCTTGCAGAGGCTGAAGAGCTATGTGATCACCA | |
| CCAGCAGGTGTCCCCAGAAGGCTGTCATCTTCAGAACCAAACTGGGCAAGGAGATCTGTGCTGACCCAAAGGAGAAGTGGGTCCAGAATTATATGAAACACCTGGGCCGGAAGCTCACACCCTGAAGACTTGAA | |
| CTCTGCTACCCCTACTGAAATCAAGCTGGAGTACGTGAAATGACTTTTCCATTCTCCTCTGGCCTCCTCTTCTATGCTTTGGAATACTTCTACCATAATTTTCAAATAGGATGCATTCGGTTTTGTGATTCAAA | |
| ATGTACTATGTGTTAAGTAATATTGGCTATTATTTGACTTGTTGCTGGTTTGGAGTTTATTTGAGTATTGCTGATCTTTTCTATAGCAAGGCCTTGAGCAAGTAGGTTGCTGTCTCTAAGCCCCCTTCCCTTCC | |
| ACTATGAGCTGCTGGCAGTGGGTTTGTATTCGGTTCCCAGGGGTTGAGAGCATGCCTGTGGGAGTCATGGACATGAAGGGATGCCGCAATGTAGGAAGGAGAGCTCTTTGTGAATGTGAGGTGTTGCTAAATAT | |
| GTTATTGTGGAAAGATGAATGCAATAGTAGGACTGCTGACATTTTGCAGAAAATACATTTTATTTAAAAATCTCCTAAAAAAAAAAAAAAAAAAAAAAAAGAAAAAAAAAAAAAGTGGCTCTACTTTCAGAAGA | |
| AAGTGTCTCTCTTCCTGCTTAAACCTCTGTCTCTGACGGTCCCTGCCAATCGCTCTGGTCGACCCCAACACACTAGGAGGACAGACACAGGCTCCAAACTCCACTAACCAGAGCTGTGATTGTGCCCGCTGAGT | |
| GGACTGCGTTGTCAGGGAGTGAGTGCTCCATCATCGGGAGAATCCAAGCAGGACCGCCATGGAGGAAGGTCAATATTCAGAGATCGAGGAGCTTCCCAGGAGGCGGTGTTGCAGGCGTGGGACTCAGATCGTGC | |
| TGCTGGGGCTGGTGACCGCCGCTCTGTGGGCTGGGCTGCTGACTCTGCTTCTCCTGTGGCACTGGGACACCACACAGAGTCTAAAACAGCTGGAAGAGAGGGCTGCCCGGAACGTCTCTCAAGTTTCCAAGAAC | |
| TTGGAAAGCCACCACGGTGACCAGATGGCGCAGAAATCCCAGTCCACGCAGATTTCACAGGAACTGGAGGAACTTCGAGCTGAACAGCAGAGATTGAAATCTCAGGACTTGGAGCTGTCCTGGAACCTGAACGG | |
| GCTTCAAGCAGATCTGAGCAGCTTCAAGTCCCAGGAATTGAACGAGAGGAACGAAGCTTCAGATTTGCTGGAAAGACTCCGGGAGGAGGTGACAAAGCTAAGGATGGAGTTGCAGGTGTCCAGCGGCTTTGTGT | |
| GCAACACGTGCCCTGAAAAGTGGATCAACTTCCAACGGAAGTGCTACTACTTCGGCAAGGGCACCAAGCAGTGGGTCCACGCCCGGTATGCCTGTGACGACATGGAAGGGGCAGCTGGTCAGCATCCACAGCCG | |
| GAGGAGCAGGACTTCCTGACCAAGCATGCCAGCCACACCGGCTCCTGGATTGGCCTTCGGAACTTGGACCTGAAGGGAGAGTTTATCTGGGTGGATGGGAGCCATGTGGACTACAGCAACTGGGCTCCAGGGGA | |
| GCCCACCAGCCGGAGCCAGGGCGAGGACTGCGTGATGATGCGGGGCTCCGGTCGCTGGACCGACGCCTTCTGCGACCGTAAGCTGGGCGCCTGGGTGTGCGACCGGCTGGCCACATGCACGCCGCCAGCCAGCG | |
| AAGGTTCCGCGGAGTCCATGGGACCTGATTCAAGACCAGACCCTGACGGCCGCCTGCCCACCCCCTCTGCCCCTCTCCACTCTTGAGCATGGATACAGCCAGGCCCAGAGCAAGACCCTGAAGACCCCCAACCA | |
| CGGCCTAAAAGCCTCTTTGTGGCTGAAAGGTCCCTGTGACATTTTCTGCCACCCAAACGGAGGCAGCTGACACATCTCCCGCTCCTCTATGGCCCCTGCCTTCCCAGGAGTACACCCCAACAGCACCCTCTCCA | |
| GATGGGAGTGCCCCCAACAGCACCCTCTCCAGATGAGAGTTACACCCCAACAGCACCCTCTCCAGATGCAGCCCCATCTCCTCAGCACCCCAGGACCTGAGTATCCCCAGCTCAGGGTGGTGAGTCCTCCTGTC | |
| CAGCCTGCATCAATAAAATGGGGCAGTGATGGCCCCATGGAGGAAGGTCAATATTCAGGTAGGAGGACTCTCTGGTTCTAACGTTGGCAGAAGCAATGACCCTTAGCTACTCCTTTCACCCAGAAGAGAAGCGG | |
| GGCTTCCCAGTCCCTCTCTGGGAAAGAGGGTGAATTTCTAAGAAAGGGACTGGTGTGAGTAAGGAGGTGAGGCCGCACTGACTTTCCTGGCACAGAGCCAGGAAGGAGTGGAAAATTGAGGGCCCCTCCTTTTT | |
| CTGATTCAACACCCTCCTGACAAAAAAAGAAAAAGAAAAAAAAAAACGGCTTCAGCTAGGGAGCGGGGACGCAATAGAGTCAGAGGCCAAATAGAACAGGAACTTGGAACAAGCAGAATTTAGCATAATGAATC | |
| CTCCAAGCCAGGGTGAGTGCAGACTCCTGCTTAAACCTCTGTCTCTGACGGTCCCTGCCAATCGCTCTGGTCGACCCCAACACACTAGGAGGACAGACACAGGCTCCAAACTCCACTAAGTGACCAGAGCTGTG | |
| ATTGTGCCCGCTGAGTGGACTGCGTTGTCAGGGAGTGAGTGCTCCATCATCGGGAGAATCCAAGCAGGACCGCCATGGAGGAAGGTCAATATTCAGAGATCGAGGAGCTTCCCAGGAGGCGGTGTTGCAGGCGT | |
| GGGACTCAGATCGTGCTGCTGGGGCTGGTGACCGCCGCTCTGTGGGCTGGGCTGCTGACTCTGCTTCTCCTGTGGCACTGGGACACCACACAGAGTCTAAAACAGCTGGAAGAGAGGGCTGCCCGGAACGTCTC | |
| TCAAGTTTCCAAGAACTTGGAAAGCCACCACGGTGACCAGATGGCGCAGAAATCCCAGTCCACGCAGATTTCACAGGAACTGGAGGAACTTCGAGCTGAACAGCAGAGATTGAAATCTCAGGACTTGGAGCTGT | |
| CCTGGAACCTGAACGGGCTTCAAGCAGATCTGAGCAGCTTCAAGTCCCAGGAATTGAACGAGAGGAACGAAGCTTCAGATTTGCTGGAAAGACTCCGGGAGGAGGTGACAAAGCTAAGGATGGAGTTGCAGGTG | |
| TCCAGCGGCTTTGTGTGCAACACGTGCCCTGAAAAGTGGATCAATTTCCAACGGAAGTGCTACTACTTCGGCAAGGGCACCAAGCAGTGGGTCCACGCCCGGTATGCCTGTGACGACATGGAAGGGCAGCTGGT | |
| CAGCATCCACAGCCCGGAGGAGCAGGACTTCCTGACCAAGCATGCCAGCCACACCGGCTCCTGGATTGGCCTTCGGAACTTGGACCTGAAGGGAGAGTTTATCTGGGTGGATGGGAGCCATGTGGACTACAGCA | |
| ACTGGGCTCCAGGGGAGCCCACCAGCCGGAGCCAGGGCGAGGACTGCGTGATGATGCGGGGCTCCGGTCGCTGGAACGACGCCTTCTGCGACCGTAAGCTGGGCGCCTGGGTGTGCGACCGGCTGGCCACATGC | |
| ACGCCGCCAGCCAGCGAAGGTTCCGCGGAGTCCATGGGACCTGATTCAAGACCAGACCCTGACGGCCGCCTGCCCACCCCCTCTGCCCCTCTCCACTCTTGAGCATGGATACAGCCAGGCCCAGAGCAAGACCC | |
| TGAAGACCCCCAACCACGGCCTAAAAGCCTCTTTGTGGCTGAAAGGTCCCTGTGACATTTTCTGCCACCCAAACGGAGGCAGCTGACACATCTCCCGCTCCTCTATGGCCCCTGCCTTCCCAGGAGTACACCCC | |
| CGCTGCCCGCACTCCTGGTCCTGCTCGGGGCTCTGTTCCCAGGACCTGGCAATGCCCAGACATCTGTGTCCCCCTCAAAAGTCATCCTGCCCCGGGGAGGCTCCGTGCTGGTGACATGCAGCACCTCCTGTGAC | |
| CAGCCCAAGTTGTTGGGCATAGAGACCCCGTTGCCTAAAAAGGAGTTGCTCCTGCCTGGGAACAACCGGAAGGTGTATGAACTGAGCAATGTGCAAGAAGATAGCCAACCAATGTGCTATTCAAACTGCCCTGA | |
| TGGGCAGTCAACAGCTAAAACCTTCCTCACCGTGTACTGGACTCCAGAACGGGTGGAACTGGCACCCCTCCCCTCTTGGCAGCCAGTGGGCAAGAACCTTACCCTACGCTGCCAGGTGGAGGGTGGGGCACCCC | |
| GGGCCAACCTCACCGTGGTGCTGCTCCGTGGGGAGAAGGAGCTGAAACGGGAGCCAGCTGTGGGGGAGCCCGCTGAGGTCACGACCACGGTGCTGGTGAGGAGAGATCACCATGGAGCCAATTTCTCGTGCCGC | |
| ACTGAACTGGACCTGCGGCCCCAAGGGCTGGAGCTGTTTGAGAACACCTCGGCCCCCTACCAGCTCCAGACCTTTGTCCTGCCAGCGACTCCCCCACAACTTGTCAGCCCCCGGGTCCTAGAGGTGGACACGCA | |
| GGGGACCGTGGTCTGTTCCCTGGACGGGCTGTTCCCAGTCTCGGAGGCCCAGGTCCACCTGGCACTGGGGGACCAGAGGTTGAACCCCACAGTCACCTATGGCAACGACTCCTTCTCGGCCAAGGCCTCAGTCA | |
| GTGTGACCGCAGAGGACGAGGGCACCCAGCGGCTGACGTGTGCAGTAATACTGGGGAACCAGAGCCAGGAGACACTGCAGACAGTGACCATCTACAGCTTTCCGGCGCCCAACGTGATTCTGACGAAGCCAGAG | |
| GTCTCAGAAGGACCGAGGTGACAGTGAAGTGTGAGGCCCACCCTAGAGCCAAGGTGACGCTGAATGGGGTTCCAGCCCAGCCACTGGGCCCGAGGGCCCAGCTCCTGCTGAAGGCCACCCCAGAGGACAACCGG | |
| GCGCAGCTTCTCCTGCTCTGCAACCCTGGAGGTGGCCGGCCAGCTTATACACAAGAACCAGACCCGGGAGCTTCGTGTCCTGTATGGCCCCCGACTGGACGAGAGGGATTGTCCGGGAAACTGGACGTGGCCAG | |
| AAAATTCCCAGCAGACTCCAATGTGCCAGGCTTGGGGGAACCCATTGCCCGAGCTCAAGTGTCTAAAGGATGGCACTTTCCCACTGCCCATCGGGGAATCAGTGACTGTCACTCGAGATCTTGAGGGCACCTAC | |
| CTCTGTCGGGCCAGGAGCACTCAAGGGGAGGTCACCCGCGAGGTGACCGTGAATGTGCTCTCCCCCCGGTATGAGATTGTCATCATCACTGTGGTAGCAGCCGCAGTCATAATGGGCACTGCAGGCCTCAGCAC | |
| GTACCTCTATAACCGCCAGCGGAAGATCAAGAAATACAGACTACAACAGGCCCAAAAAGGGACCCCCATGAAACCGAACACACAAGCCACGCCTCCCTGAACCTATCCCGGGACAGGGCCTCTTCCTCGGCCTT | |
| CCCATATTGGTGGCAGTGGTGCCACACTGAACAGAGTGGAAGACATATGCCATGCAGCTACACCTACCGGCCCTGGGACGCCGGAGGACAGGGCATTGTCCTCAGTCAGATACAACAGCATTTGGGGCCATGGT | |
| ACCTGCACACCTAAAACACTAGGCCACGCATCTGATCTGTAGTCACATGACTAAGCCAAGAGGAAGGAGCAAGACTCAAGACATGATTGATGGATGTTAAAGTCTAGCCTGATGAGAGGGGAAGTGGTGGGGGA | |
| GACATAGCCCCACCATGAGGACATACAACTGGGAAATACTGAAACTTGCTGCCTATTGGGTATGCTGAGGCCCACAGACTTACAGAAGAAGTGGCCCTCCATAGACATGTGTAGCATCAAAACACAAAGGCCCA | |
| CACTTCCTGACGGATGCCAGCTTGGGCACTGCTGTCTACTGACCCCAACCCTTGATGATATGTATTTATTCATTTGTTATTTTACCAGCTATTTATTGAGTGTCTTTTATGTAGGCTAAATGAACATAGGTCTC | |
| TGGCCTCACGGAGCTCCCAGTCCATGTCACATTCAAGGTCACCAGGTACAGTTGTACAGGTTGTACACTGCAGGAGAGTGCCTGGCAAAAAGATCAAATGGGGCTGGGACTTCTCATTGGCCAACCTGCCTTTC | |
| CCCAGAAGGAGTGATTTTTCTATCGGCACAAAAGCACTATATGGACTGGTAATGGTTCACAGGTTCAGAGATTACCCAGTGAGGCCTTATTCCTCCCTTCCCCCCAAAACTGACACCTTTGTTAGCCACCTCCC | |
| CACCCACATACATTTCTGCCAGTGTTCACAATGACACTCAGCGGTCATGTCTGGACATGAGTGCCCAGGGAATATGCCCAAGCTATGCCTTGTCCTCTTGTCCTGTTTGCATTTCACTGGGAGCTTGCACTATT | |
| GCAGCTCCAGTTTCCTGCAGTGATCAGGGTCCTGCAAGCAGTGGGGAAGGGGGCCAAGGTATTGGAGGACTCCCTCCCAGCTTTGGAAGGGTCATCCGCGTGTGTGTGTGTGTGTATGTGTAGACAAGCTCTCG | |
| CTCTGTCACCCAGGCTGGAGTGCAGTGGTGCAATCATGGTTCACTGCAGTCTTGACCTTTTGGGCTCAAGTGATCCTCCCACCTCAGCCTCCTGAGTAGCTGGGACCATAGGCTCACAACACCACACCTGGCAA | |
| ATTTGATTTTTTTTTTTTTTTTCAGAGACGGGGTCTCGCAACATTGCCCAGACTTCCTTTGTGTTAGTTAATAAAGCTTTCTCAACTGCCATGCCTGGGAAGATGGTCGTGATCCTTGGAGCCTCAAATATACT | |
| TTGGATAATGTTTGCAGCTTCTCAAGCTTTTAAAATCGAGACCACCCCAGAATCTAGATATCTTGCTCAGATTGGTGACTCCGTCTCATTGACTTGCAGCACCACAGGCTGTGAGTCCCCATTTTTCTCTTGGA | |
| GAACCCAGATAGATAGTCCACTGAATGGGAAGGTGACGAATGAGGGGACCACATCTACGCTGACAATGAATCCTGTTAGTTTTGGGAACGAACACTCTTACCTGTGCACAGCAACTTGTGAATCTAGGAAATTG | |
| GAAAAAGGAATCCAGGTGGAGATCTACTCTTTTCCTAAGGATCCAGAGATTCATTTGAGTGGCCCTCTGGAGGCTGGGAAGCCGATCACAGTCAAGTGTTCAGTTGCTGATGTATACCCATTTGACAGGCTGGA | |
| GATAGACTTACTGAAAGGAGATCATCTCATGAAGAGTCAGGAATTTCTGGAGGATGCAGACAGGAAGTCCCTGGAAACCAAGAGTTTGGAAGTAACCTTTACTCCTGTCATTGAGGATATTGGAAAAGTTCTTG | |
| TTTGCCGAGCTAAATTACACATTGATGAAATGGATTCTGTGCCCACAGTAAGGCAGGCTGTAAAAGAATTGCAAGTCTACATATCACCCAAGAATACAGTTATTTCTGTGAATCCATCCACAAAGCTGCAAGAA | |
| GGTGGCTCTGTGACCATGACCTGTTCCAGCGAGGGTCTACCAGCTCCAGAGATTTTCTGGAGTAAGAAATTAGATAATGGGAATCTACAGCACCTTTCTGGAAATGCAACTCTCACCTTAATTGCTATGAGGAT | |
| GGAAGATTCTGGAATTTATGTGTGTGAAGGAGTTAATTTGATTGGGAAAAACAGAAAAGAGGTGGAATTAATTGTTCAAGAGAAACCATTTACTGTTGAGATCTCCCCTGGACCCCGGATTGCTGCTCAGATTG | |
| GAGACTCAGTCATGTTGACATGTAGTGTCATGGGCTGTGAATCCCCATCTTTCTCCTGGAGAACCCAGATAGACAGCCCTCTGAGCGGGAAGGTGAGGAGTGAGGGGACCAATTCCACGCTGACCCTGAGCCCT | |
| GTGAGTTTTGAGAACGAACACTCTTATCTGTGCACAGTGACTTGTGGACATAAGAAACTGGAAAAGGGAATCCAGGTGGAGCTCTACTCATTCCCTAGAGATCCAGAAATCGAGATGAGTGGTGGCCTCGTGAA | |
| TGGGAGCTCTGTCACTGTAAGCTGCAAGGTTCCTAGCGTGTACCCCCTTGACCGGCTGGAGATTGAATTACTTAAGGGGGAGACTATTCTGGAGAATATAGAGTTTTTGGAGGATACGGATATGAAATCTCTAG | |
| AGAACAAAAGTTTGGAAATGACCTTCATCCCTACCATTGAAGATACTGGAAAAGCTCTTGTTTGTCAGGCTAAGTTACATATTGATGACATGGAATTCGAACCCAAACAAAGGCAGAGTACGCAAACACTTTAT | |
| GTCAATGTTGCCCCCAGAGATACAACCGTCTTGGTCAGCCCTTCCTCCATCCTGGAGGAAGGCAGTTCTGTGAATATGACATGCTTGAGCCAGGGCTTTCCTGCTCCGAAAATCCTGTGGAGCAGGCAGCTCCC | |
| TAACGGGGAGCTACAGCCTCTTTCTGAGAATGCAACTCTCACCTTAATTTCTACAAAAATGGAAGATTCTGGGGTTTATTTATGTGAAGGAATTAACCAGGCTGGAAGAAGCAGAAAGGAAGTGGAATTAATTA | |
| TCCAAGTTACTCCAAAAGACATAAAACTTACAGCTTTTCCTTCTGAGAGTGTCAAAGAAGGAGACACTGTCATCATCTCTTGTACATGTGGAAATGTTCCAGAAACATGGATAATCCTGAAGAAAAAAGCGGAG | |
| ACAGGAGACACAGTACTAAAATCTATAGATGGCGCCTATACCATCCGAAAGGCCCAGTTGAAGGATGCGGGAGTATATGAATGTGAATCTAAAAACAAAGTTGGCTCACAATTAAGAAGTTTAACACTTGATGT | |
| TCAAGGAAGAGAAAACAACAAAGACTATTTTTCTCCTGAGCTTCTCGTGCTCTATTTTGCATCCTCCTTAATAATACCTGCCATTGGAATGATAATTTACTTTGCAAGAAAAGCCAACATGAAGGGGTCATATA | |
| GTCTTGTAGAAGCACAGAAATCAAAAGTGTAGACCAGCTGACAGGTGGAGCTGCCAGTCTCCAGTGCTCAGCCCTCAGCGGGGCCTGCCTGGCAGCCCCACACACAGAGGGCATCGGGGTGGCGGGGGCACGTG | |
| TTACACGGGGGCCCTGGGTCTGAGTCATCCACTTCCTCCGAGTCTGGATGGGAGGACCCAGCGCCCCTCCTCCGCCCCCTCCTGATCTGGAAGGATAAATGGGGAGGGGAGAGCCACTGGGTAGAAGGAACAGG | |
| GAGTGGCCAGGGTAAGTCCCCACTCTCAGAGACCCTGACATCAGCGTCACCTGGAGCAGAGTGGCCCAGCCTCAGACTCAGAGCACCAAGACCCAGGCCCGCAGGCCTGGACCCACCCCGGTCCCCCCGTCCCA | |
| GCTCCATTCTTCACCCCACAATCTGTAGCCCCCAGCCCTGCCCTGTGAGGCCCGGCCAGGCCCACGATGCTCCTCCTTGCTCCCCAGATGCTGAATCTGCTGCTGCTGGCGCTGCCCGTCCTGGCGAGCCGCGC | |
| CTACGCGGCCCCTGGTGAGTCCCAGCCGGGGTCCACCCTGCCCCTCACCACATTCCACAGGTCAGGGCCTGGGTGGGTTCTGGGGAGGTCGGGCTGGCCCCCCACACAGGGAAGGGCTGGGCCCAGGCCTGGGG | |
| CTGCTTCCTGGTCCTGACCTGGCACCTGCCCCAGCCCCAGGCCAGGCCCTGCAGCGAGTGGGCATCGTCGGGGGTCAGGAGGCCCCCAGGAGCAAGTGGCCCTGGCAGGTGAGCCTGAGAGTCCACGGCCCATA | |
| CTGGATGCACTTCTGCGGGGGCTCCCTCATCCACCCCCAGTGGGTGCTGACCGCAGCGCACTGCGTGGGACCGTGAGTCTCCCGGGGCCTGGAGGGGTGGGGAAGGGCTGGATGTGAGCCCTGGCTCCCGGGTC | |
| CTCCTGGGGGCTGCCCAGGGCCCTGAGTGGGATCCTCCGCTGCCCAGGGACGTCAAGGATCTGGCCGCCCTCAGGGTGCAACTGCGGGAGCAGCACCTCTACTACCAGGACCAGCTGCTGCCGGTCAGCAGGAT | |
| CATCGTGCACCCACAGTTCTACACCGCCCAGATCGGAGCGGACATCGCCCTGCTGGAGCTGGAGGAGCCGGTGAACGTCTCCAGCCACGTCCACACGGTCACCCTGCCCCCTGCCTCAGAGACCTTCCCCCCGG | |
| GGATGCCGTGCTGGGTCACTGGCTGGGGCGATGTGGACAATGATGGTGGGTCTGGGGACAGTGGAGGTGGGGCCAGGGTCTTAGCCACAGCCCAGCCCCTGGGTCCCTCTGGGCTCCAGGTGGGGGTTGCCCGG | |
| CCCCCTCCTGAGGCTGCACCCTCTTCCCCACCTGCAGAGCGCCTCCCACCGCCATTTCCTCTGAAGCAGGTGAAGGTCCCCATAATGGAAAACCACATTTGTGACGCAAAATACCACCTTGGCGCCTACACGGG | |
| TTGGCGCCTACACGGGAGACGACGTCCGCATCGTCCGTGACGACATGCTGTGTGCCGGGAACACCCGGAGGGACTCATGCCAGGGCGACTCCGGAGGGCCCCTGGTGTGCAAGGTGAATGGCACCTGGCTGCAG | |
| GCGGGCGTGGTCAGCTGGGGCGAGGGCTGTGCCCAGCCCAACCGGCCTGGCATCTACACCCGTGTCACCTACTACTTGGACTGGATCCACCACTATGTCCCCAAAAAGCCGTGAGTCAGGCCTGGGTGTGCCAC | |
| CTGGGTCACTGGAGGACCAACCCCTGCTGTCCAAAACACCACTGCTTCCTACCCAGGTGGCGACTGCCCCCCACACCTTCCCTGCCCCGTCCTGAGTGCCCCTTCCTGTCCTAAGCCCCCTGCTCTCTTCTGAG | |
| CCCCTTCCCCTGTCCTGAGGACCCTTCCCCATCCTGAGCCCCCTTCCCTGTCCTAAGCCTGACGCCTGCACTGCTCCGGCCCTCCCCTGCCCAGGCAGCTGGTGGTGGGCGCTAATCCTCCTGAGTGCTGGACC | |
| TCATTAAAGTGCATGGAAATCACAGGCGGGCTAAGTCTCCAAGATGCCCTTGGTGGATTTCTTCTGCGAGACCTGCTCTAAGCCTTGGCTGGTGGGCTGGTGGGACCAGTTCAAAAGGATGTTGAACCGTGAGC | |
| TCACACACCTGTCAGAAATGAGCAGGTCCGGAAACCAGGTCTCAGAGTACATTCCACAACATTCCTGGACAAACAGAATGAAGTGGAGATCCCATCACCCACGATGAAGGAACGAGAAAAAACAGCAAGCGCCG | |
| CGACCAAGACCCTCCCAGCCGCCCCCGCCCCCTGTACCACACTTACAGCCCATGTCCCAAATCACAGGGTTGAAAAAGTTGATGCATAGTAACAGCCTGAACAACTCTAACATTCCCCGATTTGGGGTGAAGAC | |
| CGATCAAGAAGAGCTCCTGGCCCAAGAACTGGAGAACCTGAACAAGTGGGGCCTGAACATCTTTTGCGTGTCGGATTACGCTGGAGGCCGCTCACTCACCTGCATCATGTACATGATATTCCAGGAGCGGGACC | |
| TGCTGAAGAAATTCCGCATCCCGGTGGACACGATGGTGACATACATGCTGACGCTGGAGGATCACTACCACGCTGACGTGGCCTACCATAACAGCCTGCACGCAGCTGACGTGCTGCAGTCCACCCACGTACTG | |
| CTGGCCACGCCTGCACTAGATGCAGTGTTCACGGACCTGGAGATTCTCGCCGCCCTCTTCGCGGCTGCCATCCACGATGTGGATCACCCTGGGGTCTCCAACCAGTTCCTCATCAACACCAATTCGGAGCTGGC | |
| GCTCATGTACAACGATGAGTCGGTGCTCGAGAATCACCACCTGGCCGTGGGCTTCAAGCTGCTGCAGGAGGACAACTGCGACATCTTCCAGAACCTCAGCAAGCGCCAGCGGCAGAGCCTACGCAAGATGGTCA | |
| TCGACATGGTGCTGGCCACGGACATGTCCAAGCACATGACCCTCCTGGCTGACCTGAAGACCATGGTGGAGACCAAGAAAGTGACCAGCTCAGGGGTCCTCCTGCTAGATAACTACTCCGACCGCATCCAGGTC | |
| CTCCGGAACATGGTGCACTGTGCCGACCTCAGCAACCCCACCAAGCCGCTGGAGCTGTACCGCCAGTGGACAGACCGCATCATGGCCGAGTTCTTCCAGCAGGGTGACCGAGAGCGCGAGCGTGGCATGGAAAT | |
| CAGCCCCATGTGTGACAAGCACACTGCCTCCGTGGAGAAGTCTCAGGTGGGTTTTATTGACTACATTGTGCACCCATTGTGGGAGACCTGGGCGGACCTTGTCCACCCAGATGCCCAGGAGATCTTGGACACTT | |
| TGGAGGACAACCGGGACTGGTACTACAGCGCCATCCGGCAGAGCCCATCTCCGCCACCCGAGGAGGAGTCAAGGGGGCCAGGCCACCCACCCCTGCCTGACAAGTTCCAGTTTGAGCTGACGCTGGAGGAGGAA | |
| GAGGAGGAAGAAATATCAATGGCCCAGATACCGTGCACAGCCCAAGAGGCATTGACTGCGCAGGGATTGTCAGGAGTCGAGGAAGCTCTGGATGCAACCATAGCCTGGGAGGCATCCCCGGCCCAGGAGTCGTT | |
| GGAAGTTATGGCACAGGAAGCATCCCTGGAGGCCGAGCTGGAGGCAGTGTATTTGACACAGCAGGCACAGTCCACAGGCAGTGCACCTGTGGCTCCGGATGAGTTCTCGTCCCGGGAGGAATTCGTGGTTGCTG | |
| TAAGCCACAGCAGCCCCTCTGCCCTGGCTCTTCAAAGCCCCCTTCTCCCTGCTTGGAGGACCCTGTCTGTTTCAGAGCATGCCCCGGGCCTCCCGGGCCTCCCCTCCACGGCGGCCGAGGTGGAGGCCCAACGA | |
| GAGCACCAGGCTGCCAAGAGGGCTTGCAGTGCCTGCGCAGGGACATTTGGGGAGGACACATCCGCACTCCCAGCTCCTGGTGGCGGGGGGTCAGGTGGAGACCCTACCTGAGAATTCCTCCTCTCTTCACCCCG | |
| TTAGCTGTTTTCAATGTAATGCTGCCGTCCTTCTCTTGCACTGCCTTCTGCGCTAACACCTCCATTCCTGTTTATAACCGTGTATTTATTACTTAATGTATATAATGTAATGTTTTGTAAGTTATTAATTTATA | |
| Concatemer Nucleic Acid Sequences of All Anti-sense Oligo sequences | |
| (SEQ ID NO: 2499) | |
| CTCCACTCACTCCAGGTGCTCCACTCACTCCAGGCAGCTGCCCCATGCTGGAGAAGGCCTTGTAACCGCGCCCCTGCTCCATTCGCCTTTCTTCCAGCTGTGTGTCACCACGCCCGGCTTCTCTTCTGCCCGCC | |
| TCAGCCTCCGGCACCAGGCTGGTCTCGTGGGAGATGCCAAGGCACGCCACCCCATTGGGAGATGCAAAGCCACCCCATTGGGTTCCCAGAGCTTGCCACCTGGAGCTCGGTGCTGCAATTGGATACACGTGTGG | |
| GCTTCGGGCGCCTCCGTTGTTCTCAGGGCAGCTGCTGCCCAGCCCGGTTTCCTGGGGCCCTGGGTGGGATACGGGTTGCTCCAGTCTGCCGGGTCGTTTTCACTTTCAGGGTGCTGGCTGCGGGCCCTCACCCG | |
| TGCCCTGTCCACTCACTCCAGGTGGTGTTGTGCCACTTGGTGCTGGTGCTGCTCGAAGGGCTCCCTGGCGGCTGCGGGCTGGGTCGGGACCGCTTCTCCCACTGCTTGGCTGGTTCCTGGCCTTGGCAGCCTTG | |
| TGAGGATCTTCTTGCTGATCTCCACTGGGGTCATCCCTGCTGCTCTCTCGTACTTCCCGAAGGTGGGGTTGTCTGGACTCTGGGTGTTGCTGAAGCTGCGGTCGGGTTCTACTTCCTCCAGGTGCTCCCAGGTT | |
| TCTGGCTCCCCTGCTCCACCGCATGTGGCTTATACCCCTCTTCCCTGAGCTCTGCGGACTGCGCTGTTTCAGGTGGCCGCGGATTTACTCTTCTCTGGGGTCTGGCTTGAGCTCTGGCCCTAGTCCTCATCTGC | |
| TGCCAGCCTGGCTGCCTTCCGCGACCCAGTGCCCTCTACTGTCTGCTGCAGAAGCTGTGGGGCTCGGCTTCTAGTTCAGGTCATTCCCTTGATGGCTGTCGATTTCCCAAGGCCGCCCTTCATGTCCTCTGTTG | |
| CTCCGGCATGGGTGTTTGGCAGCGTGCCTTATGCCTGCTGTCTTTCTGTTCCACTCTCCTCAGAAACTTTATTTATACAAGGCTCCACTCACTCCAGGGCTCCACTCACTCCAGCTGGGATTATAGGCATGAGC | |
| CTGATCTCAAGTGATCTGCACAGGGAACAGGAGCCCAGAGCAGCAGGACCAGGCAGCTCGGCTCCTGCAAGACCTTCATTGTAGTCGGAGACGCAGGTGCTCGCAAGTAGAGATGCTCAGTGGGACCATTCATC | |
| TTCCAAACCAGCGTGTGGGCTTCGGAGAGCAGAAAAACCAGCTGGGATACACGTGTGGGCTTCGGACTGACCACGTCATCCATGATCCAGTGTATAGTTATCCGCGAAGGAGCCCTTCCACAGCATTCACATGC | |
| TCGCTGGGCTTGACATTGGTGTGAACTGTCAGTCAGCAGCAGAGTGTCGGAATTATACAGGTAATTGTCAGTTGACTGCATAGGTGAGATGATAGATTCTGAAATCTGCCGGGTTCTAGGTAGGTCACGTTTGG | |
| CTGCGATGCGGAGGGAGCCCTGTAGGAAATCCCAGACTGGCTGCGATGCGGAGGGAGTATAGCCCTGAGCCCAGGCCCAGGATGACAATGCAGGAAATAGCACAACAGGCAGACGGCTAATCTTGGTGATGCTG | |
| AACCTGATCCCACGTAAGAAAGATTATTATAGCCACGAGGCGGACAATTCTTCCAGTGTGGGCCGGGACCGCTTCTCCCACTGACTTGGCTGGTTCCTGGCCTTCATCCCTTTTCATGTTGTGGCCCTGGAAAG | |
| GCATCTCTTGCTCTCTGGCCAGAGGACTGACACATCGCACCACGCTGATCTCCTCCTCCTCACACTCCACAGGCGATGCACAGAAGCTCATGCCCTCCCTTCCCTCCTGTCTCTGTTAGCCGGGCCACAGAGCA | |
| GGTCCAGGAACAGGCAGGTGGAAGAAGGCATGACTGGAACTCATCCCAGGGCATGCTCCTTGGGCCCTGCACTTGGCTCCAGGTGGAGAGGCTGCCGGGCTGGCAGGAGGACTTGAGGGTTGCCTGCGATGACG | |
| AACGGTGACTGGCTCAGGGAGTGGACCCAGCTCTCTGGGACTGTCTGAAGCTGCGGTAAGCACAGTGGTTGGCTCAGAGAGGGACATTTCGGCGGAGGATCCAAACTCCTGATAGCCACTGACCGCACTGGCCT | |
| GGGTGCCGGCCTTGTAACCAGCCTCTCCTGGCAAGCAGGCTTGAGAAGAATGAGGTCTTGGAAAGGCTGTCCAGTCCAAAGGTGAACCTTGGGAGATGTGAGCTCTGGGCTCCAGACCCAGGTGCTCGGCTTTG | |
| GCATGTCCTCTACGGGTCTGTGGCCTGCTCCTGTGCCCAGGCTGTCCACAAGGGGCTGAGTAGACAATGCCACCCGCACAGGTGGCAGGTAAGTCCTCCTGGCCATGACACTGGCCACAGCAAGGACTGGCCAG | |
| ACCTGTCTCCACAGCAGCACAGACTGGCCTCCAGTGGAAGGTGCCAGGGAGGCCGGACAGGGCAGGATGGAAGGATGATGTCCCACGGAGACAAAGTTCAGAGACCCTCATGTATGTGGCAGGCATGGATAAGC | |
| CCTAGTTCCAGGAGGTGGCATTTCCGCCAATCACCTTCATACCATTCCAGTCTCTGCAGCCCAGTGTGGCGAGCCCAGCCCAATGGCCCTCTACTCTCATGGGATGAGGTGCCCAAGGGCCTCAGCAACAACTC | |
| GTTCACAAGTCGAAGCTGTGGAGGGAGCAGCAACAGGGACAGTCTGCTGCAAACATGCCTTGGGCAGTTACGGCCATGATCTGGTGGGCAAGGCAGGTGGGCCTCCACGTGCCTGAAGTTCAATTTCTCATTCT | |
| AGGCAATGACCACCCTCCGATTTCCCAAGGCCGCCCAACAGCCGGGATTCTGCCTCCTGCTTTAGTCATAGCAATTTGTTTGGCAGCAAATTGTCCCTGGCTGAGCATATTTATTCTGCCCACAGGGTGGCTGA | |
| GCACCTGCTGTCTGGATTGCCCGGCCAACATGCAGGGTAACTGCCCTAGCACCTGAGGTCTGGCAACCCAAGGTTCCCGCCTTACACACAGACGAGCATTACTCCATGCGATGAGAAGCAGCGGGGTGGCCATG | |
| CGATGAGAAGCCAATGTGCCTGGCCTGAGTCAGGCACAGGACCAATGCTACCAATGCTCAATGTGCCTAGCGTCAGGCACAGGACCTCTAGCATAGCGTCAGGCACCAGCATCTAGCATAGCGTCCTATGCTCG | |
| TGGCTGCAACCGTGTCTATGCTCGTGGCTCGTGTCTATGCTCGTGGCGTGTCTGTCGTGTCTATGCTCGGCGAGGACCGTGTCTGTCGGGCGAGGACCGTGTCTGTCAGAAGATGGCGAGGACCGTGCAGAAGA | |
| TGGCGAGGACCGCTCAACAGAAGATGGCGAGGCCAGTACTCAACAGAAGATGGTTCCGACCAGTACTCAACAGCTCTTGTTCCGACCAGTACTCTCCTCTTGTTCCGACCAGGACGATCCTCTTGTTCCGCTAC | |
| AGACGATCCTCTTGGCCTGTCTACAGACGATCCCTGTAGCCTGTCTACAGACGAATCTGTAGCCTGTCTACGATGAGTGAACATGACAGGTTACTATGAGGATTTAAGCCACGATGATCATATCCTTGCCACGA | |
| TGATCATATCCGCGCCACGATGATCATATATGCGCCACGATGATCATTGCGCCACGATGATCATAATACATGCGCCACGATGGAGTAATACATGCGCCACGGATGAGTAATACATGCGTTGCAGTATCTCAGTG | |
| GCGCTTGCAGTATCTCAGTGGTCAGCTTGCAGTATCTCGGAAGTAAGTCAGCTTGCAGTTGACAGGTGGGAGAAGTGACCAGTAACTTTAATGGTGGATCAGGATTTGGTTTCCGCTCTTGATCAGGATTTGGC | |
| CTTTGCTCTTGATCAGGGATTAACATTCCTTTGCTCCTGGTTTCATAGTCATCTTCGGTTACACATTTGCTTTCAGTGTGGAGGATGGTTACACGGTTACACATTTGCTTTCAGGTCGTTCTGCAGGATGGTCC | |
| GGTGGTCGTTCTGCAGGATGAGTGAGTGGTCGTTCTGCGCCAGTAGTGAGTGGTCGTGCTGGCCAGTAGTGAGTGGCCCAGCTGCTGGCCAGTAGTGAAGCCCAGCTGCTGGCCACAGCAGAAGCCCAGCTGCG | |
| AAGTTCAGCAGAAGCCCAGGCATGAAGTTCAGCAGAAGTTCCAGGAGACCCTGGTGTGAGGTTCCAGGAGACCCCACAATTGAGGTTCCAGGGTGTTTGTGGTGCAAGTTATGTCTTCTGTAGTGTTTGTGGGC | |
| CAGGTGCAGTGAAGGGACAAGCCAGGTGCAGTGATGCCAACAAGCCAGGTGCATCTGTGCCAACAAGCCAGGCATCTGTGCCAACAAGCCCCAAGAGCCATACCTATAGCAGTCCAAGAGCCATACCGCATTCT | |
| TCAGTCCAAGAGCCCTTGGCATTCTTCAGTCCTGCTGTATTCTTGGCATTCTTCTTTGCTGTATTCTTGGCCTCCCCAGTGTGTCTTTGCTGTTCTCCCCAGTGTGTCTTGATATTTCTCCCCAGTGTCATGCG | |
| ATATTTCTCCCCCCAGCATGCGATATTTCTGCCAGTCACGCCCTTTGCTGGCCAGTCACGCCCTTTGCAGCCGTTAACAAGCACCGGCTGGAGCCGTTAACAAGTGCTTGCTGGAGCCGTTAGCAGAGTGCTTG | |
| CTGGAGCGATAGCAGAGTGCTTGCTGGGCCTGATAGCAGAGTGCATCAATGGCGTGAAGGGCATTTGATCAATGGCGTGATGTGACATTCAGTGGAGGTCTCTGCTGTGACATTCAGTTCAATCTCTGCTGTGA | |
| CAGTTCCTTCAATCTCTGCTGCGAGTTCCTTCAATCTCTGCTGGAGAGACGAGTTCCTTCAGGATAGAGAGACGAGTTCCTCCCATTGGATAGAGAGACGGTTTCTCCCATTGGATAGAGCACTGGTTTCTCCC | |
| ATTGGGAATGCATTGGTCATCAAGAGATGAATGCATTGGTCCTTGAACATCGTACTTAGGCTCTCACTTGAACATCGTACCTGCTCTCACTTGAACATCGCTGCTGCTCTCACTTGAACTCTCTGCACATGGAG | |
| CTCCTGCCTCTCTGCACATGGGAGCCCTGCCTCTCTGCACCTCCAGAGCCCTGCCTCTCTCTCACTCCAGAGCCCTGCCCTCCACTCACTCCAGAGCCTGGCTCCACTCACTCCAGAGGGTTGGCTCCACTCAC | |
| TCCAGTGTTCATCATTTCCCACATAAAGGGCTTGTGTTCATCAAACCACTCTCTCAAGGGCTCAGTAACACTAATACCGTCAGATGGTTGCCATAATCACTGAAGCAGATGGTTGCCATCTCATAGTTAGTGGT | |
| TACGCTTTCTCATAGTTAGTGGGACCCAGCTTTCTCATAGGACTTCAATTTCCGTCTCCAGATGACTTCAATTTCCGCAACTCCAGGCTTCTCTATACAACTCCAGGCTTCTCTATGGGTCTCAACTCCAGGCT | |
| TCCCAGGGTCTCAACTCCAGGCATCCTCCAGGGTCTCAACCACAGAATCCTCCAGGGTGGATGCCAAAGTGACAGTCAGGATGCCAAAGTGACAGTCTCATCAGAGGATGCCAAAGTGTGTGAGTTCATCAGAG | |
| GATGGCATGTGTGAGTTCATCAGCTGAGGCATGTGTGAGTTCGAGCCAGCATCCCTGTTCTTGAGCCAGCATCCCCTGTTCAGCCAAGAGCCAGCATCCCTGTGCCAAGAGCCAGCATCCCTGTTAGCCAAGAG | |
| CCAGCATCCCCCTCTTAGCCAAGAGCCAGCACACCTCTTAGCCAAGAGCCTCTGAACACCTCTTAGCCAAGTTCTGAACACCTCTTAGCCTGGGTGTATTGCTTCGCAGGGATGAAGCATCCATACTTTTGAGG | |
| CGATTTGGATGAAGCGTCAACTTCCCTGCTGTAGGTGCTTGGATGAGTCAACTTCGTGCTACCCTGTACGGCATGTTGGCAGGTGAGGAGGTGCTGTCTGAGGTGAGTCAAGCACGGCACAGCCAGAAGTAACA | |
| GCCAAACGGCACAGCCAGGTGCTACAATTGGCAGCTTTGGTTCACTCCAGGCTGATGCAGTCTTGAATCCAAGTTCAGGTAGATGNTGGTCATGGTCNGAAATGGCCAGGTTAGGAAGAGCAGGTCNGAAATGT | |
| NATAAAACCCAGAGAGGACCTGTGTNATAAAACCCAGAACAAGCCTGTGTNATAAAACGCTGTACAAGCCTGTGTNATCTCGCTGTACAAGCCTGTGTAAGATCTCGCTGTACAAGCCAAAGAGGCTTGTACAG | |
| CGAGATGAAAAAGATCTCGCTGTAGGATNATGAAAAAGATCTCCAGCAGGATNATGAAAAAGCATCGTCAGCAGGATNATGAAGTCAATCGTCAGCAGGATNAACCTGTCAATCGTCAGCAGGAGGTACCTGTC | |
| AATCGTCAGGGCCAGGTACCTGTCAATCGCNATCGATTGACAGGTACCTTGGACNATCGATTGACAGGTGTGATGACACCAAAAGTGACGCTGGTGATGACACCAAAAGGATGCTGGTGATGACACCACTGTAC | |
| AAGCCTGTGNGAATNCCTGTGTAGCAGATGGTAGGCGATCACCTCNGTCACAAGGCGTAGATCACNGGGTTCCAACNAAGGCGTAGATCACGTTGGAGAGAGGTTCCGGAAGAGAGAGGTTCCGGAAGTACCGC | |
| AGGTACTTCCGGAACCTCTGNCGCAGGTACTTCCGGAATTTTAGAGGTGAGTGTGGAAGAGGTGAGTGTGGAAGGCTTAATGTGTTTGCTTCATCTCCGTTTGCTTCATCTCCTTGGTCTTCATCTCCTTGGTC | |
| CTTCTCTCCTTGGTCCTTCCTCTTTTGGTCCTTCCTCTTTAGGCCCTTCCTCTTTAGGCAATTTCTCTTTAGGCAATTTTCTGCTAGGCAATTTTCTGCATCTGAATTTTCTGCATCTGACCTACAATAGAGAG | |
| TTCCGGCTCTGAGAGTTCCGGCTCTGCTGTTTCCGGCTCTGCTGTGGATGGCTCTGCTGTGGATGGAGAGGCTGTGGATGGAGAGACAGAGGATGGAGAGACAGAGCTGGGAGAGACAGAGCTGGTTCTTACAG | |
| AGCTGGTTCTTTCCAGGCTGGTTCTTTCCAGCTTCTTTCTTTCCAGCTTCTCACTATCCAGCTTCTCACTAGGAAGCTTCTCACTAGGAAGGAATGCACTAGGAAGGAATGGGATGGGAAGGAATGGGATGTAT | |
| CTGAATGGGATGTATCTGCCCAGGATGTATCTGCCCAGGTGCTATCTGCCCAGGTGCATGAGGCCCAGGTGCATGAGCAAGTGGTGCATGAGCAAGTGCCTGATGAGCAAGTGCCTGTGGAACAAGTGCCTGTG | |
| GAAGAAGTGCCTGTGGAAGAAGTGGCGCTGGAAGAAGTGGCGCAGGTAGAAGTGGCGCAGGTACTTCCGGCGCAGGTACTTCCGGAACAGGTACTTCCGGAACCTCTCCTTCCGGAACCTCTCTCCAAGCGTAG | |
| ATCACCGGGTTCATGATCACCGGGTTCATGCAGCCCGGGTTCATGCAGCAGTGGTTCATGCAGCAGTGGGAGTAGCAGCAGTGGGAGTAGGCGAAGTGGGAGTAGGCGATCACCGAGTAGGCGATCACCTCTGT | |
| GGCGATCACCTCTGTCACCATCACCTCTGTCACCAGCATGTCTGTCACCAGCATGACCAGCACCAGCATGACCAGGTCCAGCATGACCAGGTCCAGATGCACCAGGTCCAGATGCTTGCTGTCCAGATGCTTGC | |
| TCCGCTGATGCTTGCTCCGCTCACAGTTGCTCCGCTCACAGTCATTCCGCTCACAGTCATTTCCAAATGGATTGATAGGAAGAGAGTTGATAGGAAGAGAGAAGGAAGGAAGAGAGAAGGATAGCCGAGAGAAG | |
| GATAGCCACATTAAGGATAGCCACATTGTAGGTAGCCACATTGTAGGGTGTCACATTGTAGGGTGTCCAGAAGAGCCGGATGGCCTTGTACTGGATGGCCTTGTACTTTTTTGCCTTGTACTTTTTTTTACTAC | |
| CTCAGCAGCGTTTTGATGAGCAGCGTTTTGATGATTCCCGTTTTGATGATTCCTGTGTTGATGATTCCTGTGTAGCAGATTCCTGTGTAGCAGATGGCCGAGCAGAGGGAGAACGAGAAGGTAGATGNTGGTCA | |
| TGGTCNGAAATGGCCAGGTTAGGAAGAGCAGGTCNGAAATGTNATAAAACCCAGAGAGGACCTGTGTNATAAAACCCAGAACAAGCCTGTGTNATAAAACGCTGTACAAGCCTGTGTNATCTCGCTGTACAAGC | |
| CTGTGTAAGATCTCGCTGTACAAGCCAAAGAGGCTTGTACAGCGAGATGAAAAAGATCTCGCTGTAGGATNATGAAAAAGATCTCCAGCAGGATNATGAAAAAGCATCGTCAGCAGGATNATGAAGTCAATCGT | |
| CAGCAGGATNAACCTGTCAATCGTCAGCAGGAGGTACCTGTCAATCGTCAGGGCCAGGTACCTGTCAATCGCNATCGATTGACAGGTACCTTGGACNATCGATTGACAGGTGTGATGACACCAAAAGTGACGCT | |
| GGTGATGACACCAAAAGGATGCTGGTGATGACACCACTGTACAAGCCTGTGNGAATNCCTGTGTAGCAGATGGTAGGCGATCACCTCNGTCACAAGGCGTAGATCACNGGGTTCCAACNAAGGCGTAGATCACG | |
| TTGGAGAGAGGTTCCGGAAGAGAGAGGTTCCGGAAGTACCGCAGGTACTTCCGGAACCTCTGNCGCAGGTACTTCCGGAAAGAGGGAGAACGAGACAGAAGAGAACGAGACAGAAGATGGCGAGACAGAAGATG | |
| GTCATTCAGAAGATGGTCATTCTCAGGATGGTCATTCTCAGAGTGTAACTCTTCAGTCTCATAGAAATTCAGGAAGAGCTGCTAGCCAGGCCCCAGGTGACGATGCCCCAGGTGACGATGCTGGTGGTGACGAT | |
| GCTGGTGATGACAAGTGACAGTCCGGGCTCGAACAGTCCGGGCTCGAAGGGCCAGCATGGACAATGGCCAGGTGGACAATGGCCAGGTACCTAATGGCCAGGTACCTGTCGACCAGGTACCTGTCGATTGTCTA | |
| CCTGTCGATTGTCAGCAGGTCGATTGTCAGCAGGATTAAAGATCTCGCTGTACAAGCCCTCGCTGTACAAGCCTGTGTAACCCTGAGAGGAGCTTACATGAGAGGAGCTTACACATGCGGAGCTTACACATGCC | |
| ATGGAACCCAGTTATGCCCCCTGAAGTTATGCCCCCTGACATAGTGCCCCCTGACATAGTGGATCCTGACATAGTGGATCCAGACATAGTGGATCCAGAATGGATGGATCCAGAATGGAAGGGTCCAGAATGGA | |
| AGGGTGACGAATGGAAGGGTGACGAGGAAGAGGGTGACGAGGAAGAGCAGGACGAGGAAGAGCAGGTCCGGCCAGGTTGAGCAGGTAGATGTTGAGCAGGTAGATGTTGGGCAGGTAGATGTTGGTCATAATTC | |
| GGAGCCTCCTGTATTTGAGCCTCCTGTATTTTATGATCCTGTATTTTATGAGGATCTATTTTATGAGGATCATCACTATGAGGATCATCACCACCAGGATCATCACCACCACCACAATCACCACCACCACATTG | |
| CCCACCACCACATTGCCCAAGACCACATTGCCCAAGAGGCCCTTGCCCAAGAGGCCCACAGTCAAGAGGCCCACAGTGAACAGGCCCACAGTGAACACCAGGACAGTGAACACCAGGGAGTAGAACACCAGGGA | |
| GTACAGCGCTGGGCCATCAGTGCTCTGGCCATCAGTGCTCTGGTATCAAGTGCTCTGGTATCAGCTTTTCTGGTATCAGCTTTTTCACTATCAGCTTTTTCACAGAGCGCTTTTTCACAGAGCAGGCCTTCACA | |
| GAGCAGGCCCACGTAGAGCAGGCCCACGTCATCAAGGCCCACGTCATCATAGTACACGTCATCATAGTAGGATGCATCATAGTAGGATGTGGTAGTATCTAGTGAGGTTGTCATTAGTGAGGTTGTCATTTCAC | |
| CCACTTCTTCTTGTTGGTCCAGATACTTCATGGTATTTATGGCTTTGGAGTTGTCAGGCTCTGGTTTGGTTTCCCCATGCCCTTTGGACTGCTGATATTCATGGAGGATTTGCCCACACGTGACAGGGGTGATC | |
| ATCTTTGCCAGGACCGGGTTCACAAGAACAATGACCTTCCATTTAATGAGTCACACGGTGGTTGGGACAGAAGCTGGGGTATCTTCCTATTGGCCTGATTCTCCTGTAGCTCTCTACCCACTTCTTCTTGGGGT | |
| CTGGTCCAGATACTTCATGGCTCAGGCTCTGGTTTGGTTTCCCCATGCCCTTTGGACTGTAACTGATATTCATGGAGGTTGCCCACACGTGACAGGGGATTCCAGGGAGGAAGAGTACTGATCATCTTTGCCAG | |
| GTGAGTCACACTTTGGGTTCCCCCTCAGCTCAGTGTGGGGGCTTACCTGGCCAGCTGGTTACCTTACCTTTCCTGCTGACCTAGTAGCCTGGAGGGAATTTGCAGTGAGTCTGTAGAGGCATTTCTTGTCCACC | |
| CGGCTCTTCCAGTGACTGGATGACTCGGGAGCTTTCTAGTTCCAGCGGCCCTCTTGAATGTCACTGTAAGGATAGCTGGGTCCATGCCCACAGTAAGACAGCAGATTTTCCTTCTCTCCCCTCTCTTAGCCACT | |
| GTCTCTGTCCCCTGGGACCTCGTTCTTTCTCCTAGATAACTTTCCCCAGAGTATGGGAAAATGAGTCTGGCTCAGTGGGACCGATTGTTTGTTACCATGCTATGCTGCAGAAGCCTGAGCCCATACTCCATCAG | |
| TCTAGAATCCTTCCATCAATGTTCCCCCACCCTGAGAGCTTTCTTCTGCTTGAGCCACAGTGCATTGCCTTCTTGGCCACTTTTGTTTGGGACTTGCATTTCTTGGCCACTCTCCATCCTCTGTTTTGAGCGTG | |
| GCCATGTGATGGTCCATTACTGTAGAGTGTATGGTTGGAGGCATTAATTAGTTTGCATCCTCCCCTGTGCACTGGGATGCTGTCCTGTGTCTTCGGTGGAGGGAGAATCCCTCTCACAGTTCTTGACTCTGTGC | |
| GGGAGCTGGGAGTGAACACTCCATCTTTGCTCTTGACTTGCTTGGGTTAGTTGCTGCTCTATTTTTCATGTTTGCCAGTAGAGTGCAGTGTTCCTCCCTTCAGTGTTCCTCCCTTCCTTGTTCCTCCCTTCCTT | |
| GCCTCTCCCTTCCTTGCCTCTAGAGGCCTTGCCTCTAGAGGCATGCCCTCTAGAGGCATGCTGACTAGCAGCGCCTCAGAAGCTCTCGCCTCAGAAGCTCTTCTAGCAGAAGCTCTTCTAGGCTTTGCTCTTCT | |
| AGGCTTTAGTTGAGCCTCCACCTCCTGGGTTCCCTGCCTTAGCCTCCCGAGTCTTAGCCTCCCGAGTAGCTGCCTCCCGAGTAGCTGGGATTGGATTACAGGCGTGGGCCACACAGGCGTGGGCCACCACGCCG | |
| TGGGCCACCACGCGGCTAGCAGTGGCGCGATCTCGGCTCAAGCTCCGCTCCCGGGTTCTCCGCTCCCGGGTTCACGCCCTCAGCCTCCCGAGTAGCTGCCTCCCGAGTAGCTGGGACTCGAGTAGCTGGGACTA | |
| CAGGAGCTGGGACTACAGGCGCCCGGACTACAGGCGCCCGCTACCGCCCGCTACCACGCCCGGCTTTTGTATTTTTAGTACACATTTCATCATGTTGGCCAGGCTGTCTCGAACTCCTGACCTCCGAACTCCTG | |
| ACCTCAAGTGACCTCAAGTGATCCACCCACAAGTGATCCACCCACCTTGGATCCACCCACCTTGGCCTCCTGCTGGGATTACAAGGCTGAGGATTACAAGGCTGAGCCACACAAGGCTGAGCCACCACGTGCCA | |
| CCACGTCCAGCCTGGGTGCTGCTCGTCGTGGTCAGACTCGTCGTGGTCAGAATCTGTCAGAATCTGGGCCCTTCAAATCTGGGCCCTTCAAGGAGCGGCCCTTCAAGGAGCGGGTGGGGAAGCCTCCCAAGCTA | |
| GGGGCAGATGCAGGAGCGCAGAATGCAGGAGCGCAGAGGGCATCTCCATCCTAGCTCATCTCAGTTGATGTACTCCCGAACCGAACCCATTTCTTCTCTGGGCATTTCTTCTCTGGGTTGGCCTTCTCTGGGTT | |
| GGCACACATTGGCACACACTTGGCGGTTACACACTTGGCGGTTCTTTCCTTGGCGGTTCTTTCGGGTGCTGCTGGGTTGGAGCACTTGGGGTTGGAGCACTTGCCACTTACTCCTTGATGTGGGCACGGCTGAG | |
| CCACCACGTCCAGCCTGGGTTGGCACACACTTGGTCCTGACCTCAAGTGATCCACGTGGTCAGAATCTGGGCCCATTTTCCTGCCTTAGCCTCCGCCTCCCAAGCTAGGACAAGCCAAGCTAGGACAAGAGCAA | |
| CTAGGACAAGAGCAAGCAGATTCAGGTTCAAGGACTCTCCGTTCAAGGACTCTCCATCCTAGGACTCTCCATCCTAGCTCACAGGCGCCCGCTACCACGCATGTACTCCCGAACCCATTTCTCCCGAACCCATT | |
| TCTTCTTCATGTTGGCCAGGCTGTCTTTGGCCAGGCTGTCTCGAACCAGGCTGTCTCGAACTCCTGTCCCGGGTTCACGCCATTCTGGTTCACGCCATTCTCCTGCACGCCATTCTCCTGCCTCAGATTCTCCT | |
| GCCTCAGCCTCCCCTGCCTCAGCCTCCCGAGTGGGCAGTGGGCGGGCAATGTGTGGGCGGGCAATGTAGGCAAAGCAGCAGGGTGTGGTGTCGCAGGGTGTGGTGTCCGAGGGTGTGGTGTCCGAGGAATATGG | |
| AGCGCAGAGGGCAGTAGCGCAGAGGGCAGTAGCAATGAGGGCAGTAGCAATGAGGATGGTAGCAATGAGGATGACAGCAATGAGGATGACAGCGAGGCGGATGACAGCGAGGCGTGCCACAGCGAGGCGTGCCG | |
| CGGAGAGGCGTGCCGCGGAGACCTGTGCCGCGGAGACCTTCATGGCGGAGACCTTCATGGTACCGACCTTCATGGTACCTGTGGTCATGGTACCTGTGGAGAGGGTACCTGTGGAGAGGCTGTCTGTGGACAGG | |
| CTGTCGGAGGAGAGGCATGCTGACTTCCTTCATGCTGACTTCCTTCCTTGTGACTTCCTTCCTTGTCACATCCTTCCTTGTCACAGAGCCCCTTGTCACAGAGCCCTTGCCCAGAGCTCAGAACCTAGAGGCTC | |
| AGAACCTAGAGACTTCGAACCTAGAGACTTCCTTTTTAGAGACTTCCTTTTGACAAGGGAAGCTTTTTGTTGTTGTGCTTTTTGTTGTTGTTGTTGTTGTTGTTGTTGTTGTGACGGTTGTTGTTGTGACGGAG | |
| TCTGTTGTGACGGAGTCTCACTTGACGGAGTCTCACTTTGTCGAGTCTCACTTTGTCACCCATCACTTTGTCACCCAGGCTGTTGTCACCCAGGCTGGAGTGACCCAGGCTGGAGTGAAGTGGGCTGGAGTGAA | |
| GTGGCACAGAGTGAAGTGGCACAATCTCAAGTGGCACAATCTCAGCTCCGAGTAGCTGGGATTACAGGAGCTGGGATTACAGGCGTGGGAGACGGAGTCTCGCTCTGTGGAGTCTCGCTCTGTCGCCCCTCGCT | |
| CTGTCGCCCAGGCTTCTGTCGCCCAGGCTGGAGTCGCCCAGGCTGGAGTGCAGTAGGCTGGAGTGCAGTGGCGCGGAGTGCAGTGGCGCGATCTTCTGGCTGAGCAAGTCCCTGTGCATTCATCTTTCCACAAT | |
| AGAGCTCTCCTTCCTACATTTTCCTACATTGCGGCATCCCACATTGCGGCATCCCTTCATGCGGCATCCCTTCATGTCCAATCCCTTCATGTCCATGACTTTCATGTCCATGACTCCCACGTCCATGACTCCCA | |
| CAGGCATGACTCCCACAGGCATGCTCCCCACAGGCATGCTCTCAACAGGCATGCTCTCAACCCCTGTGCTCTCAACCCCTGGGAACTGCCAGCAGCTCATAGTGGAATAGAAGAGGAGGCCAGAGGAGAGGAGG | |
| CCAGAGGAGAATAGTCATTTCACGTACTCCAGTTTCACGTACTCCAGCTTGACGTACTCCAGCTTGATTTCAATTCTGGACCCACTTCTCCTGGACCCACTTCTCCTTTGGGGGTGATCACATAGCTCTTCATC | |
| ACATAGCTCTTCAGCCTCTAGCTCTTCAGCCTCTGCAACTTCAGCCTCTGCAAGGAGAGCCTCTGCAAGGAGATCTTCACCTGCTGGTGGTGATCACACTGGTGGTGATCACATAGCTTCCAGCTTGATTTCAG | |
| TAGGGGTAGCAGAGTTCAAGTCTTCAGAGTTCAAGTCTTCAGGGTTCAAGTCTTCAGGGTGTGAGTCTTCAGGGTGTGAGCTTCCAGGGTGTGAGCTTCCGGCCTGTGAGCTTCCGGCCCAGGTGCTTCCGGCC | |
| CAGGTGTTTCCGGCCCAGGTGTTTCATATAGCAGCTCATAGTGGAAGGGAGCTTAGAGACAGCAACCTACGAGACAGCAACCTACTTGCTAGCAACCTACTTGCTCAAGGCCTACTTGCTCAAGGCCTTGTTGC | |
| TCAAGGCCTTGCTATAACATAGTACATTTTGAATCACCGAATGCATCCTATTTGAAAATTATGGTAGAAGTATTCCTGGTTCTGAAGATGACAGCCCTGAAGATGACAGCCTTCTGGGGACACCTGCTGGTGGT | |
| GATCAACCCCTGGGAACCGAATTCTCCTTCCTACATTGCGGCAATGTCAGCAGTCCTACTATCAGCAGTCCTACTATTGCATGTCCTACTATTGCATTCATCACTATTGCATTCATCTTTCCAACATATTTAGC | |
| AACACCTCATTTAGCAACACCTCACATTGCAACACCTCACATTCACAATGCAAGGAGATCTTCTTACTATGTGAAGCAGCAAGTAGATAAGCAGCAAGTAGATGGGACGCAAGTAGATGGGACGTTGATAGATG | |
| GGACGTTGAGTGCAGGGACGTTGAGTGCATCTGGGTTGAGTGCATCTGGCTGAGGTGCATCTGGCTGAGCAAGTCACTTCTCCTTTGGGTCAGCCTCCTTTGGGTCAGCACAGATTGGGTCAGCACAGATCTCC | |
| TCAGCACAGATCTCCTTGCCACAGATCTCCTTGCCCAGTTTCTCCTTGCCCAGTTTGGTTTTGCCCAGTTTGGTTCTGAACAGTTTGGTTCTGAAGATGATGGTTCTGAAGATGACAGCCCTGAAGATGACAGC | |
| CTTCTGGGGACACCTGCTGGTGGTGAACCTGCTGGTGGTGATCACACTGGTGGTGATCACATAGCTTCCAGCTTGATTTCAGTAGGGGTAGCAGAGTTCAAGTCTTCAGAGTTCAAGTCTTCAGGGTTCAAGTC | |
| TTCAGGGTGTGAGTCTTCAGGGTGTGAGCTTCCAGGGTGTGAGCTTCCGGCCTGTGAGCTTCCGGCCCAGGTGCTTCCGGCCCAGGTGTTTCCGGCCCAGGTGTTTCATATAGCAGCTCATAGTGGAAGGGAGC | |
| TTAGAGACAGCAACCTACGAGACAGCAACCTACTTGCTAGCAACCTACTTGCTCAAGGCCTACTTGCTCAAGGCCTTGTTGCTCAAGGCCTTGCTATAACATAGTACATTTTGAATCACCGAATGCATCCTATT | |
| TGAAAATTATGGTAGAAGTATTCCTCAACCCCTGGGAACCGAATTCTCCTTCCTACATTGCGGCAATGTCAGCAGTCCTACTATCAGCAGTCCTACTATTGCATGTCCTACTATTGCATTCATCACTATTGCAT | |
| TCATCTTTCCAACATATTTAGCAACACCTCATTTAGCAACACCTCACATTGCAACACCTCACATTCACAATGCAAGGAGATCTTCTTACTATGTGAAGCAGCAAGTAGATAAGCAGCAAGTAGATGGGACGCAA | |
| GTAGATGGGACGTTGATAGATGGGACGTTGAGTGCAGGGACGTTGAGTGCATCTGGGTTGAGTGCATCTGGCTGAGGTGCATCTGGCTGAGCAAGTCACTTCTCCTTTGGGTCAGCCTCCTTTGGGTCAGCACA | |
| GATTGGGTCAGCACAGATCTCCTCAGCACAGATCTCCTTGCCACAGATCTCCTTGCCCAGTTTCTCCTTGCCCAGTTTGGTTTTGCCCAGTTTGGTTCTGAACAGTTTGGTTCTGAAGATGAGCCTGTGTCTGT | |
| CCTCCTGCTTCGTTCCTCTCGTTCCTGCTTGGTGCCCTTGCCGTCCTGCTCCTCCGGGCTGTGCCGGCTGGTGGGCTCCCCTGGGTCCTCGCCCTGGCTCCGGCTCCTTCGCTGGCTGGCGGGGTCTCTTGCTC | |
| TGGGCCTGGCTGTGCTGCCTCCGTTTGGGTGGCGAAGCTCCTCGATCTCTGGGGAAGCTCCTCGATCTCTGCGCCTCCTGGGAAGCTCCTCCCTGCAACACCGCCTCCTCCTGCAACACCGCCTCCTGGGAGTC | |
| CCACGCCTGCAACGAGTCCCACGCCTGCAACACGATCTGAGTCCCACGCCTGCGCACGATCTGAGTCCCACGCAGCACGATCTGAGTCCCACGCCAGCCCCAGCAGCACGATCTGGTCACCAGCCCCAGCAGCC | |
| GGCGGTCACCAGCCCCAGCGCCCACAGAGCGGCGGTCCAGCAGCCCAGCCCACAGCAGAGTCAGCAGCCCAGCCCGAAGCAGAGTCAGCAGCCCAGCAGGAGAAGCAGAGTCAGTGCCACAGGAGAAGCAGACC | |
| CAGTGCCACAGGAGAAGCGGTGTCCCAGTGCCACAGGTGTGTGGTGTCCCAGTGCCACTGTGTGGTGTCCCAGTGCCTGTGTGGTGTCCCAGTGCGACTCTGTGTGGTGTCCCAGTGACTCTGTGTGGTGTCCC | |
| AGCTGTTTTAGACTCTGTGTCCTCTCTTCCAGCTGTTTGCAGCCCTCTCTTCCAGCTGTCCGGGCAGCCCTCTCTTCCTCCGGGCAGCCCTCTCTTGACGTTCCGGGCAGCCCTCTCGACGTTCCGGGCAGCCC | |
| TCTGACGTTCCGGGCAGCCCTCGACGTTCCGGGCAGCCCTTGAGAGACGTTCCGGGCAGCCCTTGAGAGACGTTCCGGGCCTTGGAAACTTGAGAGACGGTTCTTGGAAACTTGAGAGTTTCCAAGTTCTTGGA | |
| AACTTGTGGCTTTCCAAGTTCTTGGGTGGCTTTCCAAGTTCTTCCGTGGTGGCTTTCCAAGTTCCCGTGGTGGCTTTCCAAGGGTCACCGTGGTGGCTTTCCGGTCACCGTGGTGGCTTTCATCTGGTCACCGT | |
| GGTGGCTCATCTGGTCACCGTGGTGTGCGCCATCTGGTCACCGTGGTGCGCCATCTGGTCACCGTTTCTGCGCCATCTGGTCACCTGGGATTTCTGCGCCATCTGCTGGGATTTCTGCGCCATGTGGACTGGGA | |
| TTTCTGCGCCGTGGACTGGGATTTCTGCTCTGCGTGGACTGGGATTTCTTCTGCGTGGACTGGGATTTGAAATCTGCGTGGACTGGGTCCTGTGAAATCTGCGTGGCCAGTTCCTGTGAAATCTGCGTTCCTCC | |
| AGTTCCTGTGCGAAGTTCCTCCAGTTCCCAGCTCGAAGTTCCTCCAGTTCTGTTCAGCTCGAAGTTCCTCCTCTGCTGTTCAGCTCGAAGTTCAATCTCTGCTGTTCAGCTCGATTTCAATCTCTGCTGTTCTC | |
| CTGAGATTTCAATCTCTGCCCAAGTCCTGAGATTTCAATCCAGCTCCAAGTCCTGAGATTTCAGGACAGCTCCAAGTCCTGGGTTCCAGGACAGCTCCAAGTGTTCAGGTTCCAGGACAGCTCGCCCGTTCAGG | |
| TTCCAGGCTTGAAGCCCGTTCAGGTTCCTCTGCTTGAAGCCCGTTCAGGCTCAGATCTGCTTGAAGCCCGGCTGCTCAGATCTGCTTGCTTGAAGCTGCTCAGATCTGCTGGGACTTGAAGCTGCTCTTCCTGG | |
| GACTTGAAGCTGCTGTTCAATTCCTGGGACTTGCTCCTCCCGGAGTCTTTCCAGGTCACCTCCTCCCGGAGTCTTGCTTTGTCACCTCCTCCCGGCTTGAGCTTTGTCACCTCCTCTCCATCCTTAGCTTTGTC | |
| ACCGCAACTCCATCCTTAGCTTTGCACCTGCAACTCCATCCTTCTGGACACCTGCAACTCCATCGCCGCTGGACACCTGCAACTCCACAAAGCCGCTGGACACCTGTTGCACACAAAGCCGCTGGCGTGTTGCA | |
| CACAAAGCCGCTCCACTTTTCAGGGCACGTGTTGTTGATCCACTTTTCAGGGCTGGAAGTTGATCCACTTTTCTCCGTTGGAAGTTGATCCGCACTTCCGTTGGAAGTTGTAGTAGCACTTCCGTTGGCGAAGT | |
| AGTAGCACTTCCGCTTGCCGAAGTAGTAGCACTTGTGCCCTTGCCGAAGTAGTTGGACCCACTGCTTGGTGCCCGGGCGTGGACCCACTGCTTACCGGGCGTGGACCCACCAGGCATACCGGGCGTGGCGTCAC | |
| AGGCATACCGGGCATGTCGTCACAGGCATACCGCCTTCCATGTCGTCACAGGCGCTGCCCTTCCATGTCGTGACCAGCTGCCCTTCCATGTTGCTGACCAGCTGCCCTTCCTGTGGATGCTGACCAGCTGCGCT | |
| TGGTCAGGAAGTCCTGCGGCATGCTTGGTCAGGAAGTCTGGCTGGCATGCTTGGTCAGGCGGTGTGGCTGGCATGCTTGGGGAGCCCGGTGTGGCTGGCTCCAGGAGCCGGTGTGGCTGGGCCAATCCAGGAGC | |
| CGGTGTCCGAAGGCCAATCCAGGAGCCGGTCCAAGTTCCGAAGGCCCTTCAGGTCCAAGTTCCGTCTCCCTTCAGGTCCAAGTTCTAAACTCTCCCTTCAGGTCCCCAGATAAACTCTCCCTTCAGTCCACCCA | |
| GATAAACTCTCCCTCCCATCCACCCAGATAAACTTGGCTCCCATCCACCCAGATTGGCTCCCATCCACCCAGATGGCTCCCATCCACCCAGATTCCACATGGCTCCCATCCACCTCCACATGGCTCCCATCCTG | |
| TAGTCCACATGGCTCCCATTGTAGTCCACATGGCTCCCGCCCAGTTGCTGTAGTCCCTGGAGCCCAGTTGCTGTCTCCCCTGGAGCCCAGTTGCTGGTCCAGCGACCGGAGCCGTCGCAGAAGGCGTCGGTCCT | |
| TACGGTCGCAGAAGGCGTCCCAGCTTACGGTCGCAGAAGGGGCGCCCAGCTTACGGTCGCCCCAGGCGCCCAGCTTACGGTCGCACACCCAGGCGCCCCCGGTCGCACACCCAGGCGCGCCAGCCGGTCGCACA | |
| CCTGTGGCCAGCCGGTCGCACCGTGCATGTGGCCAGCCGGTCCGTGCATGTGGCCAGCCGTGGCTGGCGGCGTGCATGTGGGACTCCGCGGAACCTTCGCCCCATGGACTCCGCGGAACCCAGGTCCCATGGAC | |
| TCCGCGTGAATCAGGTCCCATGGACTCTGAATCAGGTCCCATGGACGGTCTTGAATCAGGTCCCATGGGTCTGGTCTTGAATCAGGTCGTCAGGGTCTGGTCTTGAATCGCAGGCGGCCGTCAGGGTCTCCATG | |
| CTCAAGAGTGGAGAGTCCATGCTCAAGAGTGGAGTGGGCCTGGCTGTATCCATGCGGTCTTCAGGGTCTTGCTCTGGGCTTTTAGGCCGTGGTTGGGCCACAAAGAGGCTTTTAGGCGGACCTTTCAGCCACAA | |
| AGAGTGTCACAGGGACCTTTCAGCCCAGAAAATGTCACAGGGACCTGGTGGCAGAAAATGTCACAGGGGTGGCAGAAAATGTCACGTTTGGGTGGCAGAAAATGTCCCTCCGTTTGGGTGGCAGTGCCTCCGTT | |
| TGGGTGGCGTGTCAGCTGCCTCCGTTTGGGTGTCAGCTGCCTCCGTTTGTGTCAGCTGCCTCCGTTGCGGGAGATGTGTCAGCTGCGAGGAGCGGGAGATGTGTCGCCATAGAGGAGCGGGAGGTACTCCTGGG | |
| AAGGCAGGGGGAGAGGGTGCTGTTGGGTCTGGAGAGGGTGCTGTTGGGTCTGGAGAGGGTGCTGTTGGTCTGGAGAGGGTGCTGTTGTCTGGAGAGGGTGCTGTTTCTGGAGAGGGTGCTGTTCTGGAGAGGGT | |
| TGGGTGGCGTGTCAGCTGCCTCCGTTTGGGTGTCAGCTGCCTCCGTTTGTGTCAGCTGCCTCCGTTGCGGGAGATGTGTCAGCTGCGAGGAGCGGGAGATGTGTCGCCATAGAGGAGCGGGAGGTACTCCTGGG | |
| AAGGCAGGGGGAGAGGGTGCTGTTGGGTCTGGAGAGGGTGCTGTTGGGTCTGGAGAGGGTGCTGTTGGTCTGGAGAGGGTGCTGTTGTCTGGAGAGGGTGCTGTTTCTGGAGAGGGTGCTGTTCTGGAGAGGGT | |
| GCTGCATCTGGAGAGGGTGCTGTTGCATCTGGAGAGGGTGCTGTCATCTGGAGAGGGTGCTCATCTGGAGAGGGTGCTCCCATCTGGAGAGGGTGCTGGGCACTCCCATCTGGAGAGGGGGCACTCCCATCTGG | |
| AGCTCTCATCTGGAGAGGGTGCGAGGACTCACCACCCTGAGCGGCTGGACAGGAGGACTCTATTGATGCAGGCTGGACAGGGGCCATCACTGCCCCATTTTGCGTCCCCGCTCCCTAGCTGTCCTGTTCTATTT | |
| GGCCTCTGGCTTGGAGGATTCATTATGCTGCAGAAGGCGTCGTTCCGGAAGTGTGGGCCTTTGTGTGCCTTTGTGTTTTGATGCTATTTGATGCTACACATGTCTACACATGTCTATGGAGGGCCATGGAGGGC | |
| CACTTCTTCTGTCTTCTTCTGTAAGTCTGTGGAAGTCTGTGGGGCCTCAGCAGGCCTCAGCATACCCAATAGTACCCAATAGGCAGCAAGTTGCAGCAAGTTTCAGTATTTCTCAGTATTTCCCAGTTGTATCC | |
| AGTTGTATGTCCTCATGGGTCCTCATGGTGGGGCTATGTGGGGCTATGTCTCCCCCACTCTCCCCCACCACTTCCCCTCACTTCCCCTCTCATCAGGCCTCATCAGGCTAGACTTTAATAGACTTTAACATCCA | |
| TCAACATCCATCAATCATGTCTTGTCATGTCTTGAGTCTTGCTCAGTCTTGCTCCTTCCTCTTGCTTCCTCTTGGCTTAGTCATGCTTAGTCATGTGACTACAGGTGACTACAGATCAGATGCGATCAGATGCG | |
| TGGCCTAGTGTGGCCTAGTGTTTTAGGTGTTTTTAGGTGTGCAGGTACCAGCAGGTACCATGGCCCCAAATGGCCCCAAATGCTGTTGTATGCTGTTGTATCTGACTGAGTCTGACTGAGGACAATGCCCGACA | |
| ATGCCCTGTCCTCCGGTGTCCTCCGGCGTCCCAGGGCGTCCCAGGGCCGGTAGGTGCCGGTAGGTGTAGCTGCATGTAGCTGCATCGCATATGTCTCCATATCTCTTCCACTCTTCTTCCACTCTCTTCACTCT | |
| GCTCAGTGTGGCACCACTGCCAACCACTGCCACCAATATGGGCCAATATGGGAAGGCCGAGGAAGGCCGAGGAAGAGGCCCTAAGAGGCCCTGTCCCGGGATGTCCCGGGATAGGTTCAGGGAGGTTCAGGGAG | |
| GATCTTCCGCTGGCTTCCGCTGGCGGTTATAGAGGGTTATAGAGGTACGTGCTGGTACGTGCTGAGGCCTGCAGAGGCCTGCAGTGCCCATTATTGCCCATTATGACTGCGGCTGACTGCGGCTGCTACCACAG | |
| GCTACCACAGTGATGATGACTGATGATGACAATCTCATACAATCTCATACCGGGGGGAGACGGGGGGAGAGCACATTCACGCACATTCACGGTCACCTTGGGTCACCTTGCGGGTGACCTCGGGTGACCTCCCC | |
| TTGAGTCCCCTTGAGTGCTCCTGGCCGCTCCTGGCCCGACAGAGGTCGACAGAGGTAGGTGCCCTCAGGTGCCCTCAAGATCTCGAAAGATCTCGAGTGACAGTCAGTGACAGTCACTGATTCCCCCTGATTCC | |
| CCGATGGGCAGTGATGGGCAGTGGGAAAGTGCGGGAAAGTGCCATCCTTTAGCATCCTTTAGACACTTGAGCACACTTGAGCTCGGGCAATGTCGGGCAATGGGTTCCCCCAGGTTCCCCCAAGCCTGGCACAG | |
| CCTGGCACATTGGAGTCTATTGGAGTCTGCTGGGAATTGCTGGGAATTTTCTGGCCACTTCTGGCCACGTCCAGTTTCGTCCAGTTTCCCGGACAATCCCGGACAATCCCTCTCGTCCCCTCTCGTCCAGTCGG | |
| GGGCAGTCGGGGGCCATACAGGACCATACAGGACACGAAGCTCCACGAAGCTCCCGGGTCTGGTCGGGTCTGGTTCTTGTGTATTCTTGTGTATAAGCTGGCCGAAGCTGGCCGGCCACCTCCAGCCACCTCCA | |
| GGGTTGCAGAGGGTTGCAGAGCAGGAGAAGGCAGGAGAAGCTGCGCCCGTCTGCGCCCGTTGTCCTCTGGTGTCCTCTGGGGTGGCCTTCGGTGGCCTTCAGCAGGAGCTAGCAGGAGCTGGGCCCTCGGGGGC | |
| CCTCGGGCCCAGTGGCGCCCAGTGGCTGGGCTGGAATGGGCTGGAACCCCATTCAGCCCCATTCAGCGTCACCTTGCGTCACCTTGGCTCTAGGGTGCTCTAGGGTGGGCCTCACAGGGCCTCACACTTCACTG | |
| TCCTTCACTGTCACCTCGGTCCACCTCGGTCCCTTCTGAGACCTTCTGAGACCTCTGGCTTCCTCTGGCTTCGTCAGAATCAGTCAGAATCACGTTGGGCGCCGTTGGGCGCCGGAAAGCTGCGGAAAGCTGTA | |
| GATGGTCATAGATGGTCACTGTCTGCAGCTGTCTGCAGTGTCTCCTGGTGTCTCCTGGCTCTGGTTCCCTCTGGTTCCCCAGTATTACCCAGTATTACTGCACACGTCTGCACACGTCAGCCGCTGGGAGCCGC | |
| TGGGTGCCCTCGTCTGCCCTCGTCCTCTGCGGTCCTCTGCGGTCACACTGACTGACACTGACTGAGGCCTTGGCAGGCCTTGGCCGAGAAGGAGCGAGAAGGAGTCGTTGCCATTCGTTGCCATAGGTGACTGT | |
| AGGTGACTGTGGGGTTCAACGGGGTTCAACCTCTGGTCCCCTCTGGTCCCCCAGTGCCAGCCAGTGCCAGGTGGACCTGGGTGGACCTGGGCCTCCGAGAGCCTCCGAGACTGGGAACAGCTGGGAACAGCCCG | |
| TCCAGGCCCGTCCAGGGAACAGACCAGAACAGACCACGGTCCCCTGCGGTCCCCTGCGTGTCCACCCGTGTCCACCTCTAGGACCCTCTAGGACCCGGGGGCTGACGGGGGCTGACAAGTTGTGGGAAGTTGTG | |
| GGGGAGTCGCTGGGAGTCGCTGGCAGGACAAAGCAGGACAAAGGTCTGGAGCGGTCTGGAGCTGGTAGGGGGTGGTAGGGGGCCGAGGTGTTCCGAGGTGTTCTCAAACAGCCTCAAACAGCTCCAGCCCTTTC | |
| CAGCCCTTGGGGCCGCAGGGGGCCGCAGGTCCAGTTCAGTCCAGTTCAGTGCGGCACGGTGCGGCACGAGAAATTGGCAGAAATTGGCTCCATGGTGATCCATGGTGATCTCTCCTCATCTCTCCTCACCAGCA | |
| CCGTCCAGCACCGTGGTCGTGACCGGTCGTGACCTCAGCGGGCTTCAGCGGGCTCCCCCACAGCCCCCCACAGCTGGCTCCCGTTGGCTCCCGTTTCAGCTCCTTTCAGCTCCTTCTCCCCACGTCTCCCCACG | |
| GAGCAGCACCGAGCAGCACCACGGTGAGGTACGGTGAGGTTGGCCCGGGGTGGCCCGGGGTGCCCCACCCTGCCCCACCCTCCACCTGGCTCCACCTGGCAGCGTAGGGTAGCGTAGGGTAAGGTTCTTGAAGG | |
| TTCTTGCCCACTGGCTCCCACTGGCTGCCAAGAGGGGCCAAGAGGGGAGGGGTGCCGAGGGGTGCCAGTTCCACCCAGTTCCACCCGTTCTGGAGTGTTCTGGAGTCCAGTACACGCCAGTACACGGTGAGGAA | |
| GGGTGAGGAAGGTTTTAGCTGTTTTTAGCTGTTGACTGCCCATGACTGCCCATCAGGGCAGTTCAGGGCAGTTTGAATAGCATTGAATAGCACATTGGTTGGCATTGGTTGGCTATCTTCTTCTATCTTCTTGC | |
| TCCCCGGGGCAGGATGACTTAGGATGACTTTTGAGGGGGATTGAGGGGGACACAGATGTCCACAGATGTCTGGGCATTGCTGGGCATTGCCAGGTCCTGGCAGGTCCTGGGAACAGAGCCGAACAGAGCCCCGA | |
| GCAGGACCGAGCAGGACCAGGAGTGCCCAGGAGTGCGGGCAGCGCGGGGCAGCGCGGGCCGGGGGCGGCCGGGGGCTGCTGGGAGCTGCTGGGAGCCATAGCGAGGCATAGCGAGGCTGAGGTTGCCTGAGGTT | |
| GCAACTCTGAGTAACTCTGAGTAGCAGAGGAGAGCAGAGGAGCTCAGCGTCGCTCAGCGTCGACTGGGGCGCTTTAGTACTGTGTCTCCTGTCTTTCTGCTTCTTCCAGCCTCTTCCAGCCTGGTTAATTCCTT | |
| TGCGTACTCTGCCTTTGTCTGCCTTTGTTTGGGTTCGATGGTAGGGATGAAGGTCATTTGTTCTCTAGAGATTTCATAAGATTTCATATCCGTATCCTCCAAAAACTCTATATTCTCCTATATTCTCCAGAATA | |
| GTCTTAATTCAATCTCCAGCCGGTCACGCTAGGAACCTTGCAGCTTCACGAGGCCACCACTCATCACCACTCATCTCGATTTCTCCGCTCAGAGGGCTGTCTATGGCTGTCTATCTGGGTTCTCCTGGGTTCTC | |
| CAGGAGAAAGATCTCAACAGTAAATGGTTTCCAGAATCTTCCATCCTCATCAGCCTGCCTTACTGTGGGCTACTGTGGGCACAGAATCCATTCACAAGTTGCTGTGCACAGCTGTGCACAGGTAAGAGTGTTCG | |
| TTCCCAAAACTAACAGTAGATTCTGGGGTGGTCTCGAGGCTCAGCATCCTGGCCACGCAGCAGGCTCAGCATCCTGCAGCAGCAGCAGGCTCAGCAAGCGCCAGCAGCAGCAGGCTCGGGCAGCGCCAGCAGCA | |
| GCCAGGACGGGCAGCGCCAGCACTCGCCAGGACGGGCAGCGCCGCGGCTCGCCAGGACGGGCGTAGGCGCGGCTCGCCAGGAGCCGCGTAGGCGCGGCTCGCCAGGGGCCGCGTAGGCGCGGTGGGGCAGGGGC | |
| CGCGTAGGTGGACTGGGGCAGGGGCCGCGGGCCTGGACTGGGGCAGGGCTGCAGGGCCTGGACTGGGGGCTTGCTGCAGGGCCTGGACTACCCGCTTGCTGCAGGGCCGACGATACCCGCTTGCTGCACCCCCG | |
| ACGATACCCGCTTGCCTGACCCCCGACGATACCCGGCCTCCTGACCCCCGACGACTGGGGGCCTCCTGACCCCCTGCTCCTGGGGGCCTCCTGACCACTTGCTCCTGGGGCCTCAGGGGCCACTTGCTCCTGGG | |
| CCTGCCAGGGCCACTTGCTCGCTCACCTGCCAGGGCCACTCTCAGGCTCACCTGCCAGGGGGACTCTCAGGCTCACCTGCGTCGCGGACTCTCAGGCTCATATCGGTCGCGGACTCTCAGTCCAGTATCGGTCG | |
| CGGACTGTGCATCCAGTATCGGTCGCCAGAAGTGCATCCAGTATCGCCCCGCAGAAGTGCATCCAGGGAGCCCCCGCAGAAGTGCAATGAGGGAGCCCCCGCAGAAGGTGGATGAGGGAGCCCCCGCTGGGGGT | |
| GGATGAGGGAGCACCCACTGGGGGTGGATGAGTCAGCACCCACTGGGGGTGGCGCGGTCAGCACCCACTGGGTGCGCCGCGGTCAGCACCCAGGCAGTGCGCCGCGGTCAGCTCCCAGGCAGTGCGCCGCGGTC | |
| CGGTCCCAGGCAGTGCGCTGACGTCCGGTCCCAGGCAGATCCTTGACGTCCGGTCCCAGCCAGATCCTTGACGTCCGGGGGTGGCCAGATCCTTGACGCCTGAGGGTGGCCAGATCCTTGCACCCTGAGGGTGG | |
| CCAGGCAGTTGCACCCTGAGGGTGCTCCCGCAGTTGCACCCTGATGCTGCTCCCGCAGTTGCACAGAGGTGCTGCTCCCGCAGTGTAGTAGAGGTGCTGCTCCCTCCTGGTAGTAGAGGTGCTGGCTGGTCCTG | |
| GTAGTAGAGGCAGCAGCTGGTCCTGGTAGTACTGGCAGCAGCTGGTCCTGTGCTGACTGGCAGCAGCTGGGATCCTGCTGACTGGCAGCAACGATGATCCTGCTGACTGGGGTGCACGATGATCCTGCTGCTGT | |
| GGGTGCACGATGATCCTAGAACTGTGGGTGCACGATTGATGTAGAACTGTGGGTGCCTGGATGATGTAGAACTGTGCCAGTCTGGATGATGTAGAACCGCTCCAGTCTGGATGATGGATATCCGCTCCAGTCTG | |
| GAAGGGCGATATCCGCTCCAGTCCAGCAGGGCGATATCCGCTCAGCTCCAGCAGGGCGATATTCCTCCAGCTCCAGCAGGGCCGGGCTCCTCCAGCTCCAGCGTTCACGGGCTCCTCCAGCTGAGATGTTCACG | |
| GGCTCCTCGGCTGGAGATGTTCACGGGCGACGCGGCTGGAGATGTTCAGTGTGGACGCGGCTGGAGATTGACCGTGTGGACGCGGCTGCAGCATGACCGTGTGGACGCGGGGGCAGCATGACCGTGTGAGGCAG | |
| GGGGCAGCATGACCCTCCGAGGCAGGGGGCAGCAAAGGTCTCCGAGGCAGGGGGGGGGGAAGGTCTCCGAGGCACCCCGGGGGGAAGGTCTCCGGGCATCCCCGGGGGGAAGGTAGCACGGCATCCCCGGGGGG | |
| GACCCAGCACGGCATCCCCGCCAGTGACCCAGCACGGCATCCCAGCCAGTGACCCAGCACATCGCCCCAGCCAGTGACCCTCCACATCGCCCCAGCCAGTCATTGTCCACATCGCCCCAGCTCATCATTGTCCA | |
| CATCGCAGGGGCTCATCATTGTCCACGTGGGAGGGGCTCATCATTGTGGCGGTGGGAGGGGCTCATGGAAATGGCGGTGGGAGGGGTCAGGGGAAATGGCGGTGGGCTGCTTCAGGGGAAATGGCGTTCACCTG | |
| CTTCAGGGGAAAGGACCTTCACCTGCTTCAGGTATGGGGACCTTCACCTGCTTCCATTATGGGGACCTTCACGGTTTTCCATTATGGGGACCAATGTGGTTTTCCATTATGGTCACAAATGTGGTTTTCCATTT | |
| GCGTCACAAATGTGGTTTGTATTTTGCGTCACAAATGTAGGTGGTATTTTGCGTCACACGCCAAGGTGGTATTTTGCGGTAGGCGCCAAGGTGGTATTCCCGTGTAGGCGCCAAGGTGCGTCTCCCGTGTAGGC | |
| GCCAGACGTCGTCTCCCGTGTAGGATGCGGACGTCGTCTCCCGTGGATGATGCGGACGTCGTCTGTCACGGATGATGCGGACGTATGTCGTCACGGATGATGCGACAGCATGTCGTCACGGATGGGCACACAGC | |
| ATGTCGTCACTTCCCGGCACACAGCATGTCGGCTGTTCCCGGCACACAGCCCTCTGGCTGTTCCCGGCACGAGTCCCTCTGGCTGTTCCCTGCAGGAGTCCCTCTGGCTGGCCCTTGCAGGAGTCCCTCTGAGT | |
| CGCCCTTGCAGGAGTCCTCCAGAGTCGCCCTTGCAGGGGCCCTCCAGAGTCGCCCTACCAGGGGCCCTCCAGAGTCTGCACACCAGGGGCCCTCCACACCTTGCACACCAGGGGCCCCATTCACCTTGCACACC | |
| AGAGGTGCCATTCACCTTGCACTAGCCAGGTGCCATTCACCTGCCTGTAGCCAGGTGCCATTCGCCCGCCTGTAGCCAGGTGGACCACGCCCGCCTGTAGCCCAGCTGACCACGCCCGCCTGCGTCCCAGCTGA | |
| CCACGCCCGCCCTCGTCCCAGCTGACCAGCACAGCCCTCGTCCCAGCTGCTGGGCACAGCCCTCGTCCGTTGGGCTGGGCACAGCCCTGGCCGGTTGGGCTGGGCACATGCCAGGCCGGTTGGGCTGGGTAGAT | |
| GCCAGGCCGGTTGGCGGGTGTAGATGCCAGGCCGTGACACGGGTGTAGATGCCAGTAGGTGACACGGGTGTAGAAAGTAGTAGGTGACACGGGTAGTCCAAGTAGTAGGTGACAGATCCAGTCCAAGTAGTAGG | |
| TGGTGGATCCAGTCCAAGTACATAGTGGTGGATCCAGTCCGGGGACATAGTGGTGGATCCTTTTTGGGGACATAGTGGTGACGGCTTTTTGGGGACATAGGACTCACGGCTTTTTGGGGAAGATTCAGCATCCT | |
| GGCCACGCAGCAGATTCAGCATCCTGCAGCAGCAGCAGATTCAGCAAGCGCCAGCAGCAGCAGATTTGGCCTGGGGCAGGGGCCGCGGGCCTGGACTGGGGCAGGGCTGCAGGGCCTGGCCTGGGGCCTCGCTG | |
| CAGGGCCTGGCCTGCCCACTCGCTGCAGGGCCAACGATGCCCACTCGCTGCACCCCCAACGATGCCCACTCGCCTGACCCCCAACGATGCCCGGCCTCCTGACCCCCAACGAGCCGTGGACTCTCAGGCTCATA | |
| TGGGCCGTGGACTCTCAGTCCAGTATGGGCCGTGGACTGTGCATCCAGTATGGGCCGTCAGAAGTGCATCCAGTATGGTGCGGTCAGCACCCACTGGGTGCGCTGCGGTCAGCACCCACGCAGTGCGCTGCGGT | |
| CAGCTCCCACGCAGTGCGCTGCGGTCCGGTCCCACGCAGTGCGCTGACGTCCGGTCCCACGCAGGGGCGGCCAGATCCTTGACGCCTGAGGGCGGCCAGATCCTTGCACCCTGAGGGCGGCCAGGCAGTTGCAC | |
| CCTGAGGGCGACCGGCAGCAGCTGGTCCTGTGCTGACCGGCAGCAGCTGGGATCCTGCTGACCGGCAGCAACGATGATCCTGCTGACCGGCGGTGTAGAACTGTGGGTGCCTGGACGGTGTAGAACTGTGCCGA | |
| TCTGGACGGTGTAGAACCGCTCCGATCTGGACGGTGGATGTCCGCTCCGATCTGGAAGGGCGATGTCCGCTCCGATCCAGCAGGGCGATGTCCGCTCAGCTCCAGCAGGGCGATGTCCGGCTCCTCCAGCTCCA | |
| GCCTTCACCGGCTCCTCCAGCTGAGACCTTCACCGGCTCCTCGGCTGGAGACCTTCACCGGCGACGTGGCTGGAGACCTTCAGTGTGGACGTGGCTGGAGACTGACCGTGTGGACGTGGCTGCAGGGTGACCGT | |
| GTGGACGTGGGGGCAGGGTGACCGTGTGAGGCAGGGGGCAGGGTGACCCTCTGAGGCAGGGGGCAGGGAAGGTCTCTGAGGCAGGGGGGGGGGAAGGTCTCTGAGGCACCCCGGGGGGAAGGTCTCTGAGGCGC | |
| TCATCATTGTCCACGTGGGAGGCGCTCATCATTGTGGCGGTGGGAGGCGCTCATGGAAATGGCGGTGGGAGGCGTCAGAGGAAATGGCGGTGGGCTGCTTCAGAGGAAATGGCGTTCACCTGCTTCAGAGGAAA | |
| GGACCTTCACCTGCTTCAGAGGACGATGCGGACGTCGTCTGTCACGGACGATGCGGACGTATGTCGTCACGGACGATGCGACAGCATGTCGTCACGGACGGGCTGTTCCCGGCACACAGCCCTCTGGCTGTTCC | |
| CGGCACGAGTCCCTCTGGCTGTTCCCTGCAGGAGTCCCTCTGGCTGGCCCTTGCAGGAGTCCCTCTGAGTCGCCCTTGCAGGAGTCCTCCAGAGTCGCCCTTGCAGGGGCCCTCCAGAGTCGCCCTACCAGGGG | |
| CCCTCCAGAGTCTGCACACCAGGGGCCCTCCATAGCCAGGTGCCATTCACCTGCCTGTAGCCAGGTGCCATTCGCCCGCCTGTAGCCAGGTGGACCACGCCCGCCTGTAGCCCGTCCCAGCTGACCACGCCCGC | |
| CCTCGTCCCAGCTGACCAGCACAGCCCTCGTCCCAGCTGCTGGGCACAGCCCTCGTCCTCAGGTAGGGTCTCCACCTGTCTCCACCTGACCCCCCGCCACCCCCCGCCACCAGGAGCTACCAGGAGCTGGGAGT | |
| GCGGGGGAGTGCGGATGTGTCCTCATGTGTCCTCCCCAAATGTCCCCAAATGTCCCTGCGCAGGCCTGCGCAGGCACTGCAAGCCACTGCAAGCCCTCTTGGCACCTCTTGGCAGCCTGGTGCTGCCTGGTGCT | |
| CTCGTTGGGCCTCGTTGGGCCTCCACCTCGCTCCACCTCGGCCGCCGTGGGCCGCCGTGGAGGGGAGGCCAGGGGAGGCCCGGGAGGCCCCGGGAGGCCCGGGGCATGCTGGGGCATGCTCTGAAACAGACTGA | |
| AACAGACAGGGTCCTCCAGGGTCCTCCAAGCAGGGACAAGCAGGGAGAAGGGGGCTGAAGGGGGCTTTGAAGAGCCTTGAAGAGCCAGGGCAGAGGAGGGCAGAGGGGCTGCTGTGGGCTGCTGTGGCTTACAG | |
| CAGCTTACAGCAACCACGAATTACCACGAATTCCTCCCGGGACCTCCCGGGACGAGAACTCACGAGAACTCATCCGGAGCCATCCGGAGCCACAGGTGCACTCAGGTGCACTGCCTGTGGACGCCTGTGGACTG | |
| TGCCTGCTTGTGCCTGCTGTGTCAAATAGTGTCAAATACACTGCCTCCCACTGCCTCCAGCTCGGCCTAGCTCGGCCTCCAGGGATGCCCAGGGATGCTTCCTGTGCCTTCCTGTGCCATAACTTCCAATAACT | |
| TCCAACGACTCCTGACGACTCCTGGGCCGGGGATGGCCGGGGATGCCTCCCAGGGCCTCCCAGGCTATGGTTGCCTATGGTTGCATCCAGAGCTATCCAGAGCTTCCTCGACTCTCCTCGACTCCTGACAATCC | |
| CTGACAATCCCTGCGCAGTCCTGCGCAGTCAATGCCTCTTAATGCCTCTTGGGCTGTGCAGGGCTGTGCACGGTATCTGGCGGTATCTGGGCCATTGATAGCCATTGATATTTCTTCCTCTTTCTTCCTCCTCT | |
| TCCTCCCTCTTCCTCCTCCAGCGTCATCCAGCGTCAGCTCAAACTGGCTCAAACTGGAACTTGTCAGAACTTGTCAGGCAGGGGTGGGCAGGGGTGGGTGGCCTGGGGTGGCCTGGCCCCCTTGACCCCCCTTG | |
| ACTCCTCCTCGGTCCTCCTCGGGTGGCGGAGAGTGGCGGAGATGGGCTCTGCTGGGCTCTGCCGGATGGCGCCGGATGGCGCTGTAGTACCATGTAGTACCAGTCCCGGTTGGTCCCGGTTGTCCTCCAAAGTC | |
| CTCCAAAGTGTCCAAGATTGTCCAAGATCTCCTGGGCACTCCTGGGCATCTGGGTGGATCTGGGTGGACAAGGTCCGCCAAGGTCCGCCCAGGTCTCCCCAGGTCTCCCACAATGGGTCACAATGGGTGCACAA | |
| TGTAGCACAATGTAGTCAATAAAAGTCAATAAAACCCACCTGAGCCCACCTGAGACTTCTCCACACTTCTCCACGGAGGCAGTGGGAGGCAGTGTGCTTGTCACTGCTTGTCACACATGGGGCTACATGGGGCT | |
| GATTTCCATGGATTTCCATGCCACGCTCGCCCACGCTCGCGCTCTCGGTCGCTCTCGGTCACCCTGCTGGACCCTGCTGGAAGAACTCGGAAGAACTCGGCCATGATGCGCCATGATGCGGTCTGTCCACGTCT | |
| GTCCACTGGCGGTACATGGCGGTACAGCTCCAGCGGGCTCCAGCGGCTTGGTGGGGCTTGGTGGGGTTGCTGAGGTTTGCTGAGGTCGGCACAGTGCGACACAGTGCACCATGTTCCACCATGTTCCGGAGGAC | |
| CTCGGAGGACCTGGATGCGGTCGGATGCGGTCGGAGTAGTTAGGAGTAGTTATCTAGCAGGATCTAGCAGGAGGACCCCTGAGGACCCCTGAGCTGGTCACTGCTGGTCACTTTCTTGGTCTTTCTTGGTCTCC | |
| ACCATGGTCCACCATGGTCTTCAGGTCACTTCAGGTCAGCCAGGAGGGGCCAGGAGGGTCATGTGCTTTCATGTGCTTGGACATGTCCGGACATGTCCGTGGCCAGCAGTGGCCAGCACCATGTCGATCCATGT | |
| CGATGACCATCTTGGACCATCTTGCGTAGGCTCTCGTAGGCTCTGCCGCTGGCGGCCGCTGGCGCTTGCTGAGGCTTGCTGAGGTTCTGGAAGATTCTGGAAGATGTCGCAGTTTGTCGCAGTTGTCCTCCTGC | |
| GTCCTCCTGCAGCAGCTTGAAGCAGCTTGAAGCCCACGGCAGCCCACGGCCAGGTGGTGACAGGTGGTGATTCTCGAGCATTCTCGAGCACCGACTCATCCCGACTCATCGTTGTACATGGTTGTACATGAGCG | |
| CCAGCTAGCGCCAGCTCCGAATTGGTCCGAATTGGTGTTGATGAGGGTTGATGAGGAACTGGTTGGAACTGGTTGGAGACCCCAGGAGACCCCAGGGTGATCCACAGTGATCCACATCGTGGATGGTCGTGGAT | |
| GGCAGCCGCGAACAGCCGCGAAGAGGGCGGCGGAGGGCGGCGAGAATCTCCAAGAATCTCCAGGTCCGTGAAGGTCCGTGAACACTGCATCTCACTGCATCTAGTGCAGGCGAGTGCAGGCGTGGCCAGCAGTG | |
| GCCAGCAGTACGTGGGTGTACGTGGGTGGACTGCAGCAGACTGCAGCACGTCAGCTGCCGTCAGCTGCGTGCAGGCTGGTGCAGGCTGTTATGGTAGGTTATGGTAGGCCACGTCAGCCCACGTCAGCGTGGTA | |
| GTGAGTGGTAGTGATCCTCCAGCGTCCTCCAGCGTCAGCATGTATCAGCATGTATGTCACCATCTGTCACCATCGTGTCCACCGGTGTCCACCGGGATGCGGAAGGATGCGGAATTTCTTCAGCTTTCTTCAGC | |
| AGGTCCCGCTAGGTCCCGCTCCTGGAATATCCTGGAATATCATGTACATGCATGTACATGATGCAGGTGAATGCAGGTGAGTGAGCGGCCGTGAGCGGCCTCCAGCGTAATCCAGCGTAATCCGACACGCTCCG | |
| ACACGCAAAAGATGTTAAAAGATGTTCAGGCCCCACCAGGCCCCACTTGTTCAGGTTTGTTCAGGTTCTCCAGTTCTCTCCAGTTCTTGGGCCAGGTTGGGCCAGGAGCTCTTCTTAGCTCTTCTTGATCGGTC | |
| TTGATCGGTCTTCACCCCAAATCACCCCAAATCGGGGAATGTCGGGGAATGTTAGAGTTGTTTAGAGTTGTTCAGGCTGTTACAGGCTGTTACTATGCATCACTATGCATCAACTTTTTCAAACTTTTTCAACC | |
| CTGTGATTCCCTGTGATTTGGGACATGGTGGGACATGGGCTGTAAGTGGCTGTAAGTGTGGTACAGGGTGGTACAGGGGGCGGGGGCGGGCGGGGGCGGCTGGGAGGGGCTGGGAGGGTCTTGGTCGCTCTTGG | |
| TCGCGGCGCTTGCTGGCGCTTGCTGTTTTTCTCGGTTTTTCTCGTTCCTTCATCTTCCTTCATCGTGGGTGATGGTGGGTGATGGGATCTCCACGGATCTCCACTTCATTCTGTTTCATTCTGTTTGTCCAGGA | |
| TTGTCCAGGAATGTTGTGGAATGTTGTGGAAATGTACTCTAATGTACTCTGAGACCTGGTGAGACCTGGTTTCCGGACCTTTCCGGACCTGCTCATTTCTGCTCATTTCTGACAGGTGTGGACAGGTGTGTGAG | |
| CTCACGTGAGCTCACGGTTCAACATCGTTCAACATCCTTTTGAACTCTTTTGAACTGGTCCCACCAGGTCCCACCAGCCCACCAGCGCCCACCAGCCAAGGCTTAGCAAGGCTTAGAGCAGGTCTCAGCAGGTC | |
| TCGCAGAAGAAAGCAGAAGAAATCCACCAAGGTCCACCAAGGGCATCTTGGAGCATCTTGGAGACTTAGCCCCCATGATGCGGTCTGTCCATCTTCAGCAGGTCCCGCTCCTGAACTGGTTGGAGACCCCAGGC | |
| TTGTCCAGGAATGTTGTGGAATGTTGTGGAAATGTACTCTAATGTACTCTGAGACCTGGTGAGACCTGGTTTCCGGACCTTTCCGGACCTGCTCATTTCTGCTCATTTCTGACAGGTGTGGACAGGTGTGTGAG | |
| CTCACGTGAGCTCACGGTTCAACATCGTTCAACATCCTTTTGAACTCTTTTGAACTGGTCCCACCAGGTCCCACCAGCCCACCAGCGCCCACCAGCCAAGGCTTAGCAAGGCTTAGAGCAGGTCTCAGCAGGTC | |
| TCGCAGAAGAAAGCAGAAGAAATCCACCAAGGTCCACCAAGGGCATCTTGGAGCATCTTGGAGACTTAGCCCCCATGATGCGGTCTGTCCATCTTCAGCAGGTCCCGCTCCTGAACTGGTTGGAGACCCCAGGC | |
| CTGCAGCAGCTTGAAGCCCACGCTGAGGTTCTGGAAGATGTGGCCAGCACCATGTCTTCTTGGTCTCCACCATGGTCTTCAGCGGCTTGGTGGGGTTGCTGTCCACTGGCGGTACAGTGCTTGTCACACATGGG | |
| GTCCACTGGCGGTACAGCTTGTGCTTGTCACACATGGGGCTTTGTCCTCCAAAGTGTCCAACCATGATGCGGTCTGTCCACTTGGTGGGGTTGCTCAGAGAGTCAGTTCAAACTGAAGACCCCATTTGTTCATC | |
| TGCCCATGTCTCCCATTTTTTTTTTTTTTTTTTTCTTTTTTTTTCTTTTTTGAGATTTTTTGAGACAGTGTCTTGCAGTGTCTTGCTCTGTCAGCCTCTGTCAGCCCCAGGCTGGCCCAGGCTGGAGTGCAGTG | |
| GAGTGCAGTGGCATGATGTCGCATGATGTCGGCTCACTGCAGCTCACTGCAACCTCCACCTACCTCCACCTCCTGAATTCACCTGAATTCAAGTGATTCTCAGTGATTCTCCTGCCTCAGCCTGCCTCAGCCTC | |
| CCCAGTACTCCCCAGTAGCTGGGATTAGCTGGGATTACAGGCACCCGCAGGCACCCGCCACCATGCCCCACCATGCCCAGCCAATTTCAGCCAATTTTTGTATTTTTTTGTATTTTTAGTAGAGATGAGTAGAG | |
| ATGGGGTTTCACCGGGTTTCACCATGTTGGCCAATGTTGGCCAGGCTGGTCTCGGCTGGTCTCGAACTCCTAAGAACTCCTAACCTCAGGTGACCTCAGGTGATCCACCTGCCTCCACCTGCCTCAGCCTCCCT | |
| CAGCCTCCCAAAGTGCTGGAAAGTGCTGGGATTATAGGCGATTATAGGCATGGGCCACTATGGGCCACTGTGCTCGGCCGTGCTCGGCCTCAGAGCCCCTCAGAGCCCCGTCTCTTTCCGTCTCTTTCCTTTCC | |
| TTCTCTTTCCTTCTCTTTTCTTTTTTTTTCTTTTTATTTTTAGACATTTTTAGACAGGATCTTGCAGGATCTTGCTGTGTTGCCCTGTGTTGCCCAGGCTGGAGTAGGCTGGAGTGCAGTGATGCGCAGTGATG | |
| CAGTCATAGCTAGTCATAGCTCTCTTCAGCCCTCTTCAGCCTCCAACTCCTTCCAACTCCTGGGCTCAAGCGGGCTCAAGCGATCCCCTTTGATCCCCTTTGTCTCAACCTGTCTCAACCTTCTGAGTAGCTCT | |
| GAGTAGCTGGGATTCTCTGGGATTCTCAGGTGCACACAGGTGCACACCACCATGCCTCACCATGCCTGGCTAATTTTGGCTAATTTTTTTTTTCAGATTTTTTCAGAGATGGTGGGGGATGGTGGGGGTCTTGC | |
| TATGTCTTGCTATGTTGCCCAGGGTTGCCCAGGCTGGTCTCAACTGGTCTCAAACTCCTGAGCACTCCTGAGCTTAAGCAGTCTTAAGCAGTCCTCCCACCTCCTCCCACCTCAGCCTCCCAAAGCCTCCCAAA | |
| GTACCGGGAAGTACCGGGATTACAGGCATTTACAGGCATAAGCCACTATAAGCCACTATGCCTTGCCCAGCCTTGCCCAGCCCTTCTTTGCCCTTCTTTTCTGCTCCTCTCTGCTCCTCTTCCTGCCCCTTCCT | |
| GCCCCCTACCGTAGTCTACCGTAGTTTCAGAAACATTCAGAAACAAAACTGGGTAAAACTGGGTATGAGTGAAGCTGAGTGAAGCTTTGGTGCTGTTTGGTGCTGAAAATTTTCCAAAATTTTCCCCACTCACA | |
| TCCACTCACATTTCCATGCTCTTCCATGCTCTTGCAGAGAGTTGCAGAGAGCCGCTTGGTACCGCTTGGTAGAGGAAGACAGAGGAAGACAGGGAGATGCCGGGAGATGCCTTTGGGATGGTTTGGGATGGTCT | |
| CCTGACTTCTCCTGACTCCCCACCCTTCCCCACCCTTTGTGCAGGGCTGTGCAGGGCTACTACAGAGTACTACAGAGGCAGAAAGCTGCAGAAAGCTGGCCCGAAGTGGCCCGAAGTAGATGAGCAAAGATGAG | |
| CAATAAATATTTGTAAATATTTGATAAAGAAGGATAAAGAAGGAAATAATTAAAAATAATTAAGTGACAGATGGTGACAGATGTGACTCAAGATGACTCAAGAGTGACCACTGGTGACCACTGGAGAGGGTGGG | |
| AGAGGGTGGACTAGAGGCTACTAGAGGCTCCAGCAGACACCAGCAGACAGCACCTCTCCGCACCTCTCCTCACAGGGATTCACAGGGATAGAAGCCCAGAGAAGCCCAGGAGAAAGACAGAGAAAGACACCAGG | |
| GCATCCCAGGGCATCGTAAGAGGCTGTAAGAGGCTGCCCCTTAGAGCCCCTTAGAGAGCTCTTTTGAGCTCTTTTAGGCAAGTCTAGGCAAGTCTAGGGTCAGAGAGGGTCAGAGTGGACCCCAGTGGACCCCA | |
| GCCAGGTGCCTCCAGGTGCCTCCAATTAGACCCAATTAGACCCTGGGAGCCCCTGGGAGCCACCTATAACTACCTATAACTAAGAGCTTGAAAGAGCTTGATTGTCTCCCTTTGTCTCCCTAAATGGGTGGAAA | |
| TGGGTGGGAAAGTGAAGGAAAGTGAAGCAGGAGCCACCAGGAGCCACATGGAGCCTCATGGAGCCTCTTCCTGGAAATTCCTGGAAAGTCTGCCTGCGTCTGCCTGCCAAGAGCCAACAAGAGCCAAAGGGCTT | |
| TACAGGGCTTTACCATCCATTGCCATCCATTGCCCCTGCAGTTCCCTGCAGTTCACGCAGGGCCACGCAGGGCTGGCCCTAAGTGGCCCTAAGTCCTCTGGTTTCCTCTGGTTGTCGAGGGGTGTCGAGGGGTA | |
| AGTCCCCAGAAGTCCCCAGGGTCTGGGCCGGTCTGGGCCGGCTTCAGGGGGCTTCAGGGGACAGGAGTTGACAGGAGTTCAGTGTCAGGCAGTGTCAGGCAACTCCAAGCAACTCCAAGGCCTCTTTGGGCCTC | |
| TTTGGCTAAAGCTGTCTAAAGCTGTCTCTTCCCCCCTCTTCCCCCTCCTCTTCTTTCCTCTTCTTCCTCCTCATCCCTCCTCATCCTCTTCCTCTCTCTTCCTCTGCCTCCTCCAGCCTCCTCCAGAGTCAGTT | |
| CGAGTCAGTTCAAACTGGAATAAACTGGAATCTGTCAGGCCCTGTCAGGCCCGTCCCGCTCCGTCCCGCTCGGGGTTGGTGGGGGTTGGTGAGGTCTGAGGAGGTCTGAGGGGACTTCGGGGACTTCGGGGGAT | |
| CTTGCTCGATCTTGCTCTGGTACCACTTGGTACCACTCTCGATTGTCCTCGATTGTCCTCCAGCGTGCTCCAGCGTGTCCAGCAGGTTCCAGCAGGTCCTGTGCATCCCTGTGCATCTGGGTGGACCTGGGTGG | |
| ACCAGGTCAGCCCAGGTCAGCCCAAGTCTCCCAAAGTCTCCCACAGTGGGTGACAGTGGGTGAGCAATGTAGTGCAATGTAGTCAATGAAACCCAATGAAACCCACCTGGGACCACCTGGGACTTCTCCACTGT | |
| TCTCCACTGAGGCCGTATGAGGCCGTATGCTTGTCACACCTTGTCACACATGGGACTGAATGGGACTGATGTCCAGGCCTGTCCAGGCCCGACTCACGCCGACTCACGCTCGCGGTCTCTCGCGGTCTCCCTGC | |
| TGGAACCTGCTGGAAGAACTCGGCCGAACTCGGCCATGATGCGGTATGATGCGGTCCGTCCACTGCCGTCCACTGGCGGTACAGGGCGGTACAGGGGCAGCGGCTGGCAGCGGCTTGGTGGGGTTTGGTGGGGT | |
| GCTGGTTGGAGACCCCAGGATACCCCAGGATGGTCCACGTCGGTCCACGTCGTGGATGGCGGTGGATGGCGCTTGCAAAGACTTGCAAAGAGGGCAGCCAGGGGCAGCCAGGATTTCCAAGGATTTCCAAGTCT | |
| TGGTTCTCCAGCACCCTCCAGCACCGAGGCGTCGTGAGGCGTCGTTGTACATAAGTGTACATAAGCGCCACGTCTCGCCACGTCTGAGTTGGTGTGAGTTGGTGTTAATCAGAAATAATCAGAAACTGGTTGGA | |
| GCTGGTTGGAGACCCCAGGATACCCCAGGATGGTCCACGTCGGTCCACGTCGTGGATGGCGGTGGATGGCGCTTGCAAAGACTTGCAAAGAGGGCAGCCAGGGGCAGCCAGGATTTCCAAGGATTTCCAAGTCT | |
| GTGAACATCTGTGAACACAGCCTCGAGCAGCCTCGAGGGCGGGCGTAGGCGGGCGTAGCCAGCAGCAGCCAGCAGCACATGCGTGGACATGCGTGGACTGGGCCACGCTGGGCCACGTCGGCGGCATTCGGCGG | |
| CATGTAGGCTGTTGTAGGCTGTTGTGGTAGGCCGTGGTAGGCCACATTGGCGTACATTGGCGTGGTAGTGACCGGTAGTGACCCTCCAGCATCCTCCAGCATCAGCAGGTAGGAGCAGGTAGGTGGCCAGTGTT | |
| GGCCAGTGTGTCTGCTGGGGTCTGCTGGGATCTGGAATGATCTGGAATGTCTTCAGCAGTCTTCAGCAGGTCCCGCTCCGTCCCGCTCCTGAAAAATGCTGAAAAATGCTGAATATGATTGAATATGATAGCTC | |
| TGAGGAGCTGTGAGGGGCCGGTTCCGGCCGGTTCCCACTTACGTCCACTTACGTCCGCCACCTTGCGCCACCTTGAACACATCAAAACACATCAAGTCCCCACTTGTCCCCACTTGTTGGTGTCTGTTGGTGTC | |
| TTCTAGCTCCTTCTAGCTCCTTGGCCAGTTGTGGCCAGTTGCTCCTCCTGGCTCCTCCTGGTCAGTCTGGATCAGTCTGGACCCCAAAGCGCCCCAAAGCGTGGGACAGTGTGGGACAGTGGCTGAGGAGAGCT | |
| GAGGAGAGGCTGGCACTGGCTGGCACTGTGGCAGAGCGTGGCAGAGCCCATGTAGGCCCATGTAGGCCACTGATCCGCACTGATCCGGGACATGGGCGGACATGGGCTGTGGGGCCTTGTGGGGCCTCCTCAGC | |
| GGTCCTCAGCGGTCACCTTGGGCCACCTTGGGCAGCTCCACCTAGCTCCACCTCGGTCTGCTGCGGTCTGCTGGTCCAGGAAGGTCCAGGAAGGTCCGGGAGAGTCCGGGAGATGTACTCGGATGTACTCGGAC | |
| ACCTGGTTCCACCTGGTTCCCGGAGCGGCCCGGAGCGGCTGGTTTCGGATGGTTTCGGACAGGTGGGTCCAGGTGGGTCAACTCCCGGTAACTCCCGGTTCAGGATCCGTCAGGATCCGCTTGAACTTGCTTGA | |
| ACTTGTTGGAGGCCATTGGAGGCCATCTCCCCCACTCTCCCCCACCGAGTGCCGGCGAGTGCCGGGTCTGCAGCGGTCTGCAGCGTCTCCAACTGTCTCCAACTGATCCAGGCACATCCAGGCACCAGTCCAGC | |
| TCAGTCCAGCTCGTCTAGCGTCGTCTAGCGTCTCCAATGCCCTCCAATGCCAGCTTCTGCCAGCTTCTGCCCCGTGTCCTCCCGTGTCCTCTGCAGGAGGGTGCAGGAGGGAGCTGATTGCAGCTGATTGCTGG | |
| ATGAAGGTGGATGAAGGGTTTCCGACGGTTTCCGACGGGTCCCTGCTGGTCCCTGCTTGGCTGCTCCTGGCTGCTCCTAGGCATTGCTAGGCATTGCTGGCGGGCAATGGCGGGCAAGGGCCGCCACGGGCCGC | |
| CACGTTGCTCCGAGTTGCTCCGAACGGTCCGCAACGGTCCGCAGACTGGCCAGGACTGGCCAGGACCTGGGCAGACCTGGGCAAAGGGCGTCAAAGGGCGTCACAATCATGTCCAATCATGTCCTCTCCATGTC | |
| TCTCCATGTAGGTCGCTGGAGGTCGCTGGCCACAGAGGACCACAGAGGAGTTCCGAGACGTTCCGAGACATGGCCTTGGATGGCCTTGGGCGAGAGTTCGCGAGAGTTCATAGTCGCTAATAGTCGCTATCTGA | |
| GCGGTTCTGAGCGGTACAGGAAGGAACAGGAAGGACTCGCGCCGCCTCGCGCCGCTGGCTGTGCGTGGCTGTGCGGGACTGGAGCGGACTGGAGCCTGCATAATCCTGCATAATCCGGCCCAGGCCGGCCCAGG | |
| CCAGGGCTGGACAGGGCTGGACTGAGGGTCCCTGAGGGTCCAGGGCCCTCCAGGGCCCTCCTCCCACACGATCCCACACGAGAGCCCATTTGAGCCCATTTTCCAGGTCAATCCAGGTCAAAGCGCCTGCAAGC | |
| GCCTGCAGGAGGAAACGGGAGGAAACGGGCCAGGAGAGGCCAGGAGAGCCGCGACTTGCCGCGACTTCCTGACCTCCCCTGAGCTCCGGCCGCGGGCGGCCGCGGGCTCAGGTCCCTTCAGGTCCCTCTCGCGG | |
| CAGCTCGCGGCAGCCCGCGGACTCCCGCGGACTTGTCCGGATCTGTCCGGATCCGAATAGAAGCGAATAGAAGCGCTGTTGGACGCTGTTGGATGCGGATGGGTGCGGATGGGGCGCCGGGGTGCGCCGGGGTT | |
| GCCGCCACATGCCGCCACAGGTGCTTCGGGGTGCTTCGGGGCTCTGGTCGGCTCTGGTCATGCTGTGGCATGCTGTGGCGGCCGCGAGAGGCCGCGAGAGCGACTCAACGCGACTCAACCTGCTGCAAGCTGCT | |
| GCAAGCCTCTGCCCCCCTCTGCCCCTTCGCCGACCTTCGCCGACCCCCAGGTTCTCCCAGGTTCTCCATGCGCCACCATGCGCCAGAGAAAGGCTGAGAAAGGCTGGATGAAGGGGGATGAAGGGTTTCCGACG | |
| GTTTCCGACGGGTCCCTGCTTGTCCCTGCTTGGCTGCTCCTGGCTGCTCCTAGGCATTGCTAGGCATTGCTGGCGGGCAAGGGCGGGCAAGGGCCGCCACGGGCCGCCACGTTGCTCCGAATTGCTCCGAACGG | |
| TCCGCAGTCTTCAGCAGGTCCCGCTCCTGAACTGGTTGGAGACCCCAGGCCTGCAGCAGCTTGAAGCCCACGCTGAGGTTCTGGAAGATGTGGCCAGCACCATGTCTTCTTGGTCTCCACCATGGTCTTCAGCG | |
| GCTTGGTGGGGTTGCTGTCCACTGGCGGTACAGTGCTTGTCACACATGGGCTTGGTGGGGTTGCTCAGAGAGTCAGTTCAAACTGAACTTGTTGGAGGCCATCTCCGGTCCGTCCACTGGCGGTACAGTGCTTG | |
| TCACACATGGGGTCCACTGGCGGTACAGCTTGTGCTTGTCACACATGGGGCTTTGTCCTCCAAAGTGTCCAAAAGACCCCATTTGTTCATCTGCCCATGTCTCCCAAACTTGTTGGAGGCCATCTCCGGTCCGT | |
| CCACTGGCGGTACAGTGCTTGTCACACATGGGATATATTAGCTCTATTATGCTAGCTCTATTATGCTTTTAATATTATGCTTTTAAATTAGTGCTTTTAAATTAGTTGGTTTAAATTAGTTGGTTTCAAATGGT | |
| TGGTTTCAAATGATGTATTTCAAATGATGTATCCTGGATGATGTATCCTGGATCAGGCATCCTGGATCAGGCCTCTGGGATCAGGCCTCTGGAGCTTGGCCTCTGGAGCTTGAGATAAGGAGCTTGAGATAATT | |
| TCTCTGAGATAATTTCTCTCTCTCAATTTCTCTCTCTCTCTCTCTCCTCTCTCTCTCTCTCTCTCTCTCTCTCTCTCTCTCTCTCTCTCTCTCTCTCTCTCTCTCTCTCTCTCTCTCTCTATACTCTCTCTCTC | |
| ATACACACACTCTCATACACACACATACACACACACACATACACAATCTAACATACACAATCTACCTAGAACAATCTACCTAGAAAAAGATACCTAGAAAAAATCATTCGAAAAAAGATCATTCTAGAATGATC | |
| ATTCTAGAATGTAGGGTCTAGAATGTAGGGGGGTGAATGTAGGGGGGTGAGGAATTGGGGGTGAGGAATTTGTGGCGAGGAATTTGTGGCTCTCACTTTGTGGCTCTCACTTGCTAGCTCTCACTTGCTATAGA | |
| AAACTTGCTATAGAAAAGACAGTATAGAAAAGACAGTCTTGAAAAGACAGTCTTGAATCCAAAGTCTTGAATCCAAGTTCATGAATCCAAGTTCATGATTCAAAGTTCATGATTCACTCCAGATGATTCACTCC | |
| AGGCTGATCACTCCAGGCTGATGCAAAAAGGCTGATGCAAAATGCTTTATGCAAAATGCTTTCTTCCTAATGCTTTCTTCCTCAGATGTTCTTCCTCAGATGGTAGCTCTCAGATGGTAGCTGAGTGCTGGTAG | |
| CTGAGTGCTACAATCTGAGTGCTACAATTGGCAGGCTACAATTGGCAGCTTAAAATTGGCAGCTTAAACAGCCAAGCTTAAACAGCCAAACGGGAACAGCCAAACGGGCACAGCCAAACGGGCACAGCCAGAAG | |
| GGCACAGCCAGAAGTAAATAGCCAGAAGTAAATACAGCTGAGTAAATACAGCTGGACGTTTACAGCTGGACGTTAGCCTTTGGACGTTAGCCTTAAAAGCTTAGCCTTAAAAGCTGTCTGTTAAAAGCTGTCTG | |
| TTGTGAGCTGTCTGTTGTGAATGAAATGTTGTGAATGAAAAGTCTGGAATGAAAAGTCTGAGGTGAAAAGTCTGAGGTGAGTCAAGTGAGGTGAGTCAAGCAAATTGAGTCAAGCAAATTGCAAAGAGCAAATT | |
| GCAAAGATTGGCTTGCAAAGATTGGCAGGTGAAGATTGGCAGGTGAGGAGGTGCAGGTGAGGAGGTGCTACCGAGGAGGTGCTACCCTGTACGTGCTACCCTGTACGGCATGCCCTGTACGGCATGGGGAGAAC | |
| GGCATGGGGAGAAAAAACTGGGGAGAAAAAACATGGCAGAAAAAACATGGCAAAATGCACATGGCAAAATGCTTGGATCAAAATGCTTGGATGAGTCAGCTTGGATGAGTCAACTTCCATGAGTCAACTTCCCT | |
| GCTGCAACTTCCCTGCTGTAGGTGCCCTGCTGTAGGTGAGGCGATGTAGGTGAGGCGATTTGGATGAGGCGATTTGGATGAAGCGATTTGGATGAAGCATCCATGATGAAGCATCCATACTTTTGCATCCATAC | |
| TTTTAAGAGAATACTTTTAAGAGATACAAGTTAAGAGATACAAGACTGGTGATACAAGACTGGTGTGTCTAGACTGGTGTGTCTGGGTGTGTGTGTCTGGGTGTATTGCTCTGGGTGTATTGCTTCGCAGGTAT | |
| TGCTTCGCAGGTAAATCTTCGCAGGTAAATTGAGTGAGGTAAATTGAGTGTTGCCTATTGAGTGTTGCCTAAATTCTGTTGCCTAAATTCTGAACACTAAATTCTGAACACCTCTTTCTGAACACCTCTTAGCC | |
| AACACCTCTTAGCCAAGAGCCATTAGCCAAGAGCCAGCATCCAAGAGCCAGCATCCCTGTTCCAGCATCCCTGTTCTTTTCACCCTGTTCTTTTCACTGAGGTCTTTTCACTGAGGCACTGACACTGAGGCACT | |
| GAGGCATGGGCACTGAGGCATGTGTGAGGAGGCATGTGTGAGTTCATCTGTGTGAGTTCATCAGAGGAAGTTCATCAGAGGATGCCAATCAGAGGATGCCAAAGTGACGATGCCAAAGTGACAGTCAAAAAGTG | |
| ACAGTCAAAACACAACAGTCAAAACACAGAATCCAAAACACAGAATCCTCCAGGCAGAATCCTCCAGGGTCTCACCTCCAGGGTCTCAACTCCAGGGTCTCAACTCCAGGCTTCCAACTCCAGGCTTCTCTATA | |
| CAGGCTTCTCTATATAACAGTCTCTATATAACAGATGACTTATAACAGATGACTTCAATTAGATGACTTCAATTTCCGTCCTTCAATTTCCGTCTCACTGTTTCCGTCTCACTGGACCCATCTCACTGGACCCA | |
| GCTTTCTGGACCCAGCTTTCTCATAGCAGCTTTCTCATAGTTAGTGTCTCATAGTTAGTGGTTACAAGTTAGTGGTTACAAAGAGATGGTTACAAAGAGATCTTTGCAAAGAGATCTTTGATATTAGATCTTTG | |
| ATATTACTTTTTTGATATTACTTTTTGGTGCTTACTTTTTGGTGCTGGAATTTTGGTGCTGGAATTGGTGGACTGGAATTGGTGGAAACAACTTGGTGGAAACAACTTGATCGAAACAACTTGATCCATAAAAC | |
| TTGATCCATAAATGACATTCCATAAATGACATATTTTAAATGACATATTTTACAGATAATATTTTACAGATAAGCGAGTACAGATAAGCGAGAGAATTTAAGCGAGAGAATTAACAAGAGAGAATTAACAAGAT | |
| GAAGTTAACAAGATGAAGCAGATGAGATGAAGCAGATGGTTGCCAGCAGATGGTTGCCATAATCTGGTTGCCATAATCACAATGCCATAATCACAATGACAAACTCACAATGACAAACCACTCTTGACAAACCA | |
| CTCTCTCAAGACCACTCTCTCAAGGGCTTGCTCTCAAGGGCTTGTGTTCAAGGGCTTGTGTTCATCATTTTGTGTTCATCATTTCCCACACATCATTTCCCACATAAATATTCCCACATAAATAGGTTGGCATA | |
| AATAGGTTGGCTCCACTAGGTTGGCTCCACTCACTCGGCTCCACTCACTCCAGAGCACTCACTCCAGAGCCCTGCCTCCAGAGCCCTGCCTCTCTGGCCCTGCCTCTCTGCACATGCCTCTCTGCACATGGAGC | |
| TCTGCACATGGAGCTCACTGCTTGGAGCTCACTGCTGCTCTCTCACTGCTGCTCTCACTTGACTGCTCTCACTTGAACATCGTCACTTGAACATCGTACTTAGAACATCGTACTTAGAAAGACGTACTTAGAAA | |
| GATCATCATAGAAAGATCATCAATTATTGATCATCAATTATTGAGATGCAATTATTGAGATGAATGCATTGAGATGAATGCATTGGTCTGAATGCATTGGTCATCAATCATTGGTCATCAATTTTTCTTCATCA | |
| ATTTTTCTATCTGCATTTTTCTATCTGCAAATATCTATCTGCAAATATCCATTCGCAAATATCCATTCCTTGTAATCCATTCCTTGTATTGTGTTCCTTGTATTGTGTATTTTTTATTGTGTATTTTTACTTCA | |
| GTATTTTTACTTCATAATCATTACTTCATAATCAAAGCAACATAATCAAAGCAATGGATTCAAAGCAATGGATTGGAAAAAATGGATTGGAAAAGCAGACTTGGAAAAGCAGACACTGGTAAGCAGACACTGGT | |
| TTCTCCACACTGGTTTCTCCCATTGGGTTTCTCCCATTGGATAGAGCCCATTGGATAGAGAGACGAGGATAGAGAGACGAGTTCCTAGAGACGAGTTCCTTCAATCGAGTTCCTTCAATCTCTGCTCTTCAATC | |
| TCTGCTGTGACATCTCTGCTGTGACATTCAGTCTGTGACATTCAGTGGAGGACATTCAGTGGAGGATTTATTGTGGAGGATTTATTTGATCAGATTTATTTGATCAATGGCGTTTGATCAATGGCGTGAAGGCA | |
| ATGGCGTGAAGGGCAAACCGTGAAGGGCAAACAGCTGAGGGCAAACAGCTGATCAAAGACAGCTGATCAAAGGGCCTGGATCAAAGGGCCTGATAGCAAGGGCCTGATAGCAGAGTGCTGATAGCAGAGTGCTT | |
| GCTGCAGAGTGCTTGCTGGAGCCGGCTTGCTGGAGCCGTTAACATGGAGCCGTTAACAAGCACCCGTTAACAAGCACCGCAAGCCAAGCACCGCAAGCCAGTCACCGCAAGCCAGTCACGCCCTGCCAGTCACG | |
| CCCTTTGCTGCACGCCCTTTGCTGAGGATACTTTGCTGAGGATAAAAGTCTGAGGATAAAAGTCCTGGGATAAAAGTCCTGGGAAACCAGTCCTGGGAAACCAGCATGCGGAAACCAGCATGCGATATTTAGCA | |
| TGCGATATTTCTCCCCCGATATTTCTCCCCAGTGTGTTCTCCCCAGTGTGTCTTTGCCAGTGTGTCTTTGCTGTATTGTCTTTGCTGTATTCTTGGTGCTGTATTCTTGGCATTCTATTCTTGGCATTCTTCAG | |
| TCGGCATTCTTCAGTCCAAGAGCTTCAGTCCAAGAGCCATACTCCAAGAGCCATACCTATAGAGCCATACCTATAGTAGAGAACCTATAGTAGAGAAAATACAGTAGAGAAAATACTGCGTGGAAAATACTGCG | |
| TGTCCTCAACTGCGTGTCCTCAGGGGCATGTCCTCAGGGGCATCTGTGCAGGGGCATCTGTGCCAACACATCTGTGCCAACAAGCCAGTGCCAACAAGCCAGGTGCAGCAAGCCAGGTGCAGTGAAGGAGGTGC | |
| AGTGAAGGGAAACTAGTGAAGGGAAACTTGGTATGGGAAACTTGGTATGACCTTCTTGGTATGACCTTAAACGTATGACCTTAAACGTGAATAATTAAACGTGAATAATTGTCTGTGAATAATTGTCTTCTGTA | |
| AATTGTCTTCTGTAGTGTTTCTTCTGTAGTGTTTGTGGTGTAGTGTTTGTGGTGCAAGTTTTGTGGTGCAAGTTAAATTCTGCAAGTTAAATTCACAACTTTAAATTCACAACTGAGGTTTCACAACTGAGGTT | |
| CCAGGACTGAGGTTCCAGGAGACCCTTTCCAGGAGACCCTGGTGGGGAGACCCTGGTGGGGCATGACTGGTGGGGCATGAAGTTCAGGGCATGAAGTTCAGCAGAAGAAGTTCAGCAGAAGCCCAGCAGCAGAA | |
| GCCCAGCTGCTGAAGCCCAGCTGCTGGCCAGTAGCTGCTGGCCAGTAGTGAGTGGCCAGTAGTGAGTGGTCGGTAGTGAGTGGTCGTTCTGCAGTGGTCGTTCTGCAGGATGCGTTCTGCAGGATGGTCCGCGC | |
| AGGATGGTCCGCACACTTTGGTCCGCACACTTGCTGAAGCACACTTGCTGAAAAGCCTTTGCTGAAAAGCCTTTGTGGAAAAGCCTTTGTGGAGGATGCTTTGTGGAGGATGGTTACAGGAGGATGGTTACACA | |
| TTTGTGGTTACACATTTGCTTTCACACATTTGCTTTCAGTGATTTGCTTTCAGTGATTCTGGTTCAGTGATTCTGGTTTCATAGTTCTGGTTTCATAGTCATCTTTTCATAGTCATCTTCTTTTAGTCATCTTC | |
| TTTTGGAGCGCTTCTTTTGGAGCGTTTATTTTGGAGCGTTTATTTTCACTCGTTTATTTTCACTTGATATTTTTCACTTGATATTCTAGACTTGATATTCTAGATTAACAATTCTAGATTAACATTCCTTGATT | |
| AACATTCCTTTGCTCTCATTCCTTTGCTCTTGATCATTTGCTCTTGATCAGGATTTCTTGATCAGGATTTGGTTTCCAGGATTTGGTTTCCATTGTTTGGTTTCCATTGTAAAAGATCCATTGTAAAAGAACTT | |
| GAGTAAAAGAACTTGAGCCAAAGAACTTGAGCCAAACCAGTAGAGCCAAACCAGTAACTTTAAACCAGTAACTTTAATGGTGTAACTTTAATGGTGAAATTGTAATGGTGAAATTGACAGGTTGAAATTGACAG | |
| GTGGGAGATGACAGGTGGGAGAAGTGAAGTGGGAGAAGTGAAATCTTTGAAGTGAAATCTTTTCATCAAAATCTTTTCATCAGGAAGTTTTCATCAGGAAGTAAGTCACAGGAAGTAAGTCAGCTTGCGTAAGT | |
| CAGCTTGCAGTATCCAGCTTGCAGTATCTCAGTGGCAGTATCTCAGTGGCCCCCTCTCAGTGGCCCCCAAAAGGTGGCCCCCAAAAGGATGAGTCCAAAAGGATGAGTAATACAGGATGAGTAATACATGCGCC | |
| GTAATACATGCGCCACGATGCATGCGCCACGATGATCATACCACGATGATCATATCCTGTTGATCATATCCTGTCTACAGTATCCTGTCTACAGACGATCGTCTACAGACGATCCTCTTGAGACGATCCTCTTG | |
| TTCCGATCCTCTTGTTCCGACCAGTATGTTCCGACCAGTACTCAACGACCAGTACTCAACAGAAGATACTCAACAGAAGATGGCGAACAGAAGATGGCGAGGACCG | |
| GCCCAGCTGCTGAAGCCCAGCTGCTGGCCAGTAGCTGCTGGCCAGTAGTGAGTGGCCAGTGAGTGAGTGGTCGGTAGTGAGTGGTCGTTCTGCAGTGGTCGTTCTGCAGGATGCGTTCTGCAGGATGGTCCGCGC | |
| TTCTCCACACTGGTTTCTCCCATTGGGTTTCTCCCATTGGATAGAGCCCATTGGATAGAGAGACGAGGTAGAGAGACGAGTTCCTAGAGACGAGTTCCTTCAATCGAGTTCCTTCAATCTCTGCTCTTCAATC | |

In one preferred embodiment, the links between neighboring mononucleotides are phosphodiester links. In another preferred, at least one mononucleotide phosphodiester residue of the oligonucleotide(s) is substituted by a methylphosphonate, phosphotriester, phosphorothioate, phosphorodithioate, boranophosphate, formacetal, thioformacetal, thioether, carbonate, carbamate, sulfate, sulfonate, sulfamate, sulfonamide, sulfone, sulfite, sulfoxide, sulfide, hydroxylamiine, methylene(methylimino), methyleneoxy(methylimino), phosphoramidate residues, and combinations thereof The STA and MTA oligos having one or more phosphodiester residues substituted by one or more of the other residues are generally longer lasting, given that these residues are more resistant to hydrolysis than the phosphodiester residue. In some cases up to about 10%, about 30%, about 50%, about 75%, and even all phosphodiester residues may be substituted (100%). Some of the examples of oligonucleotide sequence fragments target the initiation codon of the respective gene, and in some cases adenosine is substituted with a universal base adenosine analogue denoted as “B”, which lacks ability to bind to the adenosine A₁ and/or A₃ receptors. In fact, such replacement nucleotide acts as a “spacer”. Many of the examples shown below provide one such sequence and many fragments overlapping the initiation codon, preferably wherein the number of nucleotides n is about 7, about 10, about 12, about 15, about 18, about 21 and up to about 28, about 35, about 40, about 50, about 60, about 70. In one embodiment, at least one of the targets for an MTA oligo encodes a protein such as interleukin-4 receptor and interleukin-5 receptor, among others listed in this patent. In a most preferred embodiment for use in the lung, the STA and MTA oligo(s) of this invention comprise(s) a desadenosine oligo(s), whether anti-sense to a naturally occurring desthymidine or desuridine sequence, or by substitution with one or more universal bases in accordance with the invention. The methods for substituting nucleotides, as well as for synthesizing oligos of a specific sequence, and what bases to employ as universal bases are known in the art, and need not be further provided here, since they are within the knowledge of an artisan. In a further embodiment of the composition of the invention, the STA and/or MTA oligo(s) is (are) operatively linked to an agent or molecule which, itself, is internalized or up-taken by living cells. In this manner, the uptake of the agent of the invention is enhanced as is known in the art. Examples of agents or molecules suitable for use with the STA and MTA oligos of this invention are vectors, transferrin, asialoglycoprotein, and streptavidin. Others, however, are also suitable.

Although no claim is made as to any specific mechanism of action, the oligonucleotide of the invention is effective to reduce expression of a target gene or mRNA. It is believed to pass through a cell membrane and bind specifically to target gene or mRNA in the cell so as to prevent its translation. However, the gene product may be expressed on the cell membrane. Such oligo(s) may be provided as a composition with a suitable pharmaceutically or veterinarily acceptable carrier, e.g. sterile pyrogen-free saline solution, etc. The composition of the invention is also provided as a formulation with a hydrophobic carrier capable of passing through a cell membrane, e.g. with a surfactant or in a liposome(s), with the liposome(s) carried in a pharmaceutically or veterinarily acceptable aqueous carrier. The oligonucleotides may be coupled to an agent that transports them into the cell and/or inactivates mRNA, such as a ribozyme, or a vector. Such oligonucleotides may be administered to a subject in need of treatment to inhibit the activation of specific receptors, enzymes and/or proteins and/or factors, among other expression products. The formulation may also have chimeric molecules comprising oligo(s) attached to a molecule(s) that is (are) known to be internalized by cells. These conjugates utilize cellular up-take pathways to increase intracellular concentrations of the oligo. Examples of molecules used are macromolecules including eukaryotic vectors, transferrin, asialoglycoprotein (e.g. bound to oligonucleotides via polylysine), sperimine and streptavidin, among others. An inhibitor of the identified genes in this invention may be administered to a subject for the prevention or treatment of bronchoconstriction, airway inflammation and/or respiratory diseases in general. Examples of the inhibitor are those that inhibit the expression or function of the genes, e.g. dansylcadaverin, glycinamide, methylamine, n-propylamine, n-hexylamine, bacitracin, ethylamine, t-butylamine, an antibody and an oligonucleotide, among others. Chemical compounds can be prepared according to known procedures. See, Chuang, D M, J. Biol. Chem, 256:8291-8293 (1981). Other chemical compounds not disclosed in this patent may be used as long as they have a gene inhibitory activity. An antibody to an expressed gene product having inhibitory activity to the antigen may be prepared using conventional methods and comprises murine, primatized, humanized, human and chimeric antibodies. A molecule of structurally altered antibodies, for example, a single chain Fv, or a diabody, is also included in the meaning of the antibody in this invention. Once its antigen is known, it is conventional to prepare an antibody thereto and an artisan will know how to. The sequence information for protein preparation is shown in SEQ ID NOS: 1 to 12. An anti-sense oligo may be prepared using the method described in this patent.

This invention also provides a method for screening candidate compounds useful for the prevention and/or treatment of respiratory or lung diseases that binds to or inhibits formation of one or more gene(s), ETS(s), cDNA(s), mRNA(s), or gene product(s). At least one gene(s), mRNA(s), or gene product(s) may be G-alphaH, and/or other related genes, mRNAs, etc. listed in Table 1 above. Samples suspected of containing a candidate compound(s) that bind(s) to or inhibit(s) the formation of one or more of gene(s), mRNA(s), or gene product(s) are subject to the screening. Samples may be obtained from any biological source and are contacted with the protein under appropriate conditions. The genes, EST(s), cDNA(s), mRNAs, and gene products may be provided in purified form, isolated, in solution, suspension or dry form. They may also be provided in unpurified form. The genes, ESTs, cDNA, mRNAs, and gene products may be derived from an expression system, cells, tissues, plants, animals, and cell-free systems, and may be directly isolated, syntesized or used to screen candidate compounds that bind to a gene(s), EST(s), cDNA(s), mRNA(s), or gene product(s). A construct is also encompassed comprising one or more gene(s), EST(s), cDNA(s), or mRNA(s), and may be linked to a reporter gene system, e.g. for introducing a cell to express a mRNA(s), or gene product(s). These may be applied to screening and identifying candidate compounds. The gene(s), EST(s), cDNA(s), mRNA(s), or gene product(s) is (are) preferably expressed on the cell membrane. However, they may be expressed inside a cell and be exposed on the membrane or remain inside the cell. In the screening process a candidate compound may be contacted in a culture medium with cells and the binding of a candidate compound to a protein monitored and detected using known methods. This screening system may be constructed using sequence information on the proteins shown in Table 1 or known in the art. For ease of detection, the gene(s), EST(s), cDNA(s), mRNA(s), or gene product(s) may be provided in fused form, e.g. chimeric gene(s), hybrid RNA(s), fusion protein(s), etc. with other gene(s), EST(s), cDNA(s), mRNA(s), and/or gene product(s). A biological sample for use in screening may contain a candidate compound binding to a gene(s), EST(s), cDNA(s), mRNA(s), or gene product(s). Candidate compounds may be inhibitors, agonist, antagonist or reverse agonist of a target etc. The preparation of samples is not limited to biological sources. Natural compounds and libraries of synthetic compounds are known, and are suitable for this purpose. All inhibitors of a target associated with a respiratory or lung disease found by this method are useful for the prevention and/or treatment for such diseases. Marker labels may be used in these assays, such as enzymes and combinations of enzymes and proteins, for example, luciferase, or a combination of alkaline phosphatase and horse shoe crab peroxidase, fluorescent and phosphorescent labels, radio labels, etc. The detected labels may be compared to controls, and compounds showing statistically significant differences are selected to determine a desired candidate compound.

This method may be applied to the screening of a candidate compound(s) suitable for the prevention and/or treatment for respiratory and lung diseases which alters or suppresses the expression, characteristic, or function of a gene(s), EST(s), cDNA(s), mRNA(s), or gene product(s) associated with the disease. The gene(s), EST(s), cDNA(s), mRNA(s), and gene product(s) may be selected from respiratory genes shown in Table 1, or their combination with one or more thereof or with other similar molecules corresponding to other genes. Samples containing a candidate compound suspected of inhibiting one or more gene(s), EST(s), cDNA(s), mRNA(s), or gene product(s) may be subject to screening. The samples may be contacted with an expression system of a gene(s), EST(s), cDNA(s), mRNA(s), or gene product(s) under appropriate conditions as described here. The gene(s), EST(s), cDNA(s), mRNA(s), or gene product(s), in addition, may be introduced into and expressed in cells, and the cells may be used for screening candidate compounds. The inhibition of gene expression may be determined by measuring the levels or activities of gene(s), EST(s), cDNA(s), mRNA(s), or gene product(s). Transcripts from genes and cDNAs may be prepared and a regular northern blotting test employed to quantitatively assess their levels, other assays, however, may also be employed. The gene(s), EST(s), cDNA(s), or mRNA(s) may be provided as is, or in a form to be operatively linked to a reporter gene system, and the detection system may rely on a signal from the reporter gene system, executed by conventional methods. The nucleic acids may be labelled as described earlier to obtain information on the transcripts. Candidate compounds may be contacted in a culturing medium with the cells and the inhibition of expression gene may be detected using known methods. The amounts of detected label may be compared with a control, and the candidate compounds ranked based on their levels of gene expression and a cut-off value specified, to select a “hit” or “lead” compound(s). An example of the screening system and process of this invention is shown in Example 10 below.

The composition may be provided also as a pharmaceutical formulation with a surfactant, a non lipid surfactant and/or within lipid particle or vesicle, such as a liposome or microcrystal. The particles may be of any suitable structure, such as unilamellar or plurilamellar. The one preferred embodiment, the oligonucleotide is comprised within the liposome. Positively charged lipids such as N-[1-(2,3-dioleoyloxy) propyl]-N,N,N-trimethylammoniumethylsulfate, or “DOTAP,” are particularly preferred for such particles and vesicles. However, others are also suitable. The preparation of such lipid particles is well known. See, e.g., U.S. Pat. Nos. 4,880,635 to Janoff et al., U.S. Pat. No. 4,906,477 to Kurono et al., U.S. Pat. No. 4,911,928 to Wallach, U.S. Pat. No. 4,917,951 to Wallach, U.S. Pat. No. 4,920,016 to Allen et al., U.S. Pat. No. 4,921,757 to Wheatley et al., the relevant sections of all of which are herein incorporated in their entireties by reference. The composition of the invention may be administered to the airways of a patient by any suitable means, but is preferably administered through the respiratory system as a respirable, inhalable, nasal or instillable formulation, more preferably in the form of an aerosol or spray comprising respirable particles that include the agent for respiration, lung instillation or inhalation by the subject. The respirable particles may be in gaseous, liquid or solid form, and they may, optionally, contain other therapeutic ingredients and formulation components. The particles of the present invention are preferably particles of respirable size, preferably of a size sufficiently small to pass, upon inhalation, through the mouth and larynx and into the bronchi and alveoli of the lungs. In general, particles ranging from about 0.5 to about 10 microns (μm) in diameter are optimal for absorption through inhalation. Other sizes, however, may also be suitable, and preferred particles are about 0.5, about 0.8, about 1.0 to about 3, about 4, about 5 micron. Particles of non-respirable size are of considerably larger diameter, and when included in a formulation tend to deposit in the throat and may be swallowed. Accordingly, it is desirable to minimize the quantity of non-respirable particles in the aerosol. For nasal administration, a particle size in the range of about 10 micron to about 500 micron is adequate, and preferred is about 10, about 12, about 15 to about 20, about 25, about 35, about 50 micron, to ensure their retention in the nasal cavity.

Liquid compositions of the invention for producing a respirable formulation, e.g. an aerosol or spray may be prepared by combining the oligo with a suitable vehicle or carrier, such as sterile pyrogen-free water and/or other known pharmaceutically or veterinarily acceptable carrier. Other therapeutic compounds may be included as well as other formulation ingredients as is known in the art. Solid particulate compositions comprising respirable dry particles of, e.g. the micronized agent of the invention may be prepared by grinding the dry composition with a mortar and pestle, and then passing the thus ground, e.g. micronized composition through a screen, e.g. 400 mesh screen, to break up or separate large agglomerates of particles. A solid particulate composition comprising the composition may optionally also comprise a dispersant and other known agents, which serve to facilitate the formation of a mist or aerosol. A suitable dispersant is lactose, which may be blended with the composition in any suitable ratio, about 1:1 w/w. Other ratios and other dispersants may be utilized as well, as may other therapeutic and formulation agents. Aerosols of liquid particles comprising the agent may be produced by any suitable means, such as with an insufflator or nebulizer. See, e.g., U.S. Pat. No. 4,501,729. Nebulizers are commercially available devices which transform solutions or suspensions of an agent into a therapeutic aerosol mist either by means of acceleration of a compressed gas, typically air or oxygen, e.g. through a narrow venturi orifice or by means of ultrasonic agitation. Suitable formulations for use in insulators and nebulizers comprise the present agent, the agent of this invention, in an amount of about 0.01 to about 40%, preferably about 1% to less than 20% w/w in a liquid carrier which is typically water or a dilute aqueous alcoholic solution, preferably made isotonic with body fluids by the addition of, for example, sodium chloride. Other carriers and other proportions, however, are also suitable. Optional additives include preservatives if the formulation is not prepared sterile, for example, methyl hydroxybenzoate, antioxidants, flavoring agents, volatile oils, buffering agents and surfactants, among others.

The compositions provided herein comprise nucleic acid(s) comprising the oligo(s) described above and one or more surfactants. Suitable surfactants or surfactant components for enhancing the uptake of the oligos of the invention include synthetic and natural as well as full and truncated forms of lipid and non-lipid surfactants, such as surfactant proteins A, B, C, D and E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; natural and artificial lamelar bodies and liposomes as vehicles for the surfactants, omega-3 fatty acids, polyenic acid, polyenoic acid, lecithin, palmitinic acid, copolymers of ethylene or propylene oxides, polyoxypropylene, monomeric and polymeric polyoxyethylene, monomeric- and polymeric- poly(vinylamine) with dextran and/or alkanoyl side chains, Brij 35®, Triton X-100®, and synthetic surfactants ALEC®, Exosurf®, Survan®, and Atovaquone®, among others. Preferred are non-lipid and non-phosphate lipid surfactants, amongst others. The surfactants may be used either as single or part of a multiple component surfactant in a formulation, or they may be covalently bound to the 5′- and/or 3′- ends of the oligos. Although varying ranges of surfactant amounts may be added to the composition, our preferred range is about 0.001 to about 30%. Other preferred amounts are about 0.01, about 0.1, about 1, about 5, about 10, about 15 to about 15, about 20, to about 25, about 30, about 35, about 40, about 50% w/w composition. Although variable amounts of surfactant may be added, it should be understood that one preferred amount is greater than those employed as formulation ingredient. Another preferred amount is less than what would form substantial amounts of surfactant nucleic acid complexes and/or liposomes.

The composition of the invention may be administered by any means that transport the oligo and the surfactant composition to the nasal cavities and/or the lung. The composition may be administered to the respiratory tract or instilled into the lungs by any suitable means, but is preferably administered by inhalation or nasal administration of an aerosol or spray comprised of respirable or instillable particles. The respirable particles may be liquid or solid, and they may optionally contain surfactant and other therapeutic or diagnostic ingredients as well as other typical formulation ingredient. Examples of other agents are analgesics such as Acetaminophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics, among others. See, Mosby's Physician's GenRx. Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti-anxiety agents associated with mental depression, such as Chlordiazepoxide, Amitriptyline, Loxapine Maprotiline and Perphenazine, among others. Anti-inflammatory agents such as non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Sedatives including Diphenhydramine, Hydroxyzine, Methotrimeprazine, Prometazine, Propofol, Melatonin, Trimeprazine, and the like. Sedatives and agents used for treatment of petit mal and tremors, among other conditions, such as Amitriptyline HCl; Chlordiazepoxide, Amobarbital; Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g. for treatment of severe head injury; orphan status, Warner Lambert), cytoprotective agents, and agents for the treatment of menopause, menopausal symptoms (treatment), e.g. Ergotamine, Belladonna Alkaloids and Phenobarbital, for the treatment of menopausal vasomotor symptoms, e.g. Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre-menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric treatments such as Tricyclic Antidepressants, including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications.

The composition may be administered into the respiratory system as a formulation including particles of respirable size, e.g. particles of a size sufficiently small to pass through the nose, mouth and larynx upon inhalation and through the bronchi and alveoli of the lungs. The figures provided here refer to a substantial number of particles of such size and/or to an average diameter. In general, respirable particles range from about 0.5, about 1, about 1.5, about 2 to about 5, about 7, about 8, about 10 micron, and preferably about 0.5 to about 5 micron in size. Particles of non-respirable size that are included in the aerosol tend to deposit in the throat and be swallowed, and the quantity of non-respirable particles in the aerosol is thus minimized. For nasal administration and pulmonary instillation, the particle size may be in the range of about 10, about 12, about 15, about 20 to about 30, about 40, about 50, about 60, about 100, about 500 micron, and about 10 to about 50 micron is more preferred to ensure retention in the nasal cavity. Aerosols, sprays, or mists of solid particles of the composition may be produced with any device that generates solid particulate medicament aerosols or mists whether solid powdered or from liquid source. Aerosol and mist generators are suitable for administering solid particulate medicaments. These devices whether solid powdered or from a liquid source produce respirable particles, as explained above, and generate a volume of aerosol or mist containing a predetermined metered dose of a medicament at a rate suitable for human or animal administration. One illustrative type of solid particulate aerosol generator is an insufflator. Suitable formulations for administration by insufflation include finely comminuted powders that may be delivered by means of an insufflator or taken into the nasal cavity in the manner of a snuff. In the insufflator, the powder, e.g. a metered dose of the agent effective to carry out the treatments described herein, is contained in a capsule or a cartridge. These capsules or cartridges are typically made of gelatin or plastic, and may be pierced or opened in situ, and the powder delivered by air drawn through the device upon inhalation or by means of a manually-operated pump. The powder employed in the insufflator may consist either solely of the agent or of a powder blend comprising the agent, a suitable powder diluent, such as lactose, and an optional surfactant as well as other agents. The agent typically comprises from about 0.01% to about 100% w/w of the formulation. A second type of illustrative aerosol generator comprises a metered dose inhaler. Metered dose inhalers are pressurized aerosol dispensers, typically comprising a suspension or solution formulation of the active ingredient in a liquified propellant. During use these devices discharge the formulation through a valve adapted to deliver a metered volume, typically about 10 to about 150 μl, although other volumes are also suitable, to produce a fine particle spray containing the active ingredient. Suitable propellants include solvents such as certain chlorofluorocarbon compounds, for example, dichlorodifluoromethane, trichlorofluoromethane, dichlorotetrafluoroethane and/or mixtures thereof. The formulation may additionally comprise one or more co-solvents, for example, ethanol, surfactants, such as oleic acid or sorbitan trioleate, antioxidants and suitable flavoring agents. The aerosol, whether formed from solid or liquid particles, may be produced by the aerosol generator at a rate of from about 10 to about 150 liters per minute, more preferably from about 30 to about 150 liters per minute, and most preferably about 60 liters per minute. Aerosols containing greater amounts of medicament may be administered more rapidly. Instillation devices are known in the art, and are suitable for direct delivery to the lungs.

As already indicated, the composition of this invention is also provided as a pharmaceutical composition, comprising the composition of the invention, and a carrier. The carrier is preferably a biologically acceptable carrier, and more preferably a pharmaceutically or veterinarily acceptable carrier in the form of a gaseous, liquid, solid carriers, and mixtures thereof, which are suitable for the different routes of administration intended. The composition may optionally comprise other agents such as other therapeutic compounds known in the art for the treatment of the condition or disease, antioxidants, flavoring and coloring agents, fillers, volatile oils, buffering agents, dispersants, surfactants, RNA inactivating agents, antioxidants, flavoring agents, propellants and preservatives, as well as other agents known to be utilized in therapeutic compositions. An example of the mRNA inactivating agent is an enzyme, such as ribozyme.

The composition generally contains the oligonucleotide in an amount of about 0.01% to about 99.99% w/w, preferably about 1% to about 40% w/w, and more preferably about 5% to about 20% w/w of the composition. However, other ingredients, and other amounts of the agent are also suitable within the confines of this invention. The composition of the invention is also provided in various formulations that are tailored for different methods of administration and routes of delivery. The formulations that are contemplated are, for example, a transdermal formulation also containing carrier(s) and other agents suitable for delivery through the skin, mouth, nose, vagina, anus, eyes, ears, and other body cavities, intradermally, as a sustained release formulation, intracranial, intrathecally, intravascularly, by inhalation, intrapulmonarily, into an organ, by implantation, including suppositories, cremes, gels, and the like, as is known in the art. In one particular formulation, the agent is suspended or dissolved in a solvent. In another, the carrier comprises a hydrophobic carrier, such as lipid particles or vesicles, including liposomes and micro crystals. The preparation of all of these formulations, as well as the ingredients to be utilized, are known in the art, and need not be further described here. In one particularly embodiment of the vesicle formulation, the vesicles comprise lipid complexes or liposomes containing the oligonucleotide, that may comprise for example N-(1-[2,3-dioleoxyloxy]propyl)-N,N,N-trimethyl-ammonium methylsulfate as well as other lipids known in the art to provide suitable delivery of DNA to target cells. In one embodiment, this formulation is a respirable formulation, such as an aerosol or spray. The composition and formulations of the invention are provided in bulk, and in unit form as well as in the form of an implant, a solution, suspension, or emulsion, in a capsule or cartridge, which may be openable or piercable, and others known in the art.

A kit is also provided, which comprises a delivery device, and in separate containers, the agent, composition or formulation of the invention, and optionally other agents, and instructions for the use of the kit components. In one preferred embodiment, the delivery device comprises a nebulizer which delivers single or multiple metered doses of the formulation. The single metered dose nebulizer may be provided as a disposable kit which is sterilely preloaded with enough agent for one application. The nebulizer may be provided as an insufflator, and the composition in a piercable or openable capsule or cartridge. In a different embodiment, the delivery device comprises a pressurized inhaler, and the agent is in the form of a suspension or solution. The kit may optionally also comprise in a separate container an agent selected from the group consisting of other therapeutic compounds, antioxidants, flavoring and coloring agents, fillers, volatile oils, buffering agents, dispersants, surfactants, cell internalized or up taken agents, RNA inactivating agents, antioxidants, flavoring agents, propellants and preservatives, among other suitable additives for the different formulations. When a solvent for the agent or the other ingredients is added, organic solvents and organic solvents mixed with one or more co-solvents may be utilized as well as aqueous solvents as is known in the art. The composition of the invention may be provided in conjunction with a vector for delivery purposes, or for manufacturing copies thereof. The agent may be operatively linked to the vector as is known in the art. The agent may also be provided within a host cell for amplification of the MTA oligo, and for storage purposes.

The agent of this invention may be utilized by itself or in the form of a composition or various formulations in the treatment of a disease or condition associated with the mRNA corresponding to at least one target gene(s), to genomic flanking regions, initiation codon, intron-exon borders and the like, or the entire sequence of precursor RNAs, including non-coding RNA segments, the 5′-end and the 3′-end, e.g. poly-A segment and oligos targeted to the juxta-section between coding and non-coding regions, and RNA regions encoding proteins, by administration to a subject afflicted with the disease or condition of an amount of the oligonucleotide effective to reduce the production or availability, or to increase the degradation by the subject of at least one of the target mRNAs. Typically, the agent is administered in an amount effective to reduce the production or availability, or to increase the degradation of one or more, typically at least two of the target mRNAs. Optionally, the agent is administered directly to the lung(s) of the subject as a respirable aerosol or spray. An artisan will know how to titrate the amount of agent to be administered by the weight of the subject being treated in accordance with the teachings of this patent. The agent, however, is preferably administered in an amount effective to attain an intracellular concentration of about 0.05 to about 10 μM single or multi-targeted anti-sense oligonucleotide, preferably in an amount effective to attain an intracellular concentration of about 0.1 to up to about 5 μM MTA oligonucleotide. This invention is suitable for treating numerous respiratory and lung diseases and conditions and its application is solely limited by the availability of target molecules and their sequences. Examples of diseases and conditions for which this technology is particularly well suited are lung function impairing diseases or conditions, such as those associated with obstruction of the subject's airways, with asthma, etc. One of the preferred target proteins comprises interleukin-4 receptor, although various others described above, among many more, are also suitable. Depending on the target organ or tissue, the agents of this invention may be utilized by itself or in the form of a composition or various formulations in the treatment of a respiratory disease or condition associated with it. The agent(s) and composition of the invention may be delivered in one of many ways, for example by a topical or systemic route, and more specifically orally, intracavitarily, intranasally, intraanally, intravaginally, transdermally, intrabucally, intrapulmonarily, intravenously, subcutaneously, intramuscularly, intratumorously, into a gland, by inhalation, by instillation, by implantation, intradermally, and many other routes of administration. The formulation may be, in addition, an implant, slow release, transdermal release, sustained release, and/or coated with macromolecule(s) to avoid destruction of the agent prior to reaching the selected target. The subjects treated by the present agents include humans and other animals in general, and in particular vertebrates, and amongst these mammals, and more specifically humans and small and large, wild and domesticated, marine and farm animals, preferably humans and domesticated and farm animals. In one aspect of the invention, at least one of the target mRNAs and the subject are of the same species, and in a preferred case they are of human origin. However, since in one embodiment mismatched nucleotides are replaced, mismatched species may also be utilized.

The STA or MTA oligonucleotide of this invention may be administered in a broad dose range. Preferable is an amount of about 0.005 to about 150 mg/kg body weight per administration, and the agent may be administered once (acute treatment) to several doses per day, or as continuous administration to maintain the level of a specific molecule. Preferred doses are about 0.01, about 0.1, about 1.0 to about 50, about 65, about 75 mg/kg body weight, more preferably about 1 to 50 mg/kg body weight. The method may be administered as a prophylactic or therapeutic method. The composition of the invention may be produced by selecting one target, or in the case of the MTA oligonucleotides two or more targets selected from the group consisting of genes, genomic flanking regions, mRNAs and proteins known to be associated with at least one disease or condition; obtaining RNAs selected from the group consisting of RNAs corresponding to the genes, to genomic flanking regions, initiation codon, intron-exon borders and the like, or the entire sequence of RNAs, including non-coding RNA segments, the 5′-end and the 3′-end, e.g. the poly-A segment and oligos targeted to the juxta-section between coding and non-coding regions, and RNA segments encoding the target proteins; selecting a segment of a first RNA which is at least about 60% homologous to a segment of at least a segment of a second RNA; and synthesizing one or more oligonucleotide(s) to the one or more RNA segments. In one preferred embodiment, the method further comprises substituting a universal base for at least one, and in some instances all of it, non-homologous nucleotide in the oligonucleotide, and in another preferred embodiment the method further comprises substituting a methylated cytosine for cytosine in at least one CpG dinucleotide present in the oligonucleotide. The technology involved in methylation is known in the art and need not be further described here. Although the specific length of the STA or MTA oligo is determined by the target's length, and its segments containing few thymidines, the oligo(s) are preferably greater than about 7 nucleotides long, and up to about 60 nucleotides long, and longer. The specific backbone chemistry may be selected by an artisan based on the teachings provided here and the knowledge of the art at large. One factor that impinges on the selection of the nucleotide bridging residues is the level of nuclease resistance desired and other factors specific to one or the other method of administration. Another factor is the need for localization of the treatment, to minimize or fully avoid side effects which might otherwise be caused along with the therapeutic effect of the agent.

The following examples are provided to illustrate the present invention, and should not be construed as limiting thereon.

### EXAMPLES

In the following examples μM means micromolar, mM means milimolar, ml means milliliters, μm or microns means micrometers, mm means millimeters, cm means centimeters, ° C. means degrees Celsius, μg means micrograms, mg means milligrams, g means grams, kg means kilograms, M means molar, and h means hours.

### Example 1

Design and Synthesis of Oligonucleotides

Anti-sense oligonucleotides, each 16-20 nucleotides in length, are designed targeting the mRNA sequences, including 5′ and 3′ non-translation sequences, of interleukin-4 receptor, interleukin-5 receptor, chemokine receptors CCR1 and CCR3, chemokines Eotaxin-1, RANTES and MCP4, CD23, ICAM, VCAM, tryptase a and b, PDE4 (A, B, C, D subtypes). The oligonucleotides are synthesized to have phosphorothioate backbones using an Applied Biosystems 394 synthesizer (Perkin Elmer, Calif.). The sequences of oligonucleotides for each of the 6 genes are given in the sequence listing.

### Example 2

Real-Time PCR (Taqman) Analysis of Gene Expression

The RT-PCR was performed with 100 ng of total RNA using Taqman Reverse Transcription Reagents (Applied Biosystems) in Taqman 96-well plates. Each well contained 1 ul of 1× TaqMan RT Buffer, 2.2 ul of 25 mM Magnesium chloride, 0.5 ul of Random hexamers, 0.2 ul of Rnase Inhibitor and 0.25 ul of MultiScribe Reverse Transcriptase. The final volume was 10 ul. The mixture were incubated at 25 C for 10 minutes, at 48 C for 25 minutes and at 95 C for 5 minutes.

The Taqman assay was performed using gene specific and human GAPDH primers and probes The human GAPDH RNA expression was used for data normalization. The RT-PCR plates containing 10 ul of cDNA as described in RT-PCR protocol was used in a Taqman multiplex assay. To each well of microtiter plates, 12.5 ul of 2× Taqman Universal Master Mix (PE Biosystems), 0.25 ul of 10 uM forward gene specific primer, 0.25 ul of 10 uM gene specific reverse primer, 0.25 ul 20 uM gene specific probe and 1.25 ul of human GAPDH primers and probes mixture and 0.5 ul of dH2O were added. The total volume in each well was 25 ul. The real time PCR was formed in total of 40 cycles using ABI Prism 7700 or 9700 Sequence Detector.

### Example 3

CD23

A library of 213 phosophorothioate antisense oligonucleotides against CD 23 was screened using U-937 cell line (ATCC, cat. #CRL-1593.2). U937 at 2×10⁵ cells/well were transfected with 0.8 uM oligonucleotide/DOTAP (Roche, Indianopolis, Ind.) at lipid:AS ratio of 5:1 in a serum-free medium for 4 hours in a final volume of 65 ul in 96 flat bottom plates. After 4 hour of tranfection, 235 ul of RPMI medium containing 10% fetal calf serum (FBS) were added to each well. In some experiments 15 ng/ml of human IL-4 were added to the medium. The cells were harvested 16 hours post-transfection and RNA was isolated using BioRobot 3000 (Qiagen, Valencia, Calif.). Real time PCR was used to determine the RNA expression.

Twenty hours post-transfection, cells were harvested into 96-V bottom plates and were washed once with cold PBS containing 2% FBS and 0.1 % sodium azide (FACS buffer). Human CD23-phycoerythrin (PE)-labeled and PE labeled isotype control antibody (both from Pharmingen, SanDiego, Calif.) in 70 micro liter of the buffer were added to each well and were incubated for 30 minutes at 4 C in the dark . Cells were then washed three times with 0.3 ml of FACS buffer and resuspended in 0.3 ml of 1% paraformaldehyde in PBS. Cells were analyzed on a FACSCalibur (Becton and Dickinson, Moutain view, Calif.).

Treatment of U-937 cells with AS CD61,455-XO4772 resulted in 62%+16 inhibition of RNA expression and 30% of protein inhibition. RNA inhibition is expressed as percentage of inhibition of CD23 RNA in CD23 AS treated cells as compared to the cells that were incubated with a nonspecific oligonucleotide (20 mer wobble). The protein inhibition is expressed as percentage of protein expression in CD23 AS treated cells as compared to the control oligonucleotide treated cells (wobble) based on geomean fluorescence intensity, subtracting basal expression.

### Example 4

Interleukin-5 Receptor

A library of 160 phosophorothioate antisense oligonucleotides against IL-5R was screened using TF-1 cell line (ATTC cat.# CRL-2003). In a 96-V bottom plate, TF-1 at 1.5×10⁵ cells/well were transfected with 0.8 uM oligonucleotide/CellFectin (Invitrogen) at lipid:AS ratio of 2:1 in a serum-free medium for 4 hours in a final volume of 65 ul. After 4 hour of transfection, 235 ul of RPMI medium containing 10% FBS and 10 ng/ml human recombinant Il-5 (R&D Systems) were added to each well.

Protein and RNA inhibition assays were identical to example 3 except that primers and probe were based on Il-5Ra sequences in Taqman assay.

The persentage of inhibition of IL-5Ra receptors RNA expression by IL-5R AS as measured against wobble controls are presented in the Table below:

TABLE 2

Interleukin-5 receptor mRNA screening result:

| | |
|---|---|
| Seq ID | RNA Inhibition |

| | |
|---|---|
| error 173: *,EPI-06-014,,114,,GGCGAGGACCGTGTCTGT | 32% |
| error 174: *,EPI-06-015,,119,,CAGAAGATGGCGAGGACCGTG | 32% |
| error 190: *,EPI-06-031,,248,,GCGCCACGATGATCATAT | 31% |
| error 191: *,EPI-06-032,,250,,ATGCGCCACGATGATCAT | 37% |
| error 192: *,EPI-06-033,,249,,TGCGCCACGATGATCATA | 32% |
| error 198: *,EPI-06-039,,295,,GTCAGCTTGCAGTATCTC | 30% |
| error 210: *,EPI-06-051,,544,,GTCGTTCTGCAGGATGGTCCG, | 75% |
| error 211: *,EPI-06-052,,549,,GTGGTCGTTCTGCAGGATG | 59% |
| error 212: *,EPI-06-053,,555,,AGTGAGTGGTCGTTCTGC | 38% |
| error 213: *,EPI-06-054,,560,,GCCAGTAGTGAGTGGTCGT | 60% |
| error 214: *,EPI-06-055,,565,,GCTGGCCAGTAGTGAGTG | 56% |
| error 215: *,EPI-06-056,,570,,GCCCAGCTGCTGGCCAGTAGT | 58% |
| error 216: *,EPI-06-057,,575,,GAAGCCCAGCTGCTGGCCA | 53% |
| error 223: *,EPI-06-064,,644,,GTGTTTGTGGTGCAAGTTA | 31% |
| error 225: *,EPI-06-066,,703,,GCCAGGTGCAGTGAAGGG | 37% |
| error 227: *,EPI-06-068,,713,,TGCCAACAAGCCAGGTGC | 35% |
| error 229: *,EPI-06-070,,718,,GGCATCTGTGCCAACAAGCC | 33% |
| error 236: *,EPI-06-077,,804,,CTCCCCAGTGTGTCTTTGCTG | 32% |
| error 237: *,EPI-06-078,,809,,TTCTCCCCAGTGTGTCTT | 32% |
| error 241: *,EPI-06-082,,861,,GCCAGTCACGCCCTTTGCTG | 32% |
| error 248: *,EPI-06-089,,910,,GGGCCTGATAGCAGAGTGC | 30% |
| error 260: *,EPI-06-101,,1016,,CACTGGTTTCTCCCATTGG | 33% |
| error 264: *,EPI-06-105,,1158,,GCTCTCACTTGAACATCGTAC | 31% |
| error 265: *,EPI-06-106,,1161,,CTGCTCTCACTTGAACATCG | 38% |
| error 266: *,EPI-06-107,,1165,,CTGCTGCTCTCACTTGAAC | 36% |
| error 269: *,EPI-06-110,,1194,,GAGCCCTGCCTCTCTGCAC | 33% |
| error 270: *,EPI-06-111,,1198,,CTCCAGAGCCCTGCCTCTCT | 31% |

### Example 5

Interleukin-4 Receptor

A library of 156 phosophorothioate antisense oligonucleotides against IL4R was screened using A549 cells (ATTC cat.# CCL-185). In a 24 well plate, A549 cells at 1.0×10⁵ cells/well were transfected with 1.0 uM oligonucleotide/20 ul/ml LipoFectin (Invitrogen) in a serum-free medium for 4 hours in a final volume of 300 ul. After 4 hour of tranfection, 1 ml of DMEM medium containing 10% FBS and 10 ng/ml human recombinant TNF-a (R &D Systems) were added to each well.

RNA inhibition assays were identical to example 3 except that primers and probe were based on Il4Ra sequences in Taqman assay.

The persentage of expression of IL4Ra receptors RNA in cells treated with specific antisense over wobble treated control cells are presented in the Table below:

TABLE 3

Interleukin-4 receptor mRNA screening result:

| | | |
|---|---|---|
| | | % of |
| Antisense | Sequence | Expression |

| | | |
|---|---|---|
| EPI-5-m16 | GCAGCTGCCCCATGCTG | 11.44 |
| EPI-5-m17 | GAGAAGGCCTTGTAACC | 10.98 |
| EPI-5-3 | CACCACGCCCGGCTTCTCT | 3.14 |
| EPI-5-4 | TCTGCCCGCCTCAGCCTCC | 12.51 |
| EPI-5-22 | GGCGGCTGCGGGCTGGGT | 12.74 |
| EPI-5-24 | CTTGGCTGGTTCCTGGCCT | 41.80 |
| EPI-5-29 | GGTTGTCTGGACTCTGGGT | 8.38 |
| EPI-5-31 | CGGGTTCTACTTCCTCCAGG | 10.31 |
| EPI-5-32 | TGCTCCCAGGTTTCTGGCTC | 5.28 |
| EPI-5-33 | CCCTGCTCCACCGCATGT | 4.17 |
| EPI-5-36 | CTGTTTCAGGTGGCCGC | 5.84 |
| EPI-5-42 | GTCTGCTGCAGAAGCTGTGG | 33.74 |
| EPI-5-48 | GTGCCTTATGCCTGCTGTCT | 14.69 |
| EPI-5-53 | GCTGGGATTATAGGCATGAG | 48.15 |
| EPI-5-55 | ACAGGGAACAGGAGCCCAGA | 34.37 |
| EPI-5-75 | CCCTGTAGGAAATCCCAGAC | 27.25 |
| EPI-5-83 | ACAATTCTTCCAGTGTGGGC | 34.18 |
| EPI-5-89 | ACACATCGCACCACGCTGAT | 8.61 |
| EPI-5-101 | ACGGTGACTGGCTCAGGGAG | 6.90 |
| EPI-5-109 | GGCCTTGTAACCAGCCTCTC | 16.54 |
| EPI-5-123 | GGGCAGGATGGAAGGATG | 8.68 |
| EPI-5-128 | GCCAATCACCTTCATACCAT | 9.52 |
| EPI-5-129 | TCCAGTCTCTGCAGCCCAGT | 4.04 |
| EPI-5-131 | GCCCTCTACTCTCATGGGAT | 9.37 |
| EPI-5-132 | GAGGTGCCCAAGGGCCTCAG | 7.64 |
| EPI-5-134 | GAAGCTGTGGAGGGAGCAGC | 55.26 |
| EPI-5-135 | AACAGGGACAGTCTGCTGCA | 19.74 |
| EPI-5-136 | AACATGCCTTGGGCAGTTAC | 18.12 |
| EPI-5-137 | GGCCATGATCTGGTGGGC | 46.34 |
| EPI-5-140 | TCTAGGCAATGACCACCCTC | 11.01 |
| EPI-5-141 | CGATTTCCCAAGGCCGCCCA | 4.28 |
| EPI-5-145 | GCCCACAGGGTGGCTGAGCA | 17.81 |
| EPI-5-147 | GCCAACATGCAGGGTAACTG | 11.12 |
| EPI-5-148 | CCCTAGCACCTGAGGTCTGG | 5.18 |
| EPI-5-149 | CAACCCAAGGTTCCCGCCTT | 3.18 |
| EPI-5-150 | ACACACAGACGAGCATTACT | 3.90 |

### Example 6

VCAM

A library of 221 phosophorothioate antisense oligonucleotides against VCAM was screened using BEAS-2B cells (ATTC cat.# CRL-9609). In a 24 well plate, BEAS-2B cells at 1.0×10⁵ cells/well were transfected with 1.0 uM oligonucleotide/20 ul/ml LipoFectin (Invitrogen) in a serum-free medium for 4 hours in a final volume of 300 ul. After 4 hour of tranfection, 1 ml of DMEM medium containing 10% FBS and 10 ng/ml human recombinant TNF-a (R &D Systems) were added to each well.

RNA inhibition assays were identical to example 3 except that primers and probe were based on VCAM sequences in Taqman assay.

The persentage of expression of VCAM receptors RNA in cells treated with specific antisense over wobble treated control cells are presented in the Table below:

TABLE 4

VCAM mRNA screening result:

| | | |
|---|---|---|
| | | % of |
| Antisense | Sequence | Expression |

| | | |
|---|---|---|
| EPI-3-029 | TTTAETACTETETCTCCTET | 40.23 |
| EPI-3-046 | CTTTCTECTTCTTCCAECCT | 46.66 |
| EPI-3-047 | CTTCCAECCTEETTAATTCC | 30.95 |
| EPI-3-072 | TTTECETACTCTECCTTTET | 16.13 |
| EPI-3-073 | CTECCTTTETTTEEETTCEA | 33.42 |
| EPI-3-081 | TEETAEEEATEAAEETCATT | 37.72 |
| EPI-3-084 | TETTCTCTAEAEATTTCATA | 26.66 |
| EPI-3-085 | AEATTTCATATCCETATCCT | 33.12 |
| EPI-3-087 | CCAAAAACTCTATATTCTCC | 37.45 |
| EPI-3-088 | TATATTCTCCAEAATAETCT | 27.84 |
| EPI-3-091 | TAATTCAATCTCCAECCEET | 36.78 |
| EPI-3-094 | CACECTAEEAACCTTECAEC | 17.42 |
| EPI-3-098 | TTCACEAEECCACCACTCAT | 20.35 |
| EPI-3-099 | CACCACTCATCTCEATTTCT | 18.09 |
| EPI-3-116 | CCECTCAEAEEECTETCTAT | 15.36 |
| EPI-3-117 | GGCTGTCTATCTGGGTTCTC | 34.31 |
| EPI-3-118 | CTGGGTTCTCCAGGAGAAAG | 32.11 |
| EPI-3-128 | ATCTCAACAGTAAATGGTTT | 24.75 |
| EPI-3-137 | CCAGAATCTTCCATCCTCAT | 28.92 |
| EPI-3-159 | CAGCCTGCCTTACTGTGGGC | 23.24 |
| EPI-3-160 | TACTGTGGGCACAGAATCCA | 38.05 |
| EPI-3-193 | TTCACAAGTTGCTGTGCACA | 25.88 |
| EPI-3-194 | GCTGTGCACAGGTAAGAGTG | 30.65 |
| EPI-3-196 | TTCGTTCCCAAAACTAACAG | 32.19 |
| EPI-3-213 | TAGATTCTGGGGTGGTCTCG | 29.55 |

### Example 7

Tryptase a and b

A library of 248 phosophorothioate antisense oligonucleotides against Tryptase a and/or b was screened using CHO cells (ATTC cat.# CCL-61) stably express either Tryptase a or Tryptase b gene product. In a 24 well plate, cells at 1.0×10⁵ cells/well were transfected with 1.0 uM oligonucleotide/16 ul/ml CellFectin (Invitrogen) in a serum-free medium for 4 hours in a final volume of 300 ul. After 4 hour of tranfection, 1 ml of F12 medium containing 10% FB were added to each well.

RNA inhibition assays were identical to example 3 except that primers and probe were based on Tryptase sequences in Taqman assay (Taqman primer and probe recognize both isotypes).

The persentage of expression of Tryptase RNA in cells treated with specific antisense over wobble treated control cells are presented in the Table below:

TABLE 5

Tryptase mRNA screening result:

| | | | |
|---|---|---|---|
| | | % of Expression | % of Expression |
| Compound | Sequence | Tryptase b | Tryptase a |

| | | | |
|---|---|---|---|
| EPI-15-001b | agattcagcatcctggccac | 69.18 | 76.53 |
| EPI-15-004b | agcgccagcagcagcagatt | 58.62 | 120.94 |
| EPI-15-012 | tggggcaggggccgcgtagg | 49.52 | 267.78 |
| EPI-15-024 | ccacttgctcctgggggcct | 19.76 | 86.00 |
| EPI-15-113 | ttgcgtcacaaatgtggttt | 55.92 | 55.00 |
| EPI-15-118 | cccgtgtaggcgccaaggtg | 43.98 | 3.99 |
| EPI-15-119 | cgtctcccgtgtaggcgcca | 33.36 | 7.00 |
| EPI-15-126 | ggcacacagcatgtcgtcac | 50.41 | 30.00 |
| EPI-15-139 | ccattcaccttgcacaccag | 56.53 | 97.52 |
| EPI-15-145 | cagctgaccacgcccgcctg | 62.78 | 20.64 |
| EPI-15-150 | gttgggctgggcacagccct | 55.17 | 47.00 |
| EPI-15-155 | tgacacgggtgtagatgcca | 102.54 | 23.00 |
| EPI-15-161 | catagtggtggatccagtcc | 61.16 | 16.00 |
| EPI-15-162 | ggggacatagtggtggatcc | 36.11 | 23.00 |

### Examples 8 & 9

MCPA and RANTES

Eosinophils are predominant effector cells in allergic diseases, which are attracted by several CC chemokines into the inflammatory tissue. It is well documented that the human eosinophils are recruited by eotaxin, RANTES and MCP-3 and MCP4 via CCR3. These chemokines are thus a potential therapeutic target for asthma and other allergic diseases. The goal of the present studies was to determine whether antisense oligonucleotides (ASODNs) (17 to 20 bases in length) designed to hybridize to the specific sequence in the 3′- and 5′-untranslated regions as well as the coding regions of RANTES and MCP-4 mRNA, inhibited mRNA and protein expression in BEAS-2B human airway epithelial cells. Confluent monolayers of BEAS-2B cells were either treated with culture medium, or transfected with RANTES (EPI-10) or MCP4 (EPI-104) specific antisense or Wobble, a control ASODN (5 μg/ml), in the presence of lipofectin (10 μg/ml), a carrier lipid, for 4 h followed by a 4 h (for mRNA expression) or 18 h (for protein expression) treatment with the complete medium. mRNA expression was determined by TaqMan using a specific MCP-4 or RANTES probe. 54 out 123 (43%) EPI-104 ASODNs and 32 out of 100 (32%) EPI-10 ASODNs showed more than 50% inhibition of MCP-4 and RANTES mRNA expression respectively (Tables 6 & 7). The level of MCP4 or RANTES protein in the conditioned medium of the BEAS-2B cells, either untransfected or transfected with specific or control ASODNs was determined by ELISA. Our results show undetectable levels of MCP4 and low levels of RANTES expression in BEAS-2B cells treated with medium only. Treatment of BEAS-2B cells with TNFα plus IFNγ induced the levels of both chemolines. Treatment of BEAS-2B cells with antisense prior to cytoline treatment, inhibited protein expression. 10 out of 123 (8%) EPI-104 ASODNs and 15 out of 100 (15%) EPI-10 ASODNs inhibited >25% and >50% of MCP-4 and RANTES protein expression respectively (Tables 8 & 9). These findings suggest that ASODNs can inhabit RANTES and MCP4 expression. Further studies are needed to determine whether the ASODN mediated inhibition of chemokine expression could alter migration of inflammatory cells, particularly eosinophils, in airway allergic inflammation.

TABLE 6
Inhibition of MCP-4 mRNA expression by EPI-104

| ASODN | Mean* | SEM |
|---|---|---|
| EPI-104-1 | 26.67 | 9.6 |
| EPI-104-2 | 31.50 | 5.3 |
| EPI-104-3 | 66.77 | 3.7 |
| EPI-104-5 | 97.60 | |
| EPI-104-7 | 189.07 | 113.0 |
| EPI-104-8 | 189.78 | 164.5 |
| EPI-104-9 | 17.48 | 5.1 |
| EPI-104-10 | 21.53 | 5.0 |
| EPI-104-11 | 20.44 | 10.4 |
| EPI-104-12 | 69.50 | |
| EPI-104-14 | 15.22 | 2.3 |
| EPI-104-15 | 35.97 | |
| EPI-104-16 | 135.48 | 58.4 |
| EPI-104-17 | 103.15 | 38.8 |
| EPI-104-20 | 92.84 | 9.6 |
| EPI-104-21 | 59.56 | 8.2 |
| EPI-104-22 | 43.63 | 8.2 |
| EPI-104-23 | 106.58 | 5.5 |
| EPI-104-24 | 42.09 | 13.4 |
| EPI-104-25 | 59.60 | 20.2 |
| EPI-104-26 | 59.88 | 13.1 |
| EPI-104-27 | 35.87 | 12.7 |
| EPI-104-28 | 95.62 | 8.3 |
| EPI-104-29 | 112.76 | 27.2 |
| EPI-104-30 | 36.23 | 22.0 |
| EPI-104-31 | 92.03 | 17.0 |
| EPI-104-32 | 42.82 | 11.5 |
| EPI-104-33 | 36.33 | 18.4 |
| EPI-104-34 | 30.87 | 7.9 |
| EPI-104-35 | 121.19 | 78.1 |
| EPI-104-36 | 82.20 | 8.2 |
| EPI-104-37 | 20.15 | 9.0 |
| EPI-104-38 | 31.46 | 9.2 |
| EPI-104-39 | 62.34 | 6.7 |
| EPI-104-40 | 28.26 | 8.3 |
| EPI-104-41 | 61.75 | 8.7 |
| EPI-104-42 | 36.73 | 20.5 |
| EPI-104-43 | 36.35 | 19.3 |
| EPI-104-44 | 34.74 | 13.8 |
| EPI-104-45 | 125.67 | |
| EPI-104-46 | 39.18 | |
| EPI-104-47 | 46.84 | |
| EPI-104-48 | 67.88 | |
| EPI-104-49 | 96.56 | |
| EPI-104-50 | 131.56 | |
| EPI-104-51 | 35.31 | |
| EPI-104-52 | 31.56 | |
| EPI-104-53 | 187.55 | |
| EPI-104-54 | 184.24 | |
| EPI-104-55 | 51.29 | 11.0 |
| EPI-104-56 | 62.34 | 17.7 |
| EPI-104-57 | 53.33 | 20.9 |
| EPI-104-58 | 77.28 | 22.0 |
| EPI-104-59 | 16.92 | 6.7 |
| EPI-104-60 | 97.78 | 16.8 |
| EPI-104-61 | 43.02 | 8.7 |
| EPI-104-62 | 45.58 | 13.8 |
| EPI-104-63 | 27.05 | 7.7 |
| EPI-104-64 | 50.31 | 8.8 |
| EPI-104-65 | 51.38 | 17.4 |
| EPI-104-66 | 103.94 | 21.4 |
| EPI-104-67 | 63.75 | 20.5 |
| EPI-104-68 | 84.41 | 20.6 |
| EPI-104-69 | 43.27 | 15.8 |
| EPI-104-70 | 33.05 | 18.6 |
| EPI-104-71 | 56.55 | 25.3 |
| EPI-104-72 | 35.45 | 10.1 |
| EPI-104-73 | 42.12 | 15.2 |
| EPI-104-74 | 78.12 | 42.5 |
| EPI-104-75 | 137.13 | 83.5 |
| EPI-104-76 | 73.30 | 26.9 |
| EPI-104-77 | 61.51 | 21.7 |
| EPI-104-78 | 67.40 | 29.7 |
| EPI-104-79 | 108.95 | 47.3 |
| EPI-104-80 | 71.23 | 20.7 |
| EPI-104-81 | 42.99 | 19.8 |
| EPI-104-82 | 71.30 | 31.1 |
| EPI-104-83 | 47.1 | 13.2 |
| EPI-104-84 | 59.49 | 18.4 |
| EPI-104-85 | 23.30 | |
| EPI-104-86 | 15.26 | |
| EPI-104-87 | 7.63 | |
| EPI-104-88 | 12.92 | |
| EPI-104-89 | 12.48 | |
| EPI-104-90 | 17.78 | |
| EPI-104-91 | 49.01 | 15.8 |
| EPI-104-92 | 51.40 | 6.3 |
| EPI-104-93 | 66.93 | 8.6 |
| EPI-104-94 | 51.74 | 8.4 |
| EPI-104-95 | 38.61 | 7.0 |
| EPI-104-96 | 118.59 | 7.8 |
| EPI-104-97 | 38.79 | 15.5 |
| EPI-104-98 | 100.91 | 14.6 |
| EPI-104-99 | 47.32 | 11.6 |
| EPI-104-100 | 27.06 | 11.8 |
| EPI-104-101 | 70.88 | 25.6 |
| EPI-104-102 | 43.27 | 11.1 |
| EPI-104-103 | 84.89 | 29.4 |
| EPI-104-104 | 107.67 | 7.8 |
| EPI-104-105 | 133.64 | 3.7 |
| EPI-104-106 | 40.59 | 1.0 |
| EPI-104-107 | 55.78 | 8.0 |
| EPI-104-108 | 150.19 | 29.8 |
| EPI-104-109 | 85.14 | 15.7 |
| EPI-104-110 | 11.02 | |
| EPI-104-111 | 62.52 | 32.1 |
| EPI-104-112 | 31.72 | 5.3 |
| EPI-104-113 | 55.25 | 12.3 |
| EPI-104-114 | 42.69 | 2.0 |
| EPI-104-115 | 40.58 | 1.2 |
| EPI-104-116 | 82.45 | 4.5 |
| EPI-104-117 | 48.73 | 0.2 |
| EPI-104-118 | 46.17 | 10.2 |
| EPI-104-119 | 46.59 | 5.2 |
| EPI-104-120 | 39.96 | 2.9 |
| EPI-104-121 | 32.60 | 11.5 |
| EPI-104-122 | 38.63 | 0.5 |
| EPI-104-123 | 102.42 | 15.5 |

*percentage of lipid wobble control
#54/123 (43%) ASODNs inhibited >50% MCP-4

TABLE 7
Inhibition of RANTES mRNA by EPI-10

| ASODN | Mean* | SEM |
|---|---|---|
| EPI-10-1 | 1311.67 | 913.6 |
| EPI-10-2 | 301.98 | 129.9 |
| EPI-10-3 | 190.17 | 65.7 |
| EPI-10-4 | 138.06 | 16.5 |
| EPI-10-5 | 162.29 | 20.4 |
| EPI-10-6 | 213.18 | 86.8 |
| EPI-10-7 | 181.72 | 61.0 |
| EPI-10-8 | 91.16 | 17.7 |
| EPI-10-9 | 142.00 | 57.1 |
| EPI-10-10 | 215.37 | 140.4 |
| EPI-10-11 | 85.19 | 32.7 |
| EPI-10-12 | 42.42 | 26.4 |
| EPI-10-13 | 30.40 | 20.5 |
| EPI-10-14 | 37.39 | 17.0 |
| EPI-10-15 | 100.06 | 59.1 |
| EPI-10-16 | 82.52 | 40.1 |
| EPI-10-17 | 146.14 | 86.9 |
| EPI-10-18 | 50.27 | 11.5 |
| EPI-10-19 | 59.15 | 20.0 |
| EPI-10-20 | 82.24 | 20.5 |
| EPI-10-21 | 42.53 | 20.8 |
| EPI-10-22 | 49.48 | 21.0 |
| EPI-10-23 | 99.98 | 82.2 |
| EPI-10-24 | 65.47 | 39.7 |
| EPI-10-25 | 78.79 | 46.6 |
| EPI-10-26 | 80.25 | 27.2 |
| EPI-10-27 | 60.33 | 17.8 |
| EPI-10-28 | 82.93 | 20.7 |
| EPI-10-29 | 67.97 | 7.5 |
| EPI-10-30 | 47.07 | 20.8 |
| EPI-10-31 | 50.34 | 17.8 |
| EPI-10-32 | 49.23 | 19.9 |
| EPI-10-33 | 63.58 | 20.7 |
| EPI-10-34 | 57.66 | 31.8 |
| EPI-10-35 | 46.36 | 8.9 |
| EPI-10-36 | 58.47 | 16.9 |
| EPI-10-37 | 47.36 | 17.2 |
| EPI-10-38 | 170.60 | 123.6 |
| EPI-10-39 | 57.76 | 12.1 |
| EPI-10-40 | 68.47 | 5.0 |
| EPI-10-41 | 65.74 | 17.0 |
| EPI-10-42 | 39.92 | 15.9 |
| EPI-10-43 | 40.53 | 13.3 |
| EPI-10-44 | 27.89 | 11.2 |
| EPI-10-45 | 24.46 | 9.3 |
| EPI-10-46 | 33.90 | 8.6 |
| EPI-10-47 | 172.34 | 127.0 |
| EPI-10-48 | 29.41 | 2.7 |
| EPI-10-49 | 46.09 | 1.5 |
| EPI-10-50 | 49.68 | 10.2 |
| EPI-10-51 | 66.44 | 22.0 |
| EPI-10-52 | 29.28 | 3.8 |
| EPI-10-53 | 31.56 | 1.4 |
| EPI-10-54 | 33.64 | 1.2 |
| EPI-10-55 | 82.52 | 38.3 |
| EPI-10-56 | 25.33 | 3.1 |
| EPI-10-57 | 39.73 | 4.4 |
| EPI-10-58 | 103.19 | 71.1 |
| EPI-10-59 | 109.81 | 24.9 |
| EPI-10-60 | 46.42 | 13.3 |
| EPI-10-61 | 762.72 | 725.4 |
| EPI-10-62 | 33.97 | 9.8 |
| EPI-10-63 | 22.38 | 4.4 |
| EPI-10-64 | 31.42 | 14.3 |
| EPI-10-65 | 45.04 | 10.7 |
| EPI-10-66 | 91.87 | 65.6 |
| EPI-10-67 | 58.06 | 24.8 |
| EPI-10-68 | 58.29 | 35.6 |
| EPI-10-69 | 64.81 | 42.9 |
| EPI-10-70 | 65.40 | 39.7 |
| EPI-10-71 | 61.47 | 21.1 |
| EPI-10-72 | 55.34 | 16.7 |
| EPI-10-73 | 90.32 | 46.5 |
| EPI-10-74 | 29.91 | 5.1 |
| EPI-10-75 | 39.81 | 5.2 |
| EPI-10-76 | 49.87 | 8.4 |
| EPI-10-77 | 39.70 | 26.2 |
| EPI-10-78 | 144.33 | 104.3 |
| EPI-10-79 | 83.48 | 43.4 |
| EPI-10-80 | 48.82 | 16.3 |
| EPI-10-81 | 50.43 | 28.4 |
| EPI-10-82 | 41.23 | 9.4 |
| EPI-10-83 | 50.09 | 30.6 |
| EPI-10-84 | 61.16 | 34.7 |
| EPI-10-85 | 206.40 | 155.7 |
| EPI-10-86 | 130.28 | 55.0 |
| EPI-10-87 | 102.72 | 75.2 |
| EPI-10-88 | 97.09 | 51.7 |
| EPI-10-89 | 94.62 | 24.9 |
| EPI-10-90 | 96.24 | 20.5 |
| EPI-10-91 | 68.92 | 32.6 |
| EPI-10-92 | 109.33 | 72.5 |
| EPI-10-93 | 175.60 | 81.0 |
| EPI-10-94 | 113.31 | 47.8 |
| EPI-10-95 | 371.83 | 239.0 |
| EPI-10-96 | 87.55 | 46.7 |
| EPI-10-97 | 82.59 | 16.6 |
| EPI-10-98 | 98.32 | 49.7 |
| EPI-10-99 | 71.84 | 30.7 |
| EPI-10-100 | 122.34 | 46.6 |

*percentage of lipid wobble control
#32/100 ASODNs inhibited >50% RANTES

TABLE 8
Inhibition of MCP-4 protein expression

| ASODN | Mean* | SEM |
|---|---|---|
| EPI-104-1 | 121.24 | 13.1 |
| EPI-104-2 | 172.21 | 12.3 |
| EPI-104-9 | 50.62 | 5.9 |
| EPI-104-10 | 100.67 | 6.2 |
| EPI-104-14 | 73.31 | 13.5 |
| EPI-104-15 | 69.42 | 0.8 |
| EPI-104-22 | 97.35 | 16.8 |
| EPI-104-24 | 67.76 | 15.5 |
| EPI-104-27 | 52.93 | 6.2 |
| EPI-104-30 | 117.37 | 5.7 |
| EPI-104-32 | 95.94 | 1.0 |
| EPI-104-33 | 139.91 | 22.0 |
| EPI-104-34 | 88.65 | 7.1 |
| EPI-104-37 | 72.92 | 5.5 |
| EPI-104-38 | 104.96 | 10.8 |
| EPI-104-40 | 66.95 | 16.0 |
| EPI-104-42 | 66.70 | 12.4 |
| EPI-104-43 | 219.72 | 1.8 |
| EPI-104-44 | 212.68 | 17.6 |
| EPI-104-46 | 77.83 | 4.3 |
| EPI-104-47 | 95.09 | 4.3 |
| EPI-104-51 | 114.20 | |
| EPI-104-52 | 121.26 | |
| EPI-104-59 | 105.43 | 0.8 |
| EPI-104-61 | 128.53 | 0.4 |
| EPI-104-62 | 122.35 | 8.5 |
| EPI-104-63 | 61.94 | 15.8 |
| EPI-104-69 | 98.48 | 10.6 |
| EPI-104-91 | 68.51 | 8.5 |
| EPI-104-95 | 122.77 | 7.2 |
| EPI-104-97 | 99.83 | 6.9 |
| EPI-104-99 | 91.42 | 10.7 |
| EPI-104-100 | 97.47 | 26.6 |
| EPI-104-102 | 88.83 | 16.8 |
| EPI-104-107 | 74.75 | 11.8 |
| EPI-104-110 | 104.41 | 2.2 |
| EPI-104-112 | 78.39 | 12.6 |
| EPI-104-113 | 112.08 | 22.3 |
| EPI-104-114 | 86.06 | 12.2 |
| EPI-104-115 | 97.18 | 5.5 |
| EPI-104-116 | 78.94 | 11.5 |
| EPI-104-118 | 99.16 | 1.1 |
| EPI-104-119 | 92.60 | 3.1 |
| EPI-104-120 | 86.98 | 13.9 |
| EPI-104-121 | 100.87 | 8.5 |
| EPI-104-122 | 91.43 | 10.8 |

TABLE 9
Inhibition of RANTES protein expression by EPI-10

| ASODN | Mean* | SEM |
|---|---|---|
| EPI-10-12 | 53.19 | 2.3 |
| EPI-10-13 | 31.02 | 2.1 |
| EPI-10-14 | 35.81 | 3.0 |
| EPI-10-18 | 71.18 | 0.8 |
| EPI-10-19 | 61.46 | 1.5 |
| EPI-10-21 | 26.41 | 2.2 |
| EPI-10-22 | 37.10 | 2.4 |
| EPI-10-27 | 44.39 | 0.6 |
| EPI-10-30 | 37.10 | 3.3 |
| EPI-10-31 | 82.31 | 2.6 |
| EPI-10-32 | 72.89 | 2.9 |
| EPI-10-34 | 44.25 | 3.1 |
| EPI-10-35 | 84.67 | 0.9 |
| EPI-10-36 | 88.23 | 1.9 |
| EPI-10-37 | 71.37 | 1.9 |
| EPI-10-42 | 59.98 | 1.3 |
| EPI-10-43 | 46.24 | 2.6 |
| EPI-10-44 | 62.02 | 2.8 |
| EPI-10-45 | 78.42 | 1.1 |
| EPI-10-46 | 110.12 | 3.7 |
| EPI-10-48 | 45.28 | 2.1 |
| EPI-10-49 | 77.89 | 1.1 |
| EPI-10-50 | 76.71 | 1.2 |
| EPI-10-52 | 55.13 | 2.5 |
| EPI-10-53 | 88.65 | 3.7 |
| EPI-10-55 | 73.85 | 1.2 |
| EPI-10-56 | 90.24 | 4.1 |
| EPI-10-57 | 108.63 | 3.2 |
| EPI-10-60 | 45.60 | 0.6 |
| EPI-10-62 | 55.44 | 1.2 |
| EPI-10-64 | 95.77 | 5.9 |
| EPI-10-63 | 67.59 | 3.5 |
| EPI-10-65 | 67.03 | 1.5 |
| EPI-10-67 | 84.63 | 1.8 |
| EPI-10-68 | 66.45 | 2.0 |
| EPI-10-72 | 70.47 | 2.2 |
| EPI-10-74 | 34.65 | 4.1 |
| EPI-10-75 | 66.62 | 4.6 |
| EPI-10-76 | 48.40 | 2.5 |
| EPI-10-77 | 34.60 | 6.2 |
| EPI-10-80 | 137.60 | 16.9 |
| EPI-10-81 | 91.00 | 14.2 |
| EPI-10-82 | 70.72 | 3.6 |
| EPI-10-83 | 126.02 | 44.8 |
| EPI-10-84 | 68.81 | 26.1 |

*percentage of lipid wobble control
#15/100 ASODNs inhibited >50% RANTES

### Examples 10 & 11

CCR1 and CCR3

Eosinophils are predominant effector cells in allergic diseases, which are attracted by several CC chemokines into the inflammatory tissue. It is well documented that the human eosinophils predominantly express the CC chemokine receptors CCR3 and to a lesser extent CCR1. It is thus a potential therapeutic target for asthma and other allergic diseases. The goal of the present studies was to determine whether antisense oligonucleotides (ASODNs) (17 to 20 bases in length) (EPI-1) designed to hybridize to the specific sequence in the 3′- and 5′-untranslated regions as well as the coding regions of CCR1 and CCR3 mRNA, inhibited mRNA and protein expression in HOS-CD4⁺ cell line transfected with CCR1 or CCR3. Confluent monolayers of HOS-CD4⁺ cells were either treated with culture medium, or transfected with CCR1- or CCR3-specific antisense or Wobble, a control ASODN (10 μg/ml), in the presence of DOTAP (30 μg/ml) or lipofectin (20 μg/ml), a carrier lipid, for 4 h followed by a 4 h (for mRNA expression) or 1 h (CCR1) or 24 h (CCR3) (for flow cytometry) treatment with the complete medium. mRNA expression was determined by TaqMan using a specific CCR1 or CCR3 probe. 81 (47%) and 75 (44%) out of 172 EPI-1 ASODNs inhibited between 25-50% and >50% CCR3 mRNA expression respectively (Table 10). Out of 32 ASODNs against the homologous sequences of CCR1 and CCR3, 18 inhibited both CCR1 and CCR3 expression between 25-50% (Table 10 & 11). The cell surface expression of CCR1 and CCR3 on HOS-CD4⁺ cells either untransfected or transfected with specific or control ASODNs was determined by flow cytometry. Our results show constitutive expression of CCR1 or CCR3 expression, which was inhibited by the ASODNs that also inhibited mRNA expression. 30 out of the 156 EPI-1 ASODNs, which inhibited CCR3 mRNA expression by >25%, produced more than 25% inhibition of CCR3 cell surface expression (Table 12). 5 EPI-1 ASODNs inhibited cell surface CCR1 protein expression by >50%, while the rest by 25-50% (Table 11). 2-3 out of 18 ASODNs having complete homology with CCR1 and CCR3 sequence were able to inhibit both CCR1 and CCR3 cell surface expression significantly. These findings suggest that ASODNs can inhibit CCR1 and CCR3 expression. In addition, our multi target antisense approach can be used to inhibit CCR1 and CCR3 expression using ASODNs designed against homologous regions on CCR1 and CCR3. Further studies are needed to determine whether the AS inhibition of CCR1 or CCR3 expression could alter migration of HOS-CD4⁺-CCR1⁺ or CCR3⁺ cells in response to MIP-1α or eotaxin. Our data may ultimately provide new therapeutic strategies for blocking eosinophil and possibly Th2 cell infiltration and in allergic inflammation in asthma.

TABLE 10
Inhibition of CCR3 mRNA expression EPI-1

| EPI-1- | Mean | SEM |
|---|---|---|
| 1 | 66.1 | 7.2 |
| 2 | 96.0 | 6.0 |
| 3 | 52.3 | 4.1 |
| 4 | 76.7 | 8 |
| 5 | 64.3 | 5.6 |
| 6 | 54.7 | 3.4 |
| 7 | 60 | 2.7 |
| 8 | 36.7 | 2.9 |
| 9 | 31 | 3.4 |
| 10 | 43.3 | 0.9 |
| 11 | 50 | 4.2 |
| 12 | 50 | 4.5 |
| 13 | 49.7 | 13 |
| 14 | 41.3 | 12.1 |
| 15 | 49 | 8.5 |
| 16 | 62 | 7 |
| 17 | 45.5 | 7.4 |
| 18 | 51 | 10.7 |
| 19 | 29 | 4.2 |
| 20 | 32.7 | 6.3 |
| 21 | 43.5 | 11.2 |
| 22 | 37.7 | 4.5 |
| 23 | 46.8 | 10 |
| 24 | 37 | 2.5 |
| 25 | 49 | 17.1 |
| 26 | 19.5 | 4.6 |
| 27 | 26.6 | 5.6 |
| 28 | 35.3 | 8 |
| 29 | 47 | 12.6 |
| 30 | 43.3 | 16.5 |
| 31 | 39.3 | 6.1 |
| 32 | 26.7 | 3.6 |
| 33 | 23.3 | 1.9 |
| 34 | 47.7 | 14.5 |
| 35 | 27.7 | 1.1 |
| 36 | 31.7 | 4.3 |
| 37 | 32 | 5.8 |
| 38 | 30.5 | 10 |
| 39 | 34.5 | 2.5 |
| 40 | 58.3 | 7.4 |
| 41 | 54.5 | 4.6 |
| 42 | 51.3 | 9 |
| 43 | 54 | 4.8 |
| 44 | 49 | 17.7 |
| 45 | 77 | |
| 46 | 64.5 | 2.5 |
| 47 | 39 | 15.5 |
| 48 | 40 | 2.6 |
| 49 | 37 | 7.2 |
| 50 | 30.3 | 2.6 |
| 51 | 23.3 | 0.9 |
| 52 | 57.5 | 6.5 |
| 53 | 49 | 7 |
| 54 | 51 | 8 |
| 55 | 52.5 | 0.5 |
| 56 | 36.3 | 3.3 |
| 57 | 59.8 | 12.9 |
| 58 | 72.1 | 21.1 |
| 59 | 65.4 | 6.5 |
| 60 | 72.2 | 5.3 |
| 61 | 69 | 19.5 |
| 62 | 69.4 | 13 |
| 63 | 48.8 | 14 |
| 64 | 63.5 | 3.3 |
| 65 | 50.7 | 16.3 |
| 65a | 54.5 | 5.9 |
| 66 | 62.3 | 10.8 |
| 67 | 60.5 | 20.7 |
| 68 | 50.9 | 17.8 |
| 69 | 44.6 | 3.3 |
| 70 | 56.1 | 4.7 |
| 71 | 91.2 | 23 |
| 72 | 67.8 | 21.7 |
| 73 | 56.5 | 14.3 |
| 74 | 57.7 | 9 |
| 75 | 40.4 | 6.7 |
| 76 | 39.5 | 3.5 |
| 77 | 40.4 | 2.3 |
| 78 | 36.8 | 5.4 |
| 78a | 55.8 | 10 |
| 80 | 37.3 | 4.4 |
| 81 | 51.6 | 5.6 |
| 82 | 62.4 | |
| 83 | 42.9 | |
| 84 | 72.1 | 12.4 |
| 85 | 65.1 | 7.7 |
| 86 | 65.3 | 8.8 |
| 87 | 53 | 8.5 |
| 88 | 30.4 | 5.8 |
| 89 | 40.5 | 9.2 |
| 90 | 42 | 6 |
| 91 | 51.1 | 8.9 |
| 92 | 49.9 | 16.7 |
| 93 | 55.4 | 11.1 |
| 94 | 48 | |
| 95 | 47 | |
| 96 | 65 | |
| 97 | 84 | 24.7 |
| 98 | 106 | |
| 99 | 95 | |
| 100 | 68.9 | 14.7 |
| 101 | 24 | 0 |
| 102 | 51.3 | 10.1 |
| 103 | 86.7 | 23.1 |
| 104 | 76 | 7 |
| 105 | 74.7 | 10.7 |
| 106 | 84 | 4 |
| 107 | 62 | 7.6 |
| 108 | 62.7 | 14.3 |
| 109 | 74.3 | 20.3 |
| 110 | 67 | 6 |
| 111 | 64.3 | 5.9 |
| 112 | 55 | 10.6 |
| 113 | 72.3 | 18.8 |
| 114 | 28.7 | 9.8 |
| 115 | 45.7 | 6.9 |
| 116 | 69 | 20.6 |
| 117 | 57.5 | 6.5 |
| 118 | 44.3 | 12.6 |
| 119 | 86.8 | 43.7 |
| 120 | 29 | 4 |
| 121 | 38 | 9 |
| 122 | 42 | 8.6 |
| 123 | 46.5 | 3.5 |
| 124 | 53 | 14 |
| 125 | 63.5 | 25.5 |
| 126 | 18 | 1 |
| 127 | 31.5 | 11.6 |
| 128 | 40.6 | 3.3 |
| 129 | 62.1 | 8.8 |
| 130 | 63.8 | 20.8 |
| 131 | 37 | 1 |
| 132 | 67.7 | 46.6 |
| 133 | 26.6 | 3.8 |
| 133a | 73.1 | 4.7 |
| 134 | 51.7 | 17.6 |
| 135 | 48.3 | 2.3 |
| 136 | 111 | 42.2 |
| 137 | 56 | 15 |
| 138 | 50.7 | 28.2 |
| 139 | 38.5 | 1.5 |
| 140 | 36.5 | 2 |
| 141 | 67.5 | 19.1 |
| 142 | 73.7 | 10.6 |
| 143 | 57.4 | 6.2 |
| 144 | 38.6 | 9.2 |
| 145 | 40 | |
| 146 | 55.8 | |
| 147 | 66.5 | 12.6 |
| 148 | 130.4 | 53.7 |
| 149 | 91 | 2.3 |
| 150 | 68.9 | 29 |
| 150a | 96.2 | 29.2 |
| 151 | 72.9 | 7.5 |
| 152 | 60 | 6.6 |
| 153 | 79.6 | 16.2 |
| 154 | 70.9 | 17.4 |
| 155 | 61 | 1.3 |
| 156 | 61.1 | 18.1 |
| 157 | 92.3 | 52.7 |
| 158 | 56.2 | 8.2 |
| 159 | 43.3 | 2.5 |
| 160 | 71.9 | 18.5 |
| 161 | 77.4 | 19.5 |
| 162 | 65.3 | 37.7 |
| 163 | 53.9 | 23.2 |
| 164 | 61.6 | 18.9 |
| 165 | 58.2 | 18.6 |
| 166 | 51.5 | 20.3 |
| 167 | 44.7 | 14.2 |
| 168 | 68.9 | 25.3 |
| 169 | 61.4 | 41.6 |

*percentage of lipid wobble control
#81/172 (47%) & 75/172 (44%) ASODNs inhibited >25-50% & >50% CCR3 expression respectively

TABLE 11
Inhibition of CCR1 mRNA and protein expression by EPI-1

| | |
|---|---|
| mRNA | Protein |

| ASODN | Mean* | SEM | ASODN | Mean* | SEM |
|---|---|---|---|---|---|
| EPI-1-71 | 88 | 27 | EPI-1-71 | 55.5 | 13.3 |
| EPI-1-72 | 66.6 | 12.5 | EPI-1-72 | 55 | 7.4 |
| EPI-1-73 | 74.5 | 21.8 | EPI-1-73 | 54.6 | 0 |
| EPI-1-74 | 74.5 | 21.8 | EPI-1-74 | 41.4 | 19.6 |
| EPI-1-75 | 104.5 | 12.5 | EPI-1-75 | 41.2 | 10.3 |
| EPI-1-76 | 85 | 33.4 | EPI-1-76 | 37.6 | 4.6 |
| EPI-1-77 | 60.1 | 12.9 | EPI-1-77 | 67.5 | 12.7 |
| EPI-1-78 | 77.1 | 30 | EPI-1-78 | 52.6 | 12.6 |
| EPI-1-78a | 115 | 11.7 | EPI-1-78a | | |
| EPI-1-80 | 67.6 | 19.7 | EPI-1-80 | 63.3 | 2.9 |
| EPI-1-81 | 71.7 | 9 | EPI-1-81 | 71 | 23.8 |
| EPI-1-82 | 86 | 20.2 | EPI-1-82 | 69.8 | 9.5 |
| EPI-1-83 | 86.7 | 7.7 | EPI-1-83 | 52.3 | 13.8 |
| EPI-1-84 | 82.9 | 11 | EPI-1-84 | 55.6 | 16.3 |
| EPI-1-85 | 84.7 | 6.9 | EPI-1-85 | 53.9 | 9.8 |
| EPI-1-86 | 73 | 8.8 | EPI-1-86 | 45.3 | 13.1 |
| EPI-1-87 | 72.3 | 11.6 | EPI-1-87 | 39.4 | 12.4 |
| EPI-1-88 | 68.9 | 15.9 | EPI-1-88 | 34.5 | 1.2 |
| EPI-1-89 | 64.2 | 15 | EPI-1-89 | 64.7 | 2 |
| EPI-1-90 | 60.9 | 14.8 | EPI-1-90 | 51.1 | 3.4 |
| EPI-1-91 | 69.6 | 11.8 | EPI-1-91 | 42.3 | 8.2 |
| EPI-1-92 | 73.4 | 22.2 | EPI-1-92 | 66.1 | 1.3 |
| EPI-1-93 | 68.5 | 24 | EPI-1-93 | 99.3 | 1.4 |
| EPI-1-94 | 24 | | EPI-1-94 | 95.3 | 21 |
| EPI-1-95 | 68.7 | 24.4 | EPI-1-95 | 87.4 | 5 |
| EPI-1-96 | 63.3 | 19.7 | EPI-1-96 | 90.2 | 9 |
| EPI-1-97 | 87 | 26.3 | EPI-1-97 | 100.9 | 15.2 |
| EPI-1-98 | 90.5 | 22.8 | EPI-1-98 | 119.3 | 19.2 |
| EPI-1-99 | 103 | 19 | EPI-1-99 | 110 | 17.4 |
| EPI-1-100 | 93.1 | 34 | EPI-1-100 | 97.2 | |
| EPI-1-101 | 128 | 29 | EPI-1-101 | 116.5 | 10 |
| EPI-1-102 | 53.7 | 6.3 | EPI-1-102 | 112.9 | 15.6 |

TABLE 12
Inhibition of CCR3 protein expression by EPI-1

| EPI-1- | Mean* | SEM |
|---|---|---|
| 1 | 106 | 28.4 |
| 2 | 155 | 13.2 |
| 14 | 74.5 | 14.1 |
| 19 | 74.2 | 14 |
| 20 | 117.9 | 26.3 |
| 21 | 95.7 | 35.4 |
| 23 | 124 | 19.5 |
| 25 | 29.4 | 20.5 |
| 26 | 70.1 | 8.6 |
| 27 | 61.7 | 6.4 |
| 28 | 73.2 | 23.5 |
| 29 | 92.2 | 16.8 |
| 30 | 84.6 | 32.6 |
| 33 | 80.4 | 10.6 |
| 34 | 96.5 | 28.9 |
| 35 | 80.1 | 24.5 |
| 36 | 93.8 | 9.5 |
| 37 | 116.7 | 30.1 |
| 38 | 109 | 14 |
| 71 | 90 | |
| 72 | 83 | |
| 73 | 85 | |
| 74 | 79 | |
| 75 | 75 | |
| 76 | 88 | |
| 78 | 112 | |
| 78a | 90 | |
| 80 | 114 | |
| 81 | 84 | |
| 82 | 107 | |
| 83 | 88 | |
| 84 | 92.8 | |
| 85 | 93.8 | |
| 86 | 107 | |
| 87 | 98 | |
| 88 | 84.4 | |
| 89 | 139 | |
| 90 | 87 | |
| 91 | 131 | |
| 92 | 80 | |
| 93 | 83 | |
| 94 | 83 | |
| 97 | 45.2 | |
| 98 | 23 | |
| 99 | 31.8 | 15 |
| 100 | 47.1 | 10.3 |
| 101 | 94.4 | 4.2 |
| 102 | 45 | 22.2 |
| 103 | 136 | 22.4 |
| 104 | 142 | 36 |
| 105 | 144 | 63 |
| 106 | 130 | 61.8 |
| 107 | 124 | 72 |
| 108 | 138 | 68 |
| 109 | 183 | 45 |
| 110 | 165 | 42 |
| 111 | 165 | 25 |
| 112 | 126.5 | 17 |
| 113 | 149.4 | 0.5 |
| 114 | 36.5 | |
| 115 | 146 | |
| 116 | 128 | |
| 117 | 161 | |
| 118 | 65 | 31.2 |
| 119 | 193.9 | |
| 120 | 270 | |
| 121 | 255 | |
| 122 | 235 | |
| 123 | 238 | |
| 124 | 106 | |
| 125 | 79 | |
| 126 | 73.4 | |
| 127 | 144.2 | |
| 128 | 96.4 | |
| 129 | 92.5 | |
| 130 | 67.3 | 8 |
| 131 | 72.5 | 21.9 |
| 132 | 95.1 | 28 |
| 133 | 97.2 | 24.1 |
| 133a | 105 | 52 |
| 134 | 117 | 44 |
| 135 | 68.1 | 11 |
| 136 | 111.9 | 23.2 |
| 137 | 79 | 0.1 |
| 138 | 83.4 | 2.3 |
| 139 | 72.3 | 1.1 |
| 140 | 76.1 | 1.2 |
| 141 | 84 | 20.2 |
| 142 | 82.7 | 13.1 |
| 143 | 81.4 | 5.4 |
| 144 | 80.9 | 2.1 |
| 145 | 97.7 | 9 |
| 146 | 84.8 | 19.2 |
| 147 | 73.1 | 41.8 |
| 148 | 90.6 | 51.6 |
| 149 | 159.4 | 116 |
| 150 | 55.5 | 24.1 |
| 150a | 62.2 | 23.2 |
| 151 | 58.7 | 3.9 |
| 152 | 58.7 | 7 |
| 153 | 52.6 | 1.7 |
| 154 | 56.9 | 4.8 |
| 155 | 58.1 | |
| 156 | 53.1 | 20.1 |
| 157 | 58 | 17.1 |
| 158 | 72.9 | 42.6 |
| 159 | 69.2 | 0.6 |
| 160 | 64.7 | 9 |
| 161 | 59.7 | 11.2 |
| 162 | 51.9 | 9.5 |
| 163 | 58.6 | 11.3 |
| 164 | 62.1 | 9.9 |
| 165 | 67.5 | 6.2 |
| 166 | 69.1 | 0.1 |
| 167 | 65.4 | 17.4 |
| 168 | 78.5 | 20.6 |
| 169 | 42.5 | |

*percentage of lipid wobble control
#30/156 EPI-1 ASODNs inhibited CCR3 >25%

### Example 12

In vivo Testing of Oligonucleotides

Balb/c mice are used for this testing. Six to ten week old balb/c mice (Jackson Labs, ME) are sensitized by two intraperitoneal injections on the first and fifth days, respectively, with chicken ovalbumin (Sigma, MO) at the doses from 1 to 200 μg per intraperitoneal injection per mouse. From the tenth day, mice are given oligonucleotides at the doses from 1.0 to 100 mg/kg body weight for three days through either aerosol, intranasal, or intra-tracheal administration. On the 12th day, mice are challenged with ovalbumin either by aerosol of 1-10% solution, or by intranasal administration of 50 nl of 0.2-20-ng/nl solution. Pulmonary responses are tested through a wholebody plethysmography (BUXCO system; Buxco electronics, CT), cellular responses are tested by BAL (bronchoalveolar lavage) cell differential staining, and changes in transcript and protein expression for the target genes in affected tissues are tested by Taqman and ELISA, respectively.

What is claimed as novel & unobvious in United States Letters Patent is:

1. An oligonucleotide (oligo) that is anti-sense to an initiation codon, a coding region, a 5′ or 3′ intron-exon junction, an intron, a region within 2 to 10 nucleotides of the 5′-end and the 3′-end or a border section between a coding and non-coding region of a nucleic acid target comprising a gene(s) selected from interleukin-4 receptor, interleukin-5 receptor, CCR1, CCR3, Eotaxin-1, RANTES, MCP4, CD23, ICAM, VCAM, tryptase a, tryptase b, PDE4 A, PDE4 B, PDE4 C or PDE4 D gene; or anti-sense to their corresponding mRNAs; or pharmaceutically and veterinarily acceptable salts of the oligo(s); and optionally a surfactant that may be operatively linked to the oligo(s).

2. The oligo of claim 1, wherein the oligo is anti-sense to SEQ ID NOS: 1-2499.

3. The oligo of claim 1, wherein the oligo is anti-sense to at least two genes or RNAs.

4. The oligo of claim 1, wherein at least one mononucleotide is substituted or modified by one or more of phosphorothioate, chiral phosphorothioate, phosphorodithioate, phosphotriester, aminoalkylphosphotriester, methyl phosphonate, 3′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, boranophosphate, morpholino, siloxane, sulfide, sulfoxide, sulfone, formacetyl, thioformacetyl, methylene formacetyl, thioformacetyl, alkene, sulfamate, methyleneimino, methylenehydrazino, sulfonate, sulfonamide, amide, thioether, carbonate, carbamate, sulfate, sulfite, hydroxylamine, methylene(methyimino), methyleneoxy (methylimino), 2′-O-methyl, or phosphoramidate residues, or combinations thereof.

5. The oligo of claim 4, wherein all mononucleotides are substituted or modified.

6. The oligo of claim 1, wherein at least one mononucleotide is substituted or modified at the 2′ position by one or more of OH, F, O—, S—, N-alkyl, O-alkyl-O-alkyl, N-alkenyl, N-alkynyl, O[(CH₂)ₙO]ₘCH₃, O(CH₂ₙOCH₃, O(CH₂)₂ON(CH₃)₂, O(CH₂)ₙNH₂, O(CH₂)ₙCH₃, O(CH₂)ₙONH₂, or O(CH₂)ₙON[(CH₂)ₙCH₃)]₂, wherein n or m are from 1 to about 10, C₁ to C₁₀ lower alkyl, substituted lower alkyl, alkaryl, aralkyl, O-alkaryl, O-aralkyl, SH, SCH₃, OCN, Cl, Br, CN, CF₃, OC₃, SOCH₃, SO₂CH₃, ONO₂, NO₂, N₃, NH₂, heterocycloalkyl, heterocycloalkaryl, aminoalkylamino, poly-alkylamino, or substituted silyl.

7. The oligo of claim 6, wherein all mononucleotides are substituted or modified.

8. The oligo of claim 1, wherein at least one mononucleotide is substituted or modified by one or more of 5-methylcytosine (^{m}C), 5-hydroxymethyl cytosine, xanthine, hypoxanthine, 2-aminoadenine, 6methyl adenine, 6-methyl guanine, 2-propyl adenine, 2-propyl guanine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil (pseudouracil), 4-thiouracil adenine, 8-halo adenine, 8-amino adenine, 8-thiol adenine, 8-thioalkyl adenine, 8-hydroxyl adenine, 8-halo guanine, 8-amino guanine, 8-thiol guanine, 8-thioalkyl guanine, 8-hydroxyl guanine, 5-bromo uracil, 5-trifluoromethyl uracil, 5-bromo cytosine, 5-trifluoromethyl cytosine, 7-methylguanine, 7-methyladenine, 8-azaguanine, 8-azaadenine, 7-deazaguanine, 7-deazaadenine, 3-deazaguanine, 3-deazaadenine, 2-aminopropyladenine, 5-propynyluracil, 5-propynylcytosine or 5-methylcytosine.

9. The oligo of claim 8, wherein all mononucleotides are substituted or modified.

10. The oligo of claim 1, wherein a methylated cytosine (^{m}C) is substituted for an unmethylated cytosine (C) in at least one CpG dinucleotide if present in the oligo(s).

11. The oligo of claim 1, wherein if the oligo contains adenosine (A), at least one A is substituted by a universal base selected from heteroaromatic bases that bind to a thymidine base but have antagonist activity or less than about 0.3 of the adenosine base agonist activity at the adenosine A₁, A_{2b} or A₃ receptors, or heteroaromatic bases that have no activity or have agonist activity at the adenosine A₂ₐ receptor.

12. The oligo of claim 11, wherein substantially all As are substituted by a universal base(s) selected from heteroaromatic bases that bind to a thymidine base but either have antagonist activity or less than about 0.3 of the adenosine base agonist activity at the adenosine A₁, A_{2b} or A₃ receptors, or heteroaromatic bases that have no activity or have agonist activity at the adenosine A₂ₐ receptor.

13. The oligo of claim 11, wherein the heteroaromatic bases are selected from pyrimidines or purines that may be substituted by O, halo, NH₂, SH, SO, SO₂, SO₃, COOH, or branched or fused primary or secondary amino, alkyl, alkenyl, alkynyl, cycloalkyl, heterocycloalkyl, aryl, heteroaryl, alkoxy, alkenoxy, acyl, cycloacyl, arylacyl, alkynoxy, cycloalkoxy, aroyl, arylthio, arylsulfoxyl, halocycloalkyl, alkylcycloalkyl, alkenylcycloalkyl, alkynylcycloalkyl, haloaryl, alkylaryl, alkenylaryl, alkynylaryl, arylalkyl, arylalkenyl, arylalkynyl, or arylcycloalkl, which may be further substituted by O, halo, NH₂, primary, secondary or tertiary amine, SH, SO, SO₂, SO₃, cycloalkyl, heterocycloalkyl or heteroaryl.

14. The oligo of claim 13, wherein the pyrimidines are substituted at a 1, 2, 3, and/or 4 position, and the purines are substituted at a 1, 2, 3, 4, 7 and/or 8 position.

15. The oligo of claim 13, wherein the pyrimidines or purines are selected from theophylline, caffeine, dyphylline, etophylline, acephylline piperazine, bamifylline, enprofylline or xanthine.

16. The oligo of claim 11, wherein the universal base is selected from 3-nitropyrrole-2′-deoxynucleoside, 5-nitroindole, 2-deoxyribosyl-(5-nitroindole), 2-deoxyribofuranosyl-(5-nitroindole), 2′-deoxyinosine, 2′-deoxynebularine, 6H, 8H-3,4-dihydropyrimido[4,5-c]oxazine-7-one or 2-amino-6-methoxyaminopurine.

17. The oligo of claim 1, wherein the oligo consists of up to about 10% A.

18. The oligo of claim 17, wherein the oligo consists of up to about 5% A.

19. The oligo of claim 18, wherein the oligo consists of up to about 3% A.

20. The oligo of claim 19, wherein the oligo is A-free.

21. The oligo of claim 1, wherein the nucleic acid is linked to an agent that enhances cell internalization or up-take and/or a cell targeting agent.

22. The oligo of claim 21, wherein the cell internalization or up-take enhancing agent comprises transferrin, asialoglycoprotein or streptavidin.

23. The oligo of claim 21, wherein the cell targeting agent comprises a vector, and the nucleic acid is operatively linked to the vector.

24. The oligo of claim 23, wherein the vector comprises a prokaryotic or eukaryotic vector.

25. A composition comprising the oligonucleotide of claim 1, and a pharmaceutically or veterinarily acceptable carrier or diluent and optionally therapeutic agents.

26. The composition of claim 25, wherein the carrier or diluent comprises gaseous, liquid or solid carrier or diluent.

27. The composition of claim 25, wherein the therapeutic agents comprise surfactants, antioxidants, flavoring and coloring agents, fillers, volatile oils, buffering agents, dispersants, RNA inactivating agents, antioxidants, flavoring agents, propellants or preservatives.

28. The composition of claim 27, wherein the surfactants are lipid or non-lipid surfactants.

29. The composition of claim 28, wherein the surfactants comprises surfactant protein A, surfactant protein B, surfactant protein C, surfactant protein D, surfactant protein E, active fragments thereof, non-dipalmitoyl disaturated phosphatidylcholine, dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholin, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate, artificial lamellar bodies vehicles for surfactant components, omega-3 fatty acids, polyenic acid, polyenoic acid, lecithin, palmitic acid, non-ionic ethylene and/or propylene oxide block copolymers, polyoxypropylene, polyoxyethylene, poly (vinyl amine) with dextran and/or alkanoyl side chains, polyoxy ethylene 23 lauryl ether (Brij 35®), t-octyl phenoxy polyethoxy ethanol (Triton X-100®), dipalmitoyl phosphatidyl choline (DPPC), phosphatidyl glycerol (PG) (ALEC®), tyloxapol (Exosurf®), surfactant-associated proteins (Survanta®) or C₂₂H₁₉C₁₀ (Atovaquone®).

30. The composition of claim 27, wherein the RNA inactivating agent comprises an enzyme.

31. The composition of claim 30, wherein the enzyme comprises a ribozyme.

32. The composition of claim 25, further comprising a propellant.

33. The composition of claim 1, wherein the oligo is present in an amount of about 0.01 to about 99.99 w/w of the composition.

34. A formulation comprising the composition of claim 25, wherein the carrier comprises a hydrophobic carrier.

35. The formulation of claim 34, selected from intrabuccal, intrapulmonary, respirable, nasal, inhalable, intracavitary, intraorgan, or slow release formulations.

36. The formulation of claim 34, wherein the carrier is selected from a solid or liquid carrier.

37. The formulation of claim 34, which comprises a sprayable or aerosolizable powder, solution, suspension or emulsion.

38. The formulation of claim 34, which comprises a sprayable or aerosolizable aqueous or alcoholic solution or suspension, oily solution or suspension, or oil-in-water or water-in-oil emulsion.

39. A capsule or cartridge, comprising the formulation of claim 34.

40. The formulation of claim 34, which comprises a formulation of particle size about 0.5μ to about 10μ, or about 10μ to about 500μ.

41. The formulation of claim 34, which comprises a nasal formulation of particle size about 10μ to about 500μ.

42. The formulation of claim 34, which is a respirable or inhalable formulation comprising a solid powdered or liquid aerosol or spray of particle size about 0.5μ to about 10μ.

43. The formulation of claim 34, in bulk, or in single or multiple unit dose form.

44. A vector, comprising the oligonucleotide of claim 1.

45. A cell, comprising the oligonucleotide of claim 1.

46. A diagnostic or therapeutic kit for delivery of an oligonucleotide(s) (oligo(s)) comprising, in separate containers,
the delivery device;
the composition of claim 25; and
instructions for loading the composition into the device and for its use.

47. The kit of claim 46, wherein the delivery device comprises a nebulizer, a dry powder inhaler, a pressurized inhaler or insufflator.

48. The kit of claim 46, wherein the delivery device delivers single metered doses.

49. The kit of claim 46, wherein the delivery device is adapted for receiving and piercing or opening a capsule(s), blister(s) or cartridge(s) and producing a solid powdered or liquid aerosol or spray.

50. The kit of claim 46, wherein the composition is in an inhalable, respirable, nasal, intracavitary, intraorgan or intrapulmonary formulation.

51. The kit of claim 46, wherein the composition is of particle size about 0.5μ to about 10μ or about 10μ to about 500μ.

52. The kit of claim 48, wherein the composition is provided in a pierceable or openable capsule, blister or cartridge.

53. The kit of claim 48, comprising the delivery device, a surfactant, the composition and other therapeutic agents.

54. The kit of claim 48, further comprising a solvent selected from organic solvents or organic solvents mixed with one or more co-solvents.

55. A method for reducing or inhibiting expression of a gene or mRNA encoding interleukin-4 receptor, interleukin-5 receptor, CCR1, CCR3, Eotaxin-1, RANTES, MCP4, CD23, ICAM, VCAM, tryptase a, tryptase b, PDE4 A, PDE4 B, PDE4 C or PDE4 D, comprising contacting the oligonucleotide of claim 1 with cells or tissues, under conditions effective for hybridization, and allowing hybridization to occur, whereby expression is reduced or inhibited.

56. The method of claim 55, wherein the hybridization is conducted under stringent condition in vitro.

57. The method of claim 55, wherein the hybridization is conducted under semi-stringent condition in vitro.

58. The method of claim 55, wherein the hybridization is conducted under physiolosical condition in vivo.

59. A method for preventing or treating a respiratory or lung disease, comprising administering to the airways of a subject an effective amount of an inhibitor of one or more nucleic acid target(s) or expressed product(s) thereof comprising a gene(s) selected from interleukin-4 receptor, interleukin-5 receptor, CCR1, CCR3, Eotaxin-1, RANTES, MCP4, CD23, ICAM, VCAM, tryptase a, tryptase b, PDE4 A, PDE4 B, PDE4 C or PDE4 D.

60. The method of claim 59, wherein the inhibitor is administered intrapulmonary, intraorgan, intracavitarily, intrabuccally, intranasally, by inhalation or into the subject's respiratory system.

61. The method of claim 59, wherein the inhibitor is the composition of claim 25.

62. The method of claim 61, wherein the composition comprises solid powdered or liquid particles of about 0.5 to about 10μ in size.

63. The method of claim 61, wherein the composition is administered as powdered solid or liquid particles of about 10μ to about 500μ in size.

64. The method of claim 59, wherein the composition further comprises other therapeutic agents.

65. The method of claim 64, wherein the therapeutic agent(s) comprise(s) anti-adenosine A₁, A_{2b} or A₃ receptor agents or adenosine A₂ₐ receptor stimulating agents other than the nucleic acid(s).

66. The method of claim 59, further comprising administering a surfactant.

67. The method of claim 66, wherein the surfactant comprises lipid or non-lipid surfactant.

68. The method of claim 59, wherein the respiratory or lung disease comprises asthma, bronchoconstriction, impeded respiration, cystic fibrosis (CF), Chronic Obstructive Pulmonary Diseases (COPD), allergic rhinitis (AR), Acute Respiratory Distress Syndrome (ARDS), pulmonary hypertension and bronchitis.

69. The method of claim 59, wherein the the respiratory or lung disease is associated with hyper-responsiveness to and/or increased levels of, adenosine and/or levels of adenosine (A) receptor(s), and/or asthma and/or lung allergy(ies) and/or lung inflammation.

70. The method of claim 59, wherein the hyper-responsiveness to, or increased levels of, adenosine, levels of adenosine (A) receptor(s), and/or bronchoconstriction, and/or asthma, and/or lung allergy(ies) and/or lung inflammation is(are) associated with inflammation or an inflammatory disease.

71. The method of claim 60, wherein the subject is a mammal.

72. The method of claim 71, wherein the mammal is a human or a non-human mammal.

73. The method of claim 61, wherein the composition is administered in an amount of about 0.005 to about 150 mg/kg body weight.

74. The method of claim 73, wherein the composition is administered in an amount of about 0.01 to about 75 mg/kg body weight.

75. The method of claim 74, wherein the composition is administered in an amount of about 1 to about 50 mg/kg body weight.

76. The method of claim 59, which is a prophylactic or therapeutic method.

77. The method of claim 59, wherein the oligo is obtained by
(a) selecting fragments of a target nucleic acid having at least 4 contiguous bases consisting of G or C; and
(b) obtaining a second oligo 4 to 60 nucleotides long comprising a sequence that is anti-sense to the selected fragment.

78. The method of claim 59, wherein the oligo consists of up to about 10% A.

79. The method of claim 78, wherein the oligo consists of up to about 5% A.

80. The method of claim 79, wherein the oligo consists of up to about 3% A.

81. The method of claim 80, wherein the oligo is A-free.

82. The method of claim 59, wherein the inhibitor is selected from dansylcadaverin, glycinamide, methylamine, n-propylamine, n-hexylamine, bacitracin, ethylamine, t-butylamine, an antibody to the expressed product or the oligo of claim 1, or combination thereof.

83. The method of claim 59, further comprising administering a subject of interest with one or more anti-asthma agent(s).

84. The method of claim 82, wherein the oligo is anti-sense to at least two genes, ESTs or RNAs.

85. A use of the oligonucleotide of claim 1 for production of a medicament for the prevention and/or treatment of a respiratory or lung disease.

86. The use of claim 85, wherein the respiratory or luing disease comprises airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, or bronchoconstriction.

87. A method for screening a candidate compound for the prevention and/or treatment of a respiratory or lung disease that binds to one or more nucleic acid target(s) or expressed product(s) thereof comprising a gene(s) selected from interleukin-4 receptor, interleukin-5 receptor, CCR1, CCR3, Eotaxin-1, RANTES, MCP4, CD23, ICAM, VCAM, tryptase a, tryptase b, PDE4 A, PDE4 B, PDE4 C or PDE4 D.

88. The method claim 87, wherein the the nucleic acid target(s) or their expressed product(s) is(are) in a purified form from the expression system.

89. The method of claim 88, wherein the expressed product(s) is(are) expressed in or on the cell.

90. The method of claim 87, wherein the binding is detected by a label.

91. The method of claim 87, wherein the candidate compound suppresses the expression of one or more nucleic acid target(s).

92. The method of claim 87, wherein further comprising steps of contacting a candidate compound with or introducing into a cell expressing the one or more nucleic acid target(s) or their expressed product(s), and detecting the suppression, reduction or inhibition of their expression.

93. The method of claim 92, wherein the suppression, reduction or inhibition is detected by measuring the level of the transcribed mRNA of the genes.

94. The method of claim 92, wherein the cell comprises a construct comprising a nucleic acid target that is linked to a reporter gene system in a cell.
